SEQUENCE LISTING (168 sequences)

SEQ ID NO: 1 - protein, 5 aa, Artificial Sequence
  Description of Artificial Sequence Synthetic peptide
SYWIT

SEQ ID NO: 2 - protein, 17 aa, Artificial Sequence
  Description of Artificial Sequence Synthetic peptide
DIYPGSGSTNHIEKFKS

SEQ ID NO: 3 - protein, 11 aa, Artificial Sequence
  Description of Artificial Sequence Synthetic peptide
GYGSSYGYFDY

SEQ ID NO: 4 - protein, 11 aa, Artificial Sequence
  Description of Artificial Sequence Synthetic peptide
RASENIYSYLA

SEQ ID NO: 5 - protein, 7 aa, Artificial Sequence
  Description of Artificial Sequence Synthetic peptide
TAKTLAE

SEQ ID NO: 6 - protein, 9 aa, Artificial Sequence
  Description of Artificial Sequence Synthetic peptide
QHQYGPPFT

SEQ ID NO: 7 - protein, 120 aa, Artificial Sequence
  Description of Artificial Sequence Synthetic polypeptide
QVQLVQSGAEVKKPGASVKVSCKASGYTFTSYWITWVKQAPGQGLEWIGDIYPGSGSTNHIEKFKSKATLTVDTSISTAYMELSRLRSDDTAVYYCATGYGSSYGYFDYWGQGTLVTVSS

SEQ ID NO: 8 - protein, 107 aa, Artificial Sequence
  Description of Artificial Sequence Synthetic polypeptide
DIQMTQSPSSLSASVGDRVTITCRASENIYSYLAWYQQKPGKAPKLLIYTAKTLAEGVPSRFSGSGSGTDFTLTISSLQPEDFATYYCQHQYGPPFTFGQGTKLEIK

SEQ ID NO: 9 - protein, 5 aa, Artificial Sequence
  Description of Artificial Sequence Synthetic peptide
SYWMH

SEQ ID NO: 10 - protein, 17 aa, Artificial Sequence
  Description of Artificial Sequence Synthetic peptide
NIDPSDSDTHYNQKFKD

SEQ ID NO: 11 - protein, 10 aa, Artificial Sequence
  Description of Artificial Sequence Synthetic peptide
GYSKYYAMDY

SEQ ID NO: 12 - protein, 16 aa, Artificial Sequence
  Description of Artificial Sequence Synthetic peptide
RSSQSIVHSYGNTYLE

SEQ ID NO: 13 - protein, 7 aa, Artificial Sequence
  Description of Artificial Sequence Synthetic peptide
KVSNRFS

SEQ ID NO: 14 - protein, 9 aa, Artificial Sequence
  Description of Artificial Sequence Synthetic peptide
FQGSHVPYT

SEQ ID NO: 15 - protein, 119 aa, Artificial Sequence
  Description of Artificial Sequence Synthetic polypeptide
QVQLVQSGAEVKKPGASVKVSCKASGYTFTSYWMHWVRQAPGQGLEWMGNIDPSDSDTHYNQKFKDRVTMTRDTSTSTVYMELSSLRSEDTAVYYCARGYSKYYAMDYWGQGTLVTVSS

SEQ ID NO: 16 - protein, 112 aa, Artificial Sequence
  Description of Artificial Sequence Synthetic polypeptide
DIVMTQTPLSLSVTPGQPASISCRSSQSIVHSYGNTYLEWYLQKPGQSPQLLIYKVSNRFSGVPDRFSGSGSGTDFTLKISRVEAEDVGVYYCFQGSHVPYTFGQGTKLEIK

SEQ ID NO: 17 - protein, 449 aa, Artificial Sequence
  Description of Artificial Sequence Synthetic polypeptide
QVQLVQSGAEVKKPGASVKVSCKASGYTFTSYWITWVKQAPGQGLEWIGDIYPGSGSTNHIEKFKSKATLTVDTSISTAYMELSRLRSDDTAVYYCATGYGSSYGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 18 - protein, 214 aa, Artificial Sequence
  Description of Artificial Sequence Synthetic polypeptide
DIQMTQSPSSLSASVGDRVTITCRASENIYSYLAWYQQKPGKAPKLLIYTAKTLAEGVPSRFSGSGSGTDFTLTISSLQPEDFATYYCQHQYGPPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 448 aa, Artificial Sequence
  Description of Artificial Sequence Synthetic polypeptide
QVQLVQSGAEVKKPGASVKVSCKASGYTFTSYWMHWVRQAPGQGLEWMGNIDPSDSDTHYNQKFKDRVTMTRDTSTSTVYMELSSLRSEDTAVYYCARGYSKYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 20 - protein, 219 aa, Artificial Sequence
  Description of Artificial Sequence Synthetic polypeptide
DIVMTQTPLSLSVTPGQPASISCRSSQSIVHSYGNTYLEWYLQKPGQSPQLLIYKVSNRFSGVPDRFSGSGSGTDFTLKISRVEAEDVGVYYCFQGSHVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 5 aa, Artificial Sequence
  Description of Artificial Sequence Synthetic peptide
DYYIH

SEQ ID NO: 22 - protein, 17 aa, Artificial Sequence
  Description of Artificial Sequence Synthetic peptide
RIDPEDGETKYAPKFQG

SEQ ID NO: 23 - protein, 5 aa, Artificial Sequence
  Description of Artificial Sequence Synthetic peptide
GGFAY

SEQ ID NO: 24 - protein, 11 aa, Artificial Sequence
  Description of Artificial Sequence Synthetic peptide
ASSSVSSSYLY

SEQ ID NO: 25 - protein, 7 aa, Artificial Sequence
  Description of Artificial Sequence Synthetic peptide
STSNLAS

SEQ ID NO: 26 - protein, 9 aa, Artificial Sequence
  Description of Artificial Sequence Synthetic peptide
HQWSSHPYT

SEQ ID NO: 27 - protein, 114 aa, Artificial Sequence
  Description of Artificial Sequence Synthetic polypeptide
EVQLQQSGAELVKPGASVKLSCTASGFNIKDYYIHWVKQRTEQGLEWIGRIDPEDGETKYAPKFQGKATITADTSSNTAYLQLNSLTSEDTAVYSCAKGGFAYWGQGTLVTVSA

SEQ ID NO: 28 - protein, 108 aa, Artificial Sequence
  Description of Artificial Sequence Synthetic polypeptide
QIVLTQSPAIMSASPGEKVTLTCSASSSVSSSYLYWYQQKPGSSPKLWIYSTSNLASGVPARFSGSGSGTSYSLTISSMEAEDAASYFCHQWSSHPYTFGGGTKLEIK

SEQ ID NO: 29 - protein, 5 aa, Artificial Sequence
  Description of Artificial Sequence Synthetic peptide
SYWMH

SEQ ID NO: 30 - protein, 17 aa, Artificial Sequence
  Description of Artificial Sequence Synthetic peptide
NIDPSDSDTHYNQKFKD

SEQ ID NO: 31 - protein, 12 aa, Artificial Sequence
  Description of Artificial Sequence Synthetic peptide
SYGNYGENAMDY

SEQ ID NO: 32 - protein, 16 aa, Artificial Sequence
  Description of Artificial Sequence Synthetic peptide
RSSQSIVHSYGNTYLE

SEQ ID NO: 33 - protein, 7 aa, Artificial Sequence
  Description of Artificial Sequence Synthetic peptide
KVSNRFS

SEQ ID NO: 34 - protein, 9 aa, Artificial Sequence
  Description of Artificial Sequence Synthetic peptide
FQGSHVPFT

SEQ ID NO: 35 - protein, 120 aa, Artificial Sequence
  Description of Artificial Sequence Synthetic polypeptide
QVKLQESGAELVRPGSSVKLSCKASGYTFTSYWMHWVKQRPIQGLEWIGNIDPSDSDTHYNQKFKDKATLTVDNSSSTAYMQLSSLTSEDSAVYYCASYGNYGENAMDYWGQGTSVTVSS

SEQ ID NO: 36 - protein, 112 aa, Artificial Sequence
  Description of Artificial Sequence Synthetic polypeptide
DILMTQTPLSLPVSLGDQASISCRSSQSIVHSYGNTYLEWYLQKPGQSPKLLIYKVSNRFSGVPDRFSGSGSGTDFTLKISRVEAEDLGVYYCFQGSHVPFTFGSGTKLEIK

SEQ ID NO: 37 - DNA, 1347 bp, Artificial Sequence
  Description of Artificial Sequence Synthetic polynucleotide
caggttcagttggttcagtctggcgccgaagtgaagaaacctggcgcctctgtgaaggtgtcctgcaaggcttccggctacacctttaccagctactggatcacctgggtcaagcaggctcctggacagggactcgagtggatcggcgatatctatcctggctccggctccaccaaccacatcgagaagttcaagtccaaggctaccctgaccgtggacacctccatctccaccgcctacatggaactgtcccggctgagatctgacgacaccgccgtgtactattgcgctaccggctacggctcctcctacggctactttgattattggggccagggcaccctggtcaccgtgtcctctgcttctaccaagggacccagcgtgttccctctggctccttccagcaagtctacctctggcggaacagctgctctgggctgcctggtcaaggactactttcctgagcctgtgaccgtgtcttggaactctggcgctctgacatctggcgtgcacacattccctgctgtgctgcagtcctccggcctgtactctctgtcctctgtcgtgaccgtgccttccagctctctgggaacccagacctacatctgcaatgtgaaccacaagccttccaacaccaaggtggacaagaaggtggaacccaagtcctgcgacaagacccacacctgtcctccatgtcctgctccagaactgctcggcggaccttccgtgtttctgttccctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacgcctccacctacagagtggtgtccgtgctgacagtgctgcaccaggattggctgaacggcaaagagtacaagtgcaaggtgtccaacaaggccctgcctgctcctatcgaaaagaccatctccaaggccaagggccagcctagggaaccccaggtttacaccctgccacctagccgggaagagatgaccaagaaccaggtgtccctgacctgcctcgtgaagggcttctacccttccgatatcgctgtggaatgggagagcaacggccagcctgagaacaactacaagacaacccctcctgtgctggactccgacggctcattctttctgtactccaagctgactgtggacaagtccagatggcagcagggcaacgtgttctcctgcagcgtgatgcacgaggccctgcacaatcactacacacagaagtctctgtctctgagccccggc

SEQ ID NO: 38 - DNA, 642 bp, Artificial Sequence
  Description of Artificial Sequence Synthetic polynucleotide
gacatccagatgacccagtctccatcctctctgtccgcctctgtgggcgacagagtgaccatcacctgtcgggcctccgagaacatctactcctacctggcctggtatcagcagaagcctggcaaggctcccaagctgctgatctacaccgctaagacactggccgagggcgtgccctctagattttctggctctggaagcggcaccgactttaccctgacaatctccagcctgcagcctgaggacttcgccacctactactgccagcaccagtacggccctccattcacctttggccagggcaccaagctggaaatcaagcggacagtggccgctccttccgtgttcatcttcccaccttccgacgagcagctgaagtctggcacagcctctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgc

SEQ ID NO: 39 - DNA, 1344 bp, Artificial Sequence
  Description of Artificial Sequence Synthetic polynucleotide
caggttcagttggttcagtctggcgccgaagtgaagaaacctggcgcctctgtgaaggtgtcctgcaaggcttccggctacacctttaccagctactggatgcactgggtccgacaggctccaggacaaggcttggagtggatgggcaacatcgacccctctgacagcgacacccactacaaccagaaattcaaggaccgcgtgaccatgaccagagacacctccaccagcaccgtgtacatggaactgtccagcctgagatccgaggacaccgccgtgtactactgtgccagaggctactccaagtactacgccatggactactggggccagggcacactggttaccgtgtcctctgcttccaccaagggaccctctgtgttccctctggctccttccagcaagtctacctctggcggaacagctgctctgggctgcctggtcaaggactactttcctgagcctgtgaccgtgtcttggaactctggcgctctgacatctggcgtgcacacattccctgctgtgctgcagtcctccggcctgtactctctgtcctctgtcgtgaccgtgccttccagctctctgggaacccagacctacatctgcaatgtgaaccacaagccttccaacaccaaggtggacaagaaggtggaacccaagtcctgcgacaagacccacacctgtcctccatgtcctgctccagaactgctcggcggaccttccgtgtttctgttccctccaaagcctaaggacaccctgatgatctctcggacccctgaagtgacctgcgtggtggtggatgtgtcccacgaagatccagaagtgaagttcaattggtacgtggacggcgtggaagtgcacaacgccaagaccaagcctagagaggaacagtacgcctccacctacagagtggtgtccgtgctgacagtgctgcaccaggattggctgaacggcaaagagtacaagtgcaaggtgtccaacaaggccctgcctgctcctatcgaaaagaccatctccaaggccaagggccagcctagggaaccccaggtttacaccctgcctccaagccgggaagagatgaccaagaaccaggtgtccctgacctgcctcgtgaagggcttctacccttccgatatcgccgtggaatgggagagcaatggccagccagagaacaactacaagacaacccctcctgtgctggactccgacggctcattctttctgtactccaagctgaccgtggacaagtccagatggcagcagggcaacgtgttctcctgcagcgtgatgcacgaggccctgcacaatcactatacccagaagtccctgtctctgtcccctggc

SEQ ID NO: 40 - DNA, 657 bp, Artificial Sequence
  Description of Artificial Sequence Synthetic polynucleotide
gacatcgtgatgacccagacacctctgagcctgagcgtgacacctggacagcctgcctccatctcctgcagatcctctcagtccatcgtgcactcctacggcaacacctacctggaatggtatctgcagaagcccggccagtctcctcagctgctgatctacaaggtgtccaaccggttctctggcgtgcccgacagattttccggctctggctctggcaccgacttcaccctgaagatctccagagtggaagccgaggacgtgggcgtgtactactgcttccaaggctctcacgtgccctacacctttggccagggcaccaagctggaaatcaagcggacagtggccgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtccagcacactgaccctgtccaaggccgactacgagaagcacaaggtgtacgcctgcgaagtgacccatcagggcctgtctagccctgtgaccaagtctttcaaccggggcgagtgc

SEQ ID NO: 41 - DNA, 342 bp, Artificial Sequence
  Description of Artificial Sequence Synthetic polynucleotide
gaggttcagctgcagcagtctggggcagagcttgtgaagccaggggcctcagtcaagttgtcctgcacagcttctggcttcaacattaaagactactatatacactgggtgaagcagaggactgaacagggcctggagtggattggaaggattgatcctgaggatggtgaaactaaatatgccccgaaattccagggcaaggccactataacagcagacacatcctccaacacagcctacctgcagctcaacagcctgacatctgaggacactgccgtctattcctgtgctaagggggggtttgcttactggggccaagggactctggtcactgtctctgca

SEQ ID NO: 42 - DNA, 324 bp, Artificial Sequence
  Description of Artificial Sequence Synthetic polynucleotide
caaattgttctcacccagtctccagcaatcatgtctgcatctcctggggagaaggtcaccttgacctgcagtgccagttcaagtgtaagttccagctacttgtactggtaccagcagaagccaggatcctcccccaaactctggatttatagcacatccaacctggcttctggagtccctgctcgcttcagtggcagtgggtctgggacctcttactctctcacaatcagcagcatggaggctgaagatgctgcctcttatttctgccatcagtggagtagtcacccgtacacgttcggaggggggaccaagctggaaataaaa

SEQ ID NO: 43 - DNA, 360 bp, Artificial Sequence
  Description of Artificial Sequence Synthetic polynucleotide
caggtcaagctgcaggagtctggggctgagctggtgaggcctgggtcttcagtgaagctgtcctgcaaggcttctggctacaccttcaccagctactggatgcattgggtgaagcagaggcctatacaaggccttgaatggattggtaacattgacccttctgatagtgatactcactacaatcaaaagttcaaggacaaggccacattgactgtggacaactcctccagcacagcctacatgcagctcagcagcctgacctctgaggactctgcggtctattactgtgcaagctatggtaactacggggagaatgctatggactactggggtcaaggaacctcagtcaccgtctcctca

SEQ ID NO: 44 - DNA, 336 bp, Artificial Sequence
  Description of Artificial Sequence Synthetic polynucleotide
gatattttgatgacccaaactccactctccctgcctgtcagtcttggagatcaagcctccatctcttgcagatctagtcagagcattgtacatagttatggaaacacctatttagaatggtacctgcagaaaccaggccagtctccaaaactcctgatctacaaagtttccaaccgattttctggggtcccagacaggttcagtggcagtggatcaggtacagatttcacactcaagatcagcagagtggaggctgaggatctgggagtttattactgctttcaaggttcacatgttccattcacgttcggctcggggacaaagttggaaataaaa

SEQ ID NO: 45 - protein, 116 aa, Homo sapiens
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRA

SEQ ID NO: 46 - protein, 113 aa, Artificial Sequence
  Description of Artificial Sequence Synthetic polypeptide
EVQLVQSGAEVKKPGATVKISCKVSGFNIKDYYIHWVQQAPGKGLEWIGRIDPEDGETKYAPKFQDRATITADTSTDTAYMELSSLRSEDTAVYYCARWGAYWGQGTLVTVSS

SEQ ID NO: 47 - protein, 108 aa, Artificial Sequence
  Description of Artificial Sequence Synthetic polypeptide
QIVLTQSPPTLSLSPGERVTLTCSASSSVSSSYLYWYQQKPGQAPKLWIYSTSNLASGVPARFSGSGSGTSYTLTISSLQPEDFAVYFCHQWSSYPRTFGAGTKLEIK

SEQ ID NO: 48 - protein, 116 aa, Homo sapiens; identical to SEQ ID NO: 45

SEQ ID NO: 49 - protein, 115 aa, Homo sapiens
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRA

SEQ ID NO: 50 - protein, 444 aa, Artificial Sequence
  Description of Artificial Sequence Synthetic polypeptide
QVQLVQSGAEVKKPGASVKVSCKASGYTFTNYNMHWVRQAPGQRLEWMGTIYPGNDDTSYNQKFKDRVTITADTSASTAYMELSSLRSEDTAVYYCARGGYRAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 51 - protein, 219 aa, Artificial Sequence
  Description of Artificial Sequence Synthetic polypeptide
DIVMTQSPLSLPVTPGEPASISCRSSQSIVYSNGNTYLGWYLQKPGQSPQLLIYKVSNRFSGVPDRFSGSGSGTDFTLKISRVEAEDVGVYYCFQGSHVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 5 aa, Artificial Sequence
  Description of Artificial Sequence Synthetic peptide
NYNMH

SEQ ID NO: 53 - protein, 17 aa, Artificial Sequence
  Description of Artificial Sequence Synthetic peptide
TIYPGNDDTSYNQKFKD

SEQ ID NO: 54 - protein, 8 aa, Artificial Sequence
  Description of Artificial Sequence Synthetic peptide
GGYRAMDY

SEQ ID NO: 55 - protein, 15 aa, Artificial Sequence
  Description of Artificial Sequence Synthetic peptide
RSSQSIVYSNGNTYL

SEQ ID NO: 56 - protein, 7 aa, Artificial Sequence
  Description of Artificial Sequence Synthetic peptide
KVSNRFS

SEQ ID NO: 57 - protein, 9 aa, Artificial Sequence
  Description of Artificial Sequence Synthetic peptide
FQGSHVPYT

SEQ ID NO: 58 - protein, 116 aa, Artificial Sequence
  Description of Artificial Sequence Synthetic polypeptide
QVQLQQPGAELVKPGASVMMSCKASGYTFTNYNMHWVKQTPGQGLEWIGTIYPGNDDTSYNQKFKDKATLTADKSSSAAYMQLSSLTSEDSAVYYCARGGYRAMDYWGQTSVTVSS

SEQ ID NO: 59 - protein, 112 aa, Artificial Sequence
  Description of Artificial Sequence Synthetic polypeptide
DVLMTQTPLSLPVSLGDQASISCRSSQSIVYSNGNTYLGWYLQKPGQSPKLLIYKVSNRFSGVPDRFSGSGSGTDFTLKISRVEAEDLGVYHCFQGSHVPYTFGGGTKVEIK

SEQ ID NO: 60 - protein, 118 aa, Artificial Sequence
  Description of Artificial Sequence Synthetic polypeptide
EVQLVESGGGLVQPGGSLRLSCAASGFTFSGYGMSWVRQAPGKGLEWVATITSGGTYTYYPDSVKGRFTISRDNAKNSLYLQMNSLRAEDTAVYYCARSLAGNAMDYWGQGTLVTVSS

SEQ ID NO: 61 - protein, 107 aa, Artificial Sequence
  Description of Artificial Sequence Synthetic polypeptide
EIVLTQSPATLSLSPGERATLSCRASQTISDYLHWYQQKPGQAPRLLIKFASQSISGIPARFSGSGSGTDFTLTISSLEPEDFAVYYCQNGHGFPRTFGQGTKVEIK

SEQ ID NO: 62 - protein, 109 aa, Artificial Sequence
  Description of Artificial Sequence Synthetic polypeptide
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCARWGQGTLVTVSS

SEQ ID NO: 63 - protein, 107 aa, Artificial Sequence
  Description of Artificial Sequence Synthetic polypeptide
DIVMTQSPATLSVTPGDRVSLSCRASQNFSDYLHWYQQKSHESPRLLIKYVSHSISGIPSRFSGSGSGSDFTLSINSVEPEDVGVYYCQNGHSFPPTFGGGTKLEIK

SEQ ID NO: 64 - protein, 116 aa, Artificial Sequence
  Description of Artificial Sequence Synthetic polypeptide
QVQLQQSGAELVKPGASVKLSCKASGYTFTNYYIFWVKERPGQGLEWIGDINPSNGDTNFNEKFKIKATLTVDKSSSTTYMQLNSLTSEDSAVYFCTRGGYTMDYWGQGTSVTVSS

SEQ ID NO: 65 - protein, 112 aa, Artificial Sequence
  Description of Artificial Sequence Synthetic polypeptide
DVVMTQTPLSLPVSLGDQASISCRSSQSLVHSNGNTYFHWYVQKPGQSPKLLIYKVSYRFSGVPDRFSGSGSGTDFTLKISRVEAEDLGVYFCSQSTHVPRTFGGGTKLEIK

SEQ ID NO: 66 - protein, 116 aa, Artificial Sequence
  Description of Artificial Sequence Synthetic polypeptide
QVQLVQSGAEVKKPGASVKVSCKASGYTFTNYYIFWVRQAPGQGLEWIGDINPSNGDTNFNEKFKIKATLTVDKSTSTTYMELSSLRSEDTAVYYCTRGGYTMDYWGQGTLVTVSS

SEQ ID NO: 67 - protein, 116 aa, Artificial Sequence
  Description of Artificial Sequence Synthetic polypeptide
QVQLVQSGAEVKKPGASVKVSCKASGYTFTNYYIFWVRQAPGQGLEWMGDINPSNGDTNFNEKFKIRVTMTRDTSTSTVYMELSSLRSEDTAVYYCTRGGYTMDYWGQGTLVTVSS

SEQ ID NO: 68 - protein, 112 aa, Artificial Sequence
  Description of Artificial Sequence Synthetic polypeptide
DVVMTQTPLSLSVTPGQPASISCRSSQSLVHSNGNTYFHWYLQKPGQPPKLLIYKVSYRFSGVPDRFSGSGSGTDFTLKISRVEAEDVGVYFCSQSTHVPRTFGQGTKVEIK

SEQ ID NO: 69 - protein, 112 aa, Artificial Sequence
  Description of Artificial Sequence Synthetic polypeptide
DIVMTQTPLSLSVTPGQPASISCRSSQSLVHSNGNTYFHWYLQKPGQPPQLLIYKVSYRFSGVPDRFSGSGSGTDFTLKISRVEAEDVGVYYCSQSTHVPRTFGQGTKVEIK

SEQ ID NO: 70 - protein, 118 aa, Artificial Sequence
  Description of Artificial Sequence Synthetic polypeptide
EVQLQQSGAELVRSGASVKLSCTASGFNIKDYYLHWVKQRPEQGLEWIGWIDPDNGDTEFAPKFQGKATMTADTSSNTAYLQLSSLTSEDTAVYYCNAAYGSSSYPMDYWGQGTSVTV

SEQ ID NO: 71 - protein, 118 aa, Artificial Sequence
  Description of Artificial Sequence Synthetic polypeptide
EVQLVQSGAEVKKPGATVKISCKVSGFNIKDYYLHWVQQAPGKGLEWMGWIDPDNGDTEYAEKFQGRVTITADTSTDTAYMELSSLRSEDTAVYYCNAAYGSSSYPMDYWGQGTTVTV

SEQ ID NO: 72 - protein, 118 aa, Artificial Sequence
  Description of Artificial Sequence Synthetic polypeptide
QMQLVQSGAEVKKTGSSVKVSCKASGFNIKDYYLHWVRQAPGQALEWMGWIDPDNGDTEYAQKFQDRVTITRDRSMSTAYMELSSLRSEDTAMYYCNAAYGSSSYPMDYWGQGTTVTV

SEQ ID NO: 73 - protein, 118 aa, Artificial Sequence
  Description of Artificial Sequence Synthetic polypeptide
EVQLVQSGAEVKKTGSSVKVSCKASGFNIKDYYLHWVRQAPGQALEWMGWIDPDNGDTEYAQKFQDRVTITRDRSMSTAYMELSSLRSEDTAMYYCNAAYGSSSYPMDYWGQGTTVTV

SEQ ID NO: 74 - protein, 118 aa, Artificial Sequence
  Description of Artificial Sequence Synthetic polypeptide
QMQLVQSGAEVKKTGSSVKVSCKASGFNIKDYYLHWVRQAPGQALEWMGWIDPDNGDTEYAQKFQGRVTMTADTSSNTAYMELSSLRSEDTAMYYCNAAYGSSSYPMDYWGQGTTVTV

SEQ ID NO: 75 - protein, 118 aa, Artificial Sequence
  Description of Artificial Sequence Synthetic polypeptide
QMQLVQSGAEVKKTGSSVKVSCKASGFNIKDYYLHWVRQAPGQALEWMGWIDPDNGDTEYAQKFQGRVTMTEDTSTDTAYMELSSLRSEDTAMYYCNAAYGSSSYPMDYWGQGTTVTV

SEQ ID NO: 76 - protein, 118 aa, Artificial Sequence
  Description of Artificial Sequence Synthetic polypeptide
QMQLVQSGAEVKKTGSSVKVSCKASGFNIKDYYLHWVRQAPGQALEWMGWIDPDQGDTEYAQKFQDRVTITRDRSMSTAYMELSSLRSEDTAMYYCNAAYGSSSYPMDYWGQGTTVTV

SEQ ID NO: 77 - protein, 118 aa, Artificial Sequence
  Description of Artificial Sequence Synthetic polypeptide
QMQLVQSGAEVKKTGSSVKVSCKASGFNIKDYYLHWVRQAPGQALEWMGWIDPDYGDTEYAQKFQDRVTITRDRSMSTAYMELSSLRSEDTAMYYCNAAYGSSSYPMDYWGQGTTVTV

SEQ ID NO: 78 - protein, 118 aa, Artificial Sequence
  Description of Artificial Sequence Synthetic polypeptide
QMQLVQSGAEVKKTGSSVKVSCKASGFNIKDYYLHWVRQAPGQALEWMGWIDPDSGDTEYAQKFQDRVTITRDRSMSTAYMELSSLRSEDTAMYYCNAAYGSSSYPMDYWGQGTTVTV

SEQ ID NO: 79 - protein, 118 aa, Artificial Sequence
  Description of Artificial Sequence Synthetic polypeptide
QMQLVQSGAEVKKTGSSVKVSCKASGFNIKDYYLHWVRQAPGQALEWMGWIDPDNADTEYAQKFQDRVTITRDRSMSTAYMELSSLRSEDTAMYYCNAAYGSSSYPMDYWGQGTTVTV

SEQ ID NO: 80 - protein, 118 aa, Artificial Sequence
  Description of Artificial Sequence Synthetic polypeptide
QMQLVQSGAEVKKTGSSVKVSCKASGFNIKDYYLHWVRQAPGQALEWMGWIDPDNTDTEYAQKFQDRVTITRDRSMSTAYMELSSLRSEDTAMYYCNAAYGSSSYPMDYWGQGTTVTV

SEQ ID NO: 81 - protein, 118 aa, Artificial Sequence
  Description of Artificial Sequence Synthetic polypeptide
QMQLVQSGAEVKKTGSSVKVSCKASGFNIKDYYLHWVRQAPGQALEWMGWIDPDNGDTEYAQKFQDRVTITRDRSMSTAYMELSSLRSEDTAMYYCNAAYGSSPYPMDYWGQGTTVTV

SEQ ID NO: 82 - protein, 118 aa, Artificial Sequence
  Description of Artificial Sequence Synthetic polypeptide
QMQLVQSGAEVKKTGSSVKVSCKASGYTFTYYYLHWVRQAPGQALEWMGWIDPDNGDTEYAQKFQDRVTITRDRSMSTAYMELSSLRSEDTAMYYCNAAYGSSSYPMDYWGQGTTVTV

SEQ ID NO: 83 - protein, 118 aa, Artificial Sequence
  Description of Artificial Sequence Synthetic polypeptide
QMQLVQSGAEVKKTGSSVKVSCKASGFTFTYYYLHWVRQAPGQALEWMGWIDPDNGDTEYAQKFQDRVTITRDRSMSTAYMELSSLRSEDTAMYYCNAAYGSSSYPMDYWGQGTTVTV

SEQ ID NO: 84 - protein, 118 aa, Artificial Sequence
  Description of Artificial Sequence Synthetic polypeptide
QMQLVQSGAEVKKTGSSVKVSCKASGYNFTYYYLHWVRQAPGQALEWMGWIDPDNGDTEYAQKFQDRVTITRDRSMSTAYMELSSLRSEDTAMYYCNAAYGSSSYPMDYWGQGTTVTV

SEQ ID NO: 85 - protein, 118 aa, Artificial Sequence
  Description of Artificial Sequence Synthetic polypeptide
QMQLVQSGAEVKKTGSSVKVSCKASGYTITYYYLHWVRQAPGQALEWMGWIDPDNGDTEYAQKFQDRVTITRDRSMSTAYMELSSLRSEDTAMYYCNAAYGSSSYPMDYWGQGTTVTV

SEQ ID NO: 86 - protein, 118 aa, Artificial Sequence
  Description of Artificial Sequence Synthetic polypeptide
QMQLVQSGAEVKKTGSSVKVSCKASGYTFKYYYLHWVRQAPGQALEWMGWIDPDNGDTEYAQKFQDRVTITRDRSMSTAYMELSSLRSEDTAMYYCNAAYGSSSYPMDYWGQGTTVTV

SEQ ID NO: 87 - protein, 118 aa, Artificial Sequence
  Description of Artificial Sequence Synthetic polypeptide
QMQLVQSGAEVKKTGSSVKVSCKASGYTFTDYYLHWVRQAPGQALEWMGWIDPDNGDTEYAQKFQDRVTITRDRSMSTAYMELSSLRSEDTAMYYCNAAYGSSSYPMDYWGQGTTVTV

SEQ ID NO: 88 - protein, 118 aa, Artificial Sequence
  Description of Artificial Sequence Synthetic polypeptide
QMQLVQSGAEVKKTGSSVKVSCKASGFTFTDYYLHWVRQAPGQALEWMGWIDPDNGDTEYAQKFQDRVTITRDRSMSTAYMELSSLRSEDTAMYYCNAAYGSSSYPMDYWGQGTTVTV

SEQ ID NO: 89 - protein, 118 aa, Artificial Sequence
  Description of Artificial Sequence Synthetic polypeptide
QMQLVQSGAEVKKTGSSVKVSCKASGFTITDYYLHWVRQAPGQALEWMGWIDPDNGDTEYAQKFQDRVTITRDRSMSTAYMELSSLRSEDTAMYYCNAAYGSSSYPMDYWGQGTTVTV

SEQ ID NO: 90 - protein, 118 aa, Artificial Sequence
  Description of Artificial Sequence Synthetic polypeptide
QMQLVQSGAEVKKTGSSVKVSCKASGYTFKDYYLHWVRQAPGQALEWMGWIDPDNGDTEYAQKFQDRVTITRDRSMSTAYMELSSLRSEDTAMYYCNAAYGSSSYPMDYWGQGTTVTV

SEQ ID NO: 91 - protein, 118 aa, Artificial Sequence
  Description of Artificial Sequence Synthetic polypeptide
QMQLVQSGAEVKKTGSSVKVSCKASGFTFKDYYLHWVRQAPGQALEWMGWIDPDNGDTEYAQKFQDRVTITRDRSMSTAYMELSSLRSEDTAMYYCNAAYGSSSYPMDYWGQGTTVTV

SEQ ID NO: 92 - protein, 118 aa, Artificial Sequence
  Description of Artificial Sequence Synthetic polypeptide
QMQLVQSGAEVKKTGSSVKVSCKASGFNIKDYYLHWVRQAPGQALEWMGWIDPDNGDTEYAQKFQDRVTITRDRSMSTAYLQLSSLRSEDTAMYYCNAAYGSSSYPMDYWGQGTTVTV

SEQ ID NO: 93 - protein, 118 aa, Artificial Sequence
  Description of Artificial Sequence Synthetic polypeptide
QMQLVQSGAEVKKTGSSVKVSCKASGFNIKDYYLHWVRQAPGQALEWMGWIDPDNGDTEYAQKFQDRVTITRDRSMSTAYMELSSLTSEDTAVYYCNAAYGSSSYPMDYWGQGTTVTV

SEQ ID NO: 94 - protein, 118 aa, Artificial Sequence
  Description of Artificial Sequence Synthetic polypeptide
EVQLVQSGAEVKKPGATVKISCKVSGFNIKDYYLHWVRQAPGQALEWMGWIDPDNGDTEYAQKFQDRVTITRDRSMSTAYMELSSLRSEDTAMYYCNAAYGSSSYPMDYWGQGTTVTV

SEQ ID NO: 95 - protein, 118 aa, Artificial Sequence
  Description of Artificial Sequence Synthetic polypeptide
EVQLVQSGAEVKKPGATVKISCKVSGFNIKDYYLHWVQQAPGKGLEWMGWIDPDNGDTEYAQKFQDRVTITRDRSMSTAYMELSSLRSEDTAMYYCNAAYGSSSYPMDYWGQGTTVTV

SEQ ID NO: 96 - protein, 107 aa, Artificial Sequence
  Description of Artificial Sequence Synthetic polypeptide
DIKMTQSPSSLYASLGERVTITCKASQDIHRYLSWFQQKPGKSPKILIYRANRLVDGVPSRFSGSGSGQDYSLTISSLEYEDMGIYYCLQYDEFPYTFGGGTKLEMK

SEQ ID NO: 97 - protein, 107 aa, Artificial Sequence
  Description of Artificial Sequence Synthetic polypeptide
DIKMTQSPSSLYASLGERVTITCKASQDIHRYLSWFQQKPGKSPKILIYRANRLVDGVPSRFSGSGSGQDYSLTISSLEYEDMGIYYCLQYDEFPYTFGGGTKLEIK

SEQ ID NO: 98 - protein, 107 aa, Artificial Sequence
  Description of Artificial Sequence Synthetic polypeptide
DIQMTQSPSSLSASVGDRVTITCKASQDIHRYLSWYQQKPGKAPKLLIYRANRLVDGVPSRFSGSGSGTDFTFTISSLQPEDIATYYCLQYDEFPYTFGGGTKVEIK

SEQ ID NO: 99 - protein, 107 aa, Artificial Sequence
  Description of Artificial Sequence Synthetic polypeptide
DIQMTQSPSSLSASVGDRVTITCKASQDIHRYLSWFQQKPGKAPKSLIYRANRLVDGVPSRFSGSGSGTDFTLTISSLQPEDFATYYCLQYDEFPYTFGGGTKVEIK

SEQ ID NO: 100 - protein, 107 aa, Artificial Sequence
  Description of Artificial Sequence Synthetic polypeptide
NIQMTQSPSAMSASVGDRVTITCKASQDIHRYLSWFQQKPGKVPKHLIYRANRLVDGVPSRFSGSGSGTEFTLTISSLQPEDFATYYCLQYDEFPYTFGGGTKVEIK

SEQ ID NO: 101 - protein, 107 aa, Artificial Sequence
  Description of Artificial Sequence Synthetic polypeptide
DIQMTQSPSSLSASVGDRVTITCKASQDIHRYLSWYQQKPGKAPKRLIYRANRLVDGVPSRFSGSGSGTEFTLTISSLQPEDFATYYCLQYDEFPYTFGGGTKVEIK

SEQ ID NO: 102 - protein, 107 aa, Artificial Sequence
  Description of Artificial Sequence Synthetic polypeptide
DIQMTQSPSSLSASVGDRVTITCRASQDIHRYLAWYQQKPGKVPKLLIYRANRLQSGVPSRFSGSGSGTDFTLTISSLQPEDVATYYCLQYDEFPYTFGQGTKVEIK

SEQ ID NO: 103 - protein, 107 aa, Artificial Sequence
  Description of Artificial Sequence Synthetic polypeptide
EIVLTQSPATLSLSPGERATLSCRASQDIHRYLAWYQQKPGQAPRLLIYRANRRATGIPARFSGSGSGTDFTLTISSLEPEDFAVYYCLQYDEFPYTGFQGTRLEIK

SEQ ID NO: 104 - protein, 107 aa, Artificial Sequence
  Description of Artificial Sequence Synthetic polypeptide
DIQMTQSPSAMSASVGDRVTITCKASQDIHRYLSWFQQKPGKVPKHLIYRANRLVDGVPSRFSGSGSGTEFTLTISSLQPEDFATYYCLQYDEFPYTFGGGTKVEIK

SEQ ID NO: 105 - protein, 107 aa, Artificial Sequence
  Description of Artificial Sequence Synthetic polypeptide
NIQMTQSPSAMSASVGDRVTITCRARQGIHRYLSWFQQKPGKVPKHLIYRANRLVDGVPSRFSGSGSGTEFTLTISSLQPEDFATYYCLQYDEFPYTFGGGTKVEIK

SEQ ID NO: 106 - protein, 107 aa, Artificial Sequence
  Description of Artificial Sequence Synthetic polypeptide
NIQMTQSPSAMSASVGDRVTITCKASQDIHRYLSWFQQKPGKVPKILIYRANRLVDGVPSRFSGSGSGTEFTLTISSLQPEDFATYYCLQYDEFPYTFGGGTKVEIK

SEQ ID NO: 107 - protein, 107 aa, Artificial Sequence
  Description of Artificial Sequence Synthetic polypeptide
NIQMTQSPSAMSASVGDRVTITCKASQDIHRYLSWFQQKPGKVPKHLIYRANRLVSGVPSRFSGSGSGTEFTLTISSLQPEDFATYYCLQYDEFPYTFGGGTKVEIK

SEQ ID NO: 108 - protein, 107 aa, Artificial Sequence
  Description of Artificial Sequence Synthetic polypeptide
NIQMTQSPSAMSASVGDRVTITCRARQGIHRYLSWFQQKPGKVPKILIYRANRLVDGVPSRFSGSGSGTEFTLTISSLQPEDFATYYCLQYDEFPYTFGGGTKVEIK

SEQ ID NO: 109 - protein, 107 aa, Artificial Sequence
  Description of Artificial Sequence Synthetic polypeptide
NIQMTQSPSAMSASVGDRVTITCRARQGIHRYLSWFQQKPGKVPKHLIYRANRLVSGVPSRFSGSGSGTEFTLTISSLQPEDFATYYCLQYDEFPYTFGGGTKVEIK

SEQ ID NO: 110 - protein, 107 aa, Artificial Sequence
  Description of Artificial Sequence Synthetic polypeptide
NIQMTQSPSAMSASVGDRVTITCKASQDIHRYLSWFQQKPGKVPKLLIYRANRLVDGVPSRFSGSGSGTEFTLTISSLQPEDFATYYCLQYDEFPYTFGGGTKVEIK

SEQ ID NO: 111 - protein, 107 aa, Artificial Sequence
  Description of Artificial Sequence Synthetic polypeptide
NIQMTQSPSAMSASVGDRVTITCKASQDIHRYLSWFQQKPGKVPKLLIYRANRLVSGVPSRFSGSGSGTEFTLTISSLQPEDFATYYCLQYDEFPYTFGGGTKVEIK

SEQ ID NO: 112 - protein, 107 aa, Artificial Sequence
  Description of Artificial Sequence Synthetic polypeptide
NIQMTQSPSAMSASVGDRVTITCRARQGIHRYLSWFQQKPGKVPKLLIYRANRLVSGVPSRFSGSGSGTEFTLTISSLQPEDFATYYCLQYDEFPYTFGGGTKVEIK

SEQ ID NO: 113 - protein, 117 aa, Artificial Sequence
  Description of Artificial Sequence Synthetic polypeptide
QVQLVQSGAEVKKPGASVKVSCKASGYTFTNYNMHWVRQAPGQGLEWIGTIYPGNDDTSYNQKFKDKATLTADKSTSTAYMELSSLRSEDTAVYYCARGGYRAMDYWGQGTLVTVSS

SEQ ID NO: 114 - protein, 117 aa, Artificial Sequence
  Description of Artificial Sequence Synthetic polypeptide
QVQLVQSGAEVKKPGASVKVSCKASGYTFTNYNMHWVRQAPGQRLEWMGTIYPGNDDTSYNQKFKDRVTITADTSASTAYMELSSLRSEDTAVYYCARGGYRAMDYWGQGTLVTVSS

SEQ ID NO: 115 - protein, 117 aa, Artificial Sequence
  Description of Artificial Sequence Synthetic polypeptide
QVQLVQSGAEVKKPGASVKVSCKASGYTFTNYNMHWVRQAPGQRLEWIGTIYPGNDDTSYNQKFKDRATLTADKSASTAYMELSSLRSEDTAVYYCARGGYRAMDYWGQGTLVTVSS

SEQ ID NO: 116 - protein, 112 aa, Artificial Sequence
  Description of Artificial Sequence Synthetic polypeptide
DVVMTQSPLSLPVTPGEPASISCRSSQSIVYSNGNTYLGWYLQKPGQSPKLLIYKVSNRFSGVPDRFSGSGSGTDFTLKISRVEAEDVGVYHCFQGSHVPYTFGGGTKVEIK

SEQ ID NO: 117 - protein, 112 aa, Artificial Sequence
  Description of Artificial Sequence Synthetic polypeptide
DIVMTQSPLSLPVTPGEPASISCRSSQSIVYSNGNTYLGWYLQKPGQSPQLLIYKVSNRFSGVPDRFSGSGSGTDFTLKISRVEAEDVGVYYCFQGSHVPYTFGQGTKLEIK

SEQ ID NO: 118 - protein, 112 aa, Artificial Sequence
  Description of Artificial Sequence Synthetic polypeptide
DVVMTQSPLSLPVTPGEPASISCRSSQSIVYSNGNTYLGWYLQKPGQSPQLLIYKVSNRFSGVPDRFSGSGSGTDFTLKISRVEAEDVGVYHCFQGSHVPYTFGQGTKLEIK

SEQ ID NO: 119 - protein, 120 aa, Artificial Sequence
  Description of Artificial Sequence Synthetic polypeptide
QVQLQQPGAELVRPGSSVKLSCKASGYTFTSYWVHWVKQRPIQGLEWIGNIDPSDSDTHYNQKFKDKASLTVDKSSSTAYMQLSSLTFEDSAVYYCVRGGTGTMAWFAYWGQGTLVTVSA

SEQ ID NO: 120 - protein, 120 aa, Artificial Sequence
  Description of Artificial Sequence Synthetic polypeptide
EVQLVQSGAEVKKPGESLRISCKASGYTFTSYWVHWVRQMPGKGLEWIGNIDPSDSDTHYNQKFKDHVTLSVDKSISTAYLQLSSLKASDTAMYYCVRGGTGTMAWFAYWGQGTLVTVSS

SEQ ID NO: 121 - protein, 120 aa, Artificial Sequence
  Description of Artificial Sequence Synthetic polypeptide
EVQLVQSGAEVKKPGESLRISCKASGYSFTSYWVHWVRQMPGKGLEWMGNIDPSDSDTHYNQKFKDHVTLSVDKSISTAYLQLSSLKASDTAMYYCVRGGTGTMAWFAYWGQGTLVTVSS

SEQ ID NO: 122 - protein, 120 aa, Artificial Sequence
  Description of Artificial Sequence Synthetic polypeptide
EVQLVQSGAEVKKPGESLRISCKASGYSFTSYWVHWVRQMPGKGLEWMGNIDPSDSDTHYSPSFQGHVTLSVDKSISTAYLQLSSLKASDTAMYYCVRGGTGTMAWFAYWGQGTLVTVSS

SEQ ID NO: 123 - protein, 120 aa, Artificial Sequence
  Description of Artificial Sequence Synthetic polypeptide
EVQLVQSGAEVKKPGESLRISCKASGYSFTSYWVHWVRQMPGKGLEWMGNIDPSDSDTHYSPSFQGHVTLSVDKSISTAYLQLSSLKASDTAMYYCVRGGTGTLAWFAYWGQGTLVTVSS

SEQ ID NO: 124 - protein, 120 aa, Artificial Sequence
  Description of Artificial Sequence Synthetic polypeptide
EVQLVQSGAEVKKPGESLRISCKASGYSFTSYWVHWVRQMPGKGLEWMGNIDPSDSDTHYSPSFQGHVTLSVDKSISTAYLQLSSLKASDTAMYYCVRGGTGTMAYFAYWGQGTLVTVSS

SEQ ID NO: 125 - protein, 120 aa, Artificial Sequence
  Description of Artificial Sequence Synthetic polypeptide
EVQLVQSGAEVKKPGESLRISCKASGYSFTSYWVHWVRQMPGKGLEWMGNIDPSDSDTHYSPSFQGHVTLSVDKSISTAYLQLSSLKASDTAMYYCVRGGTGTLAYFAYWGQGTLVTVSS

SEQ ID NO: 126 - protein, 112 aa, Artificial Sequence
  Description of Artificial Sequence Synthetic polypeptide
DVVMTQTPLSLPVSLGDQASISCRSSQSLVHSYGNTYLYWYLQKPGQSPKLLIYRVSNRFSGVPDRFSGSGSGTDFTLKISRVEAEDLGVYFCFQGTHVPYTFGSGTKLEIK

SEQ ID NO: 127 - protein, 112 aa, Artificial Sequence
  Description of Artificial Sequence Synthetic polypeptide
DVVMTQSPLSLPVTLGQPASISCRSSQSLVHSYGNTYLYWYQQRPGQSPRLLIYRVSNRFSGVPDRFSGSGSGTDFTLKISRVEAEDVGVYFCFQGTHVPYTFGGGTKVEIK

SEQ ID NO: 128 - protein, 112 aa, Artificial Sequence
  Description of Artificial Sequence Synthetic polypeptide
DVVMTQSPLSLPVTLGQPASISCRSSQSLVHSYGNTYLYWFQQRPGQSPRLLIYRVSNRFSGVPDRFSGSGSGTDFTLKISRVEAEDVGVYYCFQGTHVPYTFGGGTKVEIK

SEQ ID NO: 129 - protein, 451 aa, Artificial Sequence
  Description of Artificial Sequence Synthetic polypeptide
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 130 - protein, 213 aa, Artificial Sequence
  Description of Artificial Sequence Synthetic polypeptide
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 131 - protein, 13 aa, Artificial Sequence
  Description of Artificial Sequence Synthetic peptide
KASGYTFTSYNMH

SEQ ID NO: 132 - protein, 10 aa, Artificial Sequence
  Description of Artificial Sequence Synthetic peptide
AIYPGNGDTS

SEQ ID NO: 133 - protein, 14 aa, Artificial Sequence
  Description of Artificial Sequence Synthetic peptide
ARSTYYGGDWYFNV

SEQ ID NO: 134 - protein, 10 aa, Artificial Sequence
  Description of Artificial Sequence Synthetic peptide
RASSSVSYIH

SEQ ID NO: 135 - protein, 8 aa, Artificial Sequence
  Description of Artificial Sequence Synthetic peptide
YATSNLAS

SEQ ID NO: 136 - protein, 9 aa, Artificial Sequence
  Description of Artificial Sequence Synthetic peptide
QQWTSNPPT

SEQ ID NO: 137 - protein, 125 aa, Artificial Sequence
  Description of Artificial Sequence Synthetic polypeptide
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTK

SEQ ID NO: 138 - protein, 94 aa, Artificial Sequence
  Description of Artificial Sequence Synthetic polypeptide
GPSVFPLAPSSKSTSGGTAALGCLVKDYFPEPVTVSWNSGALTSGVHTFPAVLQSSGLYSLSSVVTVPSSSLGTQTYICNVNHKPSNTKVDKKA

SEQ ID NO: 139 - protein, 15 aa, Artificial Sequence
  Description of Artificial Sequence Synthetic peptide
EPKSCDKTHTCPPCP

SEQ ID NO: 140 - protein, 110 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 141 - protein, 107 aa, Artificial Sequence
  Description of Artificial Sequence Synthetic polypeptide
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 142 - protein, 106 aa, Artificial Sequence [sequence omitted]

SEQ ID NO: 143 - protein, 107 aa, Artificial Sequence [sequence omitted]

SEQ ID NO: 144 - protein, 117 aa, Artificial Sequence; identical to SEQ ID NO: 114
  Description of Artificial Sequence Synthetic polypeptide

SEQ ID NO: 145 - protein, 112 aa, Artificial Sequence; identical to SEQ ID NO: 117
  Description of Artificial Sequence Synthetic polypeptide

SEQ ID NO: 146 - protein, 16 aa, Artificial Sequence
  Description of Artificial Sequence Synthetic peptide
RSSQSIVYSNGNTYLG

SEQ ID NO: 147 - protein, 8 aa, Artificial Sequence
  Description of Artificial Sequence Synthetic peptide
GYTFTNYN

SEQ ID NO: 148 - protein, 8 aa, Artificial Sequence
  Description of Artificial Sequence Synthetic peptide
IYPGNDDT

SEQ ID NO: 149 - protein, 10 aa, Artificial Sequence
  Description of Artificial Sequence Synthetic peptide
ARGGYRAMDY

SEQ ID NO: 150 - protein, 11 aa, Artificial Sequence
  Description of Artificial Sequence Synthetic peptide
QSIVYSNGNTY

SEQ ID NO: 151 - protein, 3 aa, Artificial Sequence
  Description of Artificial Sequence Synthetic peptide
KVS

SEQ ID NO: 152 - protein, 9 aa, Artificial Sequence
  Description of Artificial Sequence Synthetic peptide
FQGSHVPYT

SEQ ID NO: 153 - protein, 7 aa, Artificial Sequence
  Description of Artificial Sequence Synthetic peptide
GYTFTNY

SEQ ID NO: 154 - protein, 4 aa, Artificial Sequence
  Description of Artificial Sequence Synthetic peptide
PGND

SEQ ID NO: 155 - protein, 6 aa, Artificial Sequence
  Description of Artificial Sequence Synthetic peptide
GYRAMD

SEQ ID NO: 156 - protein, 12 aa, Artificial Sequence
  Description of Artificial Sequence Synthetic peptide
SQSIVYSNGNTY

SEQ ID NO: 157 - protein, 3 aa, Artificial Sequence
  Description of Artificial Sequence Synthetic peptide
KVS

SEQ ID NO: 158 - protein, 6 aa, Artificial Sequence
  Description of Artificial Sequence Synthetic peptide
GSHVPY

SEQ ID NO: 159 - protein, 10 aa, Artificial Sequence
  Description of Artificial Sequence Synthetic peptide
ASGYTFTNYN

SEQ ID NO: 160 - protein, 17 aa, Artificial Sequence
  Description of Artificial Sequence Synthetic peptide
IYPGNDDTSYNQKFKDR

SEQ ID NO: 161 - protein, 7 aa, Artificial Sequence
  Description of Artificial Sequence Synthetic peptide
GGYRAMD

SEQ ID NO: 162 - protein, 13 aa, Artificial Sequence
  Description of Artificial Sequence Synthetic peptide
SSQSIVYSNGNTY

SEQ ID NO: 163 - protein, 12 aa, Artificial Sequence
  Description of Artificial Sequence Synthetic peptide
KVSNRFSGVPDR

SEQ ID NO: 164 - protein, 6 aa, Artificial Sequence
  Description of Artificial Sequence Synthetic peptide
GSHVPY

SEQ ID NO: 165 - protein, 448 aa, Artificial Sequence
  Description of Artificial Sequence Synthetic polypeptide
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 166 - protein, 214 aa, Artificial Sequence
  Description of Artificial Sequence Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 167 - protein, 15 aa, Artificial Sequence
  Description of Artificial Sequence Synthetic peptide
RRCPLYISYDPVCRR

SEQ ID NO: 168 - protein, 19 aa, Artificial Sequence
  Description of Artificial Sequence Synthetic peptide
RRRRCPLYISYDPVCRRRR

(19) United States
(12) Patent Application Publication
(10) Pub. No.: US 2021/0147568 A1
(43) Pub. Date: May 20, 2021

(54) ANTI-CD47 BASED TREATMENT OF BLOOD CANCER

(71) Applicant: Forty Seven, Inc., Foster City, CA (US)

(72) Inventors: Mark Ping Chao, Mountain View, CA (US); Roy Louis Maute, Menlo Park, CA (US); Jie Huang, Menlo Park, CA (US); Chris Hidemi Mizufune Takimoto, Menlo Park, CA (US); Balaji Agoram, Menlo Park, CA (US); Irving L. Weissman, Stanford, CA (US)

(21) Appl. No.: 17/083,556

(22) Filed: Oct. 29, 2020

Related U.S. Application Data
(60) Provisional application No. 63/031,418, filed on May 28, 2020.
(60) Provisional application No. 62/928,988, filed on Oct. 31, 2019.

(51) Int. Cl. C07K 16/28; G01N 33/50; A61P 35/00

(52) U.S. Cl. CPC C07K 16/2887; C07K 16/2803; G01N 2800/52; A61P 35/00; G01N 33/5052

(57) ABSTRACT

Methods, kits, and compositions are provided herein for determining the eligibility of a subject to receive an anti-CD47 treatment based on a presence or absence of B-cells in the subject, and subsequently treating the eligible subject with the anti-CD47 treatment alone or in combination with one or more additional agent such as an anti-CD20 antibody.

### CROSS REFERENCE TO RELATED APPLICATIONS

This application claims benefit of U.S. Provisional Application No. 62/928,988, filed Oct. 31, 2019; and U.S. Provisional application No. 63/031,418, filed May 28, 2020; each of which is hereby incorporated by reference in its entirety for all purposes.

### SEQUENCE LISTING

The instant application contains a Sequence Listing which has been submitted electronically in ASCII format and is hereby incorporated by reference in its entirety. Said ASCII copy, created on Oct. 22, 2020, is named FSI-007_P2C_SL.txt and is 155,274 bytes in size.

### BACKGROUND

CD47 has been identified as a key molecule mediating cancer cell evasion of phagocytosis by the innate immune system. CD47 appears to be an important means by which cancer cells, including cancer stem cells, overcome intrinsic expression of their prophagocytic, “eat me,” signals. The progression from normal cell to cancer cell can involve changes in genes and/or gene expression that trigger programmed cell death (PCD) and programmed cell removal (PCR). Many of the steps in cancer progression subvert multiple mechanisms of PCD, and expression of anti-phagocytic signal, CD47, may represent an important checkpoint.

CD47 expression is increased on the surface of many cancer cells from a large number of diverse human tumor types including the following primary malignancies: head and neck, melanoma, breast, lung, ovarian, pancreatic, colon, bladder, prostate, leiomyosarcoma, glioblastoma, medulloblastoma, oligodendroglioma, glioma, lymphoma, leukemia, and multiple myeloma. In murine xenograft studies, it has been shown that CD47-blocking antibodies inhibit human cancer growth and metastasis by enabling phagocytosis and elimination of cancer cells from various hematologic malignancies and several solid tumors.

CD47 serves as the ligand for SIRPα, which is expressed on phagocytic cells including macrophages and dendritic cells. When SIRPα is activated by CD47 binding, it initiates a signal transduction cascade resulting in inhibition of phagocytosis. In this way, CD47 functions as an anti-phagocytic signal by delivering a dominant inhibitory signal to phagocytic cells.

Methods for effective delivery of antibodies that block CD47 in humans with cancer are of clinical interest, and are provided herein.

### SUMMARY

Disclosed herein is a method for treating a blood cancer in a subject, using a treatment comprising an anti-CD47 agent and an anti-CD20 agent (e.g., anti-CD20 antibody). In various embodiments, the subject is determined to be eligible to receive the treatment by verifying a presence of B-cells in the subject. Patients that are determined to be eligible to receive the treatment are likely to respond more favorably to the treatment than patients that are determined to be ineligible to receive the treatment. In various embodiments, the subject has a B-cell hematologic malignancy, e.g., a CD20+ cancer.

Disclosed herein is a method of treating a blood cancer in a subject comprising: (a) administering an anti-CD47 agent that inhibits binding between CD47 and SIRPα; and (b) administering an anti-CD20 antibody to the subject, wherein B-cells are determined or have been determined to be present in the subject prior to performing steps (a) and (b). Additionally disclosed herein is a method of treating a blood cancer in a subject comprising: determining or having determined that B-cells are present in the subject; and administering or having administered to the subject (i) an anti-CD47 agent that inhibits binding between CD47 and SIRPα and (ii) an anti-CD20 antibody. In various embodiments, the subject has a B-cell hematologic malignancy, e.g., a CD20+ cancer.

In various embodiments, the determination that B-cells are present in the subject comprises performing or having performed at least one assay selected from flow cytometry, B-cell resistance panel, ELISA, immunohistochemical microscopy, RNA profiling, RNA sequencing, RNA array-based detection, RT-PCR, Northern blot, immunoglobulin sequencing, Western blot, enzyme-linked immunospot, or immunofluorescent microscopy.

In various embodiments, the method further comprises prior to administering the anti-CD47 agent and the anti-CD20 antibody to the subject, determining that the subject is a candidate for treatment given the determination that B-cells are present in the subject. In various embodiments, the determination that B-cells are present in the subject comprises determining or having determined that the subject has CD19+ B-cells.

In various embodiments, determining or having determined that the subject has CD19+ B-cells comprises determining or having determined that the subject has above a threshold amount of CD19+ B-cells. In various embodiments, the threshold amount of CD19+ B-cells is a limit of detection for an assay used to determine the presence of the CD19+ B-cells. In various embodiments, the threshold amount of CD19+ B-cells is at least five percent of CD19+ B-cells out of a total population of lymphocytes. In various embodiments, the threshold amount of CD19+ B-cells is at least 1 CD19+B-cell per microliter. In various embodiments, the threshold amount of CD19+ B-cells is at least 40 CD19+ B-cells per microliter.

In various embodiments, the determination that B-cells are present in the subject comprises determining or having determined that the subject has CD20+ B-cells. In various embodiments, determining or having determined that the subject has CD20+ B-cells comprises determining or having determined that the subject has above a threshold amount of CD20+ B-cells. In various embodiments, the threshold amount of CD20+ B-cells is a limit of detection for an assay used to determine the presence of the CD20+ B-cells. In various embodiments, the threshold amount of CD20+ B-cells is at least five percent of CD20+B-cells out of a total population of lymphocytes. In various embodiments, the threshold amount of CD20+ B-cells is at least 1 CD20+B-cell per microliter. In various embodiments, the threshold amount of CD20+ B-cells is at least 40 CD20+ B-cells per microliter.

In various embodiments, the determination that B-cells are present in the subject comprises determining or having determined that the subject has both CD19+ B-cells and CD20+ B-cells. In various embodiments, determining or having determined that the subject has both CD19+ B-cells and CD20+ B-cells comprises determining or having determined that the subject has above a threshold amount of CD19+ B-cells and CD20+ B-cells. In various embodiments, the threshold amount of CD19+ B-cells is any one of a limit of detection for an assay used to determine the presence of the CD19+ B-cells, at least five percent of CD19+B-cells out of a total population of lymphocytes, at least 1 CD19+B-cell per microliter, or at least 40 CD19+ B-cells per microliter. In various embodiments, the threshold amount of CD20+ B-cells is any one of a limit of detection for an assay used to determine the presence of the CD20+ B-cells, at least five percent of CD20+ B-cells out of a total population of lymphocytes, at least 1 CD20+B-cell per microliter, or at least 40 CD20+ B-cells per microliter.

In various embodiments, the determination that B-cells are present in the subject comprises determining or having determined that the subject previously received an anti-CD20 therapy more than a threshold amount of time ago. In various embodiments, the threshold amount of time is at least 4 weeks. In various embodiments, the threshold amount of time is at least 5, 6, 7, 8, 9, 10, 11, 12, 13, 14, 15, 16, 17, 18, 19, 20, 21, 22, 23, 24, 25, 26, 27, or 28 weeks.

In various embodiments, the determination that B-cells are present in the subject comprises determining or having determined an absence of an anti-CD20 therapy in the subject. In various embodiments, determining or having determined an absence of the anti-CD20 therapy in the subject comprises determining or having determined that the subject has below a threshold concentration of the anti-CD20 therapy. In various embodiments, the threshold concentration of the anti-CD20 therapy is a limit of quantitation of a detection assay used to detect the presence of the anti-CD20 therapy. In various embodiments, the detection assay used to detect the presence of the anti-CD20 therapy is one of an immunoassay, ELIspot, fluorospot, flow cytometry based assay, Western blot, LC mass spectrometry, or surface plasmon resonance.

In various embodiments, the previously received anti-CD20 therapy comprises rituximab. In various embodiments, B-cells are determined or have been determined to be present in the subject using a sample obtained from the subject. In various embodiments, the sample obtained from the subject is a peripheral blood sample.

In various embodiments, the anti-CD47 agent comprises an isolated antibody that inhibits binding between CD47 and SIRPα. In various embodiments, the anti-CD47 agent comprises a SIRPα reagent, e.g., SIRPα-Fc fusion protein. In various embodiments, the SIRPα reagent comprises a portion of SIRPα that binds CD47. In various embodiments, the SIRPα reagent is a high affinity SIRPα reagent. In various embodiments, the anti-CD47 agent comprises an anti-CD47 antibody or an anti-SIRPα antibody. In various embodiments, the anti-CD47 agent comprises magrolimab (Hu5F9-G4). In various embodiments, the anti-CD47 agent comprises at least one of HuH9-G1, HuH9-G4, Hu3C2-G1, Hu3C2-G4, 9B11-G1, 9B11-G4, 7E11-G1, and 7E11-G4. In various embodiment, the anti-SIRPα agent is an anti-SIRPα antibody comprising at least one of FSI-189 (GS-0189), ES-004, BI765063, ADU1805, AL08, and CC-9525.

In various embodiments, the blood cancer is diffuse large B-cell lymphoma (DLBCL). In various embodiments, the subject has relapsed or refractory DLBCL. In various embodiments, the subject has previously been treated with at least two prior lines of therapy. In various embodiments, the blood cancer is follicular lymphoma (FL). In various embodiments, the blood cancer is one of non-Hodgkin's lymphoma, marginal zone lymphoma, mantle cell lymphoma, chronic lymphocytic leukemia/small lymphocytic leukemia, Waldenström's macroglobulinemia/lymphoplasmacytic lymphoma, primary mediastinal B-cell lymphoma, Burkitt's lymphoma, B-cell lymphoma unclassified, B-cell acute lymphoblastic leukemia, or post-transplant lymphoproliferative disease (PTLD).

In various embodiments, the anti-CD47 agent is administered at a dose of at least 10-30, 20-30, 10, 15, 20, 30, 46, 60 or 100 mg per kg of body weight. In various embodiments, the anti-CD47 agent is administered intravenously. In various embodiments, the anti-CD20 antibody is administered intravenously. In various embodiments, the method targets CD47 or SIRPα.

In various embodiments, the anti-CD47 agent that inhibits binding between CD47 and SIRPα is an anti-CD47 antibody, and wherein the anti-CD47 antibody is administered to the subject in a first cycle comprising a priming dose of at least 1 mg or in the range of 1 mg to 10 mg (e.g., 1 mg to 5 mg, e.g., 1 mg, 2 mg, 3 mg, 4 mg, 5 mg) of antibody per kg of body weight on day 1, and a weekly dose of at least 30 mg per kg of body weight beginning on day 8 for 4 weeks. In various embodiments, the anti-CD47 agent that inhibits binding between CD47 and SIRPα is further administered to the subject in a second cycle comprising a weekly dose of at least 30 mg per kg of body weight for 4 weeks. In various embodiments, the anti-CD47 agent that inhibits binding between CD47 and SIRPα is further administered to the subject in a third cycle comprising an every-other-week dose of at least 30 mg per kg of body weight. In various embodiments, the anti-CD47 agent that inhibits binding between CD47 and SIRPα is further administered to the subject in a subsequent cycle comprising an every-other-week dose of at least 30 mg per kg of body weight. In various embodiments, the subsequent cycle is repeated as one or more additional cycles without limit or until a clinical benefit is reduced or lost or no longer observed. In some embodiments, the anti-CD47 agent is administered intravenously. In various embodiments, the subject has a B-cell hematologic malignancy, e.g., a CD20+ cancer, e.g., an indolent or aggressive lymphoma, e.g., diffuse large B-cell lymphoma (DLBCL) (including relapsed or refractory), follicular lymphoma (FL) (including relapsed, refractory, or asymptomatic), non-Hodgkin's lymphoma (NHL) (including relapsed or refractory), marginal zone lymphoma (e.g., extranodal marginal-zone lymphoma), mantle cell lymphoma (MCL) (including relapsed or refractory), chronic lymphocytic leukemia (CLL)/small lymphocytic leukemia (including relapsed or refractory), Waldenstrom's macroglobulinemia/lymphoplasmacytic lymphoma, primary mediastinal B-cell lymphoma, Burkitt's lymphoma, double hit lymphoma (e.g., high grade B cell lymphoma with MYC and one or both of BCL2 or BCL6 rearrangement), myc-rearranged lymphoma, B-cell lymphoma unclassified, B-cell acute lymphoblastic leukemia (ALL) (e.g., Philadelphia chromosome-negative acute lymphoblastic leukemia), or post-transplant lymphoproliferative disease (PTLD). In some embodiments, the subject has diffuse large B-cell lymphoma (DLBCL), e.g., de novo or transformed DLBCL, or activated B cell (ABC), germinal center B cell (GCB), or non-germinal center B cell (non-GCB) DLBCL. In some embodiments, the subject has NHL, e.g., one or both of (i) low-grade or high risk NHL or (ii) follicular (e.g., bulky, non-bulky, or advanced follicular) or nonfollicular NHL. In some embodiments, the subject has a relapsed or refractory form of a B-cell hematologic malignancy. In various embodiments, the method targets CD47 or SIRPα.

In various embodiments, the first cycle further comprises a weekly dose of 375 mg per m² of body surface area of the anti-CD20 antibody. In various embodiments, the second cycle further comprises a monthly dose of 375 mg per m² of body surface area of the anti-CD20 antibody. In various embodiments, the third cycle further comprises a monthly dose of 375 mg per m² of body surface area of the anti-CD20 antibody. In various embodiments, the subsequent cycle further comprises an every-other-month dose of 375 mg per m² of body surface area of the anti-CD20 antibody. In various embodiments, the anti-CD20 antibody is administered to the subject at a dose of any one of 100, 125, 150, 175, 200, 225, 250, 275, 300, 325, 350, 375, 400, 425, 450, 475, or 500 mg/m². In various embodiments, the method targets CD47 or SIRPα.

In various embodiments, on days that the anti-CD47 agent and the anti-CD20 antibody are both administered to the subject, the anti-CD47 agent is administered to the subject prior to anti-CD20 antibody. In various embodiments, on days that the anti-CD47 agent and anti-CD20 antibody are both administered to the subject, the anti-CD20 antibody is administered to the subject prior to the anti-CD47 agent. In various embodiments, the method targets CD47 or SIRPα.

In various embodiments, the anti-CD47 agent that inhibits binding between CD47 and SIRPα is an anti-CD47 antibody, and wherein the anti-CD47 antibody is administered to the subject in a first cycle comprising a priming dose of at least 80 mg or in the range of 80 mg to 800 mg (e.g., 80 mg to 400 mg, e.g., 80 mg to 200 mg, e.g., 80 mg, 100 mg, 160 mg, 200 mg, 240 mg, 320 mg, 400 mg) on day 1, and a weekly dose of at least 2400 mg beginning on day 8 for 4 weeks. In various embodiments, the anti-CD47 agent that inhibits binding between CD47 and SIRPα is further administered to the subject in a second cycle comprising a weekly dose of at least 2400 mg for 4 weeks. In various embodiments, the anti-CD47 agent that inhibits binding between CD47 and SIRPα is further administered to the subject in a third cycle comprising an every-other-week dose of at least 2400 mg. In various embodiments, the anti-CD47 agent that inhibits binding between CD47 and SIRPα is further administered to the subject in a subsequent cycle comprising an every-other-week dose of at least 2400 mg. In various embodiments, the subsequent cycle is repeated as one or more additional cycles without limit or until a clinical benefit is reduced or lost or no longer observed. In some embodiments, the anti-CD47 agent is administered intravenously. In various embodiments, the subject has a B-cell hematologic malignancy, e.g., a CD20+ cancer, e.g., an indolent or aggressive lymphoma, e.g., diffuse large B-cell lymphoma (DLBCL) (including relapsed or refractory), follicular lymphoma (FL) (including relapsed, refractory, or asymptomatic), non-Hodgkin's lymphoma (NHL) (including relapsed or refractory, or asymptomatic), marginal zone lymphoma (e.g., extranodal marginal-zone lymphoma), mantle cell lymphoma (MCL) (including relapsed or refractory), chronic lymphocytic leukemia (CLL)/small lymphocytic leukemia including relapsed or refractory), Waldenstrom's macroglobulinemia/lymphoplasmacytic lymphoma, primary mediastinal B-cell lymphoma, Burkitt's lymphoma, double hit lymphoma (e.g., high grade B cell lymphoma with MYC and one or both of BCL2 or BCL6 rearrangement), myc-rearranged lymphoma, B-cell lymphoma unclassified, B-cell acute lymphoblastic leukemia (ALL) (e.g., Philadelphia chromosome-negative acute lymphoblastic leukemia), or post-transplant lymphoproliferative disease (PTLD). In some embodiments, the subject has diffuse large B-cell lymphoma (DLBCL), e.g., de novo or transformed DLBCL, or activated B cell (ABC), germinal center B cell (GCB), or non-germinal center B cell (non-GCB) DLBCL. In some embodiments, the subject has NHL, e.g., one or both of (i) low-grade or high risk NHL or (ii) follicular (e.g., bulky, non-bulky, or advanced follicular) or nonfollicular NHL. In some embodiments, the subject has a relapsed or refractory form of a B-cell hematologic malignancy. In various embodiments, the method targets CD47 or SIRPα.

In various embodiments, the first cycle further comprises a weekly dose of 375 mg per m² of body surface area of the anti-CD20 antibody. In various embodiments, the second cycle further comprises a monthly dose of 375 mg per m² of body surface area of the anti-CD20 antibody. In various embodiments, the third cycle further comprises a monthly dose of 375 mg per m² of body surface area of the anti-CD20 antibody. In various embodiments, the subsequent cycle further comprises an every-other-month dose of 375 mg per m² of body surface area of the anti-CD20 antibody. In various embodiments, the anti-CD20 antibody is administered to the subject at a dose of any one of 100, 125, 150, 175, 200, 225, 250, 275, 300, 325, 350, 375, 400, 425, 450, 475, or 500 mg/m². In various embodiments, the method targets CD47 or SIRPα.

In various embodiments, on days that the anti-CD47 agent and the anti-CD20 antibody are both administered to the subject, the anti-CD47 agent is administered to the subject prior to anti-CD20 antibody. In various embodiments, on days that the anti-CD47 agent and anti-CD20 antibody are both administered to the subject, the anti-CD20 antibody is administered to the subject prior to the anti-CD47 agent.

In various embodiments, the method further comprises administering chemotherapy to the subject. In various embodiments, the chemotherapy is gemcitabine, oxaliplatin, or a combination of gemcitabine and oxaliplatin (GEMOX).

In various embodiments, the anti-CD20 antibody comprises rituximab. In various embodiments, the anti-CD20 antibody comprises one, two, three, four, five, or six complementarity determining regions (CDRs) comprising the sequences of SEQ ID: 131-136. In various embodiments, the anti-CD20 antibody comprises a variable heavy chain sequence of SEQ ID NO: 137. In various embodiments, the anti-CD20 antibody comprises a variable light chain sequence of SEQ ID NO: 142. In various embodiments, the anti-CD20 antibody comprises an Fc region, the Fc region comprising a C_{H2} sequence of SEQ ID NO: 140 and a C_{H3} sequence of SEQ ID NO: 141. In various embodiments, the anti-CD20 antibody comprises a Fab or scFv, wherein the Fab or scFv comprises a variable heavy chain sequence of SEQ ID NO: 137 and a variable light chain sequence of SEQ ID NO: 142. In various embodiments, the anti-CD20 antibody comprises a Fab or scFv, wherein the Fab or scFv comprises the sequences of SEQ ID: 131-136.

Additionally disclosed herein is a method of treating a blood cancer in a subject, the method comprising: determining or having determined that B-cells are present in the subject, wherein the determination comprises determining or having determined that the subject has at least 5 percent of CD19+ B-cells out of a total amount of lymphocytes; administering magrolimab; and administering rituximab to the subject, wherein the subject is a human subject who has previously been treated with at least two prior lines of therapy, wherein the blood cancer, e.g., B-cell hematologic malignancy, e.g., CD20+ cancer, is relapsed or refractory DLBCL, wherein administering magrolimab comprises (1) administering a priming dose of magrolimab in the range of 1 mg to 10 mg (e.g., 1 mg to 5 mg, e.g., 1 mg, 2 mg, 3 mg, 4 mg, 5 mg) of antibody per kg of body weight on Day 1, (2) administering weekly doses of magrolimab at 30 mg per kg of body weight for 8 weeks, and (3) administering every-other-week doses of magrolimab at 30 mg per kg of body weight onwards, and wherein administering rituximab comprises (1) administering a weekly dose of rituximab at 375 mg per m² of body surface area for 4 weeks and subsequently (2) administering monthly rituximab at 375 mg per m² of body surface area. In various embodiments, the method targets CD47 or SIRPα.

Additionally disclosed herein is a method comprising: determining whether B-cells are present in a subject with a blood cancer based on whether the subject last received an anti-CD20 therapy more than a threshold amount of time ago, wherein the subject last receiving the anti-CD20 therapy more than the threshold amount of time ago indicates that the B-cells are present in the subject, wherein a presence of B-cells in the subject indicates that the subject is likely to respond to a therapy comprising 1) an anti-CD47 agent that inhibits binding between CD47 and SIRPα and 2) rituximab, wherein an absence of B-cells in the subject indicates that the subject is unlikely to respond to a therapy comprising 1) the anti-CD47 agent that inhibits binding between CD47 and SIRPα and 2) rituximab.

Additionally disclosed herein is a method comprising: obtaining a sample from a subject with a blood cancer; determining whether B-cells are present in the subject by performing an assay on the obtained sample from the subject, wherein a presence of B-cells in the subject indicates that the subject is likely to respond to a therapy comprising 1) an anti-CD47 agent that inhibits binding between CD47 and SIRPα and 2) rituximab, wherein an absence of B-cells in the subject indicates that the subject is unlikely to respond to a therapy comprising 1) the anti-CD47 agent that inhibits binding between CD47 and SIRPα and 2) rituximab. In various embodiments, the sample obtained from the subject is a peripheral blood sample.

Additionally disclosed herein is a method comprising: obtaining or having obtained a dataset comprising information indicative of presence of B-cells in a subject with a blood cancer, wherein the information indicative of presence of B-cells in the subject with the blood cancer comprises one of: a quantity of B-cells in the subject; a percentage of B-cells out of total lymphocytes in the subject; a number of days that the subject last received an anti-CD20 therapy; a presence or absence of anti-CD20 therapy in the subject; determining that B-cells are present in the subject with the blood cancer using the dataset; and administering a treatment to the subject with the blood cancer. In various embodiments, obtaining or having obtained the dataset comprises performing or having performed at least one assay selected from flow cytometry, B-cell resistance panel, ELISA, immunohistochemical microscopy, RNA profiling, RNA sequencing, RNA array-based detection, RT-PCR, Northern blot, immunoglobulin sequencing, Western blot, ELIspot, or immunofluorescent microscopy. In various embodiments, the information in the dataset comprises any one of a quantity of B-cells in a sample obtained from the subject or a percentage of B-cells in a sample obtained from the subject, and wherein determining that B-cells are present in the subject comprises comparing the information to a threshold amount of B-cells.

In various embodiments, the threshold amount of B-cells is at least five percent of B-cells out of a total population of lymphocytes. In various embodiments, the threshold amount of B-cells is at least a limit of detection for an assay used to determine the presence of the B-cells. In various embodiments, the threshold amount of B-cells is at least 1 B-cell per microliter. In various embodiments, the threshold amount of B-cells is at least at least 40 B-cells per microliter. In various embodiments, the B-cells are one of CD19+ B-cells or CD20+B-cells. In various embodiments, the B-cells are both CD19+ B-cells and CD20+ B-cells.

In various embodiments, the information in the dataset comprises an amount of time that the subject previously received an anti-CD20 therapy, and wherein determining that B-cells are present in the subject comprises determining whether the amount of time that the subject previously received an anti-CD20 therapy is above a threshold amount of time. In various embodiments, the threshold amount of time is at least 4 weeks. In various embodiments, the threshold amount of time is at least 5, 6, 7, 8, 9, 10, 11, 12, 13, 14, 15, 16, 17, 18, 19, 20, 21, 22, 23, 24, 25, 26, 27, or 28 weeks.

In various embodiments, the information in the dataset comprises a presence or absence of anti-CD20 therapy in the subject, and wherein determining that B-cells are present in the subject comprises determining that the anti-CD20 therapy is absent in the subject. In various embodiments, determining that the anti-CD20 therapy is absent in the subject comprises determining or having determined that the subject has below a threshold concentration of the anti-CD20 therapy. In various embodiments, the threshold concentration of the anti-CD20 therapy is a limit of quantitation of a detection assay used to detect the presence of the anti-CD20 therapy. In various embodiments, the detection assay used to detect the presence of the anti-CD20 therapy is one of an immunoassay, enzyme-linked immunospot, fluorospot, flow cytometry based assay, Western blot, LC mass spectrometry, or surface plasmon resonance. In various embodiments, the previously received anti-CD20 therapy comprises rituximab.

In various embodiments, the blood cancer is diffuse large B-cell lymphoma (DLBCL). In various embodiments, the blood cancer is relapsed or refractory DLBCL. In various embodiments, the subject has previously been treated with at least two prior lines of therapy. In various embodiments, the blood cancer, e.g., B-cell hematologic malignancy, e.g., a CD20+ cancer, is follicular lymphoma (FL). In various embodiments, the blood cancer is one of non-Hodgkin's lymphoma, marginal zone lymphoma, mantle cell lymphoma, chronic lymphocytic leukemia/small lymphocytic leukemia, Waldenstrom's macroglobulinemia/lymphoplasmacytic lymphoma, primary mediastinal B-cell lymphoma, Burkitt's lymphoma, B-cell lymphoma unclassified, B-cell acute lymphoblastic leukemia, or post-transplant lymphoproliferative disease (PTLD).

In various embodiments, administering the treatment comprises administering an anti-CD47 agent that inhibits binding between CD47 and SIRPα, and administering an anti-CD20 antibody to the subject. In various embodiments, the anti-CD47 agent comprises an isolated antibody that inhibits binding between CD47 and SIRPα. In various embodiments, the anti-CD47 agent comprises a SIRPα reagent. In various embodiments, the SIRPα reagent comprises a portion of SIRPα that binds CD47. In various embodiments, the SIRPα reagent is a high affinity SIRPα reagent. In various embodiments, the anti-CD47 agent comprises an isolated antibody that inhibits binding between CD47 and SIRPα. In various embodiments, the anti-CD47 agent comprises an anti-CD47 antibody or an anti-SIRPα antibody. In various embodiments, the anti-CD47 agent comprises magrolimab (Hu5F9-G4). In various embodiments, the anti-CD47 agent comprises at least one of Hu1H9-G1, Hu1H9-G4, Hu3C2-G1, Hu3C2-G4, 9B11-G, 9B11-G4, 7E11-G, and 7E11-G4. In various embodiment, the anti-SIRPα agent is an anti-SIRPα antibody comprising at least one of FSI-189 (GS-0189), ES-004, B1765063, ADU1805, and CC-9525.

In various embodiments, the subject is previously treated with an anti-CD20 therapy, and wherein the administration of the anti-CD47 agent that inhibits binding between CD47 and SIRPα and the administration of the anti-CD20 antibody to the subject each occurs no less than 28 days after the subject is previously treated with the anti-CD20 therapy. In various embodiments, the anti-CD47 agent is administered at a dose of at least 10-30, 20-30, 10, 15, 20, 30, 45, 60 or 100 mg per kg of body weight. In various embodiments, the anti-CD47 agent is administered intravenously. In various embodiments, the anti-CD20 antibody is administered intravenously. In various embodiments, the method targets CD47 or SIRPα.

In various embodiments, the anti-CD47 agent that inhibits binding between CD47 and SIRPα is an anti-CD47 antibody, and wherein the anti-CD47 antibody is administered to the subject in a first cycle comprising a priming dose of at least 1 mg or in the range of 1 mg to 10 mg (e.g., 1 mg to 5 mg, e.g., 1 mg, 2 mg, 3 mg, 4 mg, 5 mg) per kg of body weight on day 1, and a weekly dose of at least 30 mg per kg of body weight beginning on day 8 for 4 weeks. In various embodiments, the anti-CD47 agent that inhibits binding between CD47 and SIRPα is further administered to the subject in a second cycle comprising a weekly dose of at least 30 mg per kg of body weight for 4 weeks. In various embodiments, the anti-CD47 agent that inhibits binding between CD47 and SIRPα is further administered to the subject in a third cycle comprising an every-other-week dose of at least 30 mg per kg of body weight. In various embodiments, the anti-CD47 agent that inhibits binding between CD47 and SIRPα is further administered to the subject in a subsequent cycle comprising an every-other-week dose of at least 30 mg per kg of body weight. In various embodiments, the subsequent cycle is repeated as one or more additional cycles without limit or until a clinical benefit is reduced or lost or no longer observed. In some embodiments, the anti-CD47 agent is administered intravenously. In various embodiments, the subject has a B-cell hematologic malignancy, e.g., a CD20+ cancer, an indolent or aggressive lymphoma, e.g., e.g., diffuse large B-cell lymphoma (DLBCL) (including relapsed or refractory), follicular lymphoma (FL) (including relapsed, refractory, or asymptomatic), non-Hodgkin's lymphoma (NHL) (including relapsed or refractory), marginal zone lymphoma (e.g., extranodal marginal-zone lymphoma), mantle cell lymphoma (MCL) (including relapsed or refractory), chronic lymphocytic leukemia (CLL)/small lymphocytic leukemia (including relapsed or refractory), Waldenstrom's macroglobulinemia/lymphoplasmacytic lymphoma, primary mediastinal B-cell lymphoma, Burkitt's lymphoma, double hit lymphoma (e.g., high grade B cell lymphoma with MYC and one or both of BCL2 or BCL6 rearrangement), myc-rearranged lymphoma, B-cell lymphoma unclassified, B-cell acute lymphoblastic leukemia (ALL) (e.g., Philadelphia chromosome-negative acute lymphoblastic leukemia), or post-transplant lymphoproliferative disease (PTLD). In some embodiments, the subject has low Diffuse Large B-Cell Lymphoma (DLBCL), e.g., de novo or transformed DLBCL, or activated B cell (ABC), germinal center B cell (GCB), or non-germinal center B cell (non-GCB) DLBCL. In some embodiments, the subject has NHL, e.g., one or both of (i) low-grade or high risk NHL or (ii) follicular (e.g., bulky, non-bulky, or advanced follicular) or nonfollicular NHL. In some embodiments, the subject has a relapsed or refractory form of a B-cell hematologic malignancy. In various embodiments, the method targets CD47 or SIRPα.

In various embodiments, the first cycle further comprises a weekly dose of 375 mg per m² of body surface area of the anti-CD20 antibody. In various embodiments, the second cycle further comprises a monthly dose of 375 mg per m² of body surface area of the anti-CD20 antibody. In various embodiments, the third cycle further comprises a monthly dose of 375 mg per m² of body surface area of the anti-CD20 antibody. In various embodiments, the subsequent cycle further comprises an every-other-month dose of 375 mg per m² of body surface area of the anti-CD20 antibody. In various embodiments, the anti-CD20 antibody is administered to the subject at a dose of any one of 100, 125, 150, 175, 200, 225, 250, 275, 300, 325, 350, 375, 400, 425, 450, 475, or 500 mg/m². In various embodiments, on days that the anti-CD47 agent and anti-CD20 antibody are both administered to the subject, the anti-CD47 agent is administered to the subject prior to anti-CD20 antibody. In various embodiments, on days that the anti-CD47 agent and anti-CD20 antibody are both administered to the subject, the anti-CD20 antibody is administered to the subject prior to anti-CD47 agent. In various embodiments, the method further comprises administering chemotherapy to the subject. In various embodiments, the method targets CD47 or SIRPα.

In various embodiments, the anti-CD47 agent that inhibits binding between CD47 and SIRPα is an anti-SIRPα antibody. In various embodiments, the anti-SIRPα antibody is administered to the subject at a dose of any one of at least 10 mg, at least 30 mg, or at least 100 mg every two weeks for 9 months. In various embodiments, the anti-SIRPα antibody is administered to the subject at a dose of any one of at least 100 mg, at least 200 mg, at least 400 mg, or at least 800 mg every two weeks for 9 months. In various embodiments, the anti-SIRPα antibody is administered in combination with 375 mg per m² of body surface area of the anti-CD20 antibody. In some embodiments, the anti-SIRPα antibody is administered intravenously. In some embodiments, the anti-CD20 antibody is administered intravenously. In various embodiments, the subject has a B-cell hematologic malignancy, e.g., a CD20+ cancer, an indolent or aggressive lymphoma, e.g., e.g., diffuse large B-cell lymphoma (DLBCL) (including relapsed or refractory), follicular lymphoma (FL) (including relapsed, refractory, or asymptomatic), non-Hodgkin's lymphoma (NHL) (including relapsed or refractory), marginal zone lymphoma (e.g., extranodal marginal-zone lymphoma), mantle cell lymphoma (MCL) (including relapsed or refractory), chronic lymphocytic leukemia (CLL)/small lymphocytic leukemia (including relapsed or refractory), Waldenstrom's macroglobulinemia/lymphoplasmacytic lymphoma, primary mediastinal B-cell lymphoma, Burkitt's lymphoma, double hit lymphoma (e.g., high grade B cell lymphoma with MYC and one or both of BCL2 or BCL6 rearrangement), myc-rearranged lymphoma, B-cell lymphoma unclassified, B-cell acute lymphoblastic leukemia (ALL) (e.g., Philadelphia chromosome-negative acute lymphoblastic leukemia), or post-transplant lymphoproliferative disease (PTLD). In some embodiments, the subject has low Diffuse Large B-Cell Lymphoma (DLBCL), e.g., de novo or transformed DLBCL, or activated B cell (ABC), germinal center B cell (GCB), or non-germinal center B cell (non-GCB) DLBCL. In some embodiments, the subject has NHL, e.g., one or both of (i) low-grade or high risk NHL or (ii) follicular (e.g., bulky, non-bulky, or advanced follicular) or nonfollicular NHL. In some embodiments, the subject has a relapsed or refractory form of a B-cell hematologic malignancy. In various embodiments, the method targets CD47 or SIRPα.

In various embodiments, the anti-CD47 agent that inhibits binding between CD47 and SIRPα is an anti-SIRPα antibody. In various embodiments, the anti-SIRPα antibody is administered to the subject in a first cycle comprising a priming dose of at least 3 mg or at least 10 mg on day 1, and an every-other-week dose of at least 100 mg or at least 200 mg beginning on day 15 for 9 months. In various embodiments, the anti-SIRPα antibody is administered in combination with 375 mg per m² of body surface area of the anti-CD20 antibody beginning on day 15 for 9 months. In some embodiments, the anti-SIRPα antibody is administered intravenously. In some embodiments, the anti-CD20 antibody is administered intravenously. In various embodiments, the subject has a B-cell hematologic malignancy, e.g., a CD20+ cancer, an indolent or aggressive lymphoma, e.g., e.g., diffuse large B-cell lymphoma (DLBCL) (including relapsed or refractory), follicular lymphoma (FL) (including relapsed, refractory, or asymptomatic), non-Hodgkin's lymphoma (NHL) (including relapsed or refractory), marginal zone lymphoma (e.g., extranodal marginal-zone lymphoma), mantle cell lymphoma (MCL) (including relapsed or refractory), chronic lymphocytic leukemia (CLL)/small lymphocytic leukemia (including relapsed or refractory), Waldenstrom's macroglobulinemia/lymphoplasmacytic lymphoma, primary mediastinal B-cell lymphoma, Burkitt's lymphoma, double hit lymphoma (e.g., high grade B cell lymphoma with MYC and one or both of BCL2 or BCL6 rearrangement), myc-rearranged lymphoma, B-cell lymphoma unclassified, B-cell acute lymphoblastic leukemia (ALL) (e.g., Philadelphia chromosome-negative acute lymphoblastic leukemia), or post-transplant lymphoproliferative disease (PTLD). In some embodiments, the subject has low Diffuse Large B-Cell Lymphoma (DLBCL), e.g., de novo or transformed DLBCL, or activated B cell (ABC), germinal center B cell (GCB), or non-germinal center B cell (non-GCB) DLBCL. In some embodiments, the subject has NHL, e.g., one or both of (i) low-grade or high risk NHL or (ii) follicular (e.g., bulky, non-bulky, or advanced follicular) or nonfollicular NHL. In some embodiments, the subject has a relapsed or refractory form of a B-cell hematologic malignancy. In various embodiments, the method targets CD47 or SIRPα.

In various embodiments, the chemotherapy is gemcitabine, oxaliplatin, or a combination of gemcitabine and oxaliplatin (GEMOX).

In various embodiments, the anti-CD20 antibody comprises rituximab. In various embodiments, the anti-CD20 antibody comprises one, two, three, four, five, or six complementarity determining regions (CDRs) comprising the sequences of SEQ ID: 131-136. In various embodiments, the anti-CD20 antibody comprises a variable heavy chain sequence of SEQ ID NO: 137. In various embodiments, the anti-CD20 antibody comprises a variable light chain sequence of SEQ ID NO: 142. In various embodiments, the anti-CD20 antibody comprises an Fc region, the Fc region comprising a C_{H2} sequence of SEQ ID NO: 140 and a C_{H3} sequence of SEQ ID NO: 141. In various embodiments, the anti-CD20 antibody comprises a Fab or scFv, wherein the Fab or scFv comprises a variable heavy chain sequence of SEQ ID NO: 137 and a variable light chain sequence of SEQ ID NO: 142. In various embodiments, the anti-CD20 antibody comprises a Fab or scFv, wherein the Fab or scFv comprises the sequences of SEQ ID: 131-136.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 is an example flow process for determining eligibility of a blood cancer subject for receiving an anti-CD47 treatment, in accordance with an embodiment.

FIG. 2 shows a study design schema for: Phase 1b/2 Trial of Hu5F9-G4 in Combination with Rituximab in Patients with Relapsed/Refractory B-cell Non-Hodgkin's Lymphoma. A magrolimab priming dose (1 mg/kg) was utilized to mitigate on-target anemia, with dose escalation of the maintenance dose from 10 to 30 mg/kg in combination with rituximab in a standard 3+3 design.

FIG. 3 shows the use of percentage CD19+ B-cells and rituximab as a proxy for presence of CD20+ B-cells.

FIG. 4 shows identified variables that affect response rates among patients in the Phase 1b/2 trials.

FIG. 5 is a bar graph depicting the best overall response across patients that are negative for CD19 B-cells.

FIG. 6 is a plot depicting the best overall response of patients based on a percentage of CD19+ B-cells in the peripheral blood of the patients.

FIG. 7 is a plot depicting the best overall response of patients based on an absolute count of CD19+ B-cells in the peripheral blood of the patients.

FIG. 8 shows response rates of patients involved in the Phase 1b/2 trials before and after applying an eligibility criteria for presence of CD19+ B-cells.

FIG. 9 shows pie charts depicting the best overall response of patients with diffuse large B-cell lymphoma or follicular lymphoma based on a presence or absence of CD20+B-cells in the patients.

FIG. 10 shows response rates of a reduced set of patients involved in the Phase 1b/2 trials, where each of the patients in the reduced set is estimated to have a presence of CD20+B-cells.

FIGS. 11A and 11B depict results describing a CD20 H-score, which can be used as a direct measurement of the presence or absence of CD20+ B-cells.

FIGS. 12A and 12B depict the outcome of two patients with either CD20+CD19+ or CD20-CD19+ profiles confirmed using immunohistochemistry.

FIG. 13 shows the best overall response of patients based on a number of days that the patients last received an anti-CD20 treatment.

FIGS. 14A and 14B show the reduction of CD20 expression following treatment involving an anti-CD20 treatment e.g., rituximab.

FIGS. 15A and 15B show the change in CD20 expression in individual DLBCL patients at screening and post-treatment.

FIG. 16 shows a correlation between the time that a patient last received an anti-CD20 treatment and an absolute count of CD19 B-cells present in the patient.

FIG. 17 shows a correlation between the time that a patient last received an anti-CD20 treatment and a percentage of CD19 B-cells present in the patient.

FIG. 18 shows a correlation between a rituximab concentration in a patient (e.g., as a measure of rituximab pharmacokinetics) and a percentage of CD19 B-cells present in the patient.

FIG. 19 shows a correlation between a presence or absence of rituximab in a patient and a percentage of CD19 B-cells present in the patient.

FIG. 20 shows a correlation between a rituximab concentration in a patient and a presence or absence of CD19 B-cells present in the patient.

FIG. 21A shows CD47 receptor occupancy by Hu5F9-G4 in CD45+ peripheral blood cells over time after a transition from Hu5F9-G4 dosing (Q1W) to every other week Hu5F9-G4 dosing (Q2W).

FIG. 21B shows CD47 receptor occupancy by Hu5F9-G4 in CD45+ bone marrow cells over time after a transition from weekly Hu5F9-G4 dosing (Q1W) to every other week Hu5F9-G4 dosing (Q2W).

### DETAILED DESCRIPTION

Disclosed herein are methods of treating a subject with a blood cancer by determining that the subject is eligible for treatment based on a determination that B-cells are present in the subject, and further treating the subject with an anti-CD47 agent (e.g., magrolimab) alone, or in combination with one or more additional agents such as an anti-CD20 agent (e.g., rituximab).

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limit of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

The term “anti-CD47 agent” or “agent that provides for CD47 blockade” refers to any agent that reduces the binding of CD47 (e.g., on a target cell) to a CD47 ligand such as SIRPα (e.g., on a phagocytic cell). Non-limiting examples of suitable anti-CD47 reagents include SIRPα reagents, including without limitation high affinity SIRPα polypeptides, anti-SIRPα antibodies, soluble CD47 polypeptides, and anti-CD47 antibodies or antibody fragments. In some embodiments, a suitable anti-CD47 agent (e.g. an anti-CD47 antibody, a SIRPα reagent, etc.) specifically binds CD47 to reduce the binding of CD47 to SIRPα. In some embodiments, the subject anti-CD47 antibody specifically binds CD47 and reduces the interaction between CD47 on one cell (e.g., an infected cell) and SIRPα on another cell (e.g., a phagocytic cell). In some embodiments, a suitable anti-CD47 antibody does not activate CD47 upon binding. Some anti-CD47 antibodies do not reduce the binding of CD47 to SIRPα and such an antibody can be referred to as a “non-blocking anti-CD47 antibody.” A suitable anti-CD47 antibody that is an “anti-CD47 agent” can be referred to as a “CD47-blocking antibody”. Non-limiting examples of suitable antibodies include clones B6H12, 5F9, 8B6, and C3 (for example as described in International Patent Publication WO2011143624, published Jan. 19, 2012, herein specifically incorporated by reference). Suitable anti-CD47 antibodies include fully human, humanized or chimeric versions of such antibodies. Humanized antibodies (e.g., Hu5f9-G4) are especially useful for in vivo applications in humans due to their low antigenicity. Similarly caninized, felinized, etc. antibodies are especially useful for applications in dogs, cats, and other species respectively. Antibodies of interest include humanized antibodies, or caninized, felinized, equinized, bovinized, porcinized, etc., antibodies, and variants thereof.

In some embodiments, the anti-CD47 agent does not activate CD47 upon binding.

When CD47 is activated, a process akin to apoptosis (i.e., programmed cell death) may occur (Manna and Frazier, Cancer Research, 64, 1026-1036, Feb. 1 2004). Thus, in some embodiments, the anti-CD47 agent does not directly induce cell death of a CD47-expressing cell.

Some pathogens (e.g., pox viruses, Myxoma virus, Deerpox virus, swinepox virus, goatpox virus, sheeppox virus, etc.) express a CD47-analog (i.e., a CD47 mimic) (e.g., the M128L protein) that acts as a virulence factor to enable infection (Cameron et al., Virology. 2005 Jun. 20; 337(1):55-67), and some pathogens induce the expression of endogenous CD47 in the host cell. Cells infected with a pathogen that expresses a CD47-analog may therefore express the pathogen-provided CD47 analog either exclusively or in combination with endogenous CD47. This mechanism allows the pathogen to increase CD47 expression (via expression of the CD47 analog) in the infected cell with or without increasing the level of endogenous CD47. In some embodiments, an anti-CD47 agent (e.g., anti-CD47 antibody, a SIRPα reagent, a SIRPα antibody, a soluble CD47 polypeptide, etc.) can reduce the binding of a CD47 analog (i.e., a CD47 mimic) to SIRPα. In some cases, a suitable anti-CD47 agent (e.g., a SIRPα reagent, an anti-CD47 antibody, etc.) can bind a CD47 analog (i.e., a CD47 mimic) to reduce the binding of the CD47 analog to SIRPα. In some cases, a suitable anti-CD47 agent (e.g., an anti-SIRPα antibody, a soluble CD47 polypeptide, etc.) can bind to SIRPα. A suitable anti-CD47 agent that binds SIRPα does not activate SIRPα (e.g., in the SIRPα-expressing phagocytic cell). An anti-CD47 agent can be used in any of the methods provided herein when the pathogen is a pathogen that provides a CD47 analog. In other words the term “CD47,” as used herein, encompasses CD47 as well as CD47 analogs (i.e., CD47 mimics).

A SIRPα reagent comprises the portion of SIRPα that is sufficient to bind CD47 at a recognizable affinity, which normally lies between the signal sequence and the transmembrane domain, or a fragment thereof that retains the binding activity. A suitable SIRPα reagent reduces (e.g., blocks, prevents, etc.) the interaction between the native proteins SIRPα and CD47. The SIRPα reagent will usually comprise at least the dl domain of SIRPα. In some embodiments, a SIRPα reagent is a fusion protein, e.g., fused in frame with a second polypeptide. In some embodiments, the second polypeptide is capable of increasing the size of the fusion protein, e.g., so that the fusion protein will not be cleared from the circulation rapidly. In some embodiments, the second polypeptide is part or whole of an immunoglobulin Fc region. The Fc region aids in phagocytosis by providing an “eat me” signal, which enhances the block of the “don't eat me” signal provided by the high affinity SIRPα reagent. In other embodiments, the second polypeptide is any suitable polypeptide that is substantially similar to Fc, e.g., providing increased size, multimerization domains, and/or additional binding or interaction with Ig molecules.

In some embodiments, a subject anti-CD47 agent is a “high affinity SIRPα reagent”, which includes SIRPα-derived polypeptides and analogs thereof. High affinity SIRPα reagents are described in international application PCT/US13/21937 and WO2013109752A1, each of which is hereby specifically incorporated by reference. High affinity SIRPα reagents are variants of the native SIRPα protein. In some embodiments, a high affinity SIRPα reagent is soluble, where the polypeptide lacks the SIRPα transmembrane domain and comprises at least one amino acid change relative to the wild-type SIRPα sequence, and wherein the amino acid change increases the affinity of the SIRPα polypeptide binding to CD47, for example by decreasing the off-rate by at least 10-fold, at least 20-fold, at least 50-fold, at least 100-fold, at least 500-fold, or more.

A high affinity SIRPα reagent comprises the portion of SIRPα that is sufficient to bind CD47 at a recognizable affinity, e.g., high affinity, which normally lies between the signal sequence and the transmembrane domain, or a fragment thereof that retains the binding activity. The high affinity SIRPα reagent will usually comprise at least the dl domain of SIRPα with modified amino acid residues to increase affinity. In some embodiments, a SIRPα variant of the present invention is a fusion protein, e.g., fused in frame with a second polypeptide. In some embodiments, the second polypeptide is capable of increasing the size of the fusion protein, e.g., so that the fusion protein will not be cleared from the circulation rapidly. In some embodiments, the second polypeptide is part or whole of an immunoglobulin Fc region. The amino acid changes that provide for increased affinity are localized in the dl domain, and thus high affinity SIRPα reagents comprise a dl domain of human SIRPα, with at least one amino acid change relative to the wild-type sequence within the dl domain. Such a high affinity SIRPα reagent optionally comprises additional amino acid sequences, for example antibody Fc sequences; portions of the wild-type human SIRPα protein other than the dl domain, including without limitation residues 150 to 374 of the native protein or fragments thereof, usually fragments contiguous with the dl domain; and the like. High affinity SIRPα reagents may be monomeric or multimeric, i.e. dimer, trimer, tetramer, etc.

In some embodiments, a subject anti-CD47 agent is an antibody that specifically binds SIRPα (i.e., an anti-SIRPα antibody) and reduces the interaction between CD47 on one cell (e.g., an infected cell) and SIRPα on another cell (e.g., a phagocytic cell). Suitable anti-SIRPα antibodies can bind SIRPα without activating or stimulating signaling through SIRPα because activation of SIRPα would inhibit phagocytosis. Instead, suitable anti-SIRPα antibodies facilitate the preferential phagocytosis of inflicted cells over normal cells. Those cells that express higher levels of CD47 (e.g., infected cells) relative to other cells (non-infected cells) will be preferentially phagocytosed. Thus, a suitable anti-SIRPα antibody specifically binds SIRPα (without activating/stimulating enough of a signaling response to inhibit phagocytosis) and blocks an interaction between SIRPα and CD47. Suitable anti-SIRPα antibodies include fully human, humanized or chimeric versions of such antibodies. Humanized antibodies are especially useful for in vivo applications in humans due to their low antigenicity. Similarly caninized, felinized, etc. antibodies are especially useful for applications in dogs, cats, and other species respectively. Antibodies of interest include humanized antibodies, or caninized, felinized, equinized, bovinized, porcinized, etc., antibodies, and variants thereof.

As used herein, “antibody” includes reference to an immunoglobulin-based molecule immunologically reactive with a particular antigen (e.g., CD47), and includes both polyclonal and monoclonal antibodies. The term also includes genetically engineered forms such as chimeric antibodies (e.g., humanized murine antibodies) and heteroconjugate antibodies. The term “antibody” also includes antigen binding forms of antibodies, including fragments with antigen-binding capability (e.g., Fab′, F(ab′)₂, Fab, Fv and rIgG. The term also refers to recombinant single chain Fv fragments (scFv). The term antibody also includes bivalent or bispecific molecules, diabodies, triabodies, and tetrabodies. Additional description of the term antibody is found below.

As used herein, an “anti-CD47 antibody” refers to any antibody that reduces the binding of CD47 (e.g., on a target cell) to a CD47 ligand such as SIRPα (e.g., on a phagocytic cell). Non-limiting examples are described in more detail below and include but are not limited to Hu5F9-G4. In some embodiments, a subject anti-CD47 agent is an antibody that specifically binds CD47 (i.e., an anti-CD47 antibody) and reduces the interaction between CD47 on one cell (e.g., an infected cell) and SIRPα on another cell (e.g., a phagocytic cell). In some embodiments, a suitable anti-CD47 antibody does not activate CD47 upon binding. Non-limiting examples of suitable antibodies include clones B6H12, 5F9, 8B6, and C3 (for example as described in International Patent Publication WO 2011/143624, herein specifically incorporated by reference). Suitable anti-CD47 antibodies include fully human, humanized, or chimeric versions of antibodies. Humanized antibodies (e.g., hu5F9-G4) are especially useful for in vivo applications in humans due to their low antigenicity. Similarly caninized, felinized, etc. antibodies are especially useful for applications in dogs, cats, and other species respectively. Antibodies of interest include humanized antibodies, or caninized, felinized, equinized, bovinized, porcinized, etc., antibodies, and variants thereof.

As used herein, “Hu5F9-G4,” “5F9,” and “magrolimab” are used interchangeably and refer to an example of an anti-CD47 antibody that can be administered to a subject, individual, or patient, as described below, for treating a blood cancer.

A “patient” for the purposes of the present invention includes both humans and other animals, particularly mammals, including pet and laboratory animals, e.g. mice, rats, rabbits, etc. Thus the methods are applicable to both human therapy and veterinary applications. In one embodiment the patient is a mammal, preferably a primate. In other embodiments the patient is human.

The terms “subject,” “individual,” and “patient” are used interchangeably herein to refer to a mammal being assessed for treatment and/or being treated. In an embodiment, the mammal is a human. The terms “subject,” “individual,” and “patient” encompass, without limitation, individuals having cancer. Subjects may be human, but also include other mammals, particularly those mammals useful as laboratory models for human disease, e.g. mouse, rat, etc.

The term “sample” with respect to a patient encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents; washed; or enrichment for certain cell populations, such as cancer cells. The definition also includes sample that have been enriched for particular types of molecules, e.g., nucleic acids, polypeptides, etc. The term “biological sample” encompasses a clinical sample, and also includes tissue obtained by surgical resection, tissue obtained by biopsy, cells in culture, cell supernatants, cell lysates, tissue samples, organs, bone marrow, blood, plasma, serum, and the like. A “biological sample” includes a sample obtained from a patient's cancer cell, e.g., a sample comprising polynucleotides and/or polypeptides that is obtained from a patient's cancer cell (e.g., a cell lysate or other cell extract comprising polynucleotides and/or polypeptides); and a sample comprising cancer cells from a patient. A biological sample comprising a cancer cell from a patient can also include non-cancerous cells.

The term “diagnosis” is used herein to refer to the identification of a molecular or pathological state, disease or condition, such as the identification of a molecular subtype of breast cancer, prostate cancer, or other type of cancer.

The term “prognosis” is used herein to refer to the prediction of the likelihood of cancer-attributable death or progression, including recurrence, metastatic spread, and drug resistance, of a neoplastic disease, such as lymphoma. The term “prediction” is used herein to refer to the act of foretelling or estimating, based on observation, experience, or scientific reasoning. In one example, a physician may predict the likelihood that a patient will survive, following surgical removal of a primary tumor and/or chemotherapy for a certain period of time without cancer recurrence.

As used herein, the terms “treatment,” “treating,” and the like, refer to administering an agent, or carrying out a procedure, for the purposes of obtaining an effect. The effect may be therapeutic in terms of effecting a partial or complete cure for a disease and/or symptoms of the disease. “Treatment,” as used herein, may include treatment of a tumor in a mammal, particularly in a human, and includes, without limitation: inhibiting the disease, i.e., arresting its development; and relieving the disease, i.e., causing regression of the disease.

Treating may refer to any indicia of success in the treatment or amelioration of an cancer, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. The term “therapeutic effect” refers to the reduction, elimination, or prevention of the disease, symptoms of the disease, or side effects of the disease in the subject.

“In combination with”, “combination therapy” and “combination products” refer, in certain embodiments, to the concurrent administration to a patient of the agents described herein. When administered in combination, each component can be administered at the same time or sequentially in any order at different points in time. Thus, each component can be administered separately but sufficiently closely in time so as to provide the desired therapeutic effect.

“Concomitant administration” of active agents in the methods disclosed herein means administration with the reagents at such time that the agents will have a therapeutic effect at the same time. Such concomitant administration may involve concurrent (i.e. at the same time), prior, or subsequent administration of the agents.

As used herein, the term “correlates,” or “correlates with,” and like terms, refers to a statistical association between instances of two events, where events include numbers, data sets, and the like. For example, when the events involve numbers, a positive correlation (also referred to herein as a “direct correlation”) means that as one increases, the other increases as well. A negative correlation (also referred to herein as an “inverse correlation”) means that as one increases, the other decreases.

“Dosage unit” or “dose” refers to physically discrete units suited as unitary dosages for the particular individual to be treated. Each unit can contain a predetermined quantity of active compound(s) calculated to produce the desired therapeutic effect(s) in association with a pharmaceutical carrier. The specification for the dosage unit forms can be dictated by (a) the unique characteristics of the active compound(s) and the particular therapeutic effect(s) to be achieved, and (b) the limitations inherent in the art of compounding such active compound(s).

A “therapeutically effective amount” means the amount that, when administered to a subject for treating a disease, is sufficient to effect treatment for that disease.

Antibodies

The methods described herein include administration of an antibody or antibodies, i.e., administration of an anti CD47 antibody and, in some embodiments, administration of an additional antibody. Selection of antibodies may be based on a variety of criteria, including selectivity, affinity, cytotoxicity, etc. The phrase “specifically (or selectively) binds” to an antibody or “specifically (or selectively) immunoreactive with,” when referring to a protein or peptide, refers to a binding reaction that is determinative of the presence of the protein, in a heterogeneous population of proteins and other biologics. Thus, under designated immunoassay conditions, the specified antibodies bind to a particular protein sequences at least two times the background and more typically more than 10 to 100 times background. In general, antibodies of the present invention bind antigens on the surface of target cells in the presence of effector cells (such as natural killer cells or macrophages). Fc receptors on effector cells recognize bound antibodies.

An antibody immunologically reactive with a particular antigen can be generated by recombinant methods such as selection of libraries of recombinant antibodies in phage or similar vectors, or by immunizing an animal with the antigen or with DNA encoding the antigen. Methods of preparing polyclonal antibodies are known to the skilled artisan. The antibodies may, alternatively, be monoclonal antibodies. Monoclonal antibodies may be prepared using hybridoma methods. In a hybridoma method, an appropriate host animal is typically immunized with an immunizing agent to elicit lymphocytes that produce or are capable of producing antibodies that will specifically bind to the immunizing agent. Alternatively, the lymphocytes may be immunized in vitro. The lymphocytes are then fused with an immortalized cell line using a suitable fusing agent, such as polyethylene glycol, to form a hybridoma cell.

Human antibodies can be produced using various techniques known in the art, including phage display libraries. Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire.

Antibodies also exist as a number of well-characterized fragments produced by digestion with various peptidases. Thus pepsin digests an antibody below the disulfide linkages in the hinge region to produce F(ab)′₂, a dimer of Fab which itself is a light chain joined to V_{H}-C_{H1} by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region. While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries.

A “humanized antibody” is an immunoglobulin molecule which contains minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody optimally also will comprise at least a portion of an immunoglobulin constant region (Fc), typically that of a human immunoglobulin.

Antibodies of interest may be tested for their ability to induce ADCC (antibody-dependent cellular cytotoxicity), ADCP (antibody dependent cellular phagocytosis), or complement-dependent cytotoxicity (CDC). Antibody-associated ADCC activity can be monitored and quantified through detection of either the release of label or lactate dehydrogenase from the lysed cells, or detection of reduced target cell viability (e.g. Annexin assay). Assays for apoptosis may be performed by terminal deoxynucleotidyl transferase-mediated digoxigenin-11-dUTP nick end labeling (TUNEL) assay (Lazebnik et al., Nature: 371, 346 (1994). Cytotoxicity may also be detected directly by detection kits known in the art, such as Cytotoxicity Detection Kit from Roche Applied Science (Indianapolis, Ind.).

In some embodiments, the Fc region or Fc domain of the directed antibody comprise amino acid modifications that promote an increased serum half-life of the anti-binding molecule. Mutations that increase the half-life of an antibody have been described. In one embodiment, the Fc region or Fc domain of one or both of the CD3-targeting heavy chain and the HIV antigen-targeting heavy chain comprise a methionine to tyrosine substitution at position 252 (EU numbering), a serine to threonine substitution at position 254 (EU numbering), and a threonine to glutamic acid substitution at position 256 (EU numbering). See, e.g., U.S. Pat. No. 7,658,921. This type of mutant, designated as a “YTE mutant” exhibits a four-fold increased half-life relative to wild-type versions of the same antibody (Dall'Acqua, et al., J Biol Chem, 281: 23514-24 (2006); Robbie, et al., Antimicrob Agents Chemotherap., 57(12):6147-6153 (2013)). In certain embodiments, the Fc region or Fc domain of one or both of the CD3-targeting heavy chain and the HIV antigen-targeting heavy chain comprise an IgG constant domain comprising one, two, three or more amino acid substitutions of amino acid residues at positions 251-257, 285-290, 308-314, 385-389, and 428-436 (EU numbering). Alternatively, M428L and N434S (“LS”) substitutions can increase the pharmacokinetic half-life of the multi-specific antigen binding molecule. In other embodiments, the Fc region or Fc domain of one or both of the CD3-targeting heavy chain and the HIV antigen-targeting heavy chain comprise a M428L and N434S substitution (EU numbering). In other embodiments, the Fc region or Fc domain of one or both of the CD3-targeting heavy chain and the HIV antigen-targeting heavy chain comprise T250Q and M428L (EU numbering) mutations. In other embodiments, the Fc region or Fc domain of one or both of the CD3-targeting heavy chain and the HIV antigen-targeting heavy chain comprise H433K and N434F (EU numbering) mutations.

In some embodiments, the Fc region or Fc domain of the antibody comprise post-translational and/or amino acid modifications that increase effector activity, e.g., have improved FcγIIIa binding and increased antibody-dependent cellular cytotoxicity (ADCC). In some embodiments, the Fc region or Fc domain of the antibody comprises DE modifications (i.e., S239D and I332E by EU numbering) in the Fc region. In some embodiments, the Fc region or Fc domain of the antibody comprises DEL modifications (i.e., S239D, I332E and A330L by EU numbering) in the Fc region. In some embodiments, the Fc region or Fc domain of the antibody comprises DEA modifications (i.e., S239D, I332E and G236A by EU numbering) in the Fc region. In some embodiments, the Fc region or Fc domain of the antibody comprises DEAL modifications (i.e., S239D, I332E, G236A and A330L by EU numbering) in the Fc region. See, e.g., U.S. Pat. Nos. 7,317,091; 7,662,925; 8,039,592; 8,093,357; 8,093,359; 8,383,109; 8,388,955; 8,735,545; 8,858,937; 8,937,158; 9,040,041; 9,353,187; 10,184,000; and 10,584,176. Additional amino acid modifications that increase effector activity, e.g., have improved FcγIIIa binding and increased antibody-dependent cellular cytotoxicity (ADCC) include without limitation (EU numbering) F243L/R292P/Y300L/V305I/P396L; S298A/E333A/K334A; or L234Y/L235Q/G236W/S239M/H268D/D270E/S298A on a first Fc domain and D270E/K326D/A330M/K334E on a second Fc domain. Amino acid mutations that increase C1q binding and complement-dependent cytotoxicity (CDC) include without limitation (EU numbering) S267E/H268F/S324T or K326W/E333S. Fc region mutations that enhance effector activity are reviewed in, e.g., Wang, et al., Protein Cell (2018) 9(1): 63-73; and Saunders, Front Immunol. (2019) 10:1296.

In other embodiments, the antibody or antigen-binding fragment thereof has modified glycosylation, which, e.g., may be introduced post-translationally or through genetic engineering. In some embodiments, the antibody or antigen-binding fragment thereof is afucosylated, e.g., at a glycosylation site present in the antibody or antigen-binding fragment thereof. Most approved monoclonal antibodies are of the IgG1 isotype, where two N-linked biantennary complex-type oligosaccharides are bound to the Fc region. The Fc region exercises the effector function of ADCC through its interaction with leukocyte receptors of the FcγR family. Afucosylated monoclonal antibodies are monoclonal antibodies engineered so that the oligosaccharides in the Fc region of the antibody do not have any fucose sugar units.

Anti-CD47 Agents

The methods described herein include administration of therapeutic agent, such as an anti-CD47 agent. In some embodiments, the anti-CD47 agent is an anti-CD47 antibody.

CD47 is a broadly expressed transmembrane glycoprotein with a single Ig-like domain and five membrane spanning regions, which functions as a cellular ligand for SIRPα with binding mediated through the NH2-terminal V-like domain of SIRPα. SIRPα is expressed primarily on myeloid cells, including macrophages, granulocytes, myeloid dendritic cells (DCs), mast cells, and their precursors, including hematopoietic stem cells. Structural determinants on SIRPα that mediate CD47 binding are discussed by Lee et al. (2007) J. Immunol. 179:7741-7750; Hatherley et al. (2008) Mol Cell. 31(2):266-77; Hatherley et al. (2007) J.B.C. 282:14567-75; and the role of SIRPα cis dimerization in CD47 binding is discussed by Lee et al. (2010) J.B.C. 285:37953-63. In keeping with the role of CD47 to inhibit phagocytosis of normal cells, there is evidence that it is transiently upregulated on hematopoietic stem cells (HSCs) and progenitors just prior to and during their migratory phase, and that the level of CD47 on these cells determines the probability that they are engulfed in vivo.

In some embodiments an anti-CD47 antibody comprises a human IgG Fc region, e.g. an IgG1, IgG2a, IgG2b, IgG3, IgG4 constant region. In one embodiment the IgG Fc region is an IgG4 constant region. The IgG4 hinge may be stabilized by the amino acid substitution S241P (see Angal et al. (1993) Mol. Immunol. 30(1):105-108, herein specifically incorporated by reference).

In some embodiments, the anti-CD47 antibody competes for binding to CD47 with Hu5F9-G4. In some embodiments, the anti-CD47 binds to the same CD47 epitope as Hu5F9-G4.

In some embodiments, an antibody binds human CD47 with a KD of less than or equal to about 1, 1-6, 1-5, 1-4, 1-3, 2, 3, 4, 5, 6, 7, 8, 9, or 10×10\hat{ }-9 M, as measured by Biacore assay.

In some embodiments, an anti-CD47 antibody is administered at a dose of 10-30, 20-30, 10, 20, or 30 mg of antibody per kg of body weight.

In some embodiments, an anti-CD47 antibody results in greater than or equal to 90% receptor saturation, optionally 90-100, 90, 91, 92, 93, 94, 95, 96, 97, 98, 99, or 100% receptor saturation, optionally wherein receptor saturation is measured using flow cytometry or an equivalent assay.

An anti-CD47 antibody can be formulated in a pharmaceutical composition with a pharmaceutically acceptable excipient.

An anti-CD47 antibody can be administered intravenously.

An anti-CD47 agent can include a SIRPα agent that includes SIRPα or a portion thereof. For example, an anti-CD47 agent can include a SIRPα-based Fc fusion. See, e.g., Kipp Weiskopf, et al. Science 341, 88 (2013), herein incorporated by reference.

An anti-CD47 agent can include a SIRPα agent disclosed in WO2014094122, herein incorporated by reference, in its entirety, for all purposes. For example, a SIRPα agent can include the sequence of SEQ ID NO: 3, 25, or 26 as disclosed in WO2014094122; each of which is herein incorporated by reference.

An anti-CD47 agent can include a SIRPα agent disclosed in WO2017177333, herein incorporated by reference, in its entirety, for all purposes. For example, a SIRPα agent can include the sequence of SEQ ID NO: 3 or 8 as disclosed in WO2017177333; each of which is herein incorporated by reference.

An anti-CD47 agent can include a SIRPα agent disclosed in WO2016023040, herein incorporated by reference, in its entirety, for all purposes. For example, a SIRPα agent can include the sequence of SEQ ID NO: 78-85, 98-104, 107-113, 116-122, 135-137, or 152-159 as disclosed in WO2016023040; each of which is herein incorporated by reference.

An anti-CD47 agent can include a SIRPα agent disclosed in WO2017027422, herein incorporated by reference, in its entirety, for all purposes. For example, a SIRPα agent can include the sequence of SEQ ID NO: 3-34 as disclosed in WO2017027422; each of which is herein incorporated by reference.

Additional anti-CD47 agents include, without limitation, anti-CD47 mAbs (Vx-1004), anti-human CD47 mAbs (CNTO-7108), CC-90002, CC-90002-ST-001, humanized anti-CD47 antibody (Hu5F9-G4; magrolimab), NI-1701, NI-1801, RCT-1938, ALX-148, TTI-621, RRx-001, DSP-107, VT-1021, TTI-621, TTI-622, IMM-02, Lemzoparlimab, and SGN-CD47M.

In some embodiments, an anti-CD47 agent comprises a bispecific antibody. In some embodiments, an anti-CD47 agent comprises a bispecific anti-CD47 antibody. Examples of bi-specific antibodies targeting CD47 include, but are not limited to, IBI-322 (CD47/PD-L1), IMM-0306 (CD47/CD20), TJ-L1C4 (CD47/PD-L1), HX-009 (CD47/PD-1), PMC-122 (CD47/PD-L1), PT-217, (CD47/DLL3), IMM-26011 (CD47/FLT3), IMM-0207 (CD47/VEGF), IMM-2902 (CD47/HER2), BH29xx (CD47/PD-L1), IMM-0306 (CD47/CD20), IMM-2502 (CD47/PD-L1), HMBD-004B (CD47/BCMA), HMBD-004A (CD47/CD33). Additional monospecific and bispecific anti-CD47 antibodies include, but are not limited to, IBI-188, TJC-4, SHR-1603, HLX-24, LQ-001, IMC-002, ZL-1201, IMM-01, B6H12, GenSci-059, TAY-018, PT-240, 1F8-GMCSF, SY-102, and KD-015.

CD47 Antibodies

In some embodiments, the methods described herein include administration of the anti-CD47 antibody Hu5F9-G4. In some embodiments, the methods described herein include administration of an anti-CD47 antibody with sequences (light chain, heavy chain, variable light chain domain, variable heavy chain domain, and/or CDR) at least 97%, at least 98%, at least 99% or 100% identical to the sequences of Hu5f9-G4. Table 1 contains the sequence of the Hu5f9-G4 antibody heavy and light chains (SEQ ID NOs: 50 and 51, respectively), the VH and VL CDRs according to the Kabat CDR definition (SEQ ID NOs: 52-57 and 146), the VH and VL CDRs according to the IMGT CDR definition (SEQ ID NOs: 147-152), the VH and VL CDRs according to the Chothia CDR definition (SEQ ID NOs: 153-158), the VH and VL CDRs according to the Honegger CDR definition (SEQ ID NOs: 159-164), and the variable heavy and light chain sequences (SEQ ID NOs: 144 and 145). Further suitable anti CD-47 antibodies include clones B6H12, 5F9, 8B6, C3, and huC3 (for example as described in International Patent Publication WO2011143624, herein specifically incorporated by reference). The 5F9 variable heavy chain domain is provided as SEQ ID NO: 58, and the 5F9 variable light chain domain is provided as SEQ ID NO: 59. The HuB6H12 variable heavy chain domain is provided as SEQ ID NO: 60, and the HuB6H12 variable light chain domain is provided as SEQ ID NO: 61. The 8B6 variable heavy chain domain is provided as SEQ ID NO: 62, and the HuB6H12 variable light chain domain is provided as SEQ ID NO: 63. The C3 variable heavy chain domain is provided as SEQ ID NO: 64, and the C3 variable light chain domain is provided as SEQ ID NO: 65. HuC3 variable heavy chain domains are provided as SEQ ID NO: 66 and 67, and HuC3 variable light chain domains are provided as SEQ ID NO: 68 and 69. An anti-CD47 antibody can comprise: a heavy chain sequence of SEQ ID NO: 50 and a light chain of sequence of SEQ ID NO: 51. An anti-CD47 antibody can comprise: a VH sequence of SEQ ID NO: 58 and a VL sequence of SEQ ID NO: 59. An anti-CD47 antibody can comprise: a VH sequence of SEQ ID NO: 60 and a VL sequence of SEQ ID NO: 61. An anti-CD47 antibody can comprise: a VH sequence of SEQ ID NO: 62 and a VL sequence of SEQ ID NO: 63. An anti-CD47 antibody can comprise: a VH sequence of SEQ ID NO: 64 and a VL sequence of SEQ ID NO: 65. An anti-CD47 antibody can comprise: a VH sequence of SEQ ID NO: 66 or 67 and a VL sequence of SEQ ID NO: 68 or 69.

Anti-CD47 antibody heavy chain variable regions are disclosed as SEQ ID NOs: 5-30 and anti-CD47 antibody light chain variable regions are disclosed as SEQ ID NOs: 31-47 in U.S. Patent Publication US 20140140989, published May 22, 2014, and International Patent Publication WO2013119714, published Aug. 15, 2013, both of which are herein incorporated by reference in their entirety. Suitable anti-CD47 variable heavy chain domains are provided as SEQ ID NOs: 70-95 and anti-CD47 variable light chain domains are provided as SEQ ID NOs: 96-112. An anti-CD47 antibody can comprise a VH sequence of SEQ ID NO: 70-95. An anti-CD47 antibody can comprise a VL sequence of SEQ ID NO: 96-112. An anti-CD47 antibody can comprise a VH sequence of SEQ ID NO: 70-95 and a VL sequence of SEQ ID NO: 96-112.

An anti-CD47 antibody can comprise a VH sequence of SEQ ID NO: 113-115. An anti-CD47 antibody can comprise a VL sequence of SEQ ID NO: 116-118. An anti-CD47 antibody can comprise a VH sequence of SEQ ID NO: 113-115 and a VL sequence of SEQ ID NO: 116-118.

TABLE 1

| SEQ ID | | |
|---|---|---|
| NO | | Description and Sequence |
| 50 | Hu5f9-G4 | QVQLVQSGAEVKKPGASVKVSCKASGYTFTNYNMHWVRQAPGQRLEWMGTIYPGND |
| | Antibody | DTSYNQKFKDRVTITADTSASTAYMELSSLRSEDTAVYYCARGGYRAMDYWGQGTL |
| | Heavy | VTVSSASTKGPSVFPLAPCSRSTSESTAALGCLVKDYFPEPVTVSWNSGALTSGVH |
| | Chain | TFPAVLQSSGLYSLSSVVTVPSSSLGTKTYTCNVDHKPSNTKVDKRVESKYGPPCP |
| | | PCPAPEFLGGPSVFLFPPKPKDTLMISRTPEVTCVVVDVSQEDPEVQFNWYVDGVE |
| | | VHNAKTKPREEQFNSTYRVVSVLTVLHQDWLNGKEYKCKVSNKGLPSSIEKTISKA |
| | | KGQPREPQVYTLPPSQEEMTKNQVSLTCLVKGFYPSDIAVEWESNGQPENNYKTTP |
| | | PVLDSDGSFFLYSRLTVDKSRWQEGNVFSCSVMHEALHNHYTQKSISISLGK |
| 51 | Hu5f9-G4 | DIVMTQSPLSLPVTPGEPASISCRSSQSIVYSNGNTYLGWYLQKPGQSPQLLIYKV |
| | Antibody | SNRFSGVPDRFSGSGSGTDFTLKISRVEAEDVGVYYCFQGSHVPYTFGQGTKLEIK |
| | Light | RTVAAPSVFIFPPSDEQLKSGTASVVCLLNNFYPREAKVQWKVDNALQSGNSQESV |
| | chain | TEQDSKDSTYSLSSTLTLSKADYEKHKVYACEVTHQGLSSPVTKSFNRGEC |
| 52 | Hu5f9-G4 | NYNMH |
| | VH CDR1 | |
| 53 | Hu5f9-G4 | TIYPGNDDTSYNQKFKD |
| | VH CDR2 | |
| 54 | Hu5f9-G4 | GGYRAMDY |
| | VH CDR3 | |
| 55 | Hu5f9-G4 | RSSQSIVYSNGNTYL |
| | VL CDR1 | |
| 56 | Hu5f9-G4 | KVSNRFS |
| | VL CDR2 | |
| 57 | Hu5f9-G4 | FQGSHVPYT |
| | VL CDR3 | |
| 144 | Hu5f9-G4 | QVQLVQSGAEVKKPGASVKVSCKASGYTFTNYNMHWVRQAPGQRLEWMGTIYPGND |
| | VH | DTSYNQKFKDRVTITADTSASTAYMELSSLRSEDTAVYYCARGGYRAMDYWGQGTL |
| | | VTVSS |
| 145 | Hu5f9-G4 | DIVMTQSPLSLPVTPGEPASISCRSSQSIVYSNGNTYLGWYLQKPGQSPQLLIYKV |
| | VL | SNRFSGVPDRFSGSGSGTDFTLKISRVEAEDVGVYYCFQGSHVPYTFGQGTKLEIK |
| 146 | Hu5f9-G4 | RSSQSIVYSNGNTYLG |
| | VL CDR1 | |
| 147 | Hu5f9-G4 | GYTFTNYN |
| | VH CDR1 | |
| 148 | Hu5f9-G4 | IYPGNDDT |
| | VH CDR2 | |
| 149 | Hu5f9-G4 | ARGGYRAMDY |
| | VH CDR3 | |
| 150 | Hu5f9-G4 | QSIVYSNGNTY |
| | VL CDR1 | |
| 151 | Hu5f9-G4 | KVS |
| | VL CDR2 | |
| 152 | Hu5f9-G4 | FQGSHVPYT |
| | VL CDR3 | |
| 153 | Hu5f9-G4 | GYTFTNY |
| | VH CDR1 | |
| 154 | Hu5f9-G4 | PGND |
| | VH CDR2 | |
| 155 | Hu5f9-G4 | GYRAMD |
| | VH CDR3 | |
| 156 | Hu5f9-G4 | SQSIVYSNGNTY |
| | VL CDR1 | |
| 157 | Hu5f9-G4 | KVS |
| | VL CDR2 | |
| 158 | Hu5f9-G4 | GSHVPY |
| | VL CDR3 | |
| 159 | Hu5f9-G4 | ASGYTFTNYN |
| | VH CDR1 | |
| 160 | Hu5f9-G4 | IYPGNDDTSYNQKFKDR |
| | VH CDR2 | |
| 161 | Hu5f9-G4 | GGYRAMD |
| | VH CDR3 | |
| 162 | Hu5f9-G4 | SSQSIVYSNGNTY |
| | VL CDR1 | |
| 163 | Hu5f9-G4 | KVSNRFSGVPDR |
| | VL CDR2 | |
| 164 | Hu5f9-G4 | GSHVPY |
| | VL CDR3 | |

Additional CD47 antibodies are described in WO199727873, WO199940940, WO2002092784, WO2005044857, WO2009046541, WO2010070047, WO2011143624, WO2012170250, WO2013109752, WO2013119714, WO2014087248, WO2015191861, WO2016022971, WO2016023040, WO2016024021, WO2016081423, WO2016109415, WO2016141328, WO2016188449, WO2017027422, WO2017049251, WO2017053423, WO2017121771, WO2017194634, WO2017196793, WO2017215585, WO2018075857, WO2018075960, WO2018089508, WO2018095428, WO2018137705, WO2018233575, WO2019027903, WO2019034895, WO2019042119, WO2019042285, WO2019042470, WO2019086573, WO2019108733, WO2019138367, WO2019144895, WO2019157843, WO2019179366, WO2019184912, WO2019185717, WO2019201236, WO2019238012, WO2019241732, WO2020019135, WO2020036977, WO2020043188 and WO2020009725, each of which are herein incorporated by reference in their entirety.

Anti-SIRPα Agents

The methods described herein include administration of an anti-SIRPα agent.

In some embodiments, the anti-SIRPα agent is a SIRPα inhibitor. Such inhibitors include, but are not limited to, AL-008, RRx-001, and CTX-5861.

In some embodiments, the anti-SIRPα agent is an anti-SIRPα antibodies. Such antibodies include, but are not limited to, FSI-189, ES-004, B1765063, ADU1805, and CC-95251.

In some embodiments, the anti-SIRPα agent is an anti-SIRPα antibody that specifically binds to SIRPα. In some aspects, the SIRPα is human SIRPα.

In some embodiments, anti-SIRPα antibodies provided herein specifically bind to the extracellular domain of SIRPα. The SIRPα may be expressed on the surface of any suitable target cell. In some embodiments, the target cell is a professional antigen presenting cell. In some embodiments, the target cell is a macrophage. An antibody can be pan-specific for human SIRPα isotypes. An antibody can be specific for a human SIRPα isotype.

In certain embodiments an antibody is 1H9. In certain embodiments an antibody is 3C2.

In some embodiments, an antibody provided herein inhibits binding of SIRPα to one or more ligands of SIRPα.

In certain aspects, an antibody does not bind to SIRPγ. In certain aspects, an antibody does not substantially bind to SIRPγ.

In some embodiments, an antibody fragment provided herein competes for binding to SIRPα with 1H9 and/or 3C2. In some embodiments, a fragment of an antibody provided herein binds the same epitope of SIRPα as such antibody.

In some aspects, an antibody disclosed herein is pan-specific for human SIRPα isotypes. An antibody disclosed herein, such as 1H9, can bind to multiple human SIRPα isotypes including one or more of V1, V2, and V1N5. Exemplary V1 sequence shown in SEQ ID NO:48. Exemplary V2 sequence shown in SEQ ID NO:49. See also Polymorphism in Sirpa modulates engraftment of human hematopoietic stem cells. Nature Immunology, 8; 1313, 2007. An antibody disclosed herein can bind to each of human SIRPα isotypes V1 and V2. An antibody disclosed herein can bind to human SIRPα isotype V1, including homozygous. An antibody disclosed herein can bind to human SIRPα isotype V2, including homozygous. An antibody disclosed herein can bind to human SIRPα isotypes V1/V5 (heterozygous). An antibody disclosed herein, such as 1H9, can bind to multiple human SIRPα isotypes including each of V1, V2, and V1/V5. Such antibodies can include 1H9 and 3C2, including humanized and/or Fc engineered versions of such antibodies. 1H9 can bind to each of human SIRPα isotypes V1 and V2. 1H9 can bind to human SIRPα isotype V1, including homozygous. 1H9 can bind to human SIRPα isotype V2, including homozygous. 1H9 can bind to human SIRPα isotypes V1/V5 (heterozygous). 1H9 can bind to multiple human SIRPα isotypes including each of V1, V2, and V1/V5. Binding to the human SIRPα variants can be measured using assays known in the art including PCR and/or flow cytometry. For example, a given sample can be genotyped to determine SIRP status and binding to SIRP can be determined using flow cytometry.

In certain aspects, an antibody competes for binding to human SIRPα with an antibody selected from 1H9 and 3C2. In certain aspects, an antibody binds to the same human SIRPα epitope as bound by 1H9 or 3C2. In certain aspects, an antibody binds to an overlapping human SIRPα epitope as bound by 1H9 or 3C2. In certain aspects, an antibody binds to a distinct human SIRPα epitope as bound by 1H9 or 3C2.

In certain aspects, an antibody does not compete for binding to human SIRPα with KWar antibody.

In certain aspects, an antibody partially competes for binding to human SIRPα with KWar antibody.

In certain aspects, an antibody inhibits binding of human CD47 to human SIRPα.

In certain aspects, an antibody inhibits binding of human SP-A to human SIRPα.

In certain aspects, an antibody inhibits binding of human SP-D to human SIRPα.

In certain aspects, an antibody binds to rhesus monkey SIRPα.

In certain aspects, an antibody binds to cynomolgus SIRPα.

In some embodiments, a SIRPα antibody is an antibody that competes with an illustrative antibody provided herein, e.g., 1H9 and/or 3C2. In some aspects, the antibody that competes with the illustrative antibody provided herein binds the same epitope as an illustrative antibody provided herein.

In some embodiments, a subject anti-CD47 agent is a high affinity SIRPα reagent, which includes SIRPα-derived polypeptides and analogs thereof.

Additional anti-SIRPα agents, inhibitors, and antibodies are described in WO200140307, WO2002092784, WO2007133811, WO2009046541, WO2010083253, WO2011076781, WO2013056352, WO2015138600, WO2016179399, WO2016205042, WO2017178653, WO2018026600, WO2018057669, WO2018107058, WO2018190719, WO2018210793, WO2019023347, WO2019042470, WO2019175218, WO2019183266, WO2020013170 and WO02020068752, each of which are herein incorporated by reference in their entirety.

SIRPα Antibodies

In some embodiments, an antibody binds human SIRPα with a KD of less than or equal to about 1, 1-6, 1-5, 1-4, 1-3, 2, 3, 4, 5, 6, 7, 8, 9, or 10×10⁻⁹ M, as measured by Biacore assay.

An antibody can comprise: a CDR-H1 comprising the sequence set forth in SEQ ID NO:1; a CDR-H2 comprising the sequence set forth in SEQ ID NO:2; a CDR-H3 comprising the sequence set forth in SEQ ID NO:3; a CDR-L1 comprising the sequence set forth in SEQ ID NO:4; a CDR-L2 comprising the sequence set forth in SEQ ID NO:5; and a CDR-L3 comprising the sequence set forth in SEQ ID NO:6.

An antibody can comprise: a VH sequence of SEQ ID NO:7 and a VL sequence of SEQ ID NO:8.

An antibody can comprise: a heavy chain of SEQ ID NO:17 and a light chain of SEQ ID NO:18.

An antibody can comprise: a CDR-H1 comprising the sequence set forth in SEQ ID NO:9; a CDR-H2 comprising the sequence set forth in SEQ ID NO:10; a CDR-H3 comprising the sequence set forth in SEQ ID NO:11; a CDR-L1 comprising the sequence set forth in SEQ ID NO:12; a CDR-L2 comprising the sequence set forth in SEQ ID NO:13; and a CDR-L3 comprising the sequence set forth in SEQ ID NO:14.

An antibody can comprise: a VH sequence of SEQ ID NO:15 and a VL sequence of SEQ ID NO:16.

An antibody can comprise: a heavy chain of SEQ ID NO:19 and a light chain of SEQ ID NO:20.

An antibody can comprise: a CDR-H1 comprising the sequence set forth in SEQ ID NO:21; a CDR-H2 comprising the sequence set forth in SEQ ID NO:22; a CDR-H3 comprising the sequence set forth in SEQ ID NO:23; a CDR-L1 comprising the sequence set forth in SEQ ID NO:24; a CDR-L2 comprising the sequence set forth in SEQ ID NO:25; and a CDR-L3 comprising the sequence set forth in SEQ ID NO:26.

An antibody can comprise: a VH sequence of SEQ ID NO:27 and a VL sequence of SEQ ID NO:28.

An antibody can comprise: a CDR-H1 comprising the sequence set forth in SEQ ID NO:29; a CDR-H2 comprising the sequence set forth in SEQ ID NO:30; a CDR-H3 comprising the sequence set forth in SEQ ID NO:31; a CDR-L1 comprising the sequence set forth in SEQ ID NO:32; a CDR-L2 comprising the sequence set forth in SEQ ID NO:33; and a CDR-L3 comprising the sequence set forth in SEQ ID NO:34.

An antibody can comprise: a VH sequence of SEQ ID NO:35 and a VL sequence of SEQ ID NO:36.

In certain aspects, an antibody can comprise one or more CDRs of 1H9. In certain aspects, an antibody can comprise all CDRs of 1H9. In certain aspects, an antibody can comprise one or more variable sequences of 1H9. In certain aspects, an antibody can comprise each variable sequence of 1H9. In certain aspects, an antibody can comprise the heavy chain of 1H9. In certain aspects, an antibody can comprise the light chain of 1H9. In certain aspects, an antibody can comprise the heavy chain and the light chain of 1H9. In certain aspects, an antibody is 1H9.

In certain aspects, an antibody can comprise one or more CDRs of 3C2. In certain aspects, an antibody can comprise all CDRs of 3C2. In certain aspects, an antibody can comprise one or more variable sequences of 3C2. In certain aspects, an antibody can comprise each variable sequence of 3C2. In certain aspects, an antibody can comprise the heavy chain of 3C2. In certain aspects, an antibody can comprise the light chain of 3C2. In certain aspects, an antibody can comprise the heavy chain and the light chain of 3C2. In certain aspects, an antibody is 3C2.

In certain aspects, an antibody can comprise one or more CDRs of 9B11. In certain aspects, an antibody can comprise all CDRs of 9B11. In certain aspects, an antibody can comprise one or more variable sequences of 9B11. In certain aspects, an antibody can comprise each variable sequence of 9B11. In certain aspects, an antibody can comprise the heavy chain of 9B11. In certain aspects, an antibody can comprise the light chain of 9B11. In certain aspects, an antibody can comprise the heavy chain and the light chain of 9B11. In certain aspects, an antibody is 9B11.

In certain aspects, an antibody can comprise one or more CDRs of 7E11. In certain aspects, an antibody can comprise all CDRs of 7E11. In certain aspects, an antibody can comprise one or more variable sequences of 7E11. In certain aspects, an antibody can comprise each variable sequence of 7E11. In certain aspects, an antibody can comprise the heavy chain of 7E11. In certain aspects, an antibody can comprise the light chain of 7E11. In certain aspects, an antibody can comprise the heavy chain and the light chain of 7E11. In certain aspects, an antibody is 7E11.

Anti-SIRPα antibody heavy chain variable domains are also provided as SEQ ID NOs: 119-125. Anti-SIRPα antibody light chain variable domains are also provided as SEQ ID NOs: 126-128. Anti-SIRPα antibody heavy chain variable regions are disclosed as SEQ ID NOs: 24, 25, 26, 27, 28, 29, and 30 and anti-SIRP antibody light chain variable regions are disclosed as SEQ ID NOs: 31, 32 and 33 in U.S. Patent Publication US 20190127477, published May 5, 2019, herein incorporated by reference in its entirety.

Anti-SIRPα antibody heavy chain variable regions are disclosed as SEQ ID NOs: 7, 10, 14, 16, 18, 30, 75, 78, 80, 82, 84, 86, and 88 and anti-SIRP antibody light chain variable regions are disclosed as SEQ ID NOs: 8, 20, 22, 24, 26, 28, 32, 76, 90, 92, 94, 96, 98, 100, and 104 in U.S. Patent Publication US 20180312587, published Nov. 1, 2018, herein incorporated by reference in its entirety.

Anti-SIRPα antibody heavy chain variable regions are disclosed as SEQ ID NO: 26, 81, 83 and anti-SIRPα antibody light chain variable regions are disclosed as SEQ ID NOs: 25, 39-41 in International Patent Publication WO2019183266A1, published Sep. 26, 2019, herein incorporated by reference in its entirety.

In some embodiments, an antibody provided herein comprises a sequence having at least about 50%, 60%, 70%, 80%, 90%, 95%, or 99% identity to an illustrative sequence provided in SEQ ID NOs: 1-36. In some embodiments, an antibody provided herein comprises a sequence provided in SEQ ID NOs: 1-36, with up to 1, 2, 3, 4, 5, 6, 7, 8, 9, 10, 11, 12, 13, 14, 15, 16, 17, 18, 19, 20, 21, 22, 23, 24, or 25 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

Anti-CD20 Antibodies

The methods described herein include administration of an anti-CD20 antibody. In some embodiments, an anti-CD20 antibody is administered in concert with an anti-CD47 antibody or an anti-SIRPα agent as described herein. Examples of anti-CD20 agents or antibodies that can be co-administered include without limitation: IGN-002, PF-05280586; Rituximab (Rituxan/Biogen Idec), Ofatumumab (Arzerra/Genmab), Obinutuzumab (Gazyva/Roche Glycart Biotech), Alemtuzumab, Veltuzumab, IMMU-106 (Immunomedics), Ocrelizumab (Ocrevus/Biogen Idec; Genentech), Ocaratuzumab, LY2469298 (Applied Molecular Evolution) and Ublituximab, LFB-R603 (LFB Biotech.; rEVO Biologics), IGN-002, PF-05280586.

An anti-CD20 antibody can compete for binding to CD20 with rituximab.

An anti-CD20 antibody can binds to the same CD20 epitope as rituximab.

Rituximab binds amino acids 170-173 and 182-185 on CD20. See Binder et al., The epitope recognized by rituximab. Blood (2006) 108 (6): 1975-1978.

An anti-CD20 antibody can comprise or consist of rituximab.

An anti-CD20 antibody can compete for binding to CD20 with obinutuzumab, ofatumumab, ocrelizumab, veltuzumab, ocaratuzumab, ibritumomab tiuxetan, tositumomab, iodine 131 tositumumab, a rituximab biosimilar (blitzima, ritemvia, tuxella), or ublituximab.

An anti-CD20 antibody can bind to the same CD20 epitope as obinutuzumab, ofatumumab, ocrelizumab, veltuzumab, ocaratuzumab, ibritumomab tiuxetan, tositumomab, iodine 131 tositumumab, a rituximab biosimilar (blitzima, ritemvia, tuxella), or ublituximab.

An anti-CD2 antibody can comprise or consist of obinutuzumab, ofatumumab, ocrelizumab, veltuzumab, ocaratuzumab, ibritumomab tiuxetan, tositumomab, iodine 131 tositumumab, a rituximab biosimilar (blitzima, ritemvia, tuxella), or ublituximab.

An anti-CD20 antibody can comprise an Fc such as an active Fc or wild-type Fc. An anti-CD20 antibody can comprise an Fc capable of at least one of ADCC, ADCP, and CDC. An anti-CD20 antibody comprise an Fc comprising one or more modifications that results in increased ADCC, ADCP, and/or CDC activity relative to wild-type Fc. Exemplary Fc mutations are shown in the table 2 below.

TABLE 2

| Engineering and | | |
|---|---|---|
| intended function | Mutation(s) | Reference |
| Enhance ADCC | | |
| Increased FcγRIIIa | F243L/R292P/Y300L/ | (Stavenhagen |
| binding | V305I/P396L | et al., 2007) |
| Increased FcγRIIIa | S239D/I332E | (Lazar et |
| binding | | al., 2006) |
| Increased FcγRIIIa | S239D/I332E/A330L | (Lazar et |
| binding, Decreased | | al., 2006) |
| FcγRIIb binding | | |
| Increased FcγRIIIa | S298A/E333A/K334A | (Shields et |
| binding | In one heavy chain: | al., 2001) |
| | L234Y/L235Q/G236W/ | (Mimoto et |
| | S239M/H268D/ | al., 2013a) |
| | D270E/S298A | |
| | In the opposing | |
| | heavy chain: | |
| | D270E/K326D/ | |
| | A330M/K334E | |
| Enhance ADCP | | |
| Increased FcγRIIa | G236A/S239D/I332E | (Richards et |
| binding, Increased | | al., 2008) |
| FcγRIIIa binding | | |
| Enhance CDC | | |
| Increased C1q binding | K326W/E333S | (Idusogie et |
| | | al., 2001) |
| Increased C1q binding | S267E/H268F/S324T | (Moore et |
| | | al., 2010) |
| Increased C1q binding | IgG1/IgG3 cross subclass | (Natsume et |
| | | al., 2008) |
| Hexamerization | E345R/E430G/S440Y | (Diebolder et |
| | | al., 2014) |

An anti-CD20 antibody can have a higher binding affinity for CD20 relative to rituximab, obinutuzumab, ofatumumab, ocrelizumab, ibritumomab tiuxetan, tositumomab, iodine 131 tositumumab, a rituximab biosimilar (blitzima, ritemvia, tuxella), or ublituximab.

An anti-CD20 antibody can be administered to a subject at a dose of 375 mg/m² of antibody. An anti-CD20 antibody can be administered once per week, once every two weeks, once per month, once every four weeks, once every eight weeks, or once every two months, optionally at a dose of 375 mg/m² of antibody at each relevant time point.

An anti-CD47 antibody and an anti-CD20 antibody can be administered concurrently or sequentially, optionally wherein the anti-CD20 antibody is administered prior to the anti-CD47 antibody. In some embodiments, the anti-CD20 antibody is administered after administration of the anti-CD47 antibody.

An anti-CD20 antibody can be formulated in a pharmaceutical composition with a pharmaceutically acceptable excipient. An anti-CD20 antibody and an anti-CD47 antibody can be formulated together.

An anti-CD20 antibody can be administered intravenously.

In some embodiments, the anti-CD20 antibody has sequences (light chain, heavy chain, variable light chain domain, variable heavy chain domain, and/or CDR) at least 97%, at least 98%, at least 99% or 100% identical to the sequences of rituximab, shown below in Table 3. Table 3 contains the sequence of the rituximab antibody heavy and light chains (SEQ ID NOs: 129 and 130, respectively) and the VH and VL CDRs (SEQ ID NOs: 131-136). Table 3 further shows the rituximab variable heavy chain, constant heavy chain (e.g., C_{H1}, C_{H2}, C_{H3}), hinge, variable light chain, and constant light chain. An anti-CD20 antibody can comprise: a heavy chain sequence of SEQ ID NO: 129 and a light chain sequence of SEQ ID NO: 130. An anti-CD20 antibody can comprise: a VH sequence of SEQ ID NO: 137 and a VL sequence of SEQ ID NO: 142. An anti-CD20 antibody can comprise one or more of: a C_{H1} sequence of SEQ ID NO: 138, a hinge sequence of SEQ ID NO: 139 (where the hinge sequence connects the C_{H1} and C_{H2} sequences), a C_{H2} sequence of SEQ ID NO: 140, and a C_{H3} sequence of SEQ ID NO: 141. An anti-CD20 antibody can comprise a constant light chain of SEQ ID NO: 143. An anti-CD20 antibody can comprise one or more of the CDRs of the sequences set forth in SEQ ID NOs: 131-136. An anti-CD20 antibody can comprise the CDRs of the sequences set forth in SEQ ID NOs: 131-136. An anti-CD20 antibody can comprise one or more of the CDRs of the sequence set forth in SEQ ID NO:137. An anti-CD20 antibody can comprise one or more of the CDRs of the sequence set forth in SEQ ID NO:142. An anti-CD20 antibody can comprise the CDRs of the V region sequence set forth in SEQ ID NO:137. An anti-CD20 antibody can comprise the CDRs of the V region sequence set forth in SEQ ID NO:142.

In various embodiments, an anti-CD20 antibody can comprise an Fc region, which comprises a C_{H2} sequence of SEQ ID NO: 140 and a C_{H3} sequence of SEQ ID NO: 141. In various embodiments, an anti-CD20 antibody can comprise an antigen-binding fragment (Fab). In various embodiments, an anti-CD20 Fab can comprise a variable heavy chain sequence of SEQ ID NO: 137, a Cm sequence of SEQ ID NO: 138, a variable light chain sequence of SEQ ID NO: 142, and a constant light chain sequence of SEQ ID NO: 143. In various embodiments, an anti-CD20 antibody can comprise a single-chain variable fragment (scFv). In various embodiments, the scFv can comprise a variable heavy chain sequence of SEQ ID NO: 137 and a variable light chain sequence of SEQ ID NO: 142. In various embodiments, an anti-CD20 antibody can comprise a F(ab)′₂ fragment. In various embodiments, an anti-CD20 F(ab)′₂ fragment can comprise a variable heavy chain sequence of SEQ ID NO: 137, a Cm sequence of SEQ ID NO: 138, a variable light chain sequence of SEQ ID NO: 142, a constant light chain sequence of SEQ ID NO: 143, and a hinge sequence of SEQ ID NO: 139.

Table 3 contains the sequences of rituximab antibody heavy and light chains.

TABLE 3

| SEQ ID | | |
|---|---|---|
| NO | ID | Sequence |
| 129 | Rituximab heavy | QVQLQQPGAELVKPGASVKMSCKASGYTFTSYNMHWVKQTPGRGLEWIG |
| | chain chimeric | AIYPGNGDTSYNQKFKGKATLTADKSSSTAYMQLSSLTSEDSAVYYCAR |
| | | STYYGGDWYFNVWGAGTTVTVSAASTKGPSVFPLAPSSKSTSGGTAALG |
| | | CLVKDYFPEPVTVSWNSGALTSGVHTFPAVLQSSGLYSLSSVVTVPSSS |
| | | LGTQTYICNVNHKPSNTKVDKKAEPKSCDKTHTCPPCPAPELLGGPSVF |
| | | LFPPKPKDTLMISRTPEVTCVVVDVSHEDPEVKFNWYVDGVEVHNAKTK |
| | | PREEQYNSTYRVVSVLTVLHQDWLNGKEYKCKVSNKALPAPIEKTISKA |
| | | KGQPREPQVYTLPPSRDELTKNQVSLTCLVKGFYPSDIAVEWESNGQPE |
| | | NNYKTTPPVLDSDGSFFLYSKLTVDKSRWQQGNVFSCSVMHEALHNHYT |
| | | QKSLSLSPGK |
| 130 | Rituximab light | QIVLSQSPAILSASPGEKVINICRASSSVSYIHWFQQKPGSSPKPWIYA |
| | chain chimeric | TSNLASGVPVRFSGSGSGTSYSLTISRVEAEDAATYYCQQWTSNPPTFG |
| | | GGTKLEIKRTVAAPSVFIFPPSDEQLKSGTASVVCLLNNFYPREAKVQW |
| | | KVDNALQSGNSQESVTEQDSKDSTYSLSSILTLSKADYEKHKVYACEVT |
| | | HQGLSSPVTKSFNRGEC |
| 131 | Rituximab VH | KASGYTFTSYNMH |
| | CDR1 | |
| 132 | Rituximab VH | AIYPGNGDTS |
| | CDR2 | |
| 133 | Rituximab VH | ARSTYYGGDWYFNV |
| | CDR3 | |
| 134 | Rituximab VL | RASSSVSYIH |
| | CDR1 | |
| 135 | Rituximab VL | YATSNLAS |
| | CDR2 | |
| 136 | Rituximab VL | QQWTSNPPT |
| | CDR3 | |
| 137 | Rituximab | QVQLQQPGAELVKPGASVKMSCKASGYTFTSYNMHWVKQTPGRGLEWIG |
| | Variable Heavy | AIYPGNGDTSYNQKFKGKATLTADKSSSTAYMQLSSLTSEDSAVYYCAR |
| | Chain (VH) | STYYGGDWYFNVWGAGTTVTVSAASTK |
| 138 | Rituximab | GPSVFPLAPSSKSTSGGTAALGCLVKDYFPEPVTVSWNSGALTSGVHTF |
| | Constant Heavy | PAVLQSSGLYSLSSVVTVPSSSLGTQTYICNVNHKPSNTKVDKKA |
| | Chain 1 (C_{H}1) | |
| 139 | Rituximab Heavy | EPKSCDKTHTCPPCP |
| | Chain Hinge | |
| 140 | Rituximab | APELLGGPSVFLFPPKPKDTLMISRTPEVTCVVVDVSHEDPEVKFNWYV |
| | Constant Heavy | DGVEVHNAKTKPREEQYNSTYRVVSVLTVLHQDWLNGKEYKCKVSNKAL |
| | Chain 2 (C_{H}2) | PAPIEKTISKAK |
| 141 | Rituximab | GQPREPQVYTLPPSRDELTKNQVSLTCLVKGFYPSDIAVEWESNGQPEN |
| | Constant Heavy | NYKTTPPVLDSDGSFFLYSKLTVDKSRWQQGNVFSCSVMHEALHNHYTQ |
| | Chain 3 (C_{H}3) | KSLSLSPGK |
| 142 | Rituximab | QIVLSQSPAILSASPGEKVTMTCRASSSVSYIHWFQQKPGSSPKPWIYA |
| | Variable Light | TSNLASGVPVRFSGSGSGTSYSLTISRVEAEDAATYYCQQWTSNPPTFG |
| | Chain | GGTKLEIK |
| 143 | Rituximab | RTVAAPSVFIFPPSDEQLKSGTASVVCLLNNFYPREAKVQWKVDNALQS |
| | Constant Light | GNSQESVTEQDSKDSTYSLSSTLTLSKADYEKHKVYACEVTHQGLSSPV |
| | Chain | TKSFNRGEC |

Additional Agents for Combination Therapies

In various embodiments, additional agents, such as small molecules, antibodies, adoptive cellular therapies and chimeric antigen receptor T cells (CAR-T), checkpoint inhibitors, and vaccines, that are appropriate for treating hematological malignancies can be administered in combination with the anti-CD47 agents as described herein. Additional immunotherapeutic agents for hematological malignancies are described in Dong S et al, J Life Sci (Westlake Village). 2019 June; 1(1): 46-52; and Cuesta-Mateos C Et al, Front. Immunol. 8:1936. doi: 10.3389/fimmu.2017.01936, each of which are hereby incorporated by reference in their entirety.

In various embodiments, an anti-CD47 agent or an anti-SIRPα agent as described herein, is combined with one or more additional therapeutic agents, e.g., an inhibitory immune checkpoint blocker or inhibitor, a stimulatory immune checkpoint stimulator, agonist or activator, a chemotherapeutic agent, an anti-cancer agent, a radiotherapeutic agent, an anti-neoplastic agent, an anti-proliferation agent, an anti-angiogenic agent, an anti-inflammatory agent, an immunotherapeutic agent, a therapeutic antigen-binding molecule (mono- and multi-specific antibodies and fragments thereof in any format (e.g., including without limitation DARTs®, Duobodies®, BiTEs®, BiKEs, TriKEs, XmAbs®, TandAbs®, scFvs, Fabs, Fab derivatives), bi-specific antibodies, non-immunoglobulin antibody mimetics (e.g., including without limitation adnectins, affibody molecules, affilins, affimers, affitins, alphabodies, anticalins, peptide aptamers, armadillo repeat proteins (ARMs), atrimers, avimers, designed ankyrin repeat proteins (DARPins©), fynomers, knottins, Kunitz domain peptides, monobodies, and nanoCLAMPs), antibody-drug conjugates (ADC), antibody-peptide conjugate), an oncolytic virus, a gene modifier or editor, a cell comprising a chimeric antigen receptor (CAR), e.g., including a T-cell immunotherapeutic agent, an NK-cell immunotherapeutic agent, or a macrophage immunotherapeutic agent, a cell comprising an engineered T-cell receptor (TCR-T), or any combination thereof.

In various embodiments, an anti-CD47 agent or an anti-SIRPα agent as described herein, is combined with one or more additional therapeutic agents including, without limitation, an inhibitor, agonist, antagonist, ligand, modulator, stimulator, blocker, activator or suppressor of a target (e.g., polypeptide or polynucleotide) including without limitation: Abelson murine leukemia viral oncogene homolog 1 gene (ABL, such as ABL1), Acetyl-CoA carboxylase (such as ACC1/2), activated CDC kinase (ACK, such as ACK1), Adenosine deaminase, adenosine receptor (such as A2BR, A2aR, A3aR), Adenylate cyclase, ADP ribosyl cyclase-1, adrenocorticotropic hormone receptor (ACTH), Aerolysin, AKT1 gene, Alk-5 protein kinase, Alkaline phosphatase, Alpha 1 adrenoceptor, Alpha 2 adrenoceptor, Alpha-ketoglutarate dehydrogenase (KGDH), Aminopeptidase N, AMP activated protein kinase, anaplastic lymphoma kinase (ALK, such as ALK1), Androgen receptor, Angiopoietin (such as ligand-1, ligand-2), Angiotensinogen (AGT) gene, murine thymoma viral oncogene homolog 1 (AKT) protein kinase (such as AKT1, AKT2, AKT3), apolipoprotein A-I (APOA1) gene, Apoptosis inducing factor, apoptosis protein (such as 1, 2), apoptosis signal-regulating kinase (ASK, such as ASK1), Arginase (I), Arginine deiminase, Aromatase, Asteroid homolog 1 (ASTE1) gene, ataxia telangiectasia and Rad 3 related (ATR) serine/threonine protein kinase, Aurora protein kinase (such as 1, 2), Axl tyrosine kinase receptor, 4-1BB ligand (CD137L), Baculoviral IAP repeat containing 5 (BIRC5) gene, Basigin, B-cell lymphoma 2 (BCL2) gene, Bcl2 binding component 3, Bcl2 protein, BCL2L11 gene, BCR (breakpoint cluster region) protein and gene, Beta adrenoceptor, Beta-catenin, B-lymphocyte antigen CD19, B-lymphocyte antigen CD20, B-lymphocyte cell adhesion molecule, B-lymphocyte stimulator ligand, Bone morphogenetic protein-10 ligand, Bone morphogenetic protein-9 ligand modulator, Brachyury protein, Bradykinin receptor, B-Raf proto-oncogene (BRAF), Brc-Abl tyrosine kinase, Bromodomain and external domain (BET) bromodomain containing protein (such as BRD2, BRD3, BRD4), Bruton's tyrosine kinase (BTK), Calmodulin, calmodulin-dependent protein kinase (CaMK, such as CAMKII), Cancer testis antigen 2, Cancer testis antigen NY-ESO-1, cancer/testis antigen 1B (CTAG1) gene, Cannabinoid receptor (such as CB1, CB2), Carbonic anhydrase, casein kinase (CK, such as CKI, CKII), Caspase (such as caspase-3, caspase-7, Caspase-9), caspase 8 apoptosis-related cysteine peptidase CASP8-FADD-like regulator, Caspase recruitment domain protein-15, Cathepsin G, CCR5 gene, CDK-activating kinase (CAK), Checkpoint kinase (such as CHK1, CHK2), chemokine (C-C motif) receptor (such as CCR2, CCR4, CCR5, CCR8), chemokine (C—X-C motif) receptor (such as CXCR1, CXCR2, CXCR3 and CXCR4), Chemokine CC21 ligand, Cholecystokinin CCK2 receptor, Chorionic gonadotropin, c-Kit (tyrosine-protein kinase Kit or CD117), CISH (Cytokine-inducible SH2-containing protein), Claudin (such as 6, 18), cluster of differentiation (CD) such as CD4, CD27, CD29, CD30, CD33, CD37, CD40, CD40 ligand receptor, CD40 ligand, CD40LG gene, CD44, CD45, CD47, CD49b, CD51, CD52, CD55, CD58, CD66e (CEACAM6), CD70 gene, CD74, CD79, CD79b, CD79B gene, CD80, CD95, CD99, CD117, CD122, CDw123, CD134, CDw137, CD158a, CD158b1, CD158b2, CD223, CD276 antigen; clusterin (CLU) gene, Clusterin, c-Met (hepatocyte growth factor receptor (HGFR)), Complement C3, Connective tissue growth factor, COP9 signalosome subunit 5, CSF-1 (colony-stimulating factor 1 receptor), CSF2 gene, CTLA-4 (cytotoxic T-lymphocyte protein 4) receptor, C-type lectin domain protein 9A (CLEC9A), Cyclin D1, Cyclin G, cyclin-dependent kinases (CDK, such as CDK1, CDK12, CDK1B, CDK2-9), cyclooxygenase (such as COX1, COX2), CYP2B1 gene, Cysteine palmitoyltransferase porcupine, Cytochrome P450 11B2, Cytochrome P450 17, cytochrome P450 17A1, Cytochrome P450 2D6, cytochrome P450 3A4, Cytochrome P450 reductase, cytokine signalling-1, cytokine signalling-3, Cytoplasmic isocitrate dehydrogenase, Cytosine deaminase, cytosine DNA methyltransferase, cytotoxic T-lymphocyte protein-4, DDR2 gene, DEAD-box helicase 6 (DDX6), Death receptor 5 (DR5, TRAILR2), Death receptor 4 (DR4, TRAILR1), Delta-like protein ligand (such as 3, 4), Deoxyribonuclease, Deubiquitinating enzymes (DUBs), Dickkopf-1 ligand, dihydrofolate reductase (DHFR), Dihydropyrimidine dehydrogenase, Dipeptidyl peptidase IV, discoidin domain receptor (DDR, such as DDR1), Diacylglycerol kinase zeta (DGKZ), DNA binding protein (such as HU-beta), DNA dependent protein kinase, DNA gyrase, DNA methyltransferase, DNA polymerase (such as alpha), DNA primase, dUTP pyrophosphatase, L-dopachrome tautomerase, E3 ubiquitin-protein ligase (such as RNF128, CBL-B), echinoderm microtubule like protein 4, EGFR tyrosine kinase receptor, Elastase, Elongation factor 1 alpha 2, Elongation factor 2, Endoglin, Endonuclease, endoplasmic reticulum aminopeptidase (ERAP, such as ERAP 1, ERAP2), Endoplasmin, Endosialin, Endostatin, endothelin (such as ET-A, ET-B), Enhancer of zeste homolog 2 (EZH2), Ephrin (EPH) tyrosine kinase (such as Epha3, Ephb4), Ephrin B2 ligand, epidermal growth factor, epidermal growth factor receptors (EGFR), epidermal growth factor receptor (EGFR) gene, Epigen, Epithelial cell adhesion molecule (EpCAM), Erb-b2 (v-erb-b2 avian erythroblastic leukemia viral oncogene homolog 2) tyrosine kinase receptor, Erb-b3 tyrosine kinase receptor, Erb-b4 tyrosine kinase receptor, E-selectin, Estradiol 17 beta dehydrogenase, Estrogen receptor (such as alpha, beta), Estrogen related receptor, Eukaryotic translation initiation factor 5A (EIF5A) gene, Exportin 1, Extracellular signal related kinase (such as 1, 2), Extracellular signal-regulated kinases (ERK), Hypoxia-inducible factor prolyl hydroxylase (HIF-PH or EGLN), Factor (such as Xa, VIIa), famesoid x receptor (FXR), Fas ligand, Fatty acid synthase (FASN), Ferritin, FGF-2 ligand, FGF-5 ligand, fibroblast growth factor (FGF, such as FGF1, FGF2, FGF4), Fibronectin, focal adhesion kinase (FAK, such as FAK2), folate hydrolase prostate-specific membrane antigen 1 (FOLH1), Folate receptor (such as alpha), Folate, Folate transporter 1, FYN tyrosine kinase, paired basic amino acid cleaving enzyme (FURIN), Beta-glucuronidase, Galactosyltransferase, Galectin-3, Ganglioside GD2, Glucocorticoid, glucocorticoid-induced TNFR-related protein GITR receptor, Glutamate carboxypeptidase II, glutaminase, Glutathione S-transferase P, glycogen synthase kinase (GSK, such as 3-beta), Glypican 3 (GPC3), gonadotropin-releasing hormone (GNRH), Granulocyte macrophage colony stimulating factor (GM-CSF) receptor, Granulocyte-colony stimulating factor (GCSF) ligand, growth factor receptor-bound protein 2 (GRB2), Grp78 (78 kDa glucose-regulated protein) calcium binding protein, molecular chaperone groEL2 gene, Heme oxygenase 1 (HO1), Heme oxygenase 2 (H02), Heat shock protein (such as 27, 70, 90 alpha, beta), Heat shock protein gene, Heat stable enterotoxin receptor, Hedgehog protein, Heparanase, Hepatocyte growth factor, HERV-H LTR associating protein 2, Hexose kinase, Histamine H2 receptor, Histone methyltransferase (DOT1L), histone deacetylase (HDAC, such as 1, 2, 3, 6, 10, 11), Histone H1, Histone H3, HLA class I antigen (A-2 alpha), HLA class II antigen, HLA class I antigen alpha G (HLA-G), Non-classical HLA, Homeobox protein NANOG, HSPB1 gene, Human leukocyte antigen (HLA), Human papillomavirus (such as E6, E7) protein, Hyaluronic acid, Hyaluronidase, Hypoxia inducible factor-1 alpha (HIF1α), Imprinted Maternally Expressed Transcript (H19) gene, mitogen-activated protein kinase 1 (MAP4K1), tyrosine-protein kinase HCK, I-Kappa-B kinase (IKK, such as IKKbe), IL-1 alpha, IL-1 beta, IL-12, IL-12 gene, IL-15, IL-17, IL-2 gene, IL-2 receptor alpha subunit, IL-2, IL-3 receptor, IL-4, IL-6, IL-7, IL-8, immunoglobulin (such as G, G1, G2, K, M), Immunoglobulin Fc receptor, Immunoglobulin gamma Fc receptor (such as I, III, IIIA), indoleamine 2,3-dioxygenase (IDO, such as IDO1 and IDO2), indoleamine pyrrole 2,3-dioxygenase 1 inhibitor, insulin receptor, Insulin-like growth factor (such as 1, 2), Integrin alpha-4/beta-1, integrin alpha-4/beta-7, Integrin alpha-5/beta-1, Integrin alpha-V/beta-3, Integrin alpha-V/beta-5, Integrin alpha-V/beta-6, Intercellular adhesion molecule 1 (ICAM-1), interferon (such as alpha, alpha 2, beta, gamma), Interferon inducible protein absent in melanoma 2 (AIM2), interferon type I receptor, Interleukin 1 ligand, Interleukin 13 receptor alpha 2, interleukin 2 ligand, interleukin-1 receptor-associated kinase 4 (IRAK4), Interleukin-2, Interleukin-29 ligand, Interleukin 35 (IL-35), isocitrate dehydrogenase (such as IDH1, IDH2), Janus kinase (JAK, such as JAKI, JAK2), Jun N terminal kinase, kallikrein-related peptidase 3 (KLK3) gene, Killer cell Ig like receptor, Kinase insert domain receptor (KDR), Kinesin-like protein KIF11, Kirsten rat sarcoma viral oncogene homolog (KRAS) gene, Kisspeptin (KiSS-1) receptor, KIT gene, v-kit Hardy-Zuckerman 4 feline sarcoma viral oncogene homolog (KIT) tyrosine kinase, lactoferrin, Lanosterol-14 demethylase, LDL receptor related protein-1, Leukocyte immunoglobulin-like receptor subfamily B member 1 (ILT2), Leukocyte immunoglobulin-like receptor subfamily B member 2 (ILT4), Leukotriene A4 hydrolase, Listeriolysin, L-Selectin, Luteinizing hormone receptor, Lyase, lymphocyte activation gene 3 protein (LAG-3), Lymphocyte antigen 75, Lymphocyte function antigen-3 receptor, lymphocyte-specific protein tyrosine kinase (LCK), Lymphotactin, Lyn (Lck/Yes novel) tyrosine kinase, lysine demethylases (such as KDM1, KDM2, KDM4, KDM5, KDM6, A/B/C/D), Lysophosphatidate-1 receptor, lysosomal-associated membrane protein family (LAMP) gene, Lysyl oxidase homolog 2, lysyl oxidase protein (LOX), 5-Lipoxygenase (5-LOX), Hematopoietic Progenitor Kinase 1 (HPKI), Hepatocyte growth factor receptor (MET) gene, macrophage colony-stimulating factor (MCSF) ligand, Macrophage migration inhibitory fact, MAGECI gene, MAGEC2 gene, Major vault protein, MAPK-activated protein kinase (such as MK2), Mas-related G-protein coupled receptor, matrix metalloprotease (MMP, such as MMP2, MMP9), Mcl-1 differentiation protein, Mdm2 p53-binding protein, Mdm4 protein, Melan-A (MART-1) melanoma antigen, Melanocyte protein Pmel 17, melanocyte stimulating hormone ligand, melanoma antigen family A3 (MAGEA3) gene, Melanoma associated antigen (such as 1, 2, 3, 6), Membrane copper amine oxidase, Mesothelin, MET tyrosine kinase, Metabotropic glutamate receptor 1, Metalloreductase STEAP1 (six transmembrane epithelial antigen of the prostate 1), Metastin, methionine aminopeptidase-2, Methyltransferase, Mitochondrial 3 ketoacyl CoA thiolase, mitogen-activate protein kinase (MAPK), mitogen-activated protein kinase (MEK, such as MEK1, MEK2), mTOR (mechanistic target of rapamycin (serine/threonine kinase), mTOR complex (such as 1,2), mucin (such as 1, 5A, 16), mut T homolog (MTH, such as MTH1), Myc proto-oncogene protein, myeloid cell leukemia 1 (MCL1) gene, myristoylated alanine-rich protein kinase C substrate (MARCKS) protein, NAD ADP ribosyltransferase, natriuretic peptide receptor C, Neural cell adhesion molecule 1, Neurokinin 1 (NK1) receptor, Neurokinin receptor, Neuropilin 2, NF kappa B activating protein, NIMA-related kinase 9 (NEK9), Nitric oxide synthase, NK cell receptor, NK3 receptor, NKG2 A B activating NK receptor, NLRP3 (NACHT LRR PYD domain protein 3) modulators, Noradrenaline transporter, Notch (such as Notch-2 receptor, Notch-3 receptor, Notch-4 receptor), Nuclear erythroid 2-related factor 2, Nuclear Factor (NF) kappa B, Nucleolin, Nucleophosmin, nucleophosmin-anaplastic lymphoma kinase (NPM-ALK), 2 oxoglutarate dehydrogenase, 2,5-oligoadenylate synthetase, O-methylguanine DNA methyltransferase, Opioid receptor (such as delta), Ornithine decarboxylase, Orotate phosphoribosyltransferase, orphan nuclear hormone receptor NR4A1, Osteocalcin, Osteoclast differentiation factor, Osteopontin, OX-40 (tumor necrosis factor receptor superfamily member 4 TNFRSF4, or CD134) receptor, P3 protein, p38 kinase, p38 MAP kinase, p53 tumor suppressor protein, Parathyroid hormone ligand, peroxisome proliferator-activated receptors (PPAR, such as alpha, delta, gamma), P-Glycoprotein (such as 1), phosphatase and tensin homolog (PTEN), phosphatidylinositol 3-kinase (PI3K), phosphoinositide-3 kinase (PI3K such as alpha, delta, gamma), phosphorylase kinase (PK), PKN3 gene, placenta growth factor, platelet-derived growth factor (PDGF, such as alpha, beta), Platelet-derived growth factor (PDGF, such as alpha, beta), Pleiotropic drug resistance transporter, Plexin B1, PLK1 gene, polo-like kinase (PLK), Polo-like kinase 1, Poly (ADP-ribose) polymerase (PARP, such as PARP1, PARP2 and PARP3, PARP7, and mono-PARPs), Preferentially expressed antigen in melanoma (PRAME) gene, Prenyl-binding protein (PrPB), Probable transcription factor PML, Progesterone receptor, Programmed cell death 1 (PD-1), Programmed cell death ligand 1 inhibitor (PD-L1), Prosaposin (PSAP) gene, Prostanoid receptor (EP4), Prostaglandin E2 synthase, prostate specific antigen, Prostatic acid phosphatase, proteasome, Protein E7, Protein farnesyltransferase, protein kinase (PK, such as A, B, C), protein tyrosine kinase, Protein tyrosine phosphatase beta, Proto-oncogene serine/threonine-protein kinase (PIM, such as PIM-1, PIM-2, PIM-3), P-Selectin, Purine nucleoside phosphorylase, purinergic receptor P2X ligand gated ion channel 7 (P2X7), Pyruvate dehydrogenase (PDH), Pyruvate dehydrogenase kinase, Pyruvate kinase (PYK), 5-Alpha-reductase, Raf protein kinase (such as 1, B), RAF1 gene, Ras gene, Ras GTPase, RET gene, Ret tyrosine kinase receptor, retinoblastoma associated protein, retinoic acid receptor (such as gamma), Retinoid X receptor, Rheb (Ras homolog enriched in brain) GTPase, Rho (Ras homolog) associated protein kinase 2, ribonuclease, Ribonucleotide reductase (such as M2 subunit), Ribosomal protein S6 kinase, RNA polymerase (such as I, II), Ron (Recepteur d'Origine Nantais) tyrosine kinase, ROS1 (ROS proto-oncogene 1, receptor tyrosine kinase) gene, Ros1 tyrosine kinase, Runt-related transcription factor 3, Gamma-secretase, S100 calcium binding protein A9, Sarco endoplasmic calcium ATPase, Second mitochondria-derived activator of caspases (SMAC) protein, Secreted frizzled related protein-2, Secreted phospholipase A2, Semaphorin-4D, Serine protease, serine/threonine kinase (STK), serine/threonine-protein kinase (TBK, such as TBK1), signal transduction and transcription (STAT, such as STAT-1, STAT-3, STAT-5), Signaling lymphocytic activation molecule (SLAM) family member 7, six-transmembrane epithelial antigen of the prostate (STEAP) gene, SL cytokine ligand, smoothened (SMO) receptor, Sodium iodide cotransporter, Sodium phosphate cotransporter 2B, Somatostatin receptor (such as 1, 2, 3, 4, 5), Sonic hedgehog protein, Son of sevenless (SOS), Specific protein 1 (Sp1) transcription factor, Sphingomyelin synthase, Sphingosine kinase (such as 1, 2), Sphingosine-1-phosphate receptor-1, spleen tyrosine kinase (SYK), SRC gene, Src tyrosine kinase, Stabilin-1 (STAB1), STAT3 gene, Steroid sulfatase, Stimulator of interferon genes (STING) receptor, stimulator of interferon genes protein, Stromal cell-derived factor 1 ligand, SUMO (small ubiquitin-like modifier), Superoxide dismutase, Suppressor of cytokine signaling modulators (SOCS), Survivin protein, Synapsin 3, Syndecan-1, Synuclein alpha, T cell surface glycoprotein CD28, tank-binding kinase (TBK), TATA box-binding protein-associated factor RNA polymerase I subunit B (TAF1B) gene, T-cell CD3 glycoprotein zeta chain, T-cell differentiation antigen CD6, T-cell immunoglobulin and mucin-domain containing-3 (TIM-3), T-cell surface glycoprotein CD8, Tec protein tyrosine kinase, Tek tyrosine kinase receptor, telomerase, Telomerase reverse transcriptase (TERT) gene, Tenascin, Three prime repair exonuclease 1 (TREX1), Three prime repair exonuclease 2 (TREX2), Thrombopoietin receptor, Thymidine kinase, Thymidine phosphorylase, Thymidylate synthase, Thymosin (such as alpha 1), Thyroid hormone receptor, Thyroid stimulating hormone receptor, Tissue factor, TNF related apoptosis inducing ligand, TNFR1 associated death domain protein, TNF-related apoptosis-inducing ligand (TRAIL) receptor, TNFSF11 gene, TNFSF9 gene, Toll-like receptor (TLR such as 1-13), topoisomerase (such as I, II, III), Transcription factor, Transferase, transferrin (TF), transforming growth factor alpha (TGFα), transforming growth factor beta (TGFB) and isoforms thereof, TGF beta 2 ligand, Transforming growth factor TGF-β receptor kinase, Transglutaminase, Translocation associated protein, Transmembrane glycoprotein NMB, Trop-2 calcium signal transducer, trophoblast glycoprotein (TPBG) gene, Trophoblast glycoprotein, Tropomyosin receptor kinase (Trk) receptor (such as TrkA, TrkB, TrkC), tryptophan 2,3-dioxygenase (TDO), Tryptophan 5-hydroxylase, Tubulin, Tumor necrosis factor (TNF, such as alpha, beta), Tumor necrosis factor 13C receptor, tumor progression locus 2 (TPL2), Tumor protein 53 (TP53) gene, Tumor suppressor candidate 2 (TUSC2) gene, Tumor specific neoantigens, Tyrosinase, Tyrosine hydroxylase, tyrosine kinase (TK), Tyrosine kinase receptor, Tyrosine kinase with immunoglobulin-like and EGF-like domains (TIE) receptor, Tyrosine protein kinase ABL1 inhibitor, Ubiquitin, Ubiquitin carboxyl hydrolase isozyme L5, Ubiquitin thioesterase-14, Ubiquitin-conjugating enzyme E21(UBE2I, UBC9), Ubiquitin-specific-processing protease 7 (USP7), Urease, Urokinase plasminogen activator, Uteroglobin, Vanilloid VR1, Vascular cell adhesion protein 1, vascular endothelial growth factor receptor (VEGFR), V-domain Ig suppressor of T-cell activation (VISTA), VEGF-1 receptor, VEGF-2 receptor, VEGF-3 receptor, VEGF-A, VEGF-B, Vimentin, Vitamin D3 receptor, Proto-oncogene tyrosine-protein kinase, Mer (Mer tyrosine kinase receptor modulators), YAP (Yes-associated protein modulators)es, Wee-1 protein kinase, Werner Syndrome RecQ Like Helicase (WRN), Wilms' tumor antigen 1, Wilms' tumor protein, WW domain containing transcription regulator protein 1 (TAZ), X-linked inhibitor of apoptosis protein, Zinc finger protein transcription factor or any combination thereof.

In various embodiments, an anti-CD47 agent or an anti-SIRPα agent as described herein, is combined with one or more additional therapeutic agents that may be categorized by their mechanism of action into, for example, the following groups: anti-metabolites/anti-cancer agents, such as pyrimidine analogs floxuridine, capecitabine, cytarabine, CPX-351 (liposomal cytarabine, daunorubicin), and TAS-118; Alpha 1 adrenoceptor/Alpha 2 adrenoceptor antagonists, such as phenoxybenzamine hydrochloride (injectable, pheochromocytoma); Androgen receptor antagonists, such as nilutamide; anti-cadherin antibodies, such as HKT-288; anti-leucine-rich repeat containing 15 (LRRC15) antibodies, such as ABBV-085. ARGX-110; angiotensin receptor blockers, nitric oxide donors; antisense oligonucleotides, such as AEG35156, IONIS-KRAS-2.5Rx, EZN-3042, RX-0201, IONIS-AR-2.5Rx, BP-100 (prexigebersen), IONIS-STAT3-2.5Rx; anti-angiopoietin (ANG)-2 antibodies, such as MED13617, and LY3127804; anti-ANG-1/ANG-2 antibodies, such as AMG-780; anti-CSF1R antibodies, such as emactuzumab, LY3022855, AMG-820, FPA-008 (cabiralizumab); anti-endoglin antibodies, such as TRC105 (carotuximab); anti-ERBB antibodies, such as CDX-3379, HLX-02, seribantumab; anti-HER2 antibodies, such as HERCEPTIN® (trastuzumab), trastuzumab biosimimar, margetuximab, MED14276, BAT-8001, Pertuzumab (Perjeta), RG6264, ZW25 (a bispecific HER2-directed antibody targeting the extracellular domains 2 and 4; Cancer Discov. 2019 January; 9(1):8; PMID: 30504239); anti-HLA-DR antibodies, such as IMMU-114; anti-IL-3 antibodies, such as JNJ-56022473; anti-TNF receptor superfamily member 18 (TNFRSF18, GITR; NCBI Gene ID: 8784) antibodies, such as MK-4166, MED11873, FPA-154, INCAGN-1876, TRX-518, BMS-986156, MK-1248, GWN-323; and those described, e.g. in Intl. Patent Publ. Nos. WO 2017/096179, WO 2017/096276, WO 2017/096189; and WO 2018/089628; anti-EphA3 antibodies, such as KB-004; anti-CD37 antibodies, such as otlertuzumab (TRU-016); anti-FGFR-3 antibodies, such as LY3076226, B-701; anti-FGFR-2 antibodies, such as GAL-F2; anti-C5 antibodies, such as ALXN-1210; anti-EpCAM antibodies, such as VB4-845; anti-CEA antibodies, such as RG-7813; anti-Carcinoembryonic-antigen-related-cell-adhesion-molecule-6 (CEACAM6, CD66C) antibodies, such as BAY-1834942, NEO-201 (CEACAM 5/6); anti-GD2 antibodies, such as APN-301; anti-interleukin-17 (IL-17) antibodies, such as CJM-112; anti-interleukin-1 beta antibodies, such as canakinumab (ACZ885), VPM087; anti-carbonic anhydrase 9 (CA9, CAIX) antibodies, such as TX-250; anti-Mucin 1 (MUC1) antibodies, such as gatipotuzumab, Mab-AR-20.5; anti-KMA antibodies, such as MDX-1097; anti-CD55 antibodies, such as PAT-SC; anti-c-Met antibodies, such as ABBV-399; anti-PSMA antibodies, such as ATL-101; anti-CD100 antibodies, such as VX-15; anti-EPHA3 antibodies, such as fibatuzumab; anti-APRIL antibodies, such as BION-1301; anti-fibroblast activation protein (FAP)/IL-2R antibodies, such as RG7461; anti-fibroblast activation protein (FAP)/TRAIL-R2 antibodies, such as RG7386; anti-fucosyl-GM1 antibodies, such as BMS-986012; anti-IL-8 (Interleukin-8) antibodies, such as HuMax-Inflam; anti-myostatin inhibitors, such as landogrozumab; anti-delta-like protein ligand 3 (DDL3) antibodies, such as rovalpituzumab tesirine; anti-DLL4 (delta like ligand 4) antibodies, such as demcizumab; anti-clusterin antibodies, such as AB-16B5; anti-Ephrin-A4 (EFNA4) antibodies, such as PF-06647263; anti-mesothelin antibodies, such as BMS-986148, Anti-MSLN-MMAE; anti-sodium phosphate cotransporter 2B (NaP2B) antibodies, such as lifastuzumab; anti-TGFb antibodies, such as SAR439459; anti-transforming growth factor-beta (TGF-beta) antibodies, such as ABBV-151, LY3022859, NIS793, XOMA 089; purine analogs, folate antagonists (such as pralatrexate), cladribine, pentostatin, fludarabine and related inhibitors; antiproliferative/antimitotic agents including natural products, such as vinca alkaloids (vinblastine, vincristine) and microtubule disruptors such as taxane (paclitaxel, docetaxel), vinblastin, nocodazole, epothilones, vinorelbine (NAVELBINE®), and epipodophyllotoxins (etoposide, teniposide); DNA damaging agents, such as actinomycin, amsacrine, busulfan, carboplatin, chlorambucil, cisplatin, cyclophosphamide (CYTOXAN®), dactinomycin, daunorubicin, doxorubicin, DEBDOX, epirubicin, iphosphamide, melphalan, merchlorethamine, mitomycin C, mitoxantrone, nitrosourea, procarbazine, taxol, Taxotere, teniposide, etoposide, and triethylenethiophosphoramide; DNA-hypomethylating agents, such as guadecitabine (SGI-110), ASTX727; antibiotics such as dactinomycin, daunorubicin, doxorubicin, idarubicin, anthracyclines, mitoxantrone, bleomycins, plicamycin (mithramycin); enzymes such as L-asparaginase which systemically metabolizes L-asparagine and deprives cells which do not have the capacity to synthesize their own asparagine; DNAi oligonucleotides targeting Bcl-2, such as PNT2258; agents that activate or reactivate latent human immunodeficiency virus (HIV), such as panobinostat and romidepsin; asparaginase stimulators, such as crisantaspase (Erwinase®) and GRASPA (ERY-001, ERY-ASP), calaspargase pegol, pegaspargase; pan-Trk, ROS1 and ALK inhibitors, such as entrectinib, TPX-0005; anaplastic lymphoma kinase (ALK) inhibitors, such as alectinib, ceritinib, alecensa (RG7853), ALUNBRIG® (brigatinib); antiproliferative/antimitotic alkylating agents, such as nitrogen mustard cyclophosphamide and analogs (e.g., melphalan, chlorambucil, hexamethylmelamine, thiotepa), alkyl nitrosoureas (e.g., carmustine) and analogs, streptozocin, and triazenes (e.g., dacarbazine); antiproliferative/antimitotic antimetabolites, such as folic acid analogs (methotrexate); platinum coordination complexes (e.g., cisplatin, oxiloplatinim, and carboplatin), procarbazine, hydroxyurea, mitotane, and aminoglutethimide; hormones, hormone analogs (e.g., estrogen, tamoxifen, goserelin, bicalutamide, and nilutamide), and aromatase inhibitors (e.g., letrozole and anastrozole); antiplatelet agents; anticoagulants such as heparin, synthetic heparin salts, and other inhibitors of thrombin; fibrinolytic agents such as tissue plasminogen activator, streptokinase, urokinase, aspirin, dipyridamole, ticlopidine, and clopidogrel; antimigratory agents; antisecretory agents (e.g., breveldin); immunosuppressives, such as tacrolimus, sirolimus, azathioprine, and mycophenolate; growth factor inhibitors, and vascular endothelial growth factor inhibitors; fibroblast growth factor inhibitors, such as FPA14; AMP activated protein kinase stimulators, such as metformin hydrochloride; ADP ribosyl cyclase-1 inhibitors, such as daratumumab (DARZALEX®); Caspase recruitment domain protein-15 stimulators, such as mifamurtide (liposomal); CCR5 chemokine antagonists, such as MK-7690 (vicriviroc); CDCl₇ protein kinase inhibitors, such as TAK-931; Cholesterol side-chain cleavage enzyme inhibitors, such as ODM-209; Dihydropyrimidine dehydrogenase/Orotate phosphoribosyltransferase inhibitors, such as Cefesone (tegafur+gimeracil+oteracil potassium); DNA polymerase/Ribonucleotide reductase inhibitors, such as clofarabine; DNA interference oligonucleotides, such as PNT2258, AZD-9150; Estrogen receptor modulators, such as bazedoxifene; Estrogen receptor agonists/Progesterone receptor antagonists, such as TRI-CYCLEN LO (norethindrone+ethinyl estradiol); HLA class I antigen A-2 alpha modulators, such as FH-MCVA2TCR; HLA class I antigen A-2 alpha/MART-1 melanoma antigen modulators, such as MART-1 F5 TCR engineered PBMC; Human Granulocyte Colony Stimulating Factors, such as PF-06881894; GNRH receptor agonists, such as leuprorelin acetate, leuprorelin acetate sustained release depot (ATRIGEL), triptorelin pamoate, goserelin acetate; GNRH receptor antagonists, such as elagolix, relugolix, degarelix; Endoplasmin modulators, such as anlotinib; H+ K+ ATPase inhibitors, such as omeprazole, esomeprazole; ICAM-1/CD55 modulators, such as cavatak (V-937); IL-15/IL-12 modulators, such as SAR441000; Interleukin 23A inhibitors, such as guselkumab; Lysine specific histone demethylase 1 inhibitors, such as CC-90011; IL-12 Mma, such as MEDI1191; RIG-I modulators, such as RGT-100; NOD2 modulators, such as SB-9200, and IR-103; Progesterone receptor agonists, such as levonorgestrel; Protein cereblon modulators, such as CC-92480, CC-90009; Protein cereblon modulators/DNA binding protein Ikaros inhibitors/Zinc finger binding protein Aiolos inhibitors, such as iberdomide; Retinoid X receptor modulators, such as alitretinoin, bexarotene (oral formulation); RIP-1 kinase inhibitors, such as GSK-3145095; selective oestrogen receptor degraders, such as AZD9833; SUMO inhibitors, such as TAK-981; Thrombopoietin receptor agonists, such as eltrombopag; Thyroid hormone receptor agonists, such as levothyroxine sodium; TNF agonists, such as tasonermin; Tyrosine phosphatase substrate 1 inhibitors, such as CC-95251; HER2 inhibitors, such as neratinib, tucatinib (ONT-380); EGFR/ErbB2/Ephb4 inhibitors, such as tesevatinib; EGFR/HER2 inhibitors, such as TAK-788; EGFR family tyrosine kinase receptor inhibitors, such as DZD-9008; EGFR/ErbB-2 inhibitors, such as varlitinib; mutant selective EGFR inhibitors, such as PF-06747775, EGF816 (nazartinib), ASP8273, ACEA-0010, BI-1482694; epha2 inhibitors, such as MM-310; polycomb protein (EED) inhibitors, such as MAK683; DHFR inhibitor/Folate transporter 1 modulator/Folate receptor antagonist, such as pralatrexate; DHFR/GAR transformylase/Thymidylate synthase/Transferase inhibitors, such as pemetrexed disodium; p38 MAP kinase inhibitors, such as ralimetinib; PRMT inhibitors, such as MS203, PF-06939999, GSK3368715, GSK3326595; Sphingosine kinase 2 (SK2) inhibitors, such as opaganib; Nuclear erythroid 2-related factor 2 stimulators, such as omaveloxolone (RTA-408); Tropomyosin receptor kinase (TRK) inhibitors, such as LOXO-195, ONO-7579; Mucin 1 inhibitors, such as GO-203-2C; MARCKS protein inhibitors, such as BIO-11006; Folate antagonists, such as arfolitixorin; Galectin-3 inhibitors, such as GR-MD-02; Phosphorylated P68 inhibitors, such as RX-5902; CD95/TNF modulators, such as ofranergene obadenovec; pan-PIM kinase inhibitors, such as INCB-053914; IL-12 gene stimulators, such as EGEN-001, tavokinogene telseplasmid; Heat shock protein HSP90 inhibitors, such as TAS-116, PEN-866; VEGF/HGF antagonists, such as MP-0250; VEGF ligand inhibitors, such as bevacizumab biosimilar; VEGF receptor antagonists/VEGF ligand inhibitors, such as ramucirumab; VEGF-1/VEGF-2/VEGF-3 receptor antagonists; such as fruquintinib; VEGF-1/VEGF-2 receptor modulators, such as HLA-A2402/HLA-A0201 restricted epitope peptide vaccine; Placenta growth factor ligand inhibitor/VEGF-A ligand inhibitor, such as aflibercept; SYK tyrosine kinase/JAK tyrosine kinase inhibitors, such as ASN-002; Trk tyrosine kinase receptor inhibitors, such as larotrectinib sulfate; JAK3/JAKI/TBKI kinase inhibitors, such as CS-12912; IL-24 antagonist, such as AD-IL24; NLRP3 (NACHT LRR PYD domain protein 3) modulators, such as BMS-986299; RIG-I agonists, such as RGT-100; Aerolysin stimulators, such as topsalysin; P-Glycoprotein 1 inhibitors, such as HM-30181A; CSF-1 antagonists, such as ARRY-382, BLZ-945; CCR8 inhibitors, such as JTX-1811, I-309, SB-649701, HG-1013, RAP-310; anti-Mesothelin antibodies, such as SEL-403; Thymidine kinase stimulators, such as aglatimagene besadenovec; Polo-like kinase 1 inhibitors, such as PCM-075, onvansertib; NAE inhibitors, such as pevonedistat (MLN-4924), TAS-4464; Pleiotropic pathway modulators, such as avadomide (CC-122); Amyloid protein binding protein-1 inhibitorS/Ubiquitin ligase modulators, such as pevonedistat; FoxM1 inhibitors, such as thiostrepton; UBA1 inhibitors, such as TAK-243; Src tyrosine kinase inhibitors, such as VAL-201; VDAC/HK inhibitors, such as VDA-1102; Elf4a inhibitors, such as rohinitib, eFT226; TP53 gene stimulators, such as ad-p53; Retinoic acid receptor agonists, such as tretinoin; Retinoic acid receptor alpha (RARu) inhibitors, such as SY-1425; SIRT3 inhibitors, such as YC8-02; Stromal cell-derived factor 1 ligand inhibitors, such as olaptesed pegol (NOX-A12); IL-4 receptor modulators, such as MDNA-55; Arginase-I stimulators, such as pegzilarginase; Topoisomerase I inhibitors, such as innotecan hydrochloride, Onivyde; Topoisomerase I inhibitor/hypoxia inducible factor-1 alpha inhibitors, such as PEG-SN38 (firtecan pegol); Hypoxia inducible factor-1 alpha inhibitors, such as PT-2977, PT-2385; CD122 (IL-2 receptor) agonists, such as proleukin (aldesleukin, IL-2); pegylated IL-2 (eg NKTR-214); modified variants of IL-2 (eg THOR-707); TLR7/TLR8 agonist, such as NKTR-262; TLR7 agonists, such as DS-0509, GS-9620, LHC-165, TMX-101 (imiquimod); p53 tumor suppressor protein stimulators such as kevetrin; Mdm4/Mdm2 p53-binding protein inhibitors, such as ALRN-6924; kinesin spindle protein (KSP) inhibitors, such as filanesib (ARRY-520); CD80-fc fusion protein inhibitors, such as FPT-155; Menin and mixed lineage leukemia (MLL) inhibitors such as KO-539; Liver x receptor agonists, such as RGX-104; IL-10 agonists, such as Pegilodecakin (AM-0010); VEGFR/PDGFR inhibitors, such as vorolanib; IRAK4 inhibitors, such as CA-4948; anti-TLR-2 antibodies, such as OPN-305; Calmodulin modulators, such as CBP-501;

Glucocorticoid receptor antagonists, such as relacorilant (CORT-125134); Second mitochondria-derived activator of caspases (SMAC) protein inhibitors, such as BI-891065; Lactoferrin modulators, such as LTX-315; KIT proto-oncogene, receptor tyrosine kinase (KIT) inhibitors, such as PLX-9486; platelet derived growth factor receptor alpha (PDGFRA)/KIT proto-oncogene, receptor tyrosine kinase (KIT) mutant-specific antagonists/inhibitors such as BLU-285, DCC-2618; Exportin 1 inhibitors, such as eltanexor; CHST15 gene inhibitors, such as STNM-01; Somatostatin receptor antagonist, such as OPS-201; CEBPA gene stimulators, such as MTL-501; DKK3 gene modulators, such as MTG-201; Chemokine (CXCR1/CXCR2) inhibitors, such as SX-682; p70s6k inhibitors, such as MSC2363318A; methionine aminopeptidase 2 (MetAP2) inhibitors, such as M8891, APL-1202; arginine N-methyltransferase 5 inhibitors, such as GSK-3326595; CD71 modulators, such as CX-2029 (ABBV-2029); ATM (ataxia telangiectasia) inhibitors, such as AZD0156, AZD1390; CHK1 inhibitors, such as GDC-0575, LY2606368 (prexasertib), SRA737, RG7741 (CHK1/2); CXCR4 antagonists, such as BL-8040, LY2510924, burixafor (TG-0054), X4P-002, X4P-001-IO, Plerixafor; EXH2 inhibitors, such as GSK2816126; KDM1 inhibitors, such as ORY-1001, IMG-7289, INCB-59872, GSK-2879552; CXCR2 antagonists, such as AZD-5069; DNA dependent protein kinase inhibitors, such as MSC2490484A (nedisertib), VX-984, AsiDNA (DT-01); protein kinase C (PKC) inhibitors, such as LXS-196, sotrastaurin; selective estrogen receptor downregulators (SERD), such as fulvestrant (Faslodex®), RG6046, RG6047, RG6171, elacestrant (RAD-1901), SAR439859 and AZD9496; selective estrogen receptor covalent antagonists (SERCAs), such as H3B-6545; selective androgen receptor modulator (SARM), such as GTX-024, darolutamide; transforming growth factor-beta (TGF-beta) kinase antagonists, such as galunisertib, LY3200882; TGF-beta inhibitors described in WO 2019/103203; TGF beta receptor 1 inhibitors, such as PF-06952229; bispecific antibodies, such as ABT-165 (DLL4/VEGF), MM-141 (IGF-1/ErbB3), MM-111 (Erb2/Erb3), JNJ-64052781 (CD19/CD3), PRS-343 (CD-137/HER2), AFM26 (BCMA/CD16A), JNJ-61186372 (EGFR/cMET), AMG-211 (CEA/CD3), RG7802 (CEA/CD3), ERY-974 (CD3/GPC3) vancizumab (angiopoietins/VEGF), PF-06671008 (Cadherins/CD3), AFM-13 (CD16/CD30), APV0436 (CD123/CD3), flotetuzumab (CD123/CD3), REGN-1979 (CD20/CD3), MCLA-117 (CD3/CLEC12A), MCLA-128 (HER2/HER3), JNJ-0819, JNJ-7564 (CD3/heme), AMG-757 (DLL3-CD3), MGD-013 (PD-1/LAG-3), FS-118 (LAG-3/PD-L1) MGD-019 (PD-1/CTLA-4), KN-046 (PD-1/CTLA-4), MEDI-5752 (CTLA-4/PD-1), RO-7121661 (PD-1/TIM-3), XmAb-20717 (PD-1/CTLA-4), AK-104 (CTLA-4/PD-1), AMG-420 (BCMA/CD3), BI-836880 (VEFG/ANG2), JNJ-63709178 (CD123/CD3), MGD-007 (CD3/gpA33), MGD-009 (CD3/B7H3), AGEN1223, IMCgp100 (CD3/gp100), AGEN-1423, ATOR-1015 (CTLA-4/OX40), LY-3415244 (TIM-3/PDL1), INHIBRX-105 (4-1BB/PDL1), faricimab (VEGF-A/ANG-2), FAP-4-IBBL (4-1BB/FAP), XmAb-13676 (CD3/CD20), TAK-252 (PD-1/OX40L), TG-1801 (CD19/CD47), XmAb-18087 (SSTR2/CD3), catumaxomab (CD3/EpCAM), SAR-156597 (IL4/IL13), EMB-01 (EGFR/cMET), REGN-4018 (MUC16/CD3), REGN-1979 (CD20/CD3), RG-7828 (CD20/CD3), CC-93269 (CD3/BCMA), REGN-5458 (CD3/BCMA), navicixizumab (DLL4/VEGF), GRB-1302 (CD3/Erbb2), vanucizumab (VEGF-A/ANG-2), GRB-1342 (CD38/CD3), GEM-333 (CD3/CD33), IMM-0306 (CD47/CD20), RG6076, MEDI5752 (PD-1/CTLA-4), LY3164530 (MET/EGFR); Alpha-ketoglutarate dehydrogenase (KGDH) inhibitors, such as CPI-613; XPO1 inhibitors, such as selinexor (KPT-330); Isocitrate dehydrogenase 2 (IDH2) inhibitors, such as enasidenib (AG-221); IDH1 inhibitors such as AG-120, and AG-881 (IDH1 and IDH2), IDH-305, BAY-1436032; IDH1 gene inhibitors, such as ivosidenib; interleukin-3 receptor (IL-3R) modulators, such as SL-401; Arginine deiminase stimulators, such as pegargiminase (ADI-PEG-20); claudin-18 inhibitors, such as claudiximab; 0-catenin inhibitors, such as CWP-291; chemokine receptor 2 (CCR) inhibitors, such as PF-04136309, CCX-872, BMS-813160 (CCR2/CCR5); thymidylate synthase inhibitors, such as ONX-0801; ALK/ROS1 inhibitors, such as lorlatinib; tankyrase inhibitors, such as G007-LK; triggering receptor expressed on myeloid cells 1 (TREM1; NCBI Gene ID: 54210), such as PY159; triggering receptor expressed on myeloid cells 2 (TREM2; NCBI Gene ID: 54209), such as PY314; Mdm2 p53-binding protein inhibitors, such as CMG-097, HDM-201; c-PIM inhibitors, such as PIM447; sphingosine kinase-2 (SK2) inhibitors, such as Yeliva® (ABC294640); DNA polymerase inhibitors, such as sapacitabine; Cell cycle/Microtubule inhibitors, such as eribulin mesylate; c-MET inhibitors, such as AMG-337, savolitinib, tivantinib (ARQ-197), capmatinib, and tepotinib, ABT-700, AG213, AMG-208, JNJ-38877618 (OMO-1), merestinib, HQP-8361; c-Met/VEGFR inhibitors, such as BMS-817378, TAS-115; c-Met/RON inhibitors, such as BMS-777607; BCR/ABL inhibitors, such as rebastinib, asciminib, ponatinib (ICLUSIG®); MNK1/MNK2 inhibitors, such as eFT-508; Cytochrome P450 11B2/Cytochrome P450 17/AKT protein kinase inhibitors, such as LAE-201; Cytochrome P450 3A4 stimulators, such as mitotane; lysine-specific demethylase-1 (LSD1) inhibitors, such as CC-90011; CSF1R/KIT and FLT3 inhibitors, such as pexidartinib (PLX3397); Flt3 tyrosine kinase/Kit tyrosine kinase inhibitor and PDGF receptor antagonists, such as quizartinib dihydrochloride; kinase inhibitors, such as vandetanib; E selectin antagonists, such as GMI-1271; differentiation inducers, such as tretinoin; epidermal growth factor receptor (EGFR) inhibitors, such as osimertinib (AZD-9291), cetuximab; topoisomerase inhibitors, such as Adriamycin, doxorubicin, daunorubicin, dactinomycin, DaunoXome, Caelyx, eniposide, epirubicin, etoposide, idarubicin, irinotecan, mitoxantrone, pixantrone, sobuzoxane, topotecan, irinotecan, MM-398 (liposomal irinotecan), vosaroxin and GPX-150, aldoxorubicin, AR-67, mavelertinib, AST-2818, avitinib (ACEA-0010), irofulven (MGI-114); corticosteroids, such as cortisone, dexamethasone, hydrocortisone, methylprednisolone, prednisone, prednisolone; growth factor signal transduction kinase inhibitors; nucleoside analogs, such as DFP-10917; Axl inhibitors, such as BGB-324 (bemcentinib), SLC-0211; Axl/Flt3 inhibitors, such as gilteritinib; Inhibitors of bromodomain and extraterminal motif (BET) proteins, including ABBV-744, BRD2 (NCBI Gene ID: 6046), BRD3 (NCBI Gene ID: 8019), BRD4 (NCBI Gene ID: 23476), and bromodomain testis-specific protein (BRDT; NCBI Gene ID: 676), such as INCB-054329, INCB057643, TEN-010, AZD-5153, ABT-767, BMS-986158, CC-90010, GSK525762 (molibresib), NHWD-870, ODM-207, GSK-2820151, GSK-1210151A, ZBC246, ZBC260, ZEN3694, FT-1101, RG-6146, CC-90010, CC-95775, mivebresib, BI-894999, PLX-2853, PLX-51107, CPI-0610, GS-5829; PARP inhibitors, such as olaparib (MK7339), rucaparib, veliparib, talazoparib, ABT-767, BGB-290, fluzolepali (SHR-3162), niraparib (JNJ-64091742), bendamustine hydrochloride; PARP/Tankyrase inhibitors such as 2X-121 (e-7499); IMP-4297, SC-10914, IDX-1197, HWH-340, CK-102, simmiparib; Proteasome inhibitors, such as ixazomib (NINLARO®), carfilzomib (Kyprolis®), marizomib, bortezomib; Glutaminase inhibitors, such as CB-839 (telaglenastat), bis-2-(5-phenylacetamido-1,3,4-thiadiazol-2-yl)ethyl sulfide (BPTES); mitochondrial complex I inhibitors, such as metformin, phenformin; vaccines, such as peptide vaccine TG-01 (RAS), GALE-301, GALE-302, nelipepimut-s, SurVaxM, DSP-7888, TPIV-200, PVX-410, VXL-100, DPX-E7, ISA-101, 6MHP, OSE-2101, galinpepimut-S, SVN53-67/M57-KLH, IMU-131, peptide subunit vaccine (acute lymphoblastic leukemia, University Children's Hospital Tuebingen); bacterial vector vaccines such as CRS-207/GVAX, axalimogene filolisbac (ADXS11-001); adenovirus vector vaccines such as nadofaragene firadenovec; autologous Gp96 vaccine; dendritic cells vaccines, such as CVactm, tapuldencel-T, eltrapuldencel-T, SL-701, BSK01TM, rocapuldencel-T (AGS-003), DCVAC, CVactm, stapuldencel-T, eltrapuldencel-T, SL-701, BSK01TM, ADXS31-142, autologous dendritic cell vaccine (metastatic malignant melanoma, intradermal/intravenous, Universitatsklinikum Erlangen); oncolytic vaccines such as, talimogene laherparepvec, pexastimogene devacirepvec, GL-ONC1, MG1-MA3, parvovirus H-1, ProstAtak, enadenotucirev, MG1MA3, ASN-002 (TG-1042); therapeutic vaccines, such as CVAC-301, CMP-001, CreaVax-BC, PF-06753512, VBI-1901, TG-4010, ProscaVax™; tumor cell vaccines, such as Vigil® (IND-14205), Oncoquest-L vaccine; live attenuated, recombinant, serotype 1 poliovirus vaccine, such as PVS-RIPO; Adagloxad simolenin; MEDI-0457; DPV-001 a tumor-derived, autophagosome enriched cancer vaccine; RNA vaccines such as, CV-9209, LV-305; DNA vaccines, such as MEDI-0457, MVI-816, INO-5401; modified vaccinia virus Ankara vaccine expressing p53, such as MVA-p53; DPX-Survivac; BriaVax™; GI-6301; GI-6207; GI-4000; 10-103; Neoantigen peptide vaccines, such as AGEN-2017, GEN-010, NeoVax, RG-6180, GEN-009, PGV-001 (TLR-3 agonist), GRANITE-001, NEO-PV-01; Peptide vaccines that target heat shock proteins, such as PhosphoSynVax™; Vitespen (HSPPC-96-C), NANT Colorectal Cancer Vaccine containing aldoxorubicin, autologous tumor cell vaccine+systemic CpG-B+IFN-alpha (cancer), IO-120+IO-103 (PD-L1/PD-L2 vaccines), HB-201, HB-202, HB-301, TheraT®*-based vaccines; TLR-3 agonist/interferon inducers, such as Poly-ICLC (NSC-301463); STAT-3 inhibitors, such as napabucasin (BBI-608); ATPase p97 inhibitors, such as CB-5083; smoothened (SMO) receptor inhibitors, such as Odomzo® (sonidegib, formerly LDE-225), LEQ506, vismodegib (GDC-0449), BMS-833923, glasdegib (PF-04449913), LY2940680, and itraconazole; interferon alpha ligand modulators, such as interferon alpha-2b, interferon alpha-2a biosimilar (Biogenomics), ropeginterferon alfa-2b (AOP-2014, P-1101, PEG IFN alpha-2b), Multiferon (Alfanative, Viragen), interferon alpha 1b, Roferon-A (Canferon, Ro-25-3036), interferon alfa-2a follow-on biologic (Biosidus)(Inmutag, Inter 2A), interferon alfa-2b follow-on biologic (Biosidus—Bioferon, Citopheron, Ganapar, Beijing Kawin Technology—Kaferon), Alfaferone, pegylated interferon alpha-1b, peginterferon alfa-2b follow-on biologic (Amega), recombinant human interferon alpha-1b, recombinant human interferon alpha-2a, recombinant human interferon alpha-2b, veltuzumab-IFN alpha 2b conjugate, Dynavax (SD-101), and interferon alfa-n1 (Humoferon, SM-10500, Sumiferon); interferon gamma ligand modulators, such as interferon gamma (OH-6000, Ogamma 100); telomerase modulators, such as, tertomotide (GV-1001, HR-2802, Riavax) and imetelstat (GRN-163, JNJ-63935937); DNA methyltransferases inhibitors, such as temozolomide (CCRG-81045), decitabine, guadecitabine (5-110, SGI-110), KRX-0402, RX-3117, RRx-001, and azacytidine (CC-486); DNA gyrase inhibitors, such as pixantrone and sobuzoxane; DNA gyrase inhibitors/Topoisimerase II inhibitors, such as amrubicin; Bcl-2 family protein inhibitors, such as ABT-263, venetoclax (ABT-199), ABT-737, RG7601, and AT-101; Bcl-2/Bcl-XL inhibitors, such as novitoclax; Notch inhibitors, such as LY3039478 (crenigacestat), tarextumab (anti-Notch2/3), BMS-906024; hyaluronidase stimulators, such as PEGPH-20; Erbb2 tyrosine kinase receptor inhibitors/Hyaluronidase stimulators, such as Herceptin Hylecta; Wnt pathway inhibitors, such as SM-04755, PRI-724, WNT-974; gamma-secretase inhibitors, such as PF-03084014, MK-0752, RO-4929097; Grb-2 (growth factor receptor bound protein-2) inhibitors, such as BP1001; TRAIL pathway-inducing compounds, such as ONC201, ABBV-621; TRAIL modulators, such as SCB-313; Focal adhesion kinase inhibitors, such as VS-4718, defactinib, GSK2256098; hedgehog inhibitors, such as saridegib, sonidegib (LDE225), glasdegib; Aurora kinase inhibitors, such as alisertib (MLN-8237), and AZD-2811, AMG-900, barasertib, ENMD-2076; HSPB1 modulators (heat shock protein 27, HSP27), such as brivudine, apatorsen; ATR inhibitors, such as BAY-937, AZD6738, AZD6783, VX-803, VX-970 (berzosertib) and VX-970; Hsp90 inhibitors, such as AUY922, onalespib (AT13387), SNX-2112, SNX5422; murine double minute (mdm2) oncogene inhibitors, such as DS-3032b, RG7775, AMG-232, HDM201, and idasanutlin (RG7388); CD137 agonists, such as urelumab, utomilumab (PF-05082566), AGEN2373, ADG-106, BT-7480, QL1806; STING agonists, such as ADU-S100 (MIW-815), SB-11285, MK-1454, SR-8291, AdVCA0848, GSK-532, SYN-STING, MSA-1, SR-8291, GSK3745417; FGFR inhibitors, such as FGF-401, INCB-054828, BAY-1163877, AZD4547, JNJ-42756493, LY2874455, Debio-1347; fatty acid synthase (FASN) inhibitors, such as TVB-2640; CD44 binders, such as A6; protein phosphatease 2A (PP2A) inhibitors, such as LB-100; CYP17 inhibitors, such as seviteronel (VT-464), ASN-001, ODM-204, CFG920, abiraterone acetate; RXR agonists, such as IRX4204; hedgehog/smoothened (hh/Smo) antagonists, such as taladegib, patidegib, vismodegib; complement C3 modulators, such as Imprime PGG; IL-15 agonists, such as ALT-803, NKTR-255, interleukin-15/Fc fusion protein, AM-0015, NIZ-985, and hetIL-15; EZH2 (enhancer of zeste homolog 2) inhibitors, such as tazemetostat, CPI-1205, GSK-2816126, PF-06821497; oncolytic viruses, such as pelareorep, CG-0070, MV-NIS therapy, HSV-1716, DS-1647, VCN-01, ONCOS-102, TBI-1401, tasadenoturev (DNX-2401), vocimagene amiretrorepvec, RP-1, CVA21, Celyvir, LOAd-703, OBP-301, IMLYGIC®; DOT1L (histone methyltransferase) inhibitors, such as pinometostat (EPZ-5676); toxins such as Cholera toxin, ricin, Pseudomonas exotoxin, Bordetella pertussis adenylate cyclase toxin, diphtheria toxin, and caspase activators; DNA plasmids, such as BC-819; PLK inhibitors of PLK 1, 2, and 3, such as volasertib (PLK1); WEEl inhibitors, such as AZD-1775 (adavosertib); Rho kinase (ROCK) inhibitors, such as AT13148, KD025; Inhibition of Apoptosis Protein (IAP) inhibitors, such as ASTX660, debio-1143, birinapant, APG-1387, LCL-161; RNA polymerase inhibitors, such has lurbinectedin (PM-1183), CX-5461; Tubulin inhibitors, such as PM-184, BAL-101553 (lisavanbulin), and OXI-4503, fluorapacin (AC-0001), plinabulin, vinflunine; Toll-like receptor 4 (TLR-4) agonists, such as G100, GSK1795091, and PEPA-10; Elongation factor 1 alpha 2 inhibitors, such as plitidepsin; Elongation factor 2 inhibitors/Interleukin-2 ligands/NAD ADP ribosyltransferase stimulators, such as denileukin diftitox; CD95 inhibitors, such as APG-101, APO-010, asunercept; WT1 inhibitors, such as DSP-7888; splicing factor 3B subunit1 (SF3B1) inhibitors, such as H3B-8800; retinoid Z receptor gamma (RORγ) agonists, such as LYC-55716; and microbiome modulators, such as SER-401, EDP-1503, MRx-0518.

In some embodiments, an anti-CD47 agent or an anti-SIRPα agent as described herein, is co-administered with one or more additional therapeutic agents comprising an inhibitor or antagonist of: myeloid cell leukemia sequence 1 (MCL1) apoptosis regulator (NCBI Gene ID: 4170); mitogen-activated protein kinase 1 (MAP4K1) (also called Hematopoietic Progenitor Kinase 1 (HPK1), NCBI Gene ID: 11184); diacylglycerol kinase alpha (DGKA, DAGK, DAGK1 or DGK-alpha; NCBI Gene ID: 1606); 5′-nucleotidase ecto (NT5E or CD73; NCBI Gene ID: 4907); ectonucleoside triphosphate diphosphohydrolase 1 (ENTPD1 or CD39; NCBI Gene ID: 593); transforming growth factor beta 1 (TGFB1 or TGFβ; NCBI Gene ID: 7040); heme oxygenase 1 (HMOX1, HO-1 or HOl; NCBI Gene ID: 3162); heme oxygenase 2 (HMOX2, HO-2 or HO2; NCBI Gene ID: 3163); vascular endothelial growth factor A (VEGFA or VEGF; NCBI Gene ID: 7422); erb-b2 receptor tyrosine kinase 2 (ERBB2, HER2, HER2/neu or CD340; NCBI Gene ID: 2064), epidermal growth factor receptor (EGFR, ERBB, ERBB1 or HER1; NCBI Gene ID: 1956); ALK receptor tyrosine kinase (ALK, CD246; NCBI Gene ID: 238); poly(ADP-ribose) polymerase 1 (PARP1; NCBI Gene ID: 142); poly(ADP-ribose) polymerase 2 (PARP2; NCBI Gene ID: 10038); TCDD inducible poly(ADP-ribose) polymerase (TIPARP, PARP7; NCBI Gene ID: 25976); cyclin dependent kinase 4 (CDK4; NCBI Gene ID: 1019); cyclin dependent kinase 6 (CDK6; NCBI Gene ID: 1021); TNF receptor superfamily member 14 (TNFRSF14, HVEM, CD270; NCBI Gene ID: 8764); T cell immunoreceptor with Ig and ITIM domains (TIGIT; NCBI Gene ID: 201633); X-linked inhibitor of apoptosis (XIAP, BIRC4, IAP-3; NCBI Gene ID: 331); baculoviral IAP repeat containing 2 (BIRC2, cIAPI; NCBI Gene ID: 329); baculoviral IAP repeat containing 3 (BIRC3, cIAP2; NCBI Gene ID: 330); baculoviral IAP repeat containing 5 (BIRC5, surviving; NCBI Gene ID: 332); C-C motif chemokine receptor 2 (CCR2, CD192; NCBI Gene ID: 729230); C-C motif chemokine receptor 5 (CCR5, CD195; NCBI Gene ID: 1234); C-C motif chemokine receptor 8 (CCR8, CDw198; NCBI Gene ID: 1237); C—X-C motif chemokine receptor 2 (CXCR2, CD182; NCBI Gene ID: 3579); C—X-C motif chemokine receptor 3 (CXCR3, CD182, CD183; NCBI Gene ID: 2833); C—X-C motif chemokine receptor 4 (CXCR4, CD184; NCBI Gene ID: 7852); arginase (ARGI (NCBI Gene ID: 383), ARG2 (NCBI Gene ID: 384)), carbonic anhydrase (CA1 (NCBI Gene ID: 759), CA2 (NCBI Gene ID: 760), CA3 (NCBI Gene ID: 761), CA4 (NCBI Gene ID: 762), CA5A (NCBI Gene ID: 763), CA5B (NCBI Gene ID: 11238), CA6 (NCBI Gene ID: 765), CA7 (NCBI Gene ID: 766), CA8 (NCBI Gene ID: 767), CA9 (NCBI Gene ID: 768), CA10 (NCBI Gene ID: 56934), CA11 (NCBI Gene ID: 770), CA12 (NCBI Gene ID: 771), CA13 (NCBI Gene ID: 377677), CA14 (NCBI Gene ID: 23632)), prostaglandin-endoperoxide synthase 1 (PTGS1, COX-1; NCBI Gene ID: 5742), prostaglandin-endoperoxide synthase 2 (PTGS2, COX-2; NCBI Gene ID: 5743), secreted phospholipase A2, prostaglandin E synthase (PTGES, PGES; Gene ID: 9536), arachidonate 5-lipoxygenase (ALOX5, 5-LOX; NCBI Gene ID: 240) and/or soluble epoxide hydrolase 2 (EPHX2, SEH; NCBI Gene ID: 2053); a secreted phospholipase A2 (e.g., PLA2G1B (NCBI Gene ID: 5319); PLA2G7 (NCBI Gene ID: 7941), PLA2G3 (NCBI Gene ID: 50487), PLA2G2A (NCBI Gene ID: 5320); PLA2G4A (NCBI Gene ID: 5321); PLA2G12A (NCBI Gene ID: 81579); PLA2G12B (NCBI Gene ID: 84647); PLA2G10 (NCBI Gene ID: 8399); PLA2G5 (NCBI Gene ID: 5322); PLA2G2D (NCBI Gene ID: 26279); PLA2G15 (NCBI Gene ID: 23659)); indoleamine 2,3-dioxygenase 1 (IDO1; NCBI Gene ID: 3620); indoleamine 2,3-dioxygenase 2 (IDO2; NCBI Gene ID: 169355); hypoxia inducible factor 1 subunit alpha (HIF1A; NCBI Gene ID: 3091); angiopoietin 1 (ANGPT1; NCBI Gene ID: 284); Endothelial TEK tyrosine kinase (TIE-2, TEK, CD202B; NCBI Gene ID: 7010); Janus kinase 1 (JAK1; NCBI Gene ID: 3716); catenin beta 1 (CTNNBl; NCBI Gene ID: 1499); histone deacetylase 9 (HDAC9; NCBI Gene ID: 9734), and/or 5′-3′ exoribonuclease 1 (XRN1; NCBI Gene ID: 54464).

In various embodiments, additional agents, such as small molecules, antibodies, adoptive cellular therapies and chimeric antigen receptor T cells (CAR-T), checkpoint inhibitors, and vaccines, that are appropriate for treating hematological malignancies can be administered in combination with the anti-CD47 agents and the anti-CD20 agents described herein.

In various embodiments, an anti-CD47 agent or an anti-SIRPα agent as described herein, is combined with an agonist of fins related receptor tyrosine kinase 3 (FLT3); FLK2; STK1; CD135; FLK-2; NCBI Gene ID: 2322). Examples of FLT3 agonists include, but are not limited to, CDX-301 and GS-3583.

In various embodiments, an anti-CD47 agent or an anti-SIRPα agent as described herein, is combined with an anti-CD19 agent or antibody. Examples of anti-CD19 agents or antibodies that can be co-administered include without limitation: MOR00208, XmAb5574 (Xencor), AFM-11, Inebilizumab, MEDI 551 (Cellective Therapeutics); MDX-1342 (Medarexand) and blinatumomab (Amgen).

In various embodiments, an anti-CD47 agent or an anti-SIRPα agent as described herein, is combined with an anti-CD22 agent or antibody. Examples of anti-CD22 agents or antibodies that can be co-administered include without limitation: Epratuzumab, AMG-412, IMMU-103 (Immunomedics).

In various embodiments, an anti-CD47 agent or an anti-SIRPα agent as described herein, is combined with an anti-CD30 agent or antibody. Examples of anti-CD30 agents or antibodies that can be co-administered include without limitation: Brentuximab vedotin (Seattle Genetics).

In various embodiments, an anti-CD47 agent or an anti-SIRPα agent as described herein, is combined with an anti-CD33 agent or antibody. Examples of anti-CD33 agents or antibodies that can be co-administered include without limitation: CIK-CAR.CD33; CD33CART, AMG-330 (CD33/CD3), AMG-673 (CD33/CD3), GEM-333 (CD3/CD33), and IMGN-779.

In various embodiments, an anti-CD47 agent or an anti-SIRPα agent as described herein, is combined with an anti-CD37 agent or antibody. Examples of anti-CD37 agents or antibodies that can be co-administered include without limitation: B1836826 (Boehringer Ingelheim), Otlertuzumab, and TRU-016 (Trubion Pharmaceuticals).

In various embodiments, an anti-CD47 agent or an anti-SIRPα agent as described herein, is combined with an anti-CD38 agent or antibody. Examples of anti-CD38 agents or antibodies that can be co-administered include without limitation: CD38, such as T-007, UCART-38; Darzalex (Genmab), Daratumumab, JNJ-54767414 (Darzalex/Genmab), Isatuximab, SAR650984 (ImmunoGen), MOR202, MOR03087 (MorphoSys), TAK-079; and anti-CD38-attenukine, such as TAK573.

In various embodiments, an anti-CD47 agent or an anti-SIRPα agent as described herein, is combined with an anti-CD52 agent or antibody. Examples of anti-CD52 agents or antibodies that can be co-administered include without limitation: anti-CD52 antibodies, such as Alemtuzumab (Campath/University of Cambridge).

In various embodiments, an anti-CD47 agent or an anti-SIRPα agent as described herein, is combined with an anti-CD98 (4F2, FRP-1) agent or antibody. Examples of anti-CD98 agents or antibodies that can be co-administered include without limitation: IGN523 (Igenica).

In various embodiments, an anti-CD47 agent or an anti-SIRPα agent as described herein, is combined with an anti-CD157 (BST-1) agent or antibody. Examples of anti-CD157 agents or antibodies that can be co-administered include without limitation: OBT357, MEN1112 (Menarini; Oxford BioTherapeutics).

In various embodiments, an anti-CD47 agent or an anti-SIRPα agent as described herein, is combined with an anti-DKK-1 agent or antibody. Examples of anti-DKK-1 agents or antibodies that can be co-administered include without limitation: BHQ880 (MorphoSys; Novartis), and DKN-01, LY-2812176 (Eli Lilly).

In various embodiments, an anti-CD47 agent or an anti-SIRPα agent as described herein, is combined with an anti-GRP78 (BiP) agent or antibody. Examples of anti-GRP78 agents or antibodies that can be co-administered include without limitation: PAT-SM6 (OncoMab GmbH).

In various embodiments, an anti-CD47 agent or an anti-SIRPα agent as described herein, is combined with an anti-NOTCH1 agent or antibody. Examples of anti-NOTCH1 agents or antibodies that can be co-administered include without limitation: Brontictuzumab, OMP-52M51 (OncoMed Pharmaceuticals).

In various embodiments, an anti-CD47 agent or an anti-SIRPα agent as described herein, is combined with an anti-ROR1 agent or antibody. Examples of anti-ROR1 agents or antibodies that can be co-administered include without limitation: Mapatumumab, TRM1, and HGS-1012 (Cambridge Antibody Technology).

In various embodiments, an anti-CD47 agent or an anti-SIRPα agent as described herein, is combined with an anti-SLAMF7 (CS1, CD319) agent or antibody. Examples of anti-SLAMF7 agents or antibodies that can be co-administered include without limitation: Elotuzumab, HuLuc63, BMS-901608 (Empliciti/PDL BioPharma), Mogamulizumab (KW-0761).

In various embodiments, an anti-CD47 agent or an anti-SIRPα agent as described herein, is combined with an anti-TNFRSF10A (DR4; APO2; CD261; TRAILR1; TRAILR-1) agent or antibody. Examples of anti-TNFRSF10A agents or antibodies that can be co-administered include without limitation: Mapatumumab, TRM1, and HGS-1012 (Cambridge Antibody Technology).

In various embodiments, an anti-CD47 agent or an anti-SIRPα agent as described herein, is combined with an anti-Transferrin Receptor (TFRC; CD71) agent or antibody. Examples of anti-Transferrin Receptor agents or antibodies that can be co-administered include without limitation: E2.3/A27.15 (University of Arizona).

In various embodiments, an anti-CD47 agent or an anti-SIRPα agent as described herein, is combined with an anti-EPHA3 agent or antibody. Examples of anti-EPHA3 agents or antibodies that can be co-administered include without limitation: Ifabotuzumab, KB004 (Ludwig Institute for Cancer Research).

In various embodiments, an anti-CD47 agent or an anti-SIRPα agent as described herein, is combined with an anti-CCR4 agent or antibody. Examples of anti-CCR4 agents or antibodies that can be co-administered include without limitation: Mogamulizumab, KW-0761 (Poteligeo/Kyowa Hakko Kirin Co.)

In various embodiments, an anti-CD47 agent or an anti-SIRPα agent as described herein, is combined with an anti-CXCR4 agent or antibody. Examples of anti-CXCR4 agents or antibodies that can be co-administered include without limitation: Ulocuplumab, BMS-936564, MDX-1338 (Medarex), and PF-06747143 (Pfizer).

In various embodiments, an anti-CD47 agent or an anti-SIRPα agent as described herein, is combined with an anti-BAFF agent or antibody. Examples of anti-BAFF agents or antibodies that can be co-administered include without limitation: Tabalumab, LY2127399 (Eli Lilly).

In various embodiments, an anti-CD47 agent or an anti-SIRPα agent as described herein, is combined with an anti-BAFF Receptor (BAFF-R) agent or antibody. Examples of anti-BAFF-R agents or antibodies that can be co-administered include without limitation: VAY736 (MorphoSys; Novartis)

In various embodiments, an anti-CD47 agent or an anti-SIRPα agent as described herein, is combined with an anti-RANKL agent or antibody. Examples of anti-RANKL agents or antibodies that can be co-administered include without limitation: Denosumab, AMG-162 (Prolia; Ranmark; Xgeva/Amgen).

In various embodiments, an anti-CD47 agent or an anti-SIRPα agent as described herein, is combined with an anti-IL-6 agent or antibody. Examples of anti-IL-6 agents or antibodies that can be co-administered include without limitation: Siltuximab, CNTO-328 (Sylvant/Centocor).

In various embodiments, an anti-CD47 agent or an anti-SIRPα agent as described herein, is combined with an anti-IL-6 Receptor (IL-6R) agent or antibody. Examples of anti-IL-6R agents or antibodies that can be co-administered include without limitation: Tocilizumab, R-1569 (Actemra/Chugai Pharmaceutical; Osaka University), or AS-101 (CB-06-02, IVX-Q-101).

In various embodiments, an anti-CD47 agent or an anti-SIRPα agent as described herein, is combined with an anti-IL3RA (CD123) agent or antibody. Examples of anti-IL3RA (CD123) agents or antibodies that can be co-administered include without limitation: CSL360 (CSL), Talacotuzumab, JNJ-56022473, CSL362 (CSL); XmAb14045 (Xencor); KHK2823 (KyowaHakko Kirin Co.); APV0436 (CD123/CD3); flotetuzumab (CD123/CD3); JNJ-63709178 (CD123/CD3); and XmAb-14045 (CD123/CD3) (Xencor).

In various embodiments, an anti-CD47 agent or an anti-SIRPα agent as described herein, is combined with an anti-IL2RA (CD25) agent or antibody. Examples of anti-IL2RA agents or antibodies that can be co-administered include without limitation: Basiliximab, SDZ-CHI-621 (Simulect/Novartis), and Daclizumab.

In various embodiments, an anti-CD47 agent or an anti-SIRPα agent as described herein, is combined with an anti-IGF-1R (CD221) agent or antibody. Examples of anti-IGF-1R agents or antibodies that can be co-administered include without limitation: Ganitumab, AMG-479 (Amgen); Ganitumab, AMG-479 (Amgen), Dalotuzumab, MK-0646 (Pierre Fabre), and AVE1642 (ImmunoGen).

In various embodiments, an anti-CD47 agent or an anti-SIRPα agent as described herein, is combined with an anti-GM-CSF (CSF2) agent or antibody. Examples of anti-GM-CSF agents or antibodies that can be co-administered include without limitation: Lenzilumab, KB003 (KaloBios Pharmaceuticals).

In various embodiments, an anti-CD47 agent or an anti-SIRPα agent as described herein, is combined with an anti-HGF agent or antibody. Examples of anti-HGF agents or antibodies that can be co-administered include without limitation: Ficlatuzumab, AV-299 (AVEO Pharmaceuticals).

In various embodiments, an anti-CD47 agent or an anti-SIRPα agent as described herein, is combined with an anti-CD44 agent or antibody. Examples of anti-CD44 agents or antibodies that can be co-administered include without limitation: RG7356, R05429083 (Chugai Biopharmaceuticals; Roche).

In various embodiments, an anti-CD47 agent or an anti-SIRPα agent as described herein, is combined with an anti-VLA-4 (CD49d) agent or antibody. Examples of anti-VLA-4 agents or antibodies that can be co-administered include without limitation: Natalizumab, BG-0002-E (Tysabri/Elan Corporation).

In various embodiments, an anti-CD47 agent or an anti-SIRPα agent as described herein, is combined with an anti-ICAM-1 (CD54) agent or antibody. Examples of anti-ICAM-1 agents or antibodies that can be co-administered include without limitation: BI-505 (BioInvent International)

In various embodiments, an anti-CD47 agent or an anti-SIRPα agent as described herein, is combined with an anti-VEGF-A agent or antibody. Examples of anti-VEGF-A agents or antibodies that can be co-administered include without limitation: Bevacizumab (Avastin/Genentech; Hackensack University Medical Center).

In various embodiments, an anti-CD47 agent or an anti-SIRPα agent as described herein, is combined with an anti-Endosialin (CD248, TEM1) agent or antibody. Examples of antiEndosialin agents or antibodies that can be co-administered include without limitation: Ontecizumab, MORAB-004 (Ludwig Institute for Cancer Research; Morphotek).

In various embodiments, an anti-CD47 agent or an anti-SIRPα agent as described herein, is combined with an anti-CD79 agent or antibody. Examples of anti-CD79 agents or antibodies that can be co-administered include without limitation: polatuzumab, DCDS4501A, RG7596 (Genentech).

In various embodiments, an anti-CD47 agent or an anti-SIRPα agent as described herein, is combined with an anti-Isocitrate dehydrogenase (IDH) agent or antibody. Examples of anti-IDH agents or antibodies that can be co-administered include without limitation: IDH1 inhibitor ivosidenib (Tibsovo; Agios) and the IDH2 inhibitor enasidenib (Idhifa; Celgene/Agios).

In various embodiments, an anti-CD47 agent or an anti-SIRPα agent as described herein, is combined with an antibody that targets tumor associated calcium signal transducer 2 (TACSTD2) (NCBI Gene ID: 4070; EGP-1, EGP1, GA733-1, GA7331, GP50, M1S1, TROP2), such as sacituzumab.

In various embodiments, an anti-CD47 agent or an anti-SIRPα agent as described herein, is combined with an anti-major histocompatibility complex, class I, G (HLA-G; NCBI Gene ID: 3135) antibody, such as TTX-080.

In various embodiments, an anti-CD47 agent or an anti-SIRPα agent as described herein, is combined with an anti-leukocyte immunoglobulin like receptor B2 (LILRB2, a.k.a., CD85D, ILT4; NCBI Gene ID: 10288) antibody, such as JTX-8064 or MK-4830.

### TNF Receptor Superfamily (TNFRSF) Member Azonists or Activators

In various embodiments, an anti-CD47 agent or an anti-SIRPα agent as described herein, is combined with an agonist of one or more TNF receptor superfamily (TNFRSF) members, e.g., an agonist of one or more of TNFRSF1A (NCBI Gene ID: 7132), TNFRSF1B (NCBI Gene ID: 7133), TNFRSF4 (OX40, CD134; NCBI Gene ID: 7293), TNFRSF5 (CD40; NCBI Gene ID: 958), TNFRSF6 (FAS, NCBI Gene ID: 355), TNFRSF7 (CD27, NCBI Gene ID: 939), TNFRSF8 (CD30, NCBI Gene ID: 943), TNFRSF9 (4-1BB, CD137, NCBI Gene ID: 3604), TNFRSF10A (CD261, DR4, TRAILR1, NCBI Gene ID: 8797), TNFRSF10B (CD262, DR5, TRAILR2, NCBI Gene ID: 8795), TNFRSF10C (CD263, TRAILR3, NCBI Gene ID: 8794), TNFRSF10D (CD264, TRAILR4, NCBI Gene ID: 8793), TNFRSF11A (CD265, RANK, NCBI Gene ID: 8792), TNFRSF11B (NCBI Gene ID: 4982), TNFRSF12A (CD266, NCBI Gene ID: 51330), TNFRSF13B (CD267, NCBI Gene ID: 23495), TNFRSF13C (CD268, NCBI Gene ID: 115650), TNFRSF16 (NGFR, CD271, NCBI Gene ID: 4804), TNFRSF17 (BCMA, CD269, NCBI Gene ID: 608), TNFRSF18 (GITR, CD357, NCBI Gene ID: 8784), TNFRSF19 (NCBI Gene ID: 55504), TNFRSF21 (CD358, DR6, NCBI Gene ID: 27242), and TNFRSF25 (DR3, NCBI Gene ID: 8718).

Examples anti-TNFRSF4 (OX40) antibodies that can be co-administered include without limitation, MEDI6469, MEDI6383, MEDI0562 (tavolixizumab), MOXR0916, PF-04518600, RG-7888, GSK-3174998, INCAGN1949, BMS-986178, GBR-8383, ABBV-368, and those described in WO2016179517, WO2017096179, WO2017096182, WO2017096281, and WO2018089628, each of which is hereby incorporated by reference in its entirety.

Examples anti-TNF receptor superfamily member 10b (TNFRSF10B, DR5, TRAILR2) antibodies that can be co-administered include without limitation, such as DS-8273, CTB-006, INBRX-109, and GEN-1029.

Examples of anti-TNFRSF5 (CD40) antibodies that can be co-administered include, without limitation, selicrelumab (R07009789), mitazalimab (a.k.a., vanalimab), ADC-1013, JNJ-64457107), RG7876, SEA-CD40, APX-005M and ABBV-428, ABBV-927, and JNJ-64457107.

Examples of anti-TNFRSF7 (CD27) that can be co-administered include without limitation varlilumab (CDX-1127).

Examples of anti-TNFRSF9 (4-1BB, CD137) antibodies that can be co-administered include without limitation urelumab, utomilumab (PF-05082566), AGEN2373, ADG-106, BT-7480, and QL1806.

Examples of anti-TNFRSF17 (BCMA) that can be co-administered include without limitation GSK-2857916.

Examples of anti-TNFRSF18 (GITR) antibodies that can be co-administered include without limitation, MEDI1873, FPA-154, INCAGN-1876, TRX-518, BMS-986156, MK-1248, GWN-323, and those described in WO2017096179, WO2017096276, WO2017096189, and WO2018089628. In some embodiments, an antibody, or fragment thereof, co-targeting TNFRSF4 (OX40) and TNFRSF18 (GITR) is co-administered. Such antibodies are described, e.g., in WO2017096179 and WO2018089628, each of which is hereby incorporated by reference in its entirety.

Example anti-TRAILR1, anti-TRAILR2, anti-TRAILR3, anti-TRAILR4 antibodies that can be co-administered include without limitation ABBV-621.

Examples of Bi-specific antibodies targeting TNFRSF family members that can be co-administered include without limitation PRS-343 (CD-137/HER2), AFM26 (BCMA/CD16A), AFM-13 (CD16/CD30), REGN-1979 (CD20/CD3), AMG-420 (BCMA/CD3), INHIBRX-105 (4-1BB/PDL1), FAP-4-IBBL (4-1BB/FAP), XmAb-13676 (CD3/CD20), RG-7828 (CD20/CD3), CC-93269 (CD3/BCMA), REGN-5458 (CD3/BCMA), and IMM-0306 (CD47/CD20), and AMG-424 (CD38.CD3).

Examples of inhibitors of PVR related immunoglobulin domain containing (PVRIG, CD112R) that can be co-administered include without limitation: COM-701.

Examples of inhibitors of T cell immunoreceptor with Ig and ITIM domains (TIGIT; NCBI Gene ID: 201633) that can be co-administered include without limitation: BMS-986207, RG-6058, AGEN-1307, COM-902, etigilimab, tiragolumab (a.k.a., MTIG-7192A; RG-6058; RO 7092284), AGEN1777, IBI-939, AB154, MG1131 and EOS884448 (EOS-448).

Examples of inhibitors of hepatitis A virus cellular receptor 2 (HAVCR2, TIMD3, TIM-3) that can be co-administered include without limitation: TSR-022, LY-3321367, MBG-453, INCAGN-2390, RO-7121661 (PD-1/TIM-3), LY-3415244 (TIM-3/PDL1), and RG7769 (PD-1/TIM-3).

Examples of inhibitors of lymphocyte activating 3 (LAG-3, CD223) that can be co-administered include without limitation: relatlimab (ONO-4482), LAG-525, MK-4280, REGN-3767, INCAGN2385, TSR-033, MGD-013 (PD-1/LAG-3), and FS-118 (LAG-3/PD-L1).

Examples of anti-killer cell immunoglobulin like receptor, three Ig domains and long cytoplasmic tail 1 (KIR3DL1; KIR; NCBI Gene ID: 3811) monoclonal antibodies, such as lirilumab (IPH-2102), and IPH-4102.

Examples of anti-NKG2a antibodies that can be co-administered include without limitation: monalizumab.

Examples of anti-V-set immunoregulatory receptor (VSIR, B7H5, VISTA) antibodies that can be co-administered include without limitation: HMBD-002, and CA-170 (PD-L1/VISTA).

Examples of anti-CD70 antibodies that can be co-administered include without limitation: AMG-172.

Examples of anti-ICOS antibodies that can be co-administered include without limitation: JTX-2011, GSK3359609.

Examples of ICOS agonists that can be co-administered include without limitation: ICOS-L.COMP (Gariepy, J. et al. 106th Annu Meet Am Assoc Immunologists (AAI) (May 9-13, San Diego) 2019, Abst 71.5).

Immune Checkpoint Inhibitors

In some embodiments, an anti-CD47 agent or an anti-SIRPα agent as described herein, is combined with one or more immune checkpoint inhibitors. In some embodiments, the one or more immune checkpoint inhibitors is a proteinaceous (e.g., antibody or fragment thereof, or antibody mimetic) inhibitor of PD-L1 (CD274), PD-1 (PDCD1) or CTLA4. In some embodiments, the one or more immune checkpoint inhibitors comprises a small organic molecule inhibitor of PD-L1 (CD274), PD-1 (PDCD1) or CTLA4.

Examples of inhibitors of CTLA4 that can be co-administered include without limitation ipilimumab, tremelimumab, BMS-986218, AGEN1181, AGEN1884, BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, BPI-002, HBM-4003, as well as multi-specific inhibitors FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4), and AK-104 (CTLA4/PD-1).

Examples of inhibitors/antibodies of PD-L1 (CD274) or PD-1 (PDCD1) that can be co-administered include without limitation pembrolizumab, nivolumab, cemiplimab, pidilizumab, AMG-404, AMP-224, MEDI0680 (AMP-514), spartalizumab, atezolizumab, avelumab, durvalumab, BMS-936559, CK-301, PF-06801591, BGB-A317 (tislelizumab), GEN-1046 (PD-L1/4-1BB), GLS-010 (WBP-3055), AK-103 (HX-008), AK-105, CS-1003, HLX-10, MGA-012, BI-754091, AGEN-2034, JS-001 (toripalimab), JNJ-63723283, genolimzumab (CBT-501), LZM-009, BCD-100, LY-3300054, SHR-1201, SHR-1210 (camrelizumab), Sym-021, ABBV-181, PD1-PIK, BAT-1306, (MSB0010718C), CX-072, CBT-502, TSR-042 (dostarlimab), MSB-2311, JTX-4014, BGB-A333, SHR-1316, CS-1001 (WBP-3155, KN-035, IBI-308 (sintilimab), HLX-20, KL-A167, STI-A1014, STI-A1015 (IMC-001), BCD-135, FAZ-053, TQB-2450, MDX1105-01, GS-4224, GS-4416, INCB086550, MAX10181, as well as multi-specific inhibitors FPT-155 (CTLA4/PD-L1/CD28), PF-06936308 (PD-1/CTLA4), MGD-013 (PD-1/LAG-3), RO-7247669 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFβ-EC domain), CA-170 (PD-L1/VISTA), CDX-527 (CD27/PD-L1), LY-3415244 (TIM-3/PDL1), RG7769 (PD-1/TIM-3) and INBRX-105 (4-1BB/PDL1), GNS-1480 (PD-L1/EGFR), RG-7446 (Tecentriq, atezolizumab), ABBV-181, nivolumab (OPDIVO®, BMS-936558, MDX-1106), pembrolizumab (KEYTRUDA®, MK-3477, SCH-900475, lambrolizumab, CAS Reg. No. 1374853-91-4), pidilizumab, PF-06801591, BGB-A317 (tislelizumab), GLS-010 (WBP-3055), AK-103 (HX-008), CS-1003, HLX-10, MGA-012, BI-754091, REGN-2810 (cemiplimab), AGEN-2034, JS-001 (toripalimab), JNJ-63723283, genolimzumab (CBT-501), LZM-009, BCD-100, LY-3300054, SHR-1201, SHR-1210 (camrelizumab), Sym-021, ABBV-181, AK-105, PD1-PIK, BAT-1306, BMS-936559, atezolizumab (MPDL3280A), durvalumab (MEDI-4736), avelumab, CK-301, (MSB0010718C), MEDI-0680, CX-072, CBT-502, PDR-001 (spartalizumab), PDR001+Tafinlar®+Mekinist®, MSB-2311, JTX-4014, BGB-A333, SHR-1316, CS-1001 (WBP-3155, KN-035, IBI-308 (sintilimab), HLX-20, KL-A167, STI-A1014, STI-A1015 (IMC-001), BCD-135, FAZ-053, TQB-2450, MDX1105-01, and those described, e.g., in Intl. Patent Publ. Nos. WO2018195321, WO2020014643, WO2019160882, and WO2018195321.

In various embodiments, an anti-CD47 agent as described herein, is combined with an inhibitor of MCL1 apoptosis regulator, BCL2 family member (MCL1, TM; EAT; MCL1L; MCL1S; Mcl-1; BCL2L3; MCL1-ES; bcl2-L-3; mcl1/EAT; NCBI Gene ID: 4170). Examples of MCL1 inhibitors include AMG-176, AMG-397, S-64315, and AZD-5991, 483-LM, A-1210477, UMI-77, JKY-5-037, and those described in WO2018183418, WO2016033486, and WO2017147410.

Toll-Like Receptor (TLR) Agonists

In various embodiments, an anti-CD47 agent or an anti-SIRPα agent as described herein, is combined with an agonist of a toll-like receptor (TLR), e.g., an agonist of TLR1 (NCBI Gene ID: 7096), TLR2 (NCBI Gene ID: 7097), TLR3 (NCBI Gene ID: 7098), TLR4 (NCBI Gene ID: 7099), TLR5 (NCBI Gene ID: 7100), TLR6 (NCBI Gene ID: 10333), TLR7 (NCBI Gene ID: 51284), TLR8 (NCBI Gene ID: 51311), TLR9 (NCBI Gene ID: 54106), and/or TLR10 (NCBI Gene ID: 81793). Example TLR7 agonists that can be co-administered include without limitation DS-0509, GS-9620, LHC-165, TMX-101 (imiquimod), GSK-2245035, resiquimod, DSR-6434, DSP-3025, IMO-4200, MCT-465, MEDI-9197, 3M-051, SB-9922, 3M-052, Limtop, TMX-30X, TMX-202, RG-7863, RG-7795, and the compounds disclosed in US20100143301 (Gilead Sciences), US20110098248 (Gilead Sciences), and US20090047249 (Gilead Sciences), US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). An TLR7/TLR8 agonist that can be co-administered is NKTR-262. Example TLR8 agonists that can be co-administered include without limitation E-6887, IMO-4200, IMO-8400, IMO-9200, MCT-465, MEDI-9197, motolimod, resiquimod, GS-9688, VTX-1463, VTX-763, 3M-051, 3M-052, and the compounds disclosed in US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). Example TLR9 agonists that can be co-administered include without limitation AST-008, CMP-001, IMO-2055, IMO-2125, litenimod, MGN-1601, BB-001, BB-006, IMO-3100, IMO-8400, IR-103, IMO-9200, agatolimod, DIMS-9054, DV-1079, DV-1179, AZD-1419, leftolimod (MGN-1703), CYT-003, CYT-003-QbG10 and PUL-042. Examples of TLR3 agonist include rintatolimod, poly-ICLC, RIBOXXON®, Apoxxim, RIBOXXIM®, IPH-33, MCT-465, MCT-475, and ND-1.1.

Examples of TLR8 inhibitors include, but are not limited to, E-6887, IMO-8400, IMO-9200 and VTX-763.

Examples of TLR8 agonists include, but are not limited to, MCT-465, motolimod, GS-9688, and VTX-1463.

Examples of TLR9 inhibitors include but are not limited to, AST-008, IMO-2055, IMO-2125, lefitolimod, litenimod, MGN-1601, and PUL-042.

Examples of TLR7/TLR8 agonist, such as NKTR-262, IMO-4200, MEDI-9197 (telratolimod), resiquimod;

Examples of TLR agonists include without limitation: lefitolimod, tilsotolimod, rintatolimod, DSP-0509, AL-034, G-100, cobitolimod, AST-008, motolimod, GSK-1795091, GSK-2245035, VTX-1463, GS-9688, LHC-165, BDB-001, RG-7854, telratolimod.

In some embodiments, the therapeutic agent is a stimulator of interferon genes (STING) In some embodiments, the STING receptor agonist or activator is selected from the group consisting of ADU-S100 (MIW-815), SB-11285, MK-1454, SR-8291, AdVCA0848, GSK-532, SYN-STING, MSA-1, SR-8291, 5,6-dimethylxanthenone-4-acetic acid (DMXAA), cyclic-GAMP (cGAMP), and cyclic-di-AMP.

### TCR Signaling Modulators

In various embodiments, an anti-CD47 agent or an anti-SIRPα agent as described herein, is combined with one or more agonist or antagonist of T-Cell Receptor (TCR) signaling modulators. Activation of T cells through the TCR and is essential for thymocyte development and effector T cell function. TCR activation promotes signaling cascades that ultimately determine cell fate through regulating cytokine production, cell survival, proliferation, and differentiation. Examples of TCR signaling modulators include without limitation CD2 (cluster of differentiation 2, LFA-2, T11, LFA-3 receptor), CD3 (cluster of differentiation 3), CD4 (cluster of differentiation 4), CD8 (cluster of differentiation 8), CD28 (cluster of differentiation 28), CD45 (PTPRC, B220, GP180), LAT (Linker for activation of T cells, LAT1), Lck, LFA-1 (ITGB2, CD18, LAD, LCAMB), Src, Zap-70, SLP-76, DGKalpha, CBL-b, CISH, HPK1. Examples of agonist of cluster of differentiation 3 (CD3) that can be co-administered include without limitation MGD015.

In various embodiments, an anti-CD47 agent or an anti-SIRPα agent as described herein, is combined with one or more blockers or inhibitors of inhibitory immune checkpoint proteins or receptors and/or with one or more stimulators, activators or agonists of one or more stimulatory immune checkpoint proteins or receptors. Blockade or inhibition of inhibitory immune checkpoints can positively regulate T-cell or NK cell activation and prevent immune escape of cancer cells within the tumor microenvironment. Activation or stimulation of stimulatory immune check points can augment the effect of immune checkpoint inhibitors in cancer therapeutics. In various embodiments, the immune checkpoint proteins or receptors regulate T cell responses (e.g., reviewed in Xu, et al., J Exp Clin Cancer Res. (2018) 37:110). In various embodiments, the immune checkpoint proteins or receptors regulate NK cell responses (e.g., reviewed in Davis, et al., Semin Immunol. (2017) 31:64-75 and Chiossone, et al., Nat Rev Immunol. (2018) 18(11):671-688).

Examples of immune checkpoint proteins or receptors include without limitation CD27, CD70; CD40, CD40LG; CD47, CD48 (SLAMF2), transmembrane and immunoglobulin domain containing 2 (TMIGD2, CD28H), CD84 (LY9B, SLAMF5), CD96, CD160, MS4A1 (CD20), CD244 (SLAMF4); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); natural killer cell cytotoxicity receptor 3 ligand 1 (NCR3LG1, B7H6); HERV-H LTR-associating 2 (HHLA2, B7H7); inducible T cell co-stimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF8 (CD30), TNFSF8 (CD30L); TNFRSF10A (CD261, DR4, TRAILR1), TNFRSF9 (CD137), TNFSF9 (CD137L); TNFRSF10B (CD262, DR5, TRAILR2), TNFRSF10 (TRAIL); TNFRSF14 (HVEM, CD270), TNFSF14 (HVEML); CD272 (B and T lymphocyte associated (BTLA)); TNFRSF17 (BCMA, CD269), TNFSF13B (BAFF); TNFRSF18 (GITR), TNFSF18 (GITRL); MHC class I polypeptide-related sequence A (MICA); MHC class I polypeptide-related sequence B (MICB); CD274 (PDL1, PD-L1); programmed cell death 1 (PDCD1, PD-1, PD-1); cytotoxic T-lymphocyte associated protein 4 (CTLA4, CD152); CD80 (B7-1), CD28; nectin cell adhesion molecule 2 (NECTIN2, CD112); CD226 (DNAM-1); Poliovirus receptor (PVR) cell adhesion molecule (PVR, CD155); T cell immunoreceptor with Ig and ITIM domains (TIGIT); T cell immunoglobulin and mucin domain containing 4 (TIMD4; TIM4); hepatitis A virus cellular receptor 2 (HAVCR2, TIMD3, TIM-3); galectin 9 (LGALS9); lymphocyte activating 3 (LAG-3, CD223); signaling lymphocytic activation molecule family member 1 (SLAMF1, SLAM, CD150); lymphocyte antigen 9 (LY9, CD229, SLAMF3); SLAM family member 6 (SLAMF6, CD352); SLAM family member 7 (SLAMF7, CD319); UL16 binding protein 1 (ULBP1); UL16 binding protein 2 (ULBP2); UL16 binding protein 3 (ULBP3); retinoic acid early transcript 1E (RAET1E; ULBP4); retinoic acid early transcript 1G (RAET1G; ULBP5); retinoic acid early transcript 1L (RAET1L; ULBP6); lymphocyte activating 3 (CD223); killer cell immunoglobulin like receptor (KIR); killer cell lectin like receptor C1 (KLRC1, NKG2A, CD59A); killer cell lectin like receptor KI (KLRK1, NKG2D, CD314); killer cell lectin like receptor C2 (KLRC2, CD159c, NKG2C); killer cell lectin like receptor C3 (KLRC3, NKG2E); killer cell lectin like receptor C4 (KLRC4, NKG2F); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor D1 (KLRD1.

In various embodiments, an anti-CD47 agent or an anti-SIRPα agent as described herein, is combined with one or more blockers or inhibitors of one or more T-cell inhibitory immune checkpoint proteins or receptors. Illustrative T-cell inhibitory immune checkpoint proteins or receptors include without limitation CD274 (PDL1, PD-L1); programmed cell death 1 ligand 2 (PDCD1LG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CDT112R); T cell immunoreceptor with Ig and ITIM domains (TIGIT); lymphocyte activating 3 (LAG-3, CD223); hepatitis A virus cellular receptor 2 (HAVCR2, TIMD3, TIM-3); galectin 9 (LGALS9); killer cell immunoglobulin like receptor (KIR); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1). In various embodiments, an anti-CD47 agent or an anti-SIRPα agent as described herein, is combined with one or more agonist or activators of one or more T-cell stimulatory immune checkpoint proteins or receptors. Illustrative T-cell stimulatory immune checkpoint proteins or receptors include without limitation CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); CD244 (2B4, SLAMF4), Poliovirus receptor (PVR) cell adhesion molecule (PVR, CD155). See, e.g., Xu, et al., J Exp Clin Cancer Res. (2018) 37:110.

In various embodiments, an anti-CD47 agent or an anti-SIRPα agent as described herein, is combined with one or more blockers or inhibitors of one or more NK-cell inhibitory immune checkpoint proteins or receptors. Illustrative NK-cell inhibitory immune checkpoint proteins or receptors include without limitation killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor C1 (KLRC1, NKG2A, CD159A); and killer cell lectin like receptor D1 (KLRD1, CD94).

In various embodiments, an anti-CD47 agent or an anti-SIRPα agent as described herein, is combined with one or more agonist or activators of one or more NK-cell stimulatory immune checkpoint proteins or receptors. Illustrative NK-cell stimulatory immune checkpoint proteins or receptors include without limitation CD16, CD226 (DNAM-1); CD244 (2B4, SLAMF4); killer cell lectin like receptor K (KLRK1, NKG2D, CD314); SLAM family member 7 (SLAMF7). See, e.g., Davis, et al., Semin Immunol. (2017) 31:64-75; Fang, et al., Semin Immunol. (2017) 31:37-54; and Chiossone, et al., Nat Rev Immunol. (2018) 18(11):671-688.

Adenosine Generation and Signaling

In various embodiments, an anti-CD47 agent or an anti-SIRPα agent as described herein, is combined with an agonist or antagonist of AIR, A2AR, A2BR, A3R, CD73, CD39, CD26; e.g., Adenosine A3 receptor (A3R) agonists, such as namodenoson (CF102); A2aR/A2bR antagonists, such as AB928; anti-CD73 antibodies, such as MEDI-9447 (oleclumab), CPX-006, IPH-53, BMS-986179, NZV-930, CPI-006; CD73 inhibitors, such as AB-680, PSB-12379, PSB-12441, PSB-12425, CB-708, and those described in Int Patent Publication No. WO19173692; CD39/CD73 inhibitors, such as PBF-1662; anti-CD39 antibodies, such as TTX-030; adenosine A2A receptor antagonists, such as CPI-444, AZD-4635, preladenant, PBF-509; and adenosine deaminase inhibitors, such as pentostatin, cladribine.

Bi-Specific T-Cell Engagers

In various embodiments, an anti-CD47 agent or an anti-SIRPα agent as described herein, is combined with a bi-specific T-cell engager (e.g., not having an Fc) or an anti-CD3 bi-specific antibody (e.g., having an Fc). Illustrative anti-CD3 bi-specific antibodies or BiTEs that can be co-administered include AMG-160 (PSMA/CD3), AMG-212 (PSMA/CD3), AMG-330 (CD33/CD3), AMG-420 (BCMA/CD3), AMG-427 (FLT3/CD3), AMG-562 (CD19/CD3), AMG-596 (EGFRvIII/CD3), AMG-701 (BCMA/CD3), AMG-757 (DLL3/CD3), JNJ-64052781 (CD19/CD3), AMG-211 (CEA/CD3), BLINCYTO® (CD19/CD3), RG7802 (CEA/CD3), ERY-974 (CD3/GPC3), huGD2-BsAb (CD3/GD2), PF-06671008 (Cadherins/CD3), APV0436 (CD123/CD3), ERY974, flotetuzumab (CD123/CD3), GEM333 (CD3/CD33), GEMoab (CD3/PSCA), REGN-1979 (CD20/CD3), REGN-5678 (PSMA/CD28), MCLA-117 (CD3/CLEC12A), JNJ-0819, JNJ-7564 (CD3/heme), JNJ-63709178 (CD123/CD3), MGD-007 (CD3/gpA33), MGD-009 (CD3/B7H3), IMCgp100 (CD3/gp100), XmAb-14045 (CD123/CD3), XmAb-13676 (CD3/CD20), XmAb-18087 (SSTR2/CD3), catumaxomab (CD3/EpCAM), REGN-4018 (MUC16/CD3), RG6026, RG6076, RG6194, RG-7828 (CD20/CD3), CC-93269 (CD3/BCMA), REGN-5458 (CD3/BCMA), GRB-1302 (CD3/Erbb2), GRB-1342 (CD38/CD3), PF-06863135 (BCMA/CD3), SAR440234 (CD3/CDw123). As appropriate, the anti-CD3 binding bi-specific molecules may or may not have an Fc. Illustrative bi-specific T-cell engagers that can be co-administered target CD3 and a tumor-associated antigen as described herein, including, e.g., CD19 (e.g., blinatumomab); CD33 (e.g., AMG330); CEA (e.g., MEDI-565); receptor tyrosine kinase-like orphan receptor 1 (ROR1) (Gohil, et al., Oncoimmunology. (2017) May 17; 6(7):e1326437); PD-L1 (Horn, et al., Oncotarget. 2017 Aug. 3; 8(35):57964-57980); and EGFRvIII (Yang, et al., Cancer Lett. 2017 Sep. 10; 403:224-230).

Bi- and Tri-Specific Natural Killer (NK)-Cell Engagers

In various embodiments, an anti-CD47 agent or an anti-SIRPα agent as described herein, is combined with a bi-specific NK-cell engager (BiKE) or a tri-specific NK-cell engager (TriKE) (e.g., not having an Fc) or bi-specific antibody (e.g., having an Fc) against an NK cell activating receptor, e.g., CD16A, C-type lectin receptors (CD94/NKG2C, NKG2D, NKG2E/H and NKG2F), natural cytotoxicity receptors (NKp30, NKp44 and NKp46), killer cell C-type lectin-like receptor (NKp65, NKp80), Fc receptor FcγR (which mediates antibody-dependent cell cytotoxicity), SLAM family receptors (e.g., 2B4, SLAM6 and SLAM7), killer cell immunoglobulin-like receptors (KIR) (KIR-2DS and KIR-3DS), DNAM-1 and CD137 (41BB). Illustrative anti-CD16 bi-specific antibodies, BiKEs or TriKEs that can be co-administered include AFM26 (BCMA/CD16A) and AFM-13 (CD16/CD30). As appropriate, the anti-CD16 binding bi-specific molecules may or may not have an Fc. Illustrative bi-specific NK-cell engagers that can be co-administered target CD16 and one or more tumor-associated antigens as described herein, including, e.g., CD19, CD20, CD22, CD30, CD33, CD123, EGFR, EpCAM, ganglioside GD2, HER2/neu, HLA Class II and FOLR1. BiKEs and TriKEs are described, e.g., in Felices, et al., Methods Mol Biol. (2016) 1441:333-346; Fang, et al., Semin Immunol. (2017) 31:37-54.

Hematopoietic Progenitor Kinase 1 (HPK1) Inhibitors

In various embodiments, an anti-CD47 agent or an anti-SIRPα agent as described herein, is combined with an inhibitor of mitogen-activated protein kinase kinase kinase kinase 1 (MAP4K1, HPK1; NCBI Gene ID: 11184). Examples of Hematopoietic Progenitor Kinase 1 (HPK1) inhibitors include without limitation, those described in WO-2018183956, WO-2018183964, WO-2018167147, WO-2018183964, WO-2016205942, WO-2018049214, WO-2018049200, WO-2018049191, WO-2018102366, WO-2018049152, WO2020092528, WO2020092621 and WO-2016090300.

Apoptosis Signal-Regulating Kinase (ASK) Inhibitors

In various embodiments, an anti-CD47 agent or an anti-SIRPα agent as described herein, is combined with an inhibitor of an ASK inhibitor, e.g., mitogen-activated protein kinase kinase kinase 5 (MAP3K5; ASK1, MAPKKK5, MEKK5; NCBI Gene ID: 4217). Examples of ASK1 inhibitors include without limitation, those described in WO 2011/008709 (Gilead Sciences) and WO 2013/112741 (Gilead Sciences).

Bruton Tyrosine Kinase (BTK) Inhibitors

In various embodiments, an anti-CD47 agent or an anti-SIRPα agent as described herein, is combined with an inhibitor of Bruton tyrosine kinase (BTK, AGMX1, AT, ATK, BPK, IGHD3, IMD1, PSCTK1, XLA; NCBI Gene ID: 695). Examples of BTK inhibitors include without limitation, (S)-6-amino-9-(1-(but-2-ynoyl)pyrrolidin-3-yl)-7-(4-phenoxyphenyl)-7H-purin-8(9H)-one, acalabrutinib (ACP-196), BGB-3111, CB988, HM71224, ibrutinib (Imbruvica), M-2951 (evobrutinib), M7583, tirabrutinib (ONO-4059), PRN-1008, spebrutinib (CC-292), TAK-020, vecabrutinib, ARQ-531, SHR-1459, DTRMWXHS-12, TAS-5315, Calquence+AZD6738, Calquence+danvatirsen.

Cyclin-Dependent Kinase (CDK) Inhibitors

In various embodiments, an anti-CD47 agent or an anti-SIRPα agent as described herein, is combined with an inhibitor of cyclin dependent kinase 1 (CDK1, CDCl₂; CDC28A; P34CDCl₂; NCBI Gene ID: 983); cyclin dependent kinase 2 (CDK2, CDKN2; p33(CDK2); NCBI Gene ID: 1017); cyclin dependent kinase 3 (CDK3; NCBI Gene ID: 1018); cyclin dependent kinase 4 (CDK4, CMM3; PSK-J3; NCBI Gene ID: 1019); cyclin dependent kinase 6 (CDK6, MCPH12; PLSTIRE; NCBI Gene ID: 1021); cyclin dependent kinase 7 (CDK7, CAK; CAK1; HCAK; MO15; STK1; CDKN7; p39MO15; NCBI Gene ID: 1022); cyclin dependent kinase 9 (CDK9, TAK; C-2k; CTK1; CDCl₂L4; PITALRE; NCBI Gene ID: 1025). Inhibitors of CDK 1, 2, 3, 4, 6, 7 and/or 9, include without limitation abemaciclib, alvocidib (HMR-1275, flavopiridol), AT-7519, dinaciclib, ibrance, FLX-925, LEE001, palbociclib, ribociclib, rigosertib, selinexor, UCN-01, SY1365, CT-7001, SY-1365, G1T38, milciclib, trilaciclib, PF-06873600, AZD4573, and TG-02.

Discoidin Domain Receptor (DDR) Inhibitors.

In various embodiments, an anti-CD47 agent or an anti-SIRPα agent as described herein, is combined with an inhibitor of discoidin domain receptor tyrosine kinase 1 (DDR, CAK, CD167, DDR, EDDR1, HGK2, MCK10, NEP, NTRK4, PTK3, PTK3A, RTK6, TRKE; NCBI Gene ID: 780); and/or discoidin domain receptor tyrosine kinase 2 (DDR2, MIG20a, NTRKR3, TKT, TYRO10, WRCN; NCBI Gene ID: 4921). Examples of DDR inhibitors include without limitation, dasatinib and those disclosed in WO2014/047624 (Gilead Sciences), US 2009-0142345 (Takeda Pharmaceutical), US 2011-0287011 (Oncomed Pharmaceuticals), WO 2013/027802 (Chugai Pharmaceutical), and WO2013/034933 (Imperial Innovations).

Histone Deacetylase (HDAC) Inhibitors

In various embodiments, an anti-CD47 agent or an anti-SIRPα agent as described herein, is combined with an inhibitor of a histone deacetylase, e.g., histone deacetylase 9 (HDAC9, HD7, HD7b, HD9, HDAC, HDAC7, HDAC7B, HDAC9B, HDAC9FL, HDRP, MITR; Gene ID: 9734). Examples of HDAC inhibitors include without limitation, abexinostat, ACY-241, AR-42, BEBT-908, belinostat, CKD-581, CS-055 (HBI-8000), CUDC-907 (fimepinostat), entinostat, givinostat, mocetinostat, panobinostat, pracinostat, quisinostat (JNJ-26481585), resminostat, ricolinostat, SHP-141, valproic acid (VAL-001), vorinostat, tinostamustine, remetinostat, entinostat, romidepsin, tucidinostat.

Indoleamine-Pyrrole-2,3-Dioxygenase (IDO1) Inhibitors

In various embodiments, an anti-CD47 agent or an anti-SIRPα agent as described herein, is combined with an inhibitor of indoleamine 2,3-dioxygenase 1 (IDO1; NCBI Gene ID: 3620). Examples of IDO1 inhibitors include without limitation, BLV-0801, epacadostat, F-001287, GBV-1012, GBV-1028, GDC-0919, indoximod, NKTR-218, NLG-919-based vaccine, PF-06840003, pyranonaphthoquinone derivatives (SN-35837), resminostat, SBLK-200802, BMS-986205, and shIDO-ST, EOS-200271, KHK-2455, LY-3381916.

Janus Kinase (JAK) Inhibitors

In various embodiments, an anti-CD47 agent or an anti-SIRPα agent as described herein, is combined with an inhibitor of Janus kinase 1 (JAKI, JAK1A, JAK1B, JTK3; NCBI Gene ID: 3716); Janus kinase 2 (JAK2, JTK10, THCYT3; NCBI Gene ID: 3717); and/or Janus kinase 3 (JAK3, JAK-3, JAK3_HUMAN, JAKL, L-JAK, LJAK; NCBI Gene ID: 3718). Examples of JAK inhibitors include without limitation, AT9283, AZD1480, baricitinib, BMS-911543, fedratinib, filgotinib (GLPG0634), gandotinib (LY2784544), INCB039110 (itacitinib), lestaurtinib, momelotinib (CYT0387), NS-018, pacritinib (SB1518), peficitinib (ASP015K), ruxolitinib, tofacitinib (formerly tasocitinib), INCB052793, and XL019.

Matrix Metalloprotease (MAJP) Inhibitors

In various embodiments, an anti-CD47 agent or an anti-SIRPα agent as described herein, is combined with an inhibitor of a matrix metallopeptidase (MMP), e.g., an inhibitor of MMP1 (NCBI Gene ID: 4312), MMP2 (NCBI Gene ID: 4313), MMP3 (NCBI Gene ID: 4314), MMP7 (NCBI Gene ID: 4316), MMP8 (NCBI Gene ID: 4317), MMP9 (NCBI Gene ID: 4318); MMP10 (NCBI Gene ID: 4319); MMP11 (NCBI Gene ID: 4320); MMP12 (NCBI Gene ID: 4321), MMP13 (NCBI Gene ID: 4322), MMP14 (NCBI Gene ID: 4323), MMP15 (NCBI Gene ID: 4324), MMP16 (NCBI Gene ID: 4325), MMP17 (NCBI Gene ID: 4326), MMP19 (NCBI Gene ID: 4327), MMP20 (NCBI Gene ID: 9313), MMP21 (NCBI Gene ID: 118856), MMP24 (NCBI Gene ID: 10893), MMP25 (NCBI Gene ID: 64386), MMP26 (NCBI Gene ID: 56547), MMP27 (NCBI Gene ID: 64066) and/or MMP28 (NCBI Gene ID: 79148). Examples of MMP9 inhibitors include without limitation, marimastat (BB-2516), cipemastat (Ro 32-3555), GS-5745 (andecaliximab) and those described in WO 2012/027721 (Gilead Biologics).

RAS and RAS Pathway Inhibitors

In various embodiments, an anti-CD47 agent or an anti-SIRPα agent as described herein, is combined with an inhibitor of KRAS proto-oncogene, GTPase (KRAS; a.k.a., NS; NS3; CFC2; RALD; K-Ras; KRAS1; KRAS2; RASK2; KI-RAS; C-K-RAS; K-RAS2A; K-RAS2B; K-RAS4A; K-RAS4B; c-Ki-ras2; NCBI Gene ID: 3845); NRAS proto-oncogene, GTPase (NRAS; a.k.a., NS6; CMNS; NCMS; ALPS4; N-ras; NRAS1; NCBI Gene ID: 4893); HRas proto-oncogene, GTPase (HRAS; a.k.a., CTLO; KRAS; HAMSV; HRAS1; KRAS2; RASHI; RASK2; Ki-Ras; p2lras; C—H-RAS; c-K-ras; H-RASIDX; c-Ki-ras; C-BAS/HAS; C-HA-RAS1; NCBI Gene ID: 3265). The Ras inhibitors can inhibit Ras at either the polynucleotide (e.g., transcriptional inhibitor) or polypeptide (e.g., GTPase enzyme inhibitor) level. In some embodiments, the inhibitors target one or more proteins in the Ras pathway, e.g., inhibit one or more of EGFR, Ras, Raf (A-Raf, B-Raf, C-Raf), MEK (MEK1, MEK2), ERK, PI3K, AKT and mTOR.

In various embodiments, an anti-CD47 agent or an anti-SIRPα agent as described herein, is combined with an inhibitor of KRAS. Examples of KRAS inhibitors include AMG-510, COTI-219, MRTX-1257, ARS-3248, ARS-853, WDB-178, BI-3406, BI-1701963, ARS-1620 (G12C), SML-8-73-1 (G12C), Compound 3144 (G12D), Kobe0065/2602 (Ras GTP), RT11, MRTX-849 (G12C) and K-Ras(G12D)-selective inhibitory peptides, including KRpep-2 (Ac-RRCPLYISYDPVCRR-NH2) (SEQ ID NO:167) and KRpep-2d (Ac-RRRRCPLYISYDPVCRRRR-NH2) (SEQ ID NO: 168).

In various embodiments, an anti-CD47 agent or an anti-SIRPα agent as described herein, is combined with an inhibitor of KRAS mRNA. Illustrative KRAS mRNA inhibitors include anti-KRAS U1 adaptor, AZD-4785, siG12D-LODER™, and siG12D exosomes.

In various embodiments, an anti-CD47 agent or an anti-SIRPα agent as described herein, is combined with an inhibitor of MEK. Illustrative MEK inhibitors that can be co-administered include binimetinib, cobimetinib, PD-0325901, pimasertib, RG-7304, selumetinib, trametinib, and selumetinib.

In various embodiments, an anti-CD47 agent or an anti-SIRPα agent as described herein, is combined with an inhibitor of AKT. Illustrative AKT inhibitors that can be co-administered include RG7440, MK-2206, ipatasertib, afuresertib, AZD5363, and ARQ-092, capivasertib, triciribine, ABTL-0812 (PI3K/Akt/mTOR).

In various embodiments, an anti-CD47 agent or an anti-SIRPα agent as described herein, is combined with an inhibitor of Raf. Illustrative Raf inhibitors that can be co-administered BGB-283 (Raf/EGFR), HM-95573, LXH-254, LY-3009120, RG7304, TAK-580, dabrafenib, vemurafenib, encorafenib (LGX818), PLX8394. RAF-265 (Raf/VEGFR), ASN-003 (Raf/PI3K).

In various embodiments, an anti-CD47 agent or an anti-SIRPα agent as described herein, is combined with an inhibitor of ERK. Illustrative ERK inhibitors that can be co-administered include LTT-462, LY-3214996, MK-8353, ravoxertinib, GDC-0994, and ulixertinib.

In various embodiments, an anti-CD47 agent or an anti-SIRPα agent as described herein, is combined with an inhibitor of PI3K. Illustrative PI3K inhibitors that can be co-administered include idelalisib (Zydelig®), alpelisib, buparlisib, pictilisib, eganelisib (IPI-549). Illustrative PI3K/mTOR inhibitors that can be co-administered include dactolisib, omipalisib, voxtalisib, gedatolisib, GSK2141795, RG6114.

In various embodiments, an anti-CD47 agent or an anti-SIRPα agent as described herein, is combined with an inhibitor of mTOR. Illustrative mTOR inhibitors that can be co-administered include as sapanisertib, vistusertib (AZD2014), ME-344, sirolimus (oral nano-amorphous formulation, cancer), TYME-88 (mTOR/cytochrome P450 3A4).

In certain embodiments, Ras-driven cancers (e.g., NSCLC) having CDKN2A mutations can be inhibited by co-administration of the MEK inhibitor selumetinib and the CDK4/6 inhibitor palbociclib. See, e.g., Zhou, et al., Cancer Lett. 2017 Nov. 1; 408:130-137. Also, K-RAS and mutant N-RAS can be reduced by the irreversible ERBB1/2/4 inhibitor neratinib. See, e.g., Booth, et al., Cancer Biol Ther. 2018 Feb. 1; 19(2):132-137.

In various embodiments, an anti-CD47 agent or an anti-SIRPα agent as described herein, is combined with an inhibitor of RAS. Examples of RAS inhibitors include NEO-100, rigosertib;

In various embodiments, an anti-CD47 agent or an anti-SIRPα agent as described herein, is combined with an antagonist of EGFR, such as AMG-595, necitumumab, ABBV-221, depatuxizumab mafodotin (ABT-414), tomuzotuximab, ABT-806, vectibix, modotuximab, RM-1929.

In various embodiments, an anti-CD47 agent or an anti-SIRPα agent as described herein, is combined with an inhibitor of protein tyrosine phosphatase non-receptor type 11 (PTPN11; BPTP3, CFC, JMML, METCDS, NS1, PTP-1D, PTP2C, SH-PTP2, SH-PTP3, SHP2; NCBI Gene ID: 5781). Examples of SHP2 inhibitors include TNO155 (SHP-099), RMC-4550, JAB-3068, RMC-4630, SAR442720 and those described in WO2018172984 and WO2017211303.

In various embodiments, an anti-CD47 agent or an anti-SIRPα agent as described herein, is combined with an inhibitor of mitogen-activated protein kinase 7 (MAP2K7, JNKK2, MAPKK7, MEK, MEK 7, MKK7, PRKMK7, SAPKK-4, SAPKK4; NCBI Gene ID: 5609). Examples of MEK inhibitors include antroquinonol, binimetinib, CK-127, cobimetinib (GDC-0973, XL-518), MT-144, selumetinib (AZD6244), sorafenib, trametinib (GSK1120212), uprosertib+trametinib, PD-0325901, pimasertib, LTT462, AS703988, CC-90003, refametinib, TAK-733, CI-1040, RG7421.

In various embodiments, an anti-CD47 agent or an anti-SIRPα agent as described herein, is combined with an inhibitor of a phosphatidylinositol-4,5-bisphosphate 3-kinase catalytic subunit, e.g., phosphatidylinositol-4,5-bisphosphate 3-kinase catalytic subunit alpha (PIK3CA, CLAPO, CLOVE, CWS5, MCAP, MCM, MCMTC, PI3K, PI3K-alpha, p110-alpha; NCBI Gene ID: 5290); phosphatidylinositol-4,5-bisphosphate 3-kinase catalytic subunit beta (PIK3CB, P1IOBETA, PI3K, PI3KBETA, PIK3C1; NCBI Gene ID: 5291); phosphatidylinositol-4,5-bisphosphate 3-kinase catalytic subunit gamma (PIK3CG, PI3CG, PI3K, PI3Kgamma, PIK3, p110gamma, p120-PI3K; Gene ID: 5494); and/or phosphatidylinositol-4,5-bisphosphate 3-kinase catalytic subunit delta (PIK3CD, APDS, IMD14, P110DELTA, PI3K, p110D, NCBI Gene ID: 5293). In some embodiments, the PI3K inhibitor is a pan-PI3K inhibitor. Examples of PI3K inhibitors include without limitation, ACP-319, AEZA-129, AMG-319, AS252424, AZD8186, BAY 1082439, BEZ235, bimiralisib (PQR309), buparlisib (BKM120), BYL719 (alpelisib), carboxyamidotriazole orotate (CTO), CH5132799, CLR-457, CLR-1401, copanlisib (BAY 80-6946), DS-7423, dactolisib, duvelisib (IPI-145), fimepinostat (CUDC-907), gedatolisib (PF-05212384), GDC-0032, GDC-0084 (RG7666), GDC-0077, pictilisib (GDC-0941), GDC-0980, GSK2636771, GSK2269577, GSK2141795, idelalisib (Zydelig®), INCB040093, INCB50465, IPI-443, IPI-549, KAR4141, LY294002, LY3023414, NERLYNX® (neratinib), nemiralisib (GSK2269557), omipalisib (GSK2126458, GSK458), OXY111A, panulisib (P7170, AK151761), PA799, perifosine (KRX-0401), Pilaralisib (SAR245408; XL147), puquitinib mesylate (XC-302), SAR260301, seletalisib (UCB-5857), serabelisib (INK-1117, MLN-1117, TAK-117), SF1126, sonolisib (PX-866), RG6114, RG7604, rigosertib sodium (ON-01910 sodium), RP5090, tenalisib (RP6530), RV-1729, SRX3177, taselisib, TG100115, umbralisib (TGR-1202), TGX221, voxtalisib (SAR245409), VS-5584, WX-037, X-339, X-414, XL499, XL756, wortmannin, ZSTK474, and the compounds described in WO 2005/113556 (ICOS), WO 2013/052699 (Gilead Calistoga), WO 2013/116562 (Gilead Calistoga), WO 2014/100765 (Gilead Calistoga), WO 2014/100767 (Gilead Calistoga), and WO 2014/201409 (Gilead Sciences).

Spleen Tyrosine Kinase (SYK) Inhibitors

In various embodiments, an anti-CD47 agent or an anti-SIRPα agent as described herein, is combined with an inhibitor of spleen associated tyrosine kinase (SYK, p72-Syk, Gene ID: 6850). Examples of SYK inhibitors include without limitation, 6-(1H-indazol-6-yl)-N-(4-morpholinophenyl)imidazo[1,2-a]pyrazin-8-amine, BAY-61-3606, cerdulatinib (PRT-062607), entospletinib, fostamatinib (R788), HMPL-523, NVP-QAB 205 AA, R112, R343, tamatinib (R406), and those described in U.S. Pat. No. 8,450,321 (Gilead Connecticut) and those described in U.S. 2015/0175616.

Tyrosine-Kinase Inhibitors (TKIs)

In various embodiments, an anti-CD47 agent or an anti-SIRPα agent as described herein, is combined with a tyrosine kinase inhibitor (TKI). TKIs may target epidermal growth factor receptors (EGFRs) and receptors for fibroblast growth factor (FGF), platelet-derived growth factor (PDGF), and vascular endothelial growth factor (VEGF). Examples of TKIs include without limitation, axitinib, afatinib, ARQ-087 (derazantinib), asp5878, AZD3759, AZD4547, bosutinib, brigatinib, cabozantinib, cediranib, crenolanib, dacomitinib, dasatinib, dovitinib, E-6201, erdafitinib, erlotinib, gefitinib, gilteritinib (ASP-2215), FP-1039, HM61713, icotinib, imatinib, KX2-391 (Src), lapatinib, lestaurtinib, lenvatinib, midostaurin, nintedanib, ODM-203, olmutinib, osimertinib (AZD-9291), pazopanib, ponatinib, poziotinib, quizartinib, radotinib, rociletinib, sulfatinib (HMPL-012), sunitinib, famitinib L-malate, (MAC-4), tivoanib, TH-4000, tivoanib, and MEDI-575 (anti-PDGFR antibody), TAK-659, Cabozantinib.

Chemotherapeutic Agents (Standard of Care)

In various embodiments, an anti-CD47 agent or an anti-SIRPα agent as described herein, is combined with a chemotherapeutic agent or anti-neoplastic agent.

As used herein, the term “chemotherapeutic agent” or “chemotherapeutic” (or “chemotherapy” in the case of treatment with a chemotherapeutic agent) is meant to encompass any non-proteinaceous (e.g., non-peptidic) chemical compound useful in the treatment of cancer. Examples of chemotherapeutic agents include but not limited to: alkylating agents such as thiotepa and cyclophosphamide (CYTOXAN®); alkyl sulfonates such as busulfan, improsulfan, and piposulfan; aziridines such as benzodepa, carboquone, meturedepa, and uredepa; ethylenimines and methylamelamines including altretamine, triethylenemelamine, triethylenephosphoramide, triethylenethiophosphoramide, and trimemylolomelamine; acetogenins, e.g., bullatacin and bullatacinone; a camptothecin, including synthetic analog topotecan; bryostatin, callystatin; CC-1065, including its adozelesin, carzelesin, and bizelesin synthetic analogs; cryptophycins, particularly cryptophycin 1 and cryptophycin 8; dolastatin; duocarmycin, including the synthetic analogs KW-2189 and CBI-TMI; eleutherobin; 5-azacytidine; pancratistatin; a sarcodictyin; spongistatin; nitrogen mustards such as chlorambucil, chlomaphazine, cyclophosphamide, glufosfamide, evofosfamide, bendamustine, estramustine, ifosfamide, mechlorethamine, mechlorethamine oxide hydrochloride, melphalan, novembichin, phenesterine, prednimustine, trofosfamide, and uracil mustard; nitrosoureas such as carmustine, chlorozotocin, foremustine, lomustine, nimustine, and ranimustine; antibiotics such as the enediyne antibiotics (e.g., calicheamicin, especially calicheamicin gammaII and calicheamicin phiI1), dynemicin including dynemicin A, bisphosphonates such as clodronate, an esperamicin, neocarzinostatin chromophore and related chromoprotein enediyne antibiotic chromomophores, aclacinomycins, actinomycin, authramycin, azaserine, bleomycins, cactinomycin, carabicin, carminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin, and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, and zorubicin; anti-metabolites such as methotrexate and 5-fluorouracil (5-FU); folic acid analogs such as demopterin, methotrexate, pteropterin, and trimetrexate; purine analogs such as cladribine, pentostatin, fludarabine, 6-mercaptopurine, thiamiprine, and thioguanine; pyrimidine analogs such as ancitabine, azacitidine, 6-azauridine, carmofur, cytarabine, dideoxyuridine, doxifluridine, enocitabine, and floxuridine; androgens such as calusterone, dromostanolone propionate, epitiostanol, mepitiostane, and testolactone; anti-adrenals such as aminoglutethimide, mitotane, and trilostane; folic acid replinishers such as frolinic acid; radiotherapeutic agents such as Radium-223, 177-Lu-PSMA-617; trichothecenes, especially T-2 toxin, verracurin A, roridin A, and anguidine; taxoids such as paclitaxel (TAXOL®), abraxane, docetaxel (TAXOTERE®), cabazitaxel, BIND-014, tesetaxel; platinum analogs such as cisplatin and carboplatin, NC-6004 nanoplatin; aceglatone; aldophosphamide glycoside; aminolevulinic acid; eniluracil; amsacrine; hestrabucil; bisantrene; edatraxate; defofamine; demecolcine; diaziquone; elformthine; elliptinium acetate; an epothilone; etoglucid; gallium nitrate; hydroxyurea; lentinan; leucovorin; lonidamine; maytansinoids such as maytansine and ansamitocins; mitoguazone; mitoxantrone; mopidamol; nitracrine; phenamet; pirarubicin; losoxantrone; fluoropyrimidine; folinic acid; podophyllinic acid; 2-ethylhydrazide; procarbazine; polysaccharide-K (PSK); razoxane; rhizoxin; sizofiran; spirogermanium; tenuazonic acid; trabectedin, triaziquone; 2,2′, 2″-trichlorotriemylamine; urethane; vindesine; dacarbazine; mannomustine; mitobronitol; mitolactol; pipobroman; gacytosine; arabinoside (“Ara-C”); cyclophosphamide; thiopeta; chlorambucil; gemcitabine (GEMZAR®); 6-thioguanine; mercaptopurine; methotrexate; vinblastine; platinum; etoposide (VP-16); ifosfamide; mitroxantrone; vancristine; vinorelbine (NAVELBINE®); novantrone; teniposide; edatrexate; daunomycin; aminopterin; xeoloda; ibandronate; CPT-11; topoisomerase inhibitor RFS 2000; difluoromethylornithine (DFMO); retinoids such as retinoic acid; capecitabine; NUC-1031; FOLFOX (folinic acid, 5-fluorouracil, oxaliplatin); FOLFIRI (folinic acid, 5-fluorouracil, irinotecan); FOLFOXIRI (folinic acid, 5-fluorouracil, oxaliplatin, irinotecan), FOLFIRINOX (folinic acid, 5-fluorouracil, irinotecan, oxaliplatin), and pharmaceutically acceptable salts, acids, or derivatives of any of the above. Such agents can be conjugated onto an antibody or any targeting agent described herein to create an antibody-drug conjugate (ADC) or targeted drug conjugate.

Also included in the definition of “chemotherapeutic agent” are anti-hormonal agents such as anti-estrogens and selective estrogen receptor modulators (SERMs), inhibitors of the enzyme aromatase, anti-androgens, and pharmaceutically acceptable salts, acids or derivatives of any of the above that act to regulate or inhibit hormone action on tumors. Examples of anti-estrogens and SERMs include, for example, tamoxifen (including NOLVADEX™), raloxifene, droloxifene, 4-hydroxytamoxifen, trioxifene, keoxifene, LY117018, onapristone, and toremifene (FARESTON®). Inhibitors of the enzyme aromatase regulate estrogen production in the adrenal glands. Examples include 4(5)-imidazoles, aminoglutethimide, megestrol acetate (MEGACE®), exemestane, formestane, fadrozole, vorozole (RIVISOR®), letrozole (FEMARA®), and anastrozole (ARIMIDEX®). Examples of anti-androgens include apalutamide, abiraterone, enzalutamide, flutamide, galeterone, nilutamide, bicalutamide, leuprolide, goserelin, ODM-201, APC-100, ODM-204. An example progesterone receptor antagonist includes onapristone.

Anti-Angiogenic Agents

In various embodiments, an anti-CD47 agent or an anti-SIRPα agent as described herein, is combined with an anti-angiogenic agent. Anti-angiogenic agents that can be co-administered include, but are not limited to, retinoid acid and derivatives thereof, 2-methoxyestradiol, ANGIOSTATIN®, ENDOSTATIN®, regorafenib, necuparanib, suramin, squalamine, tissue inhibitor of metalloproteinase-1, tissue inhibitor of metalloproteinase-2, plasminogen activator inhibitor-1, plasminogen activator inbibitor-2, cartilage-derived inhibitor, paclitaxel (nab-paclitaxel), platelet factor 4, protamine sulphate (clupeine), sulphated chitin derivatives (prepared from queen crab shells), sulphated polysaccharide peptidoglycan complex (sp-pg), staurosporine, modulators of matrix metabolism including proline analogs such as 1-azetidine-2-carboxylic acid (LACA), cishydroxyproline, d,I-3,4-dehydroproline, thiaproline, α,α′-dipyridyl, beta-aminopropionitrile fumarate, 4-propyl-5-(4-pyridinyl)-2(3h)-oxazolone, methotrexate, mitoxantrone, heparin, interferons, 2 macroglobulin-serum, chicken inhibitor of metalloproteinase-3 (ChIMP-3), chymostatin, beta-cyclodextrin tetradecasulfate, eponemycin, fumagillin, gold sodium thiomalate, d-penicillamine, beta-1-anticollagenase-serum, alpha-2-antiplasmin, bisantrene, lobenzarit disodium, n-2-carboxyphenyl-4-chloroanthronilic acid disodium or “CCA”, thalidomide, angiostatic steroid, carboxy aminoimidazole, metalloproteinase inhibitors such as BB-94, inhibitors of S100A9 such as tasquinimod. Other anti-angiogenesis agents include antibodies, preferably monoclonal antibodies against these angiogenic growth factors: beta-FGF, alpha-FGF, FGF-5, VEGF isoforms, VEGF-C, HGF/SF, and Ang-1/Ang-2.

Anti-Fibrotic Agents

In various embodiments, an anti-CD47 agent or an anti-SIRPα agent as described herein, is combined with an anti-fibrotic agent. Anti-fibrotic agents that can be co-administered include, but are not limited to, the compounds such as beta-aminoproprionitrile (BAPN), as well as the compounds disclosed in U.S. Pat. No. 4,965,288 relating to inhibitors of lysyl oxidase and their use in the treatment of diseases and conditions associated with the abnormal deposition of collagen and U.S. Pat. No. 4,997,854 relating to compounds which inhibit LOX for the treatment of various pathological fibrotic states, which are herein incorporated by reference. Further exemplary inhibitors are described in U.S. Pat. No. 4,943,593 relating to compounds such as 2-isobutyl-3-fluoro-, chloro-, or bromo-allylamine, U.S. Pat. Nos. 5,021,456, 5,059,714, 5,120,764, 5,182,297, 5,252,608 relating to 2-(1-naphthyloxymemyl)-3-fluoroallylamine, and US 2004-0248871, which are herein incorporated by reference.

Exemplary anti-fibrotic agents also include the primary amines reacting with the carbonyl group of the active site of the lysyl oxidases, and more particularly those which produce, after binding with the carbonyl, a product stabilized by resonance, such as the following primary amines: emylenemamine, hydrazine, phenylhydrazine, and their derivatives; semicarbazide and urea derivatives; aminonitriles such as BAPN or 2-nitroethylamine; unsaturated or saturated haloamines such as 2-bromo-ethylamine, 2-chloroethylamine, 2-trifluoroethylamine, 3-bromopropylamine, and p-halobenzylamines; and selenohomocysteine lactone.

Other anti-fibrotic agents are copper chelating agents penetrating or not penetrating the cells. Exemplary compounds include indirect inhibitors which block the aldehyde derivatives originating from the oxidative deamination of the lysyl and hydroxylysyl residues by the lysyl oxidases. Examples include the thiolamines, particularly D-penicillamine, and its analogs such as 2-amino-5-mercapto-5-methylhexanoic acid, D-2-amino-3-methyl-3-((2-acetamidoethyl)dithio)butanoic acid, p-2-amino-3-methyl-3-((2-aminoethyl)dithio)butanoic acid, sodium-4-((p-1-dimethyl-2-amino-2-carboxyethyl)dithio)butane sulphurate, 2-acetamidoethyl-2-acetamidoethanethiol sulphanate, and sodium-4-mercaptobutanesulphinate trihydrate.

Anti-Inflammatory Agents

In various embodiments, an anti-CD47 agent or an anti-SIRPα agent as described herein, is combined with an anti-inflammatory agent. Example anti-inflammatory agents include without limitation inhibitors of one or more of arginase (ARGI (NCBI Gene ID: 383), ARG2 (NCBI Gene ID: 384)), carbonic anhydrase (CA1 (NCBI Gene ID: 759), CA2 (NCBI Gene ID: 760), CA3 (NCBI Gene ID: 761), CA4 (NCBI Gene ID: 762), CA5A (NCBI Gene ID: 763), CA5B (NCBI Gene ID: 11238), CA6 (NCBI Gene ID: 765), CA7 (NCBI Gene ID: 766), CA8 (NCBI Gene ID: 767), CA9 (NCBI Gene ID: 768), CA10 (NCBI Gene ID: 56934), CA11 (NCBI Gene ID: 770), CA12 (NCBI Gene ID: 771), CA13 (NCBI Gene ID: 377677), CA14 (NCBI Gene ID: 23632)), prostaglandin-endoperoxide synthase 1 (PTGS1, COX-1; NCBI Gene ID: 5742), prostaglandin-endoperoxide synthase 2 (PTGS2, COX-2; NCBI Gene ID: 5743), secreted phospholipase A2, prostaglandin E synthase (PTGES, PGES; Gene ID: 9536), arachidonate 5-lipoxygenase (ALOX5, 5-LOX; NCBI Gene ID: 240), soluble epoxide hydrolase 2 (EPHX2, SEH; NCBI Gene ID: 2053) and/or mitogen-activated protein kinase kinase kinase 8 (MAP3K8, TPL2; NCBI Gene ID: 1326). In some embodiments, the inhibitor is a dual inhibitor, e.g., a dual inhibitor of COX-2/COX-1, COX-2/SEH, COX-2/CA, COX-2/5-LOX.

Examples of inhibitors of prostaglandin-endoperoxide synthase 1 (PTGS1, COX-1; NCBI Gene ID: 5742) that can be co-administered include without limitation mofezolac, GLY-230, and TRK-700.

Examples of inhibitors of prostaglandin-endoperoxide synthase 2 (PTGS2, COX-2; NCBI Gene ID: 5743) that can be co-administered include without limitation diclofenac, meloxicam, parecoxib, etoricoxib, AP-101, celecoxib, AXS-06, diclofenac potassium, DRGT-46, AAT-076, meisuoshuli, lumiracoxib, meloxicam, valdecoxib, zaltoprofen, nimesulide, Anitrazafen, Apricoxib, Cimicoxib, Deracoxib, Flumizole, Firocoxib, Mavacoxib, NS-398, Pamicogrel, Parecoxib, Robenacoxib, Rofecoxib, Rutecarpine, Tilmacoxib, and Zaltoprofen. Examples of dual COX1/COX2 inhibitors that can be co-administered include without limitation, HP-5000, lomoxicam, ketorolac tromethamine, bromfenac sodium, ATB-346, HP-5000. Examples of dual COX-2/carbonic anhydrase (CA) inhibitors that can be co-administered include without limitation polmacoxib and imrecoxib.

Examples of inhibitors of secreted phospholipase A2, prostaglandin E synthase (PTGES, PGES; Gene ID: 9536) that can be co-administered include without limitation LY3023703, GRC 27864, and compounds described in WO2015158204, WO2013024898, WO2006063466, WO2007059610, WO2007124589, WO2010100249, WO2010034796, WO2010034797, WO2012022793, WO2012076673, WO2012076672, WO2010034798, WO2010034799, WO2012022792, WO2009103778, WO2011048004, WO2012087771, WO2012161965, WO2013118071, WO2013072825, WO2014167444, WO2009138376, WO2011023812, WO2012110860, WO2013153535, WO2009130242, WO2009146696, WO2013186692, WO2015059618, WO2016069376, WO2016069374, WO2009117985, WO2009064250, WO2009064251, WO2009082347, WO2009117987, and WO2008071173. Metformin has further been found to repress the COX2/PGE2/STAT3 axis, and can be co-administered. See, e.g., Tong, et al., Cancer Lett. (2017) 389:23-32; and Liu, et al., Oncotarget. (2016) 7(19):28235-46.

Examples of inhibitors of carbonic anhydrase (e.g., one or more of CA1 (NCBI Gene ID: 759), CA2 (NCBI Gene ID: 760), CA3 (NCBI Gene ID: 761), CA4 (NCBI Gene ID: 762), CASA (NCBI Gene ID: 763), CA5B (NCBI Gene ID: 11238), CA6 (NCBI Gene ID: 765), CA7 (NCBI Gene ID: 766), CA8 (NCBI Gene ID: 767), CA9 (NCBI Gene ID: 768), CA10 (NCBI Gene ID: 56934), CA11 (NCBI Gene ID: 770), CA12 (NCBI Gene ID: 771), CA13 (NCBI Gene ID: 377677), CA14 (NCBI Gene ID: 23632)) that can be co-administered include without limitation acetazolamide, methazolamide, dorzolamide, zonisamide, brinzolamide and dichlorphenamide. A dual COX-2/CA1/CA2 inhibitor that can be co-administered includes CG100649.

Examples of inhibitors of arachidonate 5-lipoxygenase (ALOX5, 5-LOX; NCBI Gene ID: 240) that can be co-administered include without limitation meclofenamate sodium, zileuton.

Examples of inhibitors of soluble epoxide hydrolase 2 (EPHX2, SEH; NCBI Gene ID: 2053) that can be co-administered include without limitation compounds described in WO2015148954. Dual inhibitors of COX-2/SEH that can be co-administered include compounds described in WO2012082647. Dual inhibitors of SEH and fatty acid amide hydrolase (FAAH; NCBI Gene ID: 2166) that can be co-administered include compounds described in WO2017160861.

Examples of inhibitors of mitogen-activated protein kinase kinase kinase 8 (MAP3K8, tumor progression loci-2, TPL2; NCBI Gene ID: 1326) that can be co-administered include without limitation GS-4875, GS-5290, BHM-078 and those described, e.g., in WO2006124944, WO2006124692, WO2014064215, WO2018005435, Teli, et al., J Enzyme Inhib Med Chem. (2012) 27(4):558-70; Gangwall, et al., Curr Top Med Chem. (2013) 13(9):1015-35; Wu, et al., Bioorg Med Chem Lett. (2009) 19(13):3485-8; Kaila, et al., Bioorg Med Chem. (2007) 15(19):6425-42; and Hu, et al., Bioorg Med Chem Lett. (2011) 21(16):4758-61.

Tumor Oxygenation Agents

In various embodiments, an anti-CD47 agent or an anti-SIRPα agent as described herein, is combined with an agent that promotes or increases tumor oxygenation or reoxygenation, or prevents or reduces tumor hypoxia. Illustrative agents that can be co-administered include, e.g., Hypoxia inducible factor-1 alpha (HIF-1α) inhibitors, such as PT-2977, PT-2385; VEGF inhibitors, such as bevasizumab, IMC-3C5, GNR-011, tanibirumab, LYN-00101, ABT-165; and/or an oxygen carrier protein (e.g, a heme nitric oxide and/or oxygen binding protein (HNOX)), such as OMX-302 and HNOX proteins described in WO 2007/137767, WO 2007/139791, WO 2014/107171, and WO 2016/149562.

Immunotherapeutic Agents

In various embodiments, an anti-CD47 agent or an anti-SIRPα agent as described herein, is combined with an immunotherapeutic agent. Example immunotherapeutic agents that can be co-administered include without limitation abagovomab, ABP-980, adecatumumab, afutuzumab, alemtuzumab, altumomab, amatuximab, anatumomab, arcitumomab, bavituximab, bectumomab, bevacizumab biosimilar, bivatuzumab, blinatumomab, brentuximab, cantuzumab, catumaxomab, CC49, cetuximab, citatuzumab, cixutumumab, clivatuzumab, conatumumab, dacetuzumab, dalotuzumab, daratumumab, detumomab, dinutuximab, drozitumab, duligotumab, dusigitumab, ecromeximab, emibetuzumab, ensituximab, ertumaxomab, etaracizumab, farletuzumab, figitumumab, flanvotumab, futuximab, gemtuzumab, girentuximab, glembatumumab, ibritumomab, igovomab, imgatuzumab, indatuximab, inotuzumab, intetumumab, ipilimumab (YERVOY®, MDX-010, BMS-734016, and MDX-101), iratumumab, labetuzumab, lexatumumab, lintuzumab, lorvotuzumab, lucatumumab, matuzumab, milatuzumab, minretumomab, mitumomab, moxetumomab, moxetumomab pasudotox, naptumomab, namatumab, necitumumab, nimotuzumab, nofetumomab, OBI-833, obinutuzumab, ocaratuzumab, ofatumumab, olaratumab, onartuzumab, oportuzumab, oregovomab, panitumumab, parsatuzumab, pasudotox, patritumab, pemtumomab, pertuzumab, pintumomab, pritumumab, racotumomab, radretumab, ramucirumab (Cyramza®), rilotumumab, rituximab, robatumumab, samalizumab, satumomab, sibrotuzumab, siltuximab, solitomab, simtuzumab, tacatuzumab, taplitumomab, tenatumomab, teprotumumab, tigatuzumab, tositumomab, trastuzumab, trastuzumab biosimilar, tucotuzumab, ubilituximab, veltuzumab, vorsetuzumab, votumumab, zalutumumab, and 3F8. Rituximab can be used for treating indolent B-cell cancers, including marginal-zone lymphoma, WM, CLL and small lymphocytic lymphoma. A combination of Rituximab and chemotherapy agents is especially effective.

The exemplified therapeutic antibodies may be further labeled or combined with a radioisotope particle such as indium-111, yttrium-90 (90Y-clivatuzumab), or iodine-131.

In some embodiments, the immunotherapeutic agent is an antibody-drug conjugate (ADC). Illustrative ADCs that can be co-administered include without limitation drug-conjugated antibodies, fragments thereof, or antibody mimetics targeting the proteins or antigens listed above and herein (e.g., in Table B). Example ADCs that can be co-administered include without limitation gemtuzumab, brentuximab, trastuzumab, inotuzumab, glembatumumab, anetumab, mirvetuximab, depatuxizumab, rovalpituzumab, vadastuximab, labetuzumab, sacituzumab, lifastuzumab, indusatumab, polatzumab, pinatuzumab, coltuximab, indatuximab, milatuzumab, rovalpituzumab, ABBV-011, ABBV-2029, ABBV-321, ABBV-647, MLN0264 (anti-GCC, guanylyl cyclase C), T-DM1 (trastuzumab emtansine, Kadcycla); SYD985 (anti-HER2, Duocarmycin), milatuzumab-doxorubicin (hCD74-DOX), DCDT2980S, belantamab mafodotin (GSK2857916), polatuzumab vedotin (RG-7596), SGN-CD70A, SGN-CD19A, inotuzumab ozogamicin (CMC-544), lorvotuzumab mertansine, SAR3419, isactuzumab govitecan, enfortumab vedotin (ASG-22ME), ASG-15ME, DS-8201 ((trastuzumab deruxtecan), 225Ac-lintuzumab, U3-1402, 177Lu-tetraxetan-tetuloma, tisotumab vedotin, anetumab ravtansine, CX-2009, SAR-566658, W-0101, ABBV-085, gemtuzumab ozogamicin, ABT-414, glembatumumab vedotin (CDX-011), labetuzumab govitecan (IMMU-130), sacituzumab govitecan (IMMU-132), lifastuzumab vedotin, (RG-7599), milatuzumab-doxorubicin (IMMU-110), indatuximab ravtansine (BT-062), pinatuzumab vedotin (RG-7593), SGN-LIV1A, SGN-CD33A, SAR566658, MLN2704, SAR408701, rovalpituzumab tesirine, ABBV-399, AGS-16C3F, ASG-22ME, AGS67E, AMG 172, AMG 595, AGS-15E, BAY1129980, BAY1187982, BAY94-934 (anetumab ravtansine), GSK2857916, Humax-TF-ADC (tisotumab vedotin), IMGN289, IMGN529; IMGN853 (mirvetuximab soravtansine), LOP628, PCA062, MDX-1203, MEDI-547, PF-06263507, PF-06647020, PF-06647263, PF-06664178, PF-06688992, PF-06804103, RG7450, RG7458, RG7598, SAR566658, SGN-CD33A, DS-1602 and DS-7300, DS-6157, DS-6000, TAK-164, MED12228, MED17247, AMG575. ADCs that can be co-administered are described, e.g., in Lambert, et al., Adv Ther (2017) 34:1015-1035 and in de Goeij, Current Opinion in Immunology (2016) 40:14-23.

Illustrative therapeutic agents (e.g., anticancer or antineoplastic agents) that can be conjugated to the drug-conjugated antibodies, fragments thereof, or antibody mimetics include without limitation monomethyl auristatin E (MMAE), monomethyl auristatin F (MMAF), a calicheamicin, ansamitocin, maytansine or an analog thereof (e.g., mertansine/emtansine (DM1), ravtansine/soravtansine (DM4)), an anthracyline (e.g., doxorubicin, daunorubicin, epirubicin, idarubicin), pyrrolobenzodiazepine (PBD) DNA cross-linking agent SC-DR002 (D6.5), duocarmycin, a microtubule inhibitors (MTI) (e.g., a taxane, a vinca alkaloid, an epothilone), a pyrrolobenzodiazepine (PBD) or dimer thereof, a duocarmycin (A, B1, B2, C1, C2, D, SA, CC-1065), and other anticancer or anti-neoplastic agents described herein.

Cancer Gene Therapy and Cell Therapy

In various embodiments, an anti-CD47 agent or an anti-SIRPα agent as described herein, is combined with a cancer gene therapy and cell therapy. Cancer gene therapies and cell therapies include the insertion of a normal gene into cancer cells to replace a mutated or altered gene; genetic modification to silence a mutated gene; genetic approaches to directly kill the cancer cells; including the infusion of immune cells designed to replace most of the patient's own immune system to enhance the immune response to cancer cells, or activate the patient's own immune system (T cells or Natural Killer cells) to kill cancer cells, or find and kill the cancer cells; genetic approaches to modify cellular activity to further alter endogenous immune responsiveness against cancer.

Cellular Therapies

In various embodiments, an anti-CD47 agent or an anti-SIRPα agent as described herein, is combined with one or more cellular therapies. Illustrative cellular therapies include without limitation co-administration of one or more of a population of immune cells. In some embodiments, the immune cells are natural killer (NK) cells, NK-T cells, T cells, gamma delta T cells, B-cells, cytokine-induced killer (CIK) cells, macrophage (MAC) cells, tumor infiltrating lymphocytes (TILs) a granulocyte, an innate lymphoid cell, a megakaryocyte, a monocyte, a macrophage, a platelet, a thymocyte, a myeloid cell, and/or dendritic cells (DCs). In some embodiments, the cellular therapy entails a T cell therapy, e.g., co-administering a population of alpha/beta TCR T cells, gamma/delta TCR T cells, regulatory T (Treg) cells and/or TRuC™ T cells. In some embodiments, the cellular therapy entails a NK cell therapy, e.g., co-administering NK-92 cells. As appropriate, a cellular therapy can entail the co-administration of cells that are autologous, syngeneic or allogeneic to the subject.

In some embodiments, the cellular therapy entails co-administering immune cells engineered to express chimeric antigen receptors (CARs) or T cell receptors (TCRs) TCRs. In particular embodiments, a population of immune cells is engineered to express a CAR, wherein the CAR comprises a tumor antigen-binding domain. In other embodiments, a population of immune cells is engineered to express T cell receptors (TCRs) engineered to target tumor derived peptides presented on the surface of tumor cells. In one embodiment, the immune cell engineered to express chimeric antigen receptors (CARs) or T cell receptors (TCRs) TCRs is a T cell. In another embodiment, the immune cell engineered to express chimeric antigen receptors (CARs) or T cell receptors (TCRs) TCRs is an NK cell.

With respect to the structure of a CAR, in some embodiments, the CAR comprises an antigen binding domain, a transmembrane domain, and an intracellular signaling domain. In some embodiments, the intracellular domain comprises a primary signaling domain, a costimulatory domain, or both of a primary signaling domain and a costimulatory domain. In some embodiments, the primary signaling domain comprises a functional signaling domain of one or more proteins selected from the group consisting of CD3 zeta, CD3 gamma, CD3 delta, CD3 epsilon, common FcR gamma (FCERIG), FcR beta (Fc Epsilon R1b), CD79a, CD79b, Fcgamma RIIa, DAP10, and DAP12 4-1BB/CD137, activating NK cell receptors, an Immunoglobulin protein, B7-H3, BAFFR, BLAME (SLAMF8), BTLA, CD100 (SEMA4D), CD103, CD160 (BY55), CD18, CD19, CD19a, CD2, CD247, CD27, CD276 (B7-H3), CD28, CD29, CD3 delta, CD3 epsilon, CD3 gamma, CD30, CD4, CD40, CD49a, CD49D, CD49f, CD69, CD7, CD84, CD8alpha, CD8beta, CD96 (Tactile), CDTa, CDTb, CDTc, CDTd, CDS, CEACAM1, CRT AM, cytokine receptor, DAP-10, DNAM1 (CD226), Fc gamma receptor, GADS, GITR, HVEM (LIGHTR), IA4, ICAM-1, ICAM-1, Ig alpha (CD79a), IL-2R beta, IL-2R gamma, IL-7R alpha, inducible T cell costimulator (ICOS), integrins, ITGA4, ITGA4, ITGA6, ITGAD, ITGAE, ITGAL, ITGAM, ITGAX, ITGB2, ITGB7, ITGB1, KIRDS2, LAT, LFA-1, LFA-1, ligand that binds with CD83, LIGHT, LIGHT, LTBR, Ly9 (CD229), Ly108), lymphocyte function-associated antigen-1 (LFA-1; CD1-1a/CD18), MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), Signaling Lymphocytic Activation Molecules (SLAM proteins), SLAM (SLAMF1; CD150; IPO-3), SLAMF4 (CD244; 2B4), SLAMF6 (NTB-A, SLAMF7, SLP-76, TNF receptor proteins, TNFR2, TNFSF14, a Toll ligand receptor, TRANCE/RANKL, VLA1, or VLA-6, or a fragment, truncation, or a combination thereof.

In some embodiments, the costimulatory domain comprises a functional domain of one or more proteins selected from the group consisting of CD27, CD28, 4-1BB(CD137), OX40, CD30, CD40, PD-1, ICOS, CD2, CD7, LIGHT, NKG2C, lymphocyte function-associated antigen-1 (LFA-1), MYD88, B7-H3, a ligand that specifically binds with CD83, CDS, ICAM-1, GITR, BAFFR, HVEM (LIGHTR), SLAMF7, NKp80 (KLRFI), CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, ITGAE, CD103, ITGAL, CD1A (NCBI Gene ID: 909), CDIB (NCBI Gene ID: 910), CDIC (NCBI Gene ID: 911), CDID (NCBI Gene ID: 912), CDIE (NCBI Gene ID: 913), ITGAM, ITGAX, ITGB1, CD29, ITGB2 (CD18, LFA-1), ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D.

In some embodiments, the transmembrane domain comprises a transmembrane domain derived from a protein selected from the group consisting of the alpha, beta or zeta chain of the T-cell receptor, CD28, CD3 epsilon, CD3 delta, CD3 gamma, CD45, CD4, CD5, CD7, CD8 alpha, CD8 beta, CD9, CD11a, CD11b, CD11c, CD11d, CD16, CD18, CD22, CD33, CD37, CD64, CD80, CD86, CD134, CD137, CD154, KIRDS2, OX40, CD2, CD27, ICOS (CD278), 4-1BB(CD137), GITR, CD40, BAFFR, HVEM (LIGHTR), SLAMF7, NKp80 (KLRF1), CD19, CD19a, IL2R beta, IL2R gamma, IL7R alpha, ITGA1, VLA1, CD49a, ITGA4, IA4, CD49D, ITGA6, VLA-6, CD49f, ITGAD, CD1A, CD1B, CD1C, CD1D, CD1E, ITGAE, CD103, ITGAL, ITGAM, ITGAX, ITGB1, ITGB2, ITGB7, CD29, ITGB2 (LFA-1, CD18), ITGB7, TNFR2, DNAM1 (CD226), SLAMF4 (CD244, 2B4), CD84, CD96 (TACTILE), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and NKG2C activating NK cell receptors, an Immunoglobulin protein, BTLA, CD247, CD276 (B7-H3), CD30, CD84, CDS, cytokine receptor, Fc gamma receptor, GADS, ICAM-1, Ig alpha (CD79a), integrins, LAT, a ligand that binds with CD83, LIGHT, MHC class 1 molecule, PAG/Cbp, TNFSF14, a Toll ligand receptor, TRANCE/RANKL, or a fragment, truncation, or a combination thereof.

In some embodiments, the CAR comprises a hinge domain. A hinge domain may be derived from a protein selected from the group consisting of the CD2, CD3 delta, CD3 epsilon, CD3 gamma, CD4, CD7, CD8.alpha., CD8.beta., CD11a (ITGAL), CD11b (ITGAM), CD11c (ITGAX), CD11d (ITGAD), CD18 (ITGB2), CD19 (B4), CD27 (TNFRSF7), CD28, CD28T, CD29 (ITGB1), CD30 (TNFRSF8), CD40 (TNFRSF5), CD48 (SLAMF2), CD49a (ITGA1), CD49d (ITGA4), CD49f (ITGA6), CD66a (CEACAM1), CD66b (CEACAM8), CD66c (CEACAM6), CD66d (CEACAM3), CD66e (CEACAM5), CD69 (CLEC2), CD79A (B-cell antigen receptor complex-associated alpha chain), CD79B (B-cell antigen receptor complex-associated beta chain), CD84 (SLAMF5), CD96 (Tactile), CD100 (SEMA4D), CD103 (ITGAE), CD134 (OX40), CD137 (4-1BB), CD150 (SLAMF1), CD158A (KIR2DL1), CD158B1 (KIR2DL2), CD158B2 (KIR2DL3), CD158C (KIR3DP1), CD158D (KIRDL4), CD158F1 (KIR2DL5A), CD158F2 (KIR2DL5B), CD158K (KIR3DL2), CD160 (BY55), CD162 (SELPLG), CD226 (DNAM1), CD229 (SLAMF3), CD244 (SLAMF4), CD247 (CD3-zeta), CD258 (LIGHT), CD268 (BAFFR), CD270 (TNFSF14), CD272 (BTLA), CD276 (B7-H3), CD279 (PD-1), CD314 (NKG2D), CD319 (SLAMF7), CD335 (NK-p46), CD336 (NK-p44), CD337 (NK-p30), CD352 (SLAMF6), CD353 (SLAMF8), CD355 (CRTAM), CD357 (TNFRSF18), inducible T cell co-stimulator (ICOS), LFA-1 (CD11a/CD18), NKG2C, DAP-10, ICAM-1, NKp80 (KLRF1), IL-2R beta, IL-2R gamma, IL-7R alpha, LFA-1, SLAMF9, LAT, GADS (GrpL), SLP-76 (LCP2), PAG1/CBP, a CD83 ligand, Fc gamma receptor, MHC class 1 molecule, MHC class 2 molecule, a TNF receptor protein, an immunoglobulin protein, a cytokine receptor, an integrin, activating NK cell receptors, or Toll ligand receptor, IgG1, IgG2, IgG3, IgG4, IgA, IgD, IgE, IgM or fragment or combination thereof.

In some embodiments, the TCR or CAR antigen binding domain or the immunotherapeutic agent described herein (e.g., monospecific or multi-specific antibody or antigen-binding fragment thereof or antibody mimetic) binds a tumor-associated antigen (TAA). In some embodiments, the tumor-associated antigen is selected from the group consisting of: CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (αNeuSAc(2-8)αNeuSAc(2-3)βDGaip(1-4)bDGIcp(1-1)Cer); ganglioside GM3 (αNeuSAc(2-3)βDGalp(1-4)βDGlcp(1-1)Cer); GM-CSF receptor; TNF receptor superfamily member 17 (TNFRSF17, BCMA); B-lymphocyte cell adhesion molecule; Tn antigen ((Tn Ag) or (GaINAcu-Ser/Thr)); prostate-specific membrane antigen (PSMA); Receptor tyrosine kinase-like orphan receptor 1 (ROR1); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); HLA class I antigen A-2 alpha; HLA antigen; Lewis(Y) antigen; CD24; Platelet-derived growth factor receptor beta (PDGFR-beta); Stage-specific embryonic antigen-4 (SSEA-4); CD20; delta like 3 (DLL3); Folate receptor alpha; Folate receptor beta, GDNF alpha 4 receptor, Receptor tyrosine-protein kinase, ERBB2 (Her2/neu); Mucin 1, cell surface associated (MUC1); APRIL receptor; ADP ribosyl cyclase-1; Ephb4 tyrosine kinase receptor, DCAMKL1 serine threonine kinase, Aspartate beta-hydroxylase, epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); ephrin type-A receptor 3 (EphA3), Fucosyl GM1; sialyl Lewis adhesion molecule (sLe); transglutaminase 5 (TGS5); high molecular weight-melanomaassociatedantigen (HMWMAA); o-acetyl-GD2 ganglioside (OAcGD2); Folate receptor beta; tumor endothelial marker 1 (TEM1/CD248); tumor endothelial marker 7-related (TEM7R); six transmembrane epithelial antigen of the prostate I (STEAP1); claudin 6 (CLDN6); thyroid stimulating hormone receptor (TSHR); G protein-coupled receptor class C group 5, member D (GPRCSD); IL-15 receptor (IL-15); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GoboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (ORS IE2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma associated antigen 1 (MAGE-A1); Melanoma associated antigen 3 (MAGE-A3); Melanoma associated antigen 4 (MAGE-A4); T cell receptor beta 2 chain C; ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MADCT-1); melanoma cancer testis antigen-2 (MAD-CT-2); Fos-related antigen 1; tumor protein p53, (p53); p53 mutant; prostein; survivin; telomerase; prostate carcinoma tumor antigen-1 (PCTA-1 or Galectin 8), melanoma antigen recognized by T cells 1 (MelanA or MARTI); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin-A1; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1(CYP IBI); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES I); lymphocyte-specific protein tyrosine kinase (LCK); A kinase anchor protein 4 (AKAP-4); Peptidoglycan recognition protein, synovial sarcoma, X breakpoint 2 (SSX2); Receptor for Advanced Glycation Endproducts (RAGE-I); renal ubiquitous 1 (RUI); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIRI); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module containing mucin-like hormone receptor-like 2 (EMR2); lymphocyte antigen 75 (LY75); Glypican-2 (GPC2); Glypican-3 (GPC3); Fc receptor-like 5 (FCRL5); and immunoglobulin lambda-like polypeptide 1 (IGLL1). In some embodiments, the target is an epitope of the tumor associated antigen presented in an MHC.

In some embodiments, the tumor antigen is selected from CD150, 5T4, ActRIIA, B7, TNF receptor superfamily member 17 (TNFRSF17, BCMA), CA-125, CCNA1, CD123, CD126, CD138, CD14, CD148, CD15, CD19, CD20, CD200, CD21, CD22, CD23, CD24, CD25, CD26, CD261, CD262, CD30, CD33, CD362, CD37, CD38, CD4, CD40, CD40L, CD44, CD46, CD5, CD52, CD53, CD54, CD56, CD66a-d, CD74, CD8, CD80, CD92, CE7, CS-1, CSPG4, ED-B fibronectin, EGFR, EGFRvIII, EGP-2, EGP-4, EPHa2, ErbB2, ErbB3, ErbB4, FBP, HER1-HER2 in combination, HER2-HER3 in combination, HERV-K, HIV-1 envelope glycoprotein gp120, HIV-1 envelope glycoprotein gp41, HLA-DR, HLA class I antigen alpha G, HM1.24, K-Ras GTPase, HMW-MAA, Her2, Her2/neu, IGF-1R, IL-11Ralpha, IL-13R-alpha2, IL-2, IL-22R-alpha, IL-6, IL-6R, Ia, Ii, L1-CAM, L1-cell adhesion molecule, Lewis Y, L1-CAM, MAGE A3, MAGE-A1, MART-1, MUC1, NKG2C ligands, NKG2D Ligands, NYESO-1, OEPHa2, PIGF, PSCA, PSMA, ROR1, T101, TAC, TAG72, TIM-3, TRAIL-R1, TRAIL-RI (DR4), TRAIL-R2 (DR5), VEGF, VEGFR2, WT-I, a G-protein coupled receptor, alphafetoprotein (AFP), an angiogenesis factor, an exogenous cognate binding molecule (ExoCBM), oncogene product, anti-folate receptor, c-Met, carcinoembryonic antigen (CEA), cyclin (D 1), ephrinB2, epithelial tumor antigen, estrogen receptor, fetal acetylcholine e receptor, folate binding protein, gp100, hepatitis B surface antigen, Epstein-Barr nuclear antigen 1, Latent membrane protein 1, Secreted protein BARF1, P2X7 purinoceptor, Syndecan-1, kappa chain, kappa light chain, kdr, lambda chain, livin, melanoma-associated antigen, mesothelin, mouse double minute 2 homolog (MDM2), mucin 16 (MUC16), mutated p53, mutated ras, necrosis antigens, oncofetal antigen, ROR2, progesterone receptor, prostate specific antigen, tEGFR, tenascin, P2-Microgiobuiin, Fc Receptor-like 5 (FcRL5).

Examples of cell therapies include without limitation: AMG-119, Algenpantucel-L, ALOFISEL®, Sipuleucel-T, (BPX-501) rivogenlecleucel U.S. Pat. No. 9,089,520, WO2016100236, AU-105, ACTR-087, activated allogeneic natural killer cells CNDO-109-AANK, MG-4101, AU-101, BPX-601, FATE-NK100, LFU-835 hematopoietic stem cells, Imilecleucel-T, baltaleucel-T, PNK-007, UCARTCS1, ET-1504, ET-1501, ET-1502, ET-190, CD19-ARTEMIS, ProHema, FT-1050-treated bone marrow stem cell therapy, CD4CARNK-92 cells, SNK-01, NEXI-001, CryoStim, AlloStim, lentiviral transduced huCART-meso cells, CART-22 cells, EGFRt/19-28z/4-1BBL CAR T cells, autologous 4H11-28z/fnL-12/EFGRt T cell, CCR5-SBC-728-HSPC, CAR4-1BBZ, CH-296, dnTGFbRII-NY-ESOc259T, Ad-RTS-IL-12, IMA-101, IMA-201, CARMA-0508, TT-18, CMD-501, CMD-503, CMD-504, CMD-502, CMD-601, CMD-602, CSG-005, LAAP T-cell therapy, PD-1 knockout T cell therapy (esophageal cancer/NSCLC), anti-MUC1 CAR T-cell therapy (esophageal cancer/NSCLC), anti-MUC1 CAR T-cell therapy+PD-1 knockout T cell therapy (esophageal cancer/NSCLC), anti-KRAS G12D mTCR PBL, anti-CD123 CAR T-cell therapy, anti-mutated neoantigen TCR T-cell therapy, tumor lysate/MUC1/survivin PepTivator-loaded dendritic cell vaccine, autologous dendritic cell vaccine (metastatic malignant melanoma, intradermal/intravenous), anti-LeY-scFv-CD28-zeta CAR T-cells, PRGN-3005, iC9-GD2-CAR-IL-15 T-cells, HSC-100, ATL-DC-101, MIDRIX4-LUNG, MIDRIXNEO, FCR-001, PLX stem cell therapy, MDR-101, GeniusVac-Mel4, ilixadencel, allogeneic mesenchymal stem cell therapy, romyelocel L, CYNK-001, ProTrans, ECT-100, MSCTRAIL, dilanubicel, FT-516, ASTVAC-2, E-CEL UVEC, CK-0801, allogenic alpha/beta CD3+ T cell and CD19+B cell depleted stem cells (hematologic diseases, TBX-1400, HLCN-061, umbilical cord derived Hu-PHEC cells (hematological malignancies/aplastic anemia), AP-011, apceth-201, apceth-301, SENTI-101, stem cell therapy (pancreatic cancer), ICOVIR15-cBiTE, CD33HSC/CD33 CAR-T, PLX-Immune, SUBCUVAX, CRISPR allogeneic gamma-delta T-cell based gene therapy (cancer), ex vivo CRISPR allogeneic healthy donor NK-cell based gene therapy (cancer), ex-vivo allogeneic induced pluripotent stem cell-derived NK-cell based gene therapy (solid tumor), and anti-CD20 CAR T-cell therapy (non-Hodgkin's lymphoma).

Additional Agents for Targeting Tumors

Additional agents for targeting tumors include without limitation: Alpha-fetoprotein modulators, such as ET-1402, and AFP-TCR; Anthrax toxin receptor 1 modulator, such as anti-TEM8 CAR T-cell therapy; TNF receptor superfamily member 17 (TNFRSF17, BCMA), such as bb-2121 (ide-cel), bb-21217, JCARH125, UCART-BCMA, ET-140, MCM-998, LCAR-B38M, CART-BCMA, SEA-BCMA, BB212, ET-140, P-BCMA-101, AUTO-2 (APRIL-CAR), JNJ-68284528; Anti-CLL-1 antibodies, (see, for example, PCT/US2017/025573); Anti-PD-L-CAR tank cell therapy, such as KD-045; Anti-PD-L1 t-haNK, such as PD-L1 t-haNK; anti-CD45 antibodies, such as 131I-BC8 (lomab-B); anti-HER3 antibodies, such as LJM716, GSK2849330; APRIL receptor modulator, such as anti-BCMA CAR T-cell therapy, Descartes-011; ADP ribosyl cyclase-1/APRIL receptor modulator, such as dual anti-BCMA/anti-CD38 CAR T-cell therapy; CART-ddBCMA; B7 homolog 6, such as CAR-NKp30 and CAR-B7H6; B-lymphocyte antigen CD19, such as TBI-1501, CTL-119 huCART-19 T cells, l iso-cel, JCAR-015 U.S. Pat. No. 7,446,190, JCAR-014, JCAR-017, (WO2016196388, WO2016033570, WO2015157386), axicabtagene ciloleucel (KTE-C19, Yescarta®), KTE-X19, U.S. Pat. Nos. 7,741,465, 6,319,494, UCART-19, EBV-CTL, T tisagenlecleucel-T (CTL019), WO2012079000, WO2017049166, CD19CAR-CD28-CD3zeta-EGFRt-expressing T cells, CD19/4-1BBL armored CAR T cell therapy, C-CAR-011, CIK-CAR.CD19, CD19CAR-28-zeta T cells, PCAR-019, MatchCART, DSCAR-01, IM19 CAR-T, TC-110; anti-CD19 CAR T-cell therapy (B-cell acute lymphoblastic leukemia, Universiti Kebangsaan Malaysia); anti-CD19 CAR T-cell therapy (acute lymphoblastic leukemia/Non-Hodgkin's lymphoma, University Hospital Heidelberg), anti-CD19 CAR T-cell therapy (silenced IL-6 expression, cancer, Shanghai Unicar-Therapy Bio-medicine Technology), MB-CART2019.1 (CD19/CD20), GC-197 (CD19/CD7), CLIC-1901, ET-019003, anti-CD19-STAR-T cells, AVA-001, BCMA-CD19 cCAR (CD19/APRIL), ICG-134, ICG-132 (CD19/CD20), CTA-101, WZTL-002, dual anti-CD19/anti-CD20 CAR T-cells (chronic lymphocytic leukemia/B-cell lymphomas), HY-001, ET-019002, YTB-323, GC-012 (CD19/APRIL), GC-022 (CD19/CD22), CD19CAR-CD28-CD3zeta-EGFRt-expressing Tn/mem; UCAR-011, ICTCAR-014, GC-007F, PTG-01, CC-97540; allogeneic anti-CD19 CART cells, such as GC-007G; APRIL receptor modulator; SLAM family member 7 modulator, BCMA-CS1 cCAR; autologous dendritic cell tumor antigen (ADCTA), such as ADCTA-SSI-G; B-lymphocyte antigen CD20, such as ACTR707 ATTCK-20, PBCAR-20A; allogenic T cells expressing CD20 CAR, such as LB-1905; B-lymphocyte antigen CD19/B-lymphocyte antigen 22, such as TC-310; B-lymphocyte antigen 22 cell adhesion, such as UCART-22, JCAR-018 WO2016090190; NY-ESO-1 modulators, such as GSK-3377794, TBI-1301, GSK3537142; Carbonic anhydrase, such as DC-Ad-GMCAIX; Caspase 9 suicide gene, such as CaspaCIDe DLI, BPX-501; CCR5, such as SB-728; CCR5 gene inhibitor/TAT gene/TRIM5 gene stimulator, such as lentivirus vector CCR5 shRNA/TRIM5alpha/TAR decoy-transduced autologous CD34-positive hematopoietic progenitor cells; CDw123, such as MB-102, IM-23, JEZ-567, UCART-123; CD4, such as ICG-122; CD5 modulators, such as CD5.28z CART cells; Anti-CD22, such as anti-CD22 CART; Anti-CD30, such as TT-11; Dual anti-CD33/anti-CLL1, such as LB-1910; CD40 ligand, such as BPX-201, MEDI5083; CD56, such as allogeneic CD56-positive CD3-negative natural killer cells (myeloid malignancies); CD19/CD7 modulator, such as GC-197; T-cell antigen CD7 modulator, such as anti-CD7 CAR T-cell therapy (CD7-positive hematological malignancies); CD123 modulator, such as UniCAR02-T-CD123; Anti-CD276, such as anti-CD276 CART; CEACAM protein 5 modulators, such as MG7-CART; Claudin 6, such as CSG-002; Claudin 18.2, such as LB-1904; Chlorotoxin, such as CLTX-CART; EBV targeted, such as CMD-003; MUC16EGFR, such as autologous 4H11-28z/fIL-12/EFGRt T cell; Endonuclease, such as PGN-514, PGN-201; Epstein-Barr virus specific T-lymphocytes, such as TT-10; Epstein-Barr nuclear antigen 1/Latent membrane protein 1/Secreted protein BARF1 modulator, such as TT-10X; Erbb2, such as CST-102, CIDeCAR; Ganglioside (GD2), such as 4SCAR-GD2; Gamma delta T cells, such as ICS-200; folate hydrolase 1 (FOLH1, Glutamate carboxypeptidase II, PSMA; NCBI Gene ID: 2346), such as CIK-CAR.PSMA, CART-PSMA-TGFβRDN, P-PSMA-101; Glypican-3(GPC3), such as TT-16, GLYCAR; Hemoglobin, such as PGN-236; Hepatocyte growth factor receptor, such as anti-cMet RNA CAR T; HLA class I antigen A-2 alpha modulator, such as FH-MCVA2TCR; HLA class I antigen A-2 alpha/Melanoma associated antigen 4 modulator, such as ADP-A2M4CD8; HLA antigen modulator, such as FIT-001, NeoTCR-P1; Human papillomavirus E7 protein, such as KITE-439 (see, for example, PCT/US2015/033129); ICAM-1 modulator, such as AIC-100; Immunoglobulin gamma Fc receptor III, such as ACTR087; IL-12, such as DC-RTS-IL-12; IL-12 agonist/mucin 16, such as JCAR-020; IL-13 alpha 2, such as MB-101; IL-15 receptor agonist, such as PRGN-3006, ALT-803; interleukin-15/Fc fusion protein (e.g., XmAb24306); recombinant interleukin-15 (e.g., AM0015, NIZ-985); pegylated IL-15 (e.g., NKTR-255); IL-2, such as CST-101; Interferon alpha ligand, such as autologous tumor cell vaccine+systemic CpG-B+IFN-alpha (cancer); K-Ras GTPase, such as anti-KRAS G12V mTCR cell therapy; Neural cell adhesion molecule L1 LICAM (CD171), such as JCAR-023; Latent membrane protein 1/Latent membrane protein 2, such as Ad5f35-LMPd1-2-transduced autologous dendritic cells; MART-1 melanoma antigen modulator, such as MART-1 F5 TCR engineered PBMC; Melanoma associated antigen 10, such as MAGE-A10C796T MAGE-A10 TCR; Melanoma associated antigen 3/Melanoma associated antigen 6 (MAGE A3/A6) such as KITE-718 (see, for example, PCT/US2013/059608); Mesothelin, such as CSG-MESO, TC-210; Mucin 1 modulator, such as ICTCAR-052, Tn MUC-1 CAR-T, ICTCAR-053; Anti-MICA/MICB, such as CYAD-02; NKG2D, such as NKR-2; Ntrkr1 tyrosine kinase receptor, such as JCAR-024; PRAMET cell receptor, such as BPX-701; Prostate stem cell antigen modulator, such as MB-105; Roundabout homolog 1 modulator, such as ATCG-427; Peptidoglycan recognition protein modulator, such as Tag-7 gene modified autologous tumor cell vaccine; PSMA, such as PSMA-CAR T-cell therapy (lentiviral vector, castrate-resistant prostate cancer); SLAM family member 7 modulator, such as IC9-Luc90-CD828Z; TGF beta receptor modulator, such as DNR.NPC T-cells; T-lymphocyte, such as TT-12; T-lymphocyte stimulator, such as ATL-001; TSH receptor modulator, such as ICTCAR-051; Tumor infiltrating lymphocytes, such as LN-144, LN-145; and/or Wilms tumor protein, such as JTCR-016, WT1-CTL, ASP-7517.

MCL1 Apoptosis Regulator, BCL2 Family Member (MCLI) Inhibitors

In various embodiments, an anti-CD47 agent or an anti-SIRPα agent as described herein, is combined with an inhibitor of MCL1 apoptosis regulator, BCL2 family member (MCL1, TM; EAT; MCL1L; MCL1S; Mcl-1; BCL2L3; MCL1-ES; bcl2-L-3; mcl1/EAT; NCBI Gene ID: 4170). Examples of MCL1 inhibitors include AMG-176, AMG-397, S-64315, and AZD-5991, 483-LM, A-1210477, UMI-77, JKY-5-037, and those described in WO2018183418, WO2016033486, WO2019222112 and WO2017147410.

Cytokine Inducible SH2 Containing Protein (CISH) Inhibitors

In various embodiments, an anti-CD47 agent or an anti-SIRPα agent as described herein, is combined with an inhibitor of cytokine inducible SH2 containing protein (CISH; CIS; G18; SOCS; CIS-1; BACTS2; NCBI Gene ID: 1154). Examples of CISH inhibitors include those described in WO2017100861, WO2018075664 and WO2019213610.

Gene Editors

In various embodiments, an anti-CD47 agent or an anti-SIRPα agent as described herein, is combined with gene editor. Illustrative gene editing system that can be co-administered include without limitation a CRISPR/Cas9 system, a zinc finger nuclease system, a TALEN system, a homing endonucleases system (e.g., an ARCUS), and a homing meganuclease system.

Others Drugs with Unspecified Targets

In various embodiments, an anti-CD47 agent or an anti-SIRPα agent as described herein, is combined with human immunoglobulin (10% liquid formulation), Cuvitru (human immunoglobulin (20% solution), levofolinate disodium, IMSA-101, BMS-986288, IMUNO BGC Moreau RJ, R-OKY-034F, GP-2250, AR-23, calcium levofolinate, porfimer sodium, RG6160, ABBV-155, CC-99282, polifeprosan 20 with carmustine, Veregen, gadoxetate disodium, gadobutrol, gadoterate meglumine, gadoteridol, 99mTc-sestamibi, pomalidomide, pacibanil, and/or valrubicin,

### Exemplified Combination Therapies

Lymphoma or Leukemia Combination Therapy

Some chemotherapy agents are suitable for treating lymphoma or leukemia. These agents include aldesleukin, alvocidib, amifostine trihydrate, aminocamptothecin, antineoplaston A10, antineoplaston AS2-1, anti-thymocyte globulin, arsenic trioxide, Bcl-2 family protein inhibitor ABT-263, beta alethine, BMS-345541bortezomib (VELCADE®, PS-341), bryostatin 1, bulsulfan, campath-1H, carboplatin, carfilzomib (Kyprolis®), carmustine, caspofungin acetate, CC-5103, chlorambucil, CHOP (cyclophosphamide, doxorubicin, vincristine, and prednisone), cisplatin, cladribine, clofarabine, curcumin, CVP (cyclophosphamide, vincristine, and prednisone), cyclophosphamide, cyclosporine, cytarabine, denileukin diftitox, dexamethasone, docetaxel, dolastatin 10, doxorubicin, doxorubicin hydrochloride, DT-PACE (dexamethasone, thalidomide, cisplatin, doxorubicin, cyclophosphamide, and etoposide), enzastaurin, epoetin alfa, etoposide, everolimus (RAD001), FCM (fludarabine, cyclophosphamide, and mitoxantrone), FCR (fludarabine, cyclophosphamide, and rituximab), fenretinide, filgrastim, flavopiridol, fludarabine, FR (fludarabine and rituximab), geldanamycin (17 AAG), hyperCVAD (hyperfractionated cyclophosphamide, vincristine, doxorubicin, dexamethasone, methotrexate, and cytarabine), ICE (iphosphamide, carboplatin, and etoposide), ifosfamide, irinotecan hydrochloride, interferon alpha-2b, ixabepilone, lenalidomide (REVLIMID®, CC-5013), pomalidomide (POMALYST®/IMNOVID®)lymphokine-activated killer cells, MCP (mitoxantrone, chlorambucil, and prednisolone), melphalan, mesna, methotrexate, mitoxantrone hydrochloride, motexafin gadolinium, mycophenolate mofetil, nelarabine, obatoclax (GX15-070), oblimersen, octreotide acetate, omega-3 fatty acids, Omr-IgG-am (WNIG, Omrix), oxaliplatin, paclitaxel, palbociclib (PD0332991), pegfilgrastim, PEGylated liposomal doxorubicin hydrochloride, perifosin, prednisolone, prednisone, recombinant flt3 ligand, recombinant human thrombopoietin, recombinant interferon alfa, recombinant interleukin-11, recombinant interleukin-12, rituximab, R-CHOP (rituximab and CHOP), R-CVP (rituximab and CVP), R-FCM (rituximab and FCM), R-ICE (rituximab and ICE), and R MCP (rituximab and MCP), R-roscovitine (seliciclib, CYC202), sargramostim, sildenafil citrate, simvastatin, sirolimus, styryl sulphones, tacrolimus, tanespimycin, temsirolimus (CCl-779), thalidomide, therapeutic allogeneic lymphocytes, thiotepa, tipifarnib, vincristine, vincristine sulfate, vinorelbine ditartrate, SAHA (suberanilohydroxamic acid, or suberoyl, anilide, and hydroxamic acid), vemurafenib (Zelboraf®), venetoclax (ABT-199).

One modified approach is radioimmunotherapy, wherein a monoclonal antibody is combined with a radioisotope particle, such as indium-111, yttrium-90, and iodine-131. Examples of combination therapies include, but are not limited to, iodine-131 tositumomab (BEXXAR®), yttrium-90 ibritumomab tiuxetan (ZEVALIN®), and BEXXAR® with CHOP.

The abovementioned therapies can be supplemented or combined with stem cell transplantation or treatment. Therapeutic procedures include peripheral blood stem cell transplantation, autologous hematopoietic stem cell transplantation, autologous bone marrow transplantation, antibody therapy, biological therapy, enzyme inhibitor therapy, total body irradiation, infusion of stem cells, bone marrow ablation with stem cell support, in vitro-treated peripheral blood stem cell transplantation, umbilical cord blood transplantation, immunoenzyme technique, low-LET cobalt-60 gamma ray therapy, bleomycin, conventional surgery, radiation therapy, and nonmyeloablative allogeneic hematopoietic stem cell transplantation.

Non-Hodgkin's Lymphomas Combination Therapy

Treatment of non-Hodgkin's lymphomas (NHL), especially those of B cell origin, includes using monoclonal antibodies, standard chemotherapy approaches (e.g., CHOP (cyclophosphamide, doxorubicin, vincristine, and prednisone), CVP (cyclophosphamide, vincristine, and prednisone), FCM (fludarabine, cyclophosphamide, and mitoxantrone), MCP (Mitoxantrone, Chlorambucil, Prednisolone), all optionally including rituximab (R) and the like), radioimmunotherapy, and combinations thereof, especially integration of an antibody therapy with chemotherapy.

Examples of unconjugated monoclonal antibodies for the treatment of NHL/B-cell cancers include rituximab, alemtuzumab, human or humanized anti-CD20 antibodies, lumiliximab, anti-TNF-related apoptosis-inducing ligand (anti-TRAIL), bevacizumab, galiximab, epratuzumab, SGN-40, and anti-CD74.

Examples of experimental antibody agents used in treatment of NHL/B-cell cancers include ofatumumab, ha20, PRO131921, alemtuzumab, galiximab, SGN-40, CHIR-12.12, epratuzumab, lumiliximab, apolizumab, milatuzumab, and bevacizumab.

Examples of standard regimens of chemotherapy for NHL/B-cell cancers include CHOP, FCM, CVP, MCP, R-CHOP (rituximab, cyclophosphamide, doxorubicin, vincristine, and prednisone), R-FCM, R-CVP, and R MCP.

Examples of radioimmunotherapy for NHL/B-cell cancers include yttrium-90 ibritumomab tiuxetan (ZEVALIN®) and iodine-131 tositumomab (BEXXAR®).

Mantle Cell Lymphoma Combination Therapy

Therapeutic treatments for mantle cell lymphoma (MCL) include combination chemotherapies such as CHOP, hyperCVAD, and FCM. These regimens can also be supplemented with the monoclonal antibody rituximab to form combination therapies R-CHOP, hyperCVAD-R, and R-FCM. Any of the abovementioned therapies may be combined with stem cell transplantation or ICE in order to treat MCL.

An alternative approach to treating MCL is immunotherapy. One immunotherapy uses monoclonal antibodies like rituximab. Another uses cancer vaccines, such as GTOP-99, which are based on the genetic makeup of an individual patient's tumor.

A modified approach to treat MCL is radioimmunotherapy, wherein a monoclonal antibody is combined with a radioisotope particle, such as iodine-131 tositumomab (BEXXAR®) and yttrium-90 ibritumomab tiuxetan (ZEVALIN®). In another example, BEXXAR® is used in sequential treatment with CHOP.

Other approaches to treating MCL include autologous stem cell transplantation coupled with high-dose chemotherapy, administering proteasome inhibitors such as bortezomib (VELCADE® or PS-341), or administering antiangiogenesis agents such as thalidomide, especially in combination with rituximab.

Another treatment approach is administering drugs that lead to the degradation of Bcl-2 protein and increase cancer cell sensitivity to chemotherapy, such as oblimersen, in combination with other chemotherapeutic agents.

A further treatment approach includes administering mTOR inhibitors, which can lead to inhibition of cell growth and even cell death. Non-limiting examples are sirolimus, temsirolimus (TORISEL®, CCI-779), CC-115, CC-223, SF-1126, PQR-309 (bimiralisib), voxtalisib, GSK-2126458, and temsirolimus in combination with RITUXAN®, VELCADE®, or other chemotherapeutic agents.

Other recent therapies for MCL have been disclosed. Such examples include flavopiridol, palbociclib (PD0332991), R-roscovitine (selicicilib, CYC202), styryl sulphones, obatoclax (GX15-070), TRAIL, Anti-TRAIL death receptors DR4 and DR5 antibodies, temsirolimus (TORISEL®, CCl-779), everolimus (RAD001), BMS-345541, curcumin, SAHA, thalidomide, lenalidomide (REVLIMID®, CC-5013), and geldanamycin (17 AAG).

### Waldenström's Macroglobulinemia Combination Therapy

Therapeutic agents used to treat Waldenstrom's Macroglobulinemia (WM) include aldesleukin, alemtuzumab, alvocidib, amifostine trihydrate, aminocamptothecin, antineoplaston A10, antineoplaston AS2-1, anti-thymocyte globulin, arsenic trioxide, autologous human tumor-derived HSPPC-96, Bcl-2 family protein inhibitor ABT-263, beta alethine, bortezomib (VELCADE®), bryostatin 1, busulfan, campath-1H, carboplatin, carmustine, caspofungin acetate, CC-5103, cisplatin, clofarabine, cyclophosphamide, cyclosporine, cytarabine, denileukin diftitox, dexamethasone, docetaxel, dolastatin 10, doxorubicin hydrochloride, DT-PACE, enzastaurin, epoetin alfa, epratuzumab (hLL2-anti-CD22 humanized antibody), etoposide, everolimus, fenretinide, filgrastim, fludarabine, ibrutinib, ifosfamide, indium-111 monoclonal antibody MN-14, iodine-131 tositumomab, irinotecan hydrochloride, ixabepilone, lymphokine-activated killer cells, melphalan, mesna, methotrexate, mitoxantrone hydrochloride, monoclonal antibody CD19 (such as tisagenlecleucel-T, CART-19, CTL-019), monoclonal antibody CD20, motexafin gadolinium, mycophenolate mofetil, nelarabine, oblimersen, octreotide acetate, omega-3 fatty acids, oxaliplatin, paclitaxel, pegfilgrastim, PEGylated liposomal doxorubicin hydrochloride, pentostatin, perifosine, prednisone, recombinant flt3 ligand, recombinant human thrombopoietin, recombinant interferon alfa, recombinant interleukin-11, recombinant interleukin-12, rituximab, sargramostim, sildenafil citrate (VIAGRA®), simvastatin, sirolimus, tacrolimus, tanespimycin, thalidomide, therapeutic allogeneic lymphocytes, thiotepa, tipifarnib, tositumomab, ulocuplumab, veltuzumab, vincristine sulfate, vinorelbine ditartrate, vorinostat, WTT 126-134 peptide vaccine, WT-1 analog peptide vaccine, yttrium-90 ibritumomab tiuxetan, yttrium-90 humanized epratuzumab, and any combination thereof.

Examples of therapeutic procedures used to treat WM include peripheral blood stem cell transplantation, autologous hematopoietic stem cell transplantation, autologous bone marrow transplantation, antibody therapy, biological therapy, enzyme inhibitor therapy, total body irradiation, infusion of stem cells, bone marrow ablation with stem cell support, in vitro-treated peripheral blood stem cell transplantation, umbilical cord blood transplantation, immunoenzyme techniques, low-LET cobalt-60 gamma ray therapy, bleomycin, conventional surgery, radiation therapy, and nonmyeloablative allogeneic hematopoietic stem cell transplantation.

Diffuse Large B-Cell Lymphoma Combination Therapy

Therapeutic agents used to treat diffuse large B-cell lymphoma (DLBCL) include cyclophosphamide, doxorubicin, vincristine, prednisone, anti-CD20 monoclonal antibodies, etoposide, bleomycin, many of the agents listed for WM, and any combination thereof, such as ICE and RICE.

Chronic Lymphocytic Leukemia Combination Therapy

Examples of therapeutic agents used to treat chronic lymphocytic leukemia (CLL) include chlorambucil, cyclophosphamide, fludarabine, pentostatin, cladribine, doxorubicin, vincristine, prednisone, prednisolone, alemtuzumab, many of the agents listed for WM, and combination chemotherapy and chemoimmunotherapy, including the following common combination regimens: CVP, R-CVP, ICE, R-ICE, FCR, and FR.

Myelofibrosis Combination Therapy

Myelofibrosis inhibiting agents include, but are not limited to, hedgehog inhibitors, histone deacetylase (HDAC) inhibitors, and tyrosine kinase inhibitors. Non-limiting examples of hedgehog inhibitors are saridegib and vismodegib. Examples of HDAC inhibitors include, but are not limited to, pracinostat and panobinostat. Non-limiting examples of tyrosine kinase inhibitors are lestaurtinib, bosutinib, imatinib, radotinib, and cabozantinib.

Hyperproliferative Disorder Combination Therapy

Gemcitabine, nab-paclitaxel, and gemcitabine/nab-paclitaxel may be used with a JAK inhibitor and/or PI3Kδ inhibitor to treat hyperproliferative disorders.

Cancer

The terms “cancer,” “neoplasm,” and “tumor” are used interchangeably herein to refer to cells which exhibit autonomous, unregulated growth, such that they exhibit an aberrant growth phenotype characterized by a significant loss of control over cell proliferation. Cells of interest for detection, analysis, or treatment in the present application include precancerous (e.g., benign), malignant, pre-metastatic, metastatic, and non-metastatic cells. Cancers of virtually every tissue are known. The phrase “cancer burden” refers to the quantum of cancer cells or cancer volume in a subject. Reducing cancer burden accordingly refers to reducing the number of cancer cells or the cancer volume in a subject. The term “cancer cell” as used herein refers to any cell that is a cancer cell or is derived from a cancer cell e.g. clone of a cancer cell. Many types of cancers are known to those of skill in the art, including solid tumors such as carcinomas, sarcomas, glioblastomas, melanomas, lymphomas, myelomas, etc., and circulating cancers such as leukemias.

The “pathology” of cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, pre-malignancy, malignancy, invasion of surrounding or distant tissues or organs, such as lymph nodes, etc.

As used herein, the terms “cancer recurrence” and “tumor recurrence,” and grammatical variants thereof, refer to further growth of neoplastic or cancerous cells after diagnosis of cancer. Particularly, recurrence may occur when further cancerous cell growth occurs in the cancerous tissue. “Tumor spread,” similarly, occurs when the cells of a tumor disseminate into local or distant tissues and organs; therefore tumor spread encompasses tumor metastasis. “Tumor invasion” occurs when the tumor growth spread out locally to compromise the function of involved tissues by compression, destruction, or prevention of normal organ function.

As used herein, the term “metastasis” refers to the growth of a cancerous tumor in an organ or body part, which is not directly connected to the organ of the original cancerous tumor. Metastasis will be understood to include micrometastasis, which is the presence of an undetectable amount of cancerous cells in an organ or body part which is not directly connected to the organ of the original cancerous tumor. Metastasis can also be defined as several steps of a process, such as the departure of cancer cells from an original tumor site, and migration and/or invasion of cancer cells to other parts of the body.

In some embodiments, the patient has a low mutation burden. In some embodiments, the patient has a high mutation burden. As is known in the art, cancer types can vary in the average or specific degree of mutation, where higher levels of mutation are associated with increased expression of neoantigens. See, for example, Vogelstein et al., (2013), supra. A low mutation burden can be a cancer type with an average per tumor, or specific number for an individual tumor, of up to about 10, up to about 20, up to about 30, up to about 40, up to about 50 non-synonymous mutations per tumor. A high mutation burden can be a cancer type with greater than about 50, greater than about 75, greater than about 100, greater than about 125, greater than about 150 non-synonymous mutations per tumor.

CD20+ Cancer

Provided herein are methods for treating individuals having a CD20+ cancer or reducing the size of such cancer in the subject, comprising administering: a therapeutically effective amount of an anti-CD47 antibody to the subject; and, optionally a therapeutically effective amount of at least one additional agent to the subject such as an anti-CD20 agent.

In some embodiments, a CD20+ cancer is a B cell cancer. In some embodiments, a subject has a B-cell hematologic malignancy. In some embodiments, a CD20+ cancer is an indolent or aggressive lymphoma. In some embodiments, the subject has a relapsed or refractory form of a B-cell cancer. B cell cancers can include Non-Hodgkin's lymphoma (NHL). In some embodiments, the NHL is low-grade or high risk NHL. In some embodiments, the NHL is follicular (e.g., bulky, non-bulky, or advanced follicular) or nonfollicular NHL.

NHL can include indolent lymphoma. Indolent lymphoma can include follicular lymphoma (FL). Indolent lymphoma can include marginal zone lymphoma.

NHL can include diffuse large B cell lymphoma (DLBCL). NHL can further include DLBCL subtypes such as de novo DLBCL or transformed DLBCL. DLBCL can be from different cells of origin including activated B cell, germinal center B cell, and double hit lymphoma.

A CD20+ cancer can include diffuse large B-cell lymphoma (DLBCL) (including relapsed or refractory), follicular lymphoma (FL) (including relapsed, refractory, or asymptomatic), non-Hodgkin's lymphoma (NHL) (including relapsed or refractory), marginal zone lymphoma (e.g., extranodal marginal-zone lymphoma), mantle cell lymphoma (MCL) (including relapsed or refractory), chronic lymphocytic leukemia (CLL)/small lymphocytic leukemia (including relapsed or refractory), Waldenstrom's macroglobulinemia/lymphoplasmacytic lymphoma, primary mediastinal B-cell lymphoma, Burkitt's lymphoma, double hit lymphoma (e.g., high grade B cell lymphoma with MYC and one or both of BCL2 or BCL6 rearrangement), myc-rearranged lymphoma, B-cell lymphoma unclassified, B-cell acute lymphoblastic leukemia (ALL) (e.g., Philadelphia chromosome-negative acute lymphoblastic leukemia), or post-transplant lymphoproliferative disease (PTLD). A given CD20+ cancer sub-type, such as those disclosed herein, can be classified based on histopathology, flow cytometry, molecular classification, one or more equivalent assays, or a combination thereof.

A CD20+ cancer can include double hit lymphoma (e.g., high grade C cell lymphoma with MYC and BCL2 and/or BCL6 rearrangement). A CD20+ cancer can include a myc-rearranged lymphoma.

Assays

In some aspects, the presence or absence of B-cells (e.g., CD19 or CD20 B-cells) can be determined by an assay. Presence of absence of B-cells can be detected using assays that detect CD19/CD20 specific proteins (e.g., CD19 and CD20). Additionally, serum levels of antibodies (e.g., antibody treatments such as anti-CD47 antibody (e.g., magrolimab) and anti-CD20 antibody (e.g., rituximab)) can also be quantified through assays.

Example assays can include immunohistochemistry, flow cytometry, mass cytometry (CyTOF), or gene expression by RNA profiling or RNA sequencing, microarray analysis or other gene expression profiling method. Additional examples of assays that can be used to measure presence/absence of B-cells include DNA assays (including whole genome or exome sequencing), microarrays, polymerase chain reaction (PCR), RT-PCR, Southern blots, Northern blots, antibody-binding assays, enzyme-linked immunosorbent assays (ELISAs), protein assays, Western blots, nephelometry, turbidimetry, chromatography, mass spectrometry, immunoassays, including, by way of example, but not limitation, RIA, immunofluorescence, immunochemiluminescence, immunoelectrochemiluminescence, or competitive immunoassays, and immunoprecipitation. Further examples of assays can include a B-cell resistance panel, immunoglobulin sequencing, or enzyme-linked immunospot (ELIspot) assay. The information from the assay can be quantitative and sent to a computer system of the invention. The information can also be qualitative, such as observing patterns or fluorescence, which can be translated into a quantitative measure by a user or automatically by a reader or computer system. In an embodiment, the subject can also provide information other than assay information to a computer system, such as race, height, weight, age, gender, eye color, hair color, family medical history and any other information that may be useful to a user, such as a clinical factor.

Protein detection assays are assays used to detect the expression level of a given protein (e.g., an anti-CD47 antibody or anti-CD20 antibody) from a sample. Protein detection assays are generally known in the art and can include an immunoassay, a protein-binding assay, an antibody-based assay, an antigen-binding protein-based assay, a protein-based array, an enzyme-linked immunosorbent assay (ELISA), flow cytometry, a protein array, a blot, a Western blot, nephelometry, turbidimetry, chromatography, mass spectrometry, enzymatic activity, and an immunoassays selected from RIA, immunofluorescence, immunochemiluminescence, immunoelectrochemiluminescence, immunoelectrophoretic, a competitive immunoassay, and immunoprecipitation. Exemplary example assays that can be used to measure serum levels of antibodies include ELISAs, immunoassays, ELIspot, Fluorospot, flow cytometry, Western Blot, spectrometry (e.g., liquid chromatography-mass spectrometry), or surface plasmon resonance.

Protein based analysis, using an antibody as described above that specifically binds to a polypeptide encoded by an altered nucleic acid or an antibody that specifically binds to a polypeptide encoded by a non-altered nucleic acid, or an antibody that specifically binds to a particular splicing variant encoded by a nucleic acid, can be used to identify the presence in a test sample of a particular splicing variant or of a polypeptide encoded by a polymorphic or altered nucleic acid, or the absence in a test sample of a particular splicing variant or of a polypeptide encoded by a non-polymorphic or non-altered nucleic acid. The presence of a polypeptide encoded by a polymorphic or altered nucleic acid, or the absence of a polypeptide encoded by a non-polymorphic or non-altered nucleic acid, is diagnostic for a susceptibility to coronary artery disease.

In one aspect, the level or amount of polypeptide encoded by a nucleic acid in a test sample is compared with the level or amount of the polypeptide encoded by the nucleic acid in a control sample. A level or amount of the polypeptide in the test sample that is higher or lower than the level or amount of the polypeptide in the control sample, such that the difference is statistically significant, is indicative of an alteration in the expression of the polypeptide encoded by the nucleic acid, and is diagnostic. Alternatively, the composition of the polypeptide encoded by a nucleic acid in a test sample is compared with the composition of the polypeptide encoded by the nucleic acid in a control sample (e.g., the presence of different splicing variants). A difference in the composition of the polypeptide in the test sample, as compared with the composition of the polypeptide in the control sample, is diagnostic. In another aspect, both the level or amount and the composition of the polypeptide can be assessed in the test sample and in the control sample. A difference in the amount or level of the polypeptide in the test sample, compared to the control sample; a difference in composition in the test sample, compared to the control sample; or both a difference in the amount or level, and a difference in the composition, is indicative of whether a subject should be treated with an anti-CD47 antibody, either increased or decreased.

In addition, one of skill in the art will also understand that the above described methods can also generally be used to detect markers that do not include a polymorphism. In some aspects the subject from whom a sample is taken for an assay has activated B-cell (ABC) DLBCL. In some aspects the subject from whom a sample is taken for an assay has non-germinal center B cell (GCB) DLBCL. In some aspects, the subject has increased expression of CD47 relative to (normal) control and the anti-CD47 antibody is administered to the subject, optionally the subject has ABC or non-germinal center B cell (GCB) DLBCL. Determination of ABC or GCB status can be performed, e.g., by gene expression profiling.

Assays can further be performed to determine effective doses of a therapeutic (e.g., anti-CD47 antibody or anti-CD20 antibody) to be provided to a subject. One example of such an assay is a Receptor occupancy (RO) assay, which measures the level of occupancy by binding agents, e.g., anti-CD47 antibody (Ab). The purpose of measuring the level of CD47 RO is to determine the relationship between the dose of a CD47 binding agent, the CD47 receptor saturation, and pharmacologic effect. The percent of receptor occupancy over time may provide useful information regarding the amount of drug or duration of exposure needed to produce the desired pharmacological effect. This assay can be used to determine the overall RO in the body by measuring the CD47 RO on surrogate cells, e.g. on CD45 negative (−) red blood cells (RBCs) and CD45 positive (+) white blood cells (WBCs), or other cell populations, e.g. bone marrow or tissue cells obtained through tissue biopsies. The RO assay can also be used to determine CD47 RO on target cells, e.g. RBC, leukemia cells or solid tumor cells, for CD47 binding and or blocking therapies.

Of interest is the use of this assay to determine the threshold of CD47 receptor occupancy that is correlated with the desired pharmacological effect. This threshold can be determined by assays performed ex vivo (in vitro) or by analysis of samples during in vivo dosing/treatment.

In one embodiment of the assay, a CD47 binding standard curve on a cell of interest cells is made by using fluorochrome-conjugated antibody at various concentrations. Receptor occupancy is measured by incubating the target cells with unlabeled antibody under different concentrations, and then the cells were either assayed in in vitro phagocytosis or incubated with a saturating concentration of labeled antibody based on the standard curve and analyzed for binding by flow cytometry. Receptor occupancy was calculated as follows:

% RO=100−((MFIₜₑₛₜ−MFI_{unstained})/(MFI_{saturated STD}−MFI_{unstained}))×100

In other embodiments the assay is performed by infusing a patient with a defined dose of antibody, obtaining a tissue sample, e.g. a blood sample, from the patient, usually before and after infusion of the antibody. The tissue sample is incubated with a saturating concentration of labeled antibody, and analyzed by flow cytometry. The analysis may be gated, for example, on red blood cells, white blood cells, cancer cells, etc.

It has been found that a priming dose that achieves at least about 80% saturation of CD47 on RBCs is sufficient to induce compensation for anemia and reduce degree of anemia on subsequent doses. In humans, the priming dose has been found to be as discussed above, i.e. from about 0.5 mg/kg to about 5 mg/kg, e.g., 1 mg/kg. In some embodiments, a receptor occupancy assay is performed with a candidate CD47 bind agent to determine the level of priming dose that provides for at least about 50% saturation on RBC, at least about 60% saturation, at least about 70% saturation, at least about 80% saturation, at least about 90% saturation, at least about 95% saturation, at least about 99% saturation, or more.

In some embodiments, a receptor occupancy assay is performed to determine the appropriate priming dose for a candidate anti-CD47 agent, e.g. an antibody that binds to CD47, a SIRPα polypeptide, etc.

Methods of Use

Methods are provided for treating a subject with a therapeutic dose of anti-CD47 agent. For example, a method can include treating a human subject having a CD20+ cancer or reducing the size of the CD20+ cancer in the human subject, comprising: (a) administering an anti-CD47 antibody to the subject at a dose of greater than or equal to 10 mg of antibody per kg of body weight; and (b) administering an anti-CD20 antibody to the subject. In various embodiments, prior to administering the anti-CD47 antibody and anti-CD20 antibody to the subject, the methods further comprise determining of having determined that B-cells are present in the subject, which can mean that the subject is eligible to receive the antibody treatments.

Methods can include a step of administering a primer agent to subject, followed by a step of administering a therapeutically effective dose of an anti-CD47 agent to the subject. In some embodiments, the step of administering a therapeutically effective dose is performed after at least about 3 days (e.g., at least about 4 days, at least about 5 days, at least about 6 days, at least about 7 days, at least about 8 days, at least about 9 days, or at least about 10 days) after beginning the administration of a primer agent. This period of time is, for example, sufficient to provide for enhanced reticulocyte production by the individual.

The administration of a therapeutically effective dose of an anti-CD47 agent can be achieved in a number of different ways. In some cases, two or more therapeutically effective doses are administered after a primer agent is administered. Suitable administration of a therapeutically effective dose can entail administration of a single dose, or can entail administration of doses daily, semi-weekly, weekly, once every two weeks, once a month, annually, etc. In some cases, a therapeutically effective dose is administered as two or more doses of escalating concentration (i.e., increasing doses), where (i) all of the doses are therapeutic doses, or where (ii) a sub-therapeutic dose (or two or more sub-therapeutic doses) is initially given and therapeutic doses are achieved by said escalation. As one non-limiting example to illustrate escalating concentration (i.e., increasing doses), a therapeutically effective dose can be administered weekly, beginning with a sub-therapeutic dose (e.g., a dose of less than 10 mg/kg, e.g., a dose of 5 mg/kg, 4 mg/kg, 3 mg/kg, 2 mg/kg, 1 mg/kg), and each subsequent dose can be increased by a particular increment (e.g., by 5 mg/kg, 10 mg/kg, 15 mg/kg), or by variable increments, until a therapeutic dose (e.g., 15 mg/kg, 30 mg/kg, 45 mg/kg, 60 mg/kg) is reached, at which point administration may cease or may continue with one or more additional therapeutic doses (e.g., continued therapeutic doses escalated therapeutic doses, e.g., doses of 15 mg/kg, 30 mg/kg, 45 mg/kg, 60 mg/kg). As another non-limiting example to illustrate escalating concentration (i.e., increasing doses), a therapeutically effective dose can be administered weekly, beginning with one or more relatively lower therapeutic doses (e.g., a dose of 10 mg/kg, 15 mg/kg, 30 mg/kg), and each subsequent dose can be increased by a particular increment (e.g., by 10 mg/kg or 15 mg/kg), or by variable increments, until a relatively higher therapeutic dose (e.g., 30 mg/kg, 45 mg/kg, 60 mg/kg, 100 mg/kg, etc.) is reached, at which point administration may cease or may continue (e.g., one or more continued therapeutic doses or escalating, e.g., doses of 30 mg/kg, 45 mg/kg, 60 mg/kg, 100 mg/kg, etc.). In some embodiments, administration of a therapeutically effective dose can be a continuous infusion and the dose can be altered (e.g., escalated) over time.

Dosage and frequency may vary depending on the half-life of the anti-CD47 agent in the patient. It will be understood by one of skill in the art that such guidelines will be adjusted for the molecular weight of the active agent, e.g. in the use of antibody fragments, in the use of antibody conjugates, in the use of SIRPα reagents, in the use of soluble CD47 peptides etc. The dosage may also be varied for localized administration, e.g. intranasal, inhalation, etc., or for systemic administration, e.g. intramuscular (i.m.), intraperitoneal (i.p.), intravenous (i.v.), subcutaneous (s.c.), and the like.

An initial dose of a CD47 binding agent, including but not limited to a priming dose, may lead to hemagglutination for a period of time immediately following infusion. Without being bound by the theory, it is believed that the initial dose of a multivalent CD47 binding agent may cause cross-linking of RBC bound to the agent. In certain embodiments of the invention, a CD47 binding agent is infused to a patient in an initial dose, and optionally in subsequent doses, over a period of time and/or concentration that reduces the possibility of hematologic microenvironments where there is a high local concentration of RBC and the agent.

In some embodiments, an initial dose of a CD47 binding agent is infused over a period of at least about 2 hours, at least about 2.5 hours, at least about 3 hours, at least about 3.5 hours, at least about 4 hours, at least about 4.5 hours, at least about 5 hours, at least about 6 hours or more. In some embodiments an initial dose is infused over a period of time from about 2.5 hours to about 6 hours; for example from about 3 hours to about 4 hours. In some such embodiments, the dose of agent in the infusate is from about 0.05 mg/ml to about 0.5 mg/ml; for example from about 0.1 mg/ml to about 0.25 mg/ml.

In other embodiments, an initial dose of a CD47 binding agent, e.g. a priming dose, is administered by continuous fusion, e.g. as an osmotic pump, delivery patch, etc., where the dose is administered over a period of at least about 6 hours, at least about 12 hours, at least about 24 hours, at least about 2 days, at least about 3 days. Many such systems are known in the art. For example DUROS technology, provides a bi-compartment system separated by a piston. One of the compartments consists of osmotic engine specifically formulated with an excess of solid NaCl, such that it remains present throughout the delivery period and results in a constant osmotic gradient. It also consists of a semi permeable membrane on one end through which water is drawn into the osmotic engine and establishes a large and constant osmotic gradient between the tissue water and the osmotic engine. Other compartment consists of a drug solution with an orifice from which the drug is released due to the osmotic gradient. This helps to provide site specific and systemic drug delivery when implanted in humans. The preferred site of implantation is subcutaneous placement in the inside of the upper arm.

Following administration of the priming agent, and allowing a period of time effective for an increase in reticulocyte production, a therapeutic dose of an anti-CD47 agent is administered. The therapeutic dose can be administered in number of different ways. In some embodiments, two or more therapeutically effective doses are administered after a primer agent is administered, e.g. in a weekly dosing schedule. In some embodiments a therapeutically effective dose of an anti-CD47 agent is administered as two or more doses of escalating concentration, in others the doses are equivalent. There is reduced hemagglutination after the priming dose.

Additional agents can enhance the efficacy of anti-CD47 agents. The anti-CD47 antibody can be administered in combination or prior to the additional agent.

A combination of an anti-CD47 antibody with an additional agent described herein is given to patients with tumors subtypes that are responsive to these therapies. These tumors may be defined by a higher frequency of mutations, resulting in more tumor antigens, therefore being more immunogenic, as described herein. In some embodiments patients treated with combination therapy are responsive to treatment with an immune activator or checkpoint inhibitor; however this represents a subset of approximately 25% of patients within a specific potentially responsive tumor subtype. In some embodiments, the individuals may be platinum therapy sensitive or resistant.

In some embodiments, the subject methods include a step of administering a primer agent to subject, followed by a step of administering a therapeutically effective dose of an anti-CD47 antibody and an additional agent to the subject. In some embodiments, the step of administering a therapeutically effective dose is performed after at least about 3 days (e.g., at least about 4 days, at least about 5 days, at least about 6 days, at least about 7 days, at least about 8 days, at least about 9 days, or at least about 10 days) after beginning the administration of a primer agent. This period of time is, for example, sufficient to provide for enhanced reticulocyte production by the individual.

The administration of a therapeutically effective dose of an anti-CD47 antibody and/or an additional agent can be achieved in a number of different ways. In some cases, two or more therapeutically effective doses are administered after a primer agent is administered. Suitable administration of a therapeutically effective dose can entail administration of a single dose, or can entail administration of doses daily, semi-weekly, weekly, once every two weeks, once a month, annually, etc. In some cases, a therapeutically effective dose is administered as two or more doses of escalating concentration (i.e., increasing doses), where (i) all of the doses are therapeutic doses, or where (ii) a sub-therapeutic dose (or two or more sub-therapeutic doses) is initially given and therapeutic doses are achieved by said escalation. As one non-limiting example to illustrate escalating concentration (i.e., increasing doses), a therapeutically effective dose can be administered weekly, beginning with a sub-therapeutic dose (e.g., a dose of 5 mg/kg), and each subsequent dose can be increased by a particular increment (e.g., by 5 mg/kg), or by variable increments, until a therapeutic dose (e.g., 30 mg/kg) is reached, at which point administration may cease or may continue (e.g., continued therapeutic doses, e.g., doses of 30 mg/kg). As another non-limiting example to illustrate escalating concentration (i.e., increasing doses), a therapeutically effective dose can be administered weekly, beginning with a therapeutic dose (e.g., a dose of 10 mg/kg), and each subsequent dose can be increased by a particular increment (e.g., by 10 mg/kg), or by variable increments, until a therapeutic dose (e.g., 30 mg/kg, 100 mg/kg, etc.) is reached, at which point administration may cease or may continue (e.g., continued therapeutic doses, e.g., doses of 30 mg/kg, 100 mg/kg, etc.). In some embodiments, administration of a therapeutically effective dose can be a continuous infusion and the dose can be altered (e.g., escalated) over time.

Dosage and frequency may vary depending on the half-life of the anti-CD47 antibody and/or the additional agent in the patient. It will be understood by one of skill in the art that such guidelines will be adjusted for the molecular weight of the active agent, e.g. in the use of antibody fragments, in the use of antibody conjugates, in the use of SIRPα reagents, in the use of soluble CD47 peptides etc. The dosage may also be varied for localized administration, e.g. intranasal, inhalation, etc., or for systemic administration, e.g. i.m., i.p., i.v., s.c., and the like.

In certain embodiments of the invention, the anti-CD47 antibody is infused to a patient in an initial dose, and optionally in subsequent doses, over a period of time and/or concentration that reduces the possibility of hematologic microenvironments where there is a high local concentration of RBC and the agent.

In some embodiments of the invention, an initial dose of the anti-CD47 antibody is infused over a period of at least about 2 hours, at least about 2.5 hours, at least about 3 hours, at least about 3.5 hours, at least about 4 hours, at least about 4.5 hours, at least about 5 hours, at least about 6 hours or more. In some embodiments an initial dose is infused over a period of time from about 2.5 hours to about 6 hours; for example from about 3 hours to about 4 hours. In some such embodiments, the dose of agent in the infusate is from about 0.05 mg/ml to about 0.5 mg/ml; for example from about 0.1 mg/ml to about 0.25 mg/ml.

Further Combination Therapies

In some embodiments, an antibody provided herein is administered with at least one additional therapeutic agent. Any suitable additional therapeutic agent may be administered with an antibody provided herein.

In some embodiments, the additional therapeutic agent comprises an immunostimulatory agent. In some embodiments, the immunostimulatory agent is an agent that blocks signaling of an inhibitory receptor of an immune cell, or a ligand thereof. In some aspects, the inhibitory receptor or ligand is PD-1 or PD-L1. In some aspects, the agent is selected from an anti-PD-1 antibody (e.g., pembrolizumab or nivolumab), and anti-PD-L1 antibody (e.g., atezolizumab), and combinations thereof. In some aspects, the agent is pembrolizumab. In some aspects, the agent is nivolumab. In some aspects, the agent is atezolizumab.

Table 4 contains the heavy and light chain sequences of atezolizumab.

TABLE 4
Atezolizumab sequences

| SEQ ID | |
|---|---|
| NO | Description and Sequence |
| 165 | >Heavy Chain Sequence |
| | EVQLVESGGGLVQPGGSLRLSCAASGFTFSDSWIHWVRQAPG |
| | KGLEWVAWISPYGGSTYYADSVKGRFTISADTSKNTAYLQMN |
| | SLRAEDTAVYYCARRHWPGGFDYWGQGTLVTVSSASTKGPSV |
| | FPLAPSSKSTSGGTAALGCLVKDYFPEPVTVSWNSGALTSGV |
| | HTFPAVLQSSGLYSLSSVVTVPSSSLGTQTYICNVNHKPSNT |
| | KVDKKVEPKSCDKTHTCPPCPAPELLGGPSVFLFPPKPKDTL |
| | MISRTPEVTCVVVDVSHEDPEVKFNWYVDGVEVHNAKTKPRE |
| | EQYASTYRVVSVLTVLHQDWLNGKEYKCKVSNKALPAPIEKT |
| | ISKAKGQPREPQVYTLPPSREEMTKNQVSLTCLVKGFYPSDI |
| | AVEWESNGQPENNYKTTPPVLDSDGSFFLYSKLTVDKSRWQQ |
| | GNVFSCSVMHEALHNHYTQKSLSLSPGK |
| 166 | >Light Chain Sequence |
| | DIQMTQSPSSLSASVGDRVTITCRASQDVSTAVAWYQQKPGK |
| | APKLLIYSASFLYSGVPSRFSGSGSGTDFTLTISSLQPEDFA |
| | TYYCQQYLYHPATFGQGTKVEIKRTVAAPSVFIFPPSDEQLK |
| | SGTASVVCLLNNFYPREAKVQWKVDNALQSGNSQESVTEQDS |
| | KDSTYSLSSTLTLSKADYEKHKVYACEVTHQGLSSPVTKSFN |
| | RGEC |

In some embodiments, the additional therapeutic agent is an agent that inhibits the interaction between PD-1 and PD-L1. In some aspects, the additional therapeutic agent that inhibits the interaction between PD-1 and PD-L1 is selected from an antibody, a peptidomimetic and a small molecule. In some aspects, the additional therapeutic agent that inhibits the interaction between PD-1 and PD-L1 is selected from pembrolizumab, nivolumab, atezolizumab, avelumab, durvalumab, tislelizumab, cemiplimab, BMS-936559, sulfamonomethoxine 1, sulfamethizole 2, and combinations thereof. In some embodiments, the additional therapeutic agent that inhibits the interaction between PD-1 and PD-L1 is any therapeutic known in the art to have such activity, for example as described in Weinmann et al., Chem Med Chem, 2016, 14:1576 (DOI: 10.1002/cmdc.201500566), incorporated by reference in its entirety. In some embodiments, the agent that inhibits the interaction between PD-1 and PD-L1 is formulated in the same pharmaceutical composition and an antibody provided herein. In some embodiments, the agent that inhibits the interaction between PD-1 and PD-L1 is formulated in a different pharmaceutical composition from an antibody provided herein. In some embodiments, the agent that inhibits the interaction between PD-1 and PD-L1 is administered prior to administration of an antibody provided herein. In some embodiments, the agent that inhibits the interaction between PD-1 and PD-L1 is administered after administration of an antibody provided herein. In some embodiments, the agent that inhibits the interaction between PD-1 and PD-L1 is administered contemporaneously with an antibody provided herein, but the agent and antibody are administered in separate pharmaceutical compositions.

In some embodiments, the additional therapeutic agent comprises a Bcl-2/Bcl-xL inhibitor. The Bcl-2/Bcl-xL inhibitor can include venetoclax, navitoclax, and/or AZD0466, or others. In some embodiments, the Bcl-2/Bcl-xL inhibitor is formulated in the same pharmaceutical composition and an antibody provided herein. In some embodiments, the Bcl-2/Bcl-xL inhibitor is formulated in a different pharmaceutical composition from an antibody provided herein. In some embodiments, the Bcl-2/Bcl-xL inhibitor is administered prior to administration of an antibody provided herein. In some embodiments, the Bcl-2/Bcl-xL inhibitor is administered after administration of an antibody provided herein. In some embodiments, the Bcl-2/Bcl-xL inhibitor is administered contemporaneously with an antibody provided herein, but the Bcl-2/Bcl-xL inhibitor and antibody are administered in separate pharmaceutical compositions.

In some embodiments, additional therapeutic agents include one or more chemotherapeutics. Example chemotherapeutics include antimetabolite antineoplastic agents (e.g., fluorouracil, cladribine, methotrexate, mercaptopurine, pemetrexed, gemcitabine, capecitabine, hydroxyurea, fludarabine, pralatrexate, nelarabine, clofarabine, decitabine, cytarabine, and floxuridine), alkylating agents (e.g., bendamustine, chlorambucil, cylclophosphamide, ifosfamide, carmustine, lomustine, busulfan, dacarbazine, temozolomide, altretamine, and thitepa), and platinum antineoplastic drugs (e.g., cisplatin, carboplatin, and oxaliplatin).

Subject Status, Eligibility, and Treatment

A subject with cancer that is administered an anti-CD47 agent and an anti-CD20 agent can have a certain status. The status can be used to determine the eligibility of the subject to receive the administration of the therapeutic agents. In some embodiments, a subject that is determined to be eligible is more likely to benefit from administration of both agents in comparison to a different subject that is determined to be ineligible.

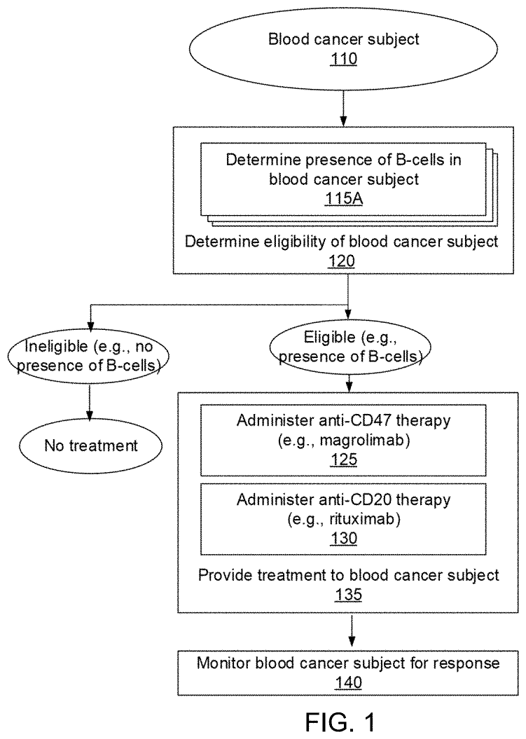

Reference is made to FIG. 1, which is an example flow process for determining eligibility of a blood cancer subject 110 for receiving a treatment, in accordance with an embodiment. The blood cancer subject 110 is evaluated for his/her status in order to determine 120 the eligibility of the blood cancer subject to receive a treatment.

General examples of the subject's status can include whether the subject has a presence or absence of B-cells, the type of cancer that the subject currently has, the number of prior therapies that the subject has undergone, whether the subject is relapsed or refractory to certain therapies, whether the subject can receive a CAR-T treatment, and an amount of time since the subject last received a treatment (e.g., an anti-CD20 treatment).

As one specific example, a blood cancer subject's status can be that the subject has relapsed or is refractory to at least 1, 2, 3, 4, 5, 6, 7, 8, 9, 10 or greater than 10 prior lines of cancer therapy. Additional examples of subject status include that a subject can be refractory to rituximab. A subject can be resistant to rituximab. Rituximab refractory status can be a failure to respond to, or progression during, any previous rituximab-containing regimen, or progression within 6 months of the last rituximab dose. Rituximab refractory status can be a failure to respond to, or progression during, last previous rituximab-containing regimen, or progression within 6 months of the last rituximab dose.

In some aspects, a subject's status includes that the subject has follicular lymphoma (FL) and/or has received at least two prior systemic therapies. In some aspects, a subject has follicular lymphoma (FL) and relapsed after, or is refractory to, a rituximab-containing regimen.

In some aspects, a subject's status includes that a subject has relapsed or refractory large-B cell lymphoma and/or has received at two or more lines of systemic therapy. In some aspects, a subject has de novo or transformed large-B cell lymphoma refractory to frontline therapy, or relapsed or refractory to second line salvage regimens or autologous hematopoietic cell transplantation. In some aspects, a subject has large-B cell lymphoma and relapsed after, or is refractory after two or more lines of systemic therapy including a rituximab-containing regimen.

In some aspects, the status of the subject is a presence or absence of B-cells in the subject. In some embodiments, the status of the subject is a presence of CD19+ B-cells. In some embodiments, the status of the subject is a presence of CD20+ B-cells. In some embodiments, the status of the subject is a presence of both CD19+ and CD20+ B-cells.

Returning to FIG. 1, the status of the blood cancer subject is used to determine 120 eligibility of the blood cancer subject. FIG. 1 depicts one embodiment where determining 120 the eligibility of the blood cancer subject includes determining 115A a presence of B-cells in the blood cancer subject. In one embodiment, if the subject is determined to have a presence of B-cells, then the subject is eligible to receive the treatment. In contrast, if the subject is determined to not have a presence of B-cells, then the subject is ineligible to receive the treatment. In some embodiments, determining 120 the eligibility of the blood cancer subject can additionally include determining whether other parts of the subject's status (e.g., subject's type of cancer, the number of prior therapies, whether the subject is relapsed or refractory to certain therapies) meet established eligibility criteria (e.g., criteria for enrolling in a clinical trial). As an example, in addition to meeting the eligibility criterion of having a presence of B-cells, an eligible patient has received at least 2 prior lines of therapy, has DLBCL, and last received an anti-CD20 treatment more than 4 weeks ago.

In one embodiment, the presence or absence of B-cells can be determined by obtaining a sample from the subject and performing an assay, as described in above, on the obtained sample. Such assays can directly measure the number of B-cells in the sample obtained from the subject. The quantity of B-cells can be expressed as a total quantity of B-cells in the subject, a percentage of B-cells out of the total quantity of lymphocytes, or a quantity of B-cells per microliter of the sample. In some embodiments, the presence or absence of B-cells can be determined by performing a tissue biopsy (e.g., biopsy of the cancer tissue) and performing a qualitative analysis of the presence or absence of B-cells. As an example, immunohistochemistry (IHC) staining for B-cells (e.g., CD19 or CD20 B-cells) can be performed. IHC stained tissue slices can be qualitatively analyzed (e.g., by a pathologist) and a score, hereafter referred to as a H-score, can be assigned, the score indicating a presence or absence of B-cells.

The quantity of B-cells can then be used to make a determination as to the presence or absence of B-cells in the subject. In one embodiment, the quantity of B-cells is compared to a threshold value. If the quantity of B-cells is above the threshold value, then B-cells are determined to be present in the subject. If the quantity of B-cells is below the threshold value, then B-cells are determined to be absent from the subject. As one example, if the quantity of B-cells is expressed as a percentage of B-cells out of the total quantity of lymphocytes, the threshold value can be 5 percent. Total quantity of lymphocytes can be measured through a marker, such as CD45. As another example, if the quantity of B-cells is expressed as a quantity of B-cells per microliter of the sample, the threshold value can be one B-cell per microliter. In some scenarios, the threshold value can be forty B-cells per microliter. In some embodiments, the threshold value is set based on a limit of detection or a limit of quantitation of an assay used to determine the presence of the B-cells. Therefore, in these embodiments, if the assay is able to reliably detect B-cells (e.g., above the limit of detection or above the limit of quantitation), then B-cells are deemed to be present in the subject.

In some embodiments, the presence or absence of B-cells is not directly measured. Instead, a surrogate for the presence or absence of B-cells is measured. Such a surrogate measurement is informative for determining 115A the presence or absence of B-cells in the blood cancer subject 110. Examples of a surrogate for the presence or absence of B-cells include an amount of time that the subject last received an anti-CD20 therapy, the concentration of the anti-CD20 therapy that the subject last received, and a concentration of the anti-CD20 therapy currently in the subject.

To determine whether B-cells are present or absent in the subject using a surrogate marker, the measurement of the surrogate is compared to a threshold value. Depending on the particular surrogate measurement and whether the surrogate measurement is above or below the threshold, the presence or absence of B-cells in the subject is determined.

For example, the surrogate measurement is an amount of time that the subject last received an anti-CD20 therapy and therefore, if the subject last received the anti-CD20 therapy more than a threshold amount of time ago, then B-cells are present in the subject. If the subject last received the anti-CD20 therapy less than a threshold amount of time ago, then B-cells are absent in the subject. If the subject had not previously received an anti-CD20 therapy, then B-cells are present in the subject or a different measurement is taken to determine whether B-cells are present. In various embodiments, a threshold amount of time is at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, or at least 28 weeks. In various embodiments, a threshold amount of time is 2, 3, 4, 5, 6, 7, 8, 9, 10, 11, 12, 13, 14, 15, 16, 17, 18, 19, 20, 21, 22, 23, 24, 25, 26, 27, or 28 weeks. In some embodiments, the threshold amount of time is between 2 and 28 weeks, between 4 and 24 weeks, between 6 and 22 weeks, between 8 and 20 weeks, between 9 and 19 weeks, between 10 and 18 weeks, between 11 and 17 weeks, between 12 and 18 weeks, between 13 and 17 weeks, or between 14 and 16 weeks.

As another example, the surrogate measurement is the concentration of the anti-CD20 therapy currently in the subject and therefore, if the subject has a concentration of anti-CD20 therapy that is above a threshold, then B-cells are not present in the subject. If the subject has a concentration of anti-CD20 therapy that is below a threshold, then B-cells are present in the subject. In various embodiments, a threshold amount of concentration of anti-CD20 therapy in the subject is based on a limit of detection or a limit of quantitation of an assay used to determine the concentration of anti-CD20 therapy. Therefore, in these embodiments, if the assay is able to reliably detect the anti-CD20 therapy (e.g., concentration of anti-CD20 therapy is above the limit of detection or above the limit of quantitation), then the anti-CD20 therapy is deemed to be present in the subject.

Returning to FIG. 1, eligible subjects (e.g., subjects that are determined to have a presence of B-cells) are treated. In various embodiments, providing 135 a treatment to the blood cancer subject includes administering 125 an anti-CD47 therapy (e.g., magrolimab). In some embodiments, providing 135 a treatment to the blood cancer subject 110 includes administering 130 an anti-CD20 therapy (e.g., rituximab). In some embodiments, providing 135 a treatment to the blood cancer subject 110 includes both administering 125 an anti-CD47 therapy and administering 130 an anti-CD20 therapy. Such therapeutics can be further administered according to particular dosing cycles, as is discussed in further detail below. Following treatment, the subject is monitored 140 for response and if needed, can undergo additional cycles of therapy.

In various embodiments, ineligible subjects (e.g., subjects that are determined to not have a presence of B-cells) do not undergo treatment. In some embodiments, ineligible subjects undergo an alternative treatment that does not involve administering an anti-CD47 and/or anti-CD20 antibody.

Immunohistochemical Analysis for Presence of B-Cells

In various embodiments, presence or absence of B-cells in a subject is determined through an immunohistochemical analysis. A tissue biopsy (e.g., a cancer biopsy) can be obtained from a patient and immunostained for a B-cell marker such as CD19 or CD20. Immunostained tissue slices can be imaged for the B-cell marker to determine the presence or absence of B-cells in the subject's tissue.

In various embodiments, the immunostained tissue slices are analyzed to calculate a score (also referred to as a H-score) representing the presence or absence of B-cells. In various embodiments, the scoring of the immunostained tissue slices is performed by a pathologist.

In various embodiments, the H-score can be scored based on an intensity of the B-cell staining in the tissue. For example, a cell in the tissue can be assigned a higher value if the staining intensity is higher in comparison to a lower value assigned to a different cell in the tissue with a lower staining intensity. In various embodiments, the percentage of cells with each value is calculated and then the percentage of cells is weighted by the value to generate a score for the percentage of cells for that value. The scores across the different values can be combined to generate the H-score for the subject.

As an example, cells can be assigned a value of 0, 1, 2, or 3, where 0 represents absent B-cell staining with 3 representing maximal B-cell staining intensity. The percentage of cells at each staining intensity level is calculated and an H-score is assigned using the following formula: [1×(% cells scored as 1)+2×(% cells scored as 2)+3×(% cells scored as 3)]. Here, an H-score can range from 0-300.

The H-score can be used to determine whether the subject has a presence or absence of B-cells. In one embodiment, the H-score for the subject is compared to a threshold H-score. If the H-score for the subject is above the threshold H-score, then the subject is deemed to have a presence of B-cells. If the H-score for the subject is below the threshold H-score, the subject is deemed to have an absence of B-cells. In various embodiments, the threshold H-score is 10, 20, 30, 40, 50, 60, 70, 80, or 90% of the maximum possible H-score value. For example if the H-score can range from 0-300, where 300 is the maximum possible H-score value, then the threshold H-score can be 30, 60, 90, 120, 150, 180, 210, 240, or 270.

Dosing

The methods described herein include administration of a therapeutically effective dose of compositions, i.e., a therapeutically effective dose of an anti-CD47 antibody (e.g., magrolimab) and, optionally, an additional agent such as an anti-CD20 antibody (e.g., rituximab). In various embodiments, the methods target one or both of CD47 or SIRPα.

Compositions are administered to a patient in an amount sufficient to substantially ablate targeted cells, as described above. An amount adequate to accomplish this is defined as a “therapeutically effective dose”, which may provide for an improvement in overall survival rates. Single or multiple administrations of the compositions may be administered depending on the dosage and frequency as needed and tolerated by the patient. The particular dose used for a treatment will depend upon the medical condition and history of the mammal, as well as other factors such as age, weight, gender, administration route, efficiency, etc.

Effective doses of the combined agents of the present invention for the treatment of cancer vary depending upon many different factors, including means of administration, target site, physiological state of the patient, whether the patient is human or an animal, other medications administered, and whether treatment is prophylactic or therapeutic. Usually, the patient is a human, but nonhuman mammals may also be treated, e.g. companion animals such as dogs, cats, horses, etc., laboratory mammals such as rabbits, mice, rats, etc., and the like. Treatment dosages can be titrated to optimize safety and efficacy.

A therapeutically effective dose of the anti-CD47 antibody can depend on the specific agent used, but is usually about 20 mg/kg body weight or more (e.g., about 20 mg/kg or more, about 25 mg/kg or more, about 30 mg/kg or more, about 35 mg/kg or more, about 40 mg/kg or more, about 45 mg/kg or more, about 50 mg/kg or more, or about 55 mg/kg or more, or about 60 mg/kg or more, or about 65 mg/kg or more, or about 70 mg/kg or more), or from about 20 mg/kg to about 70 mg/kg (e.g., from about 20 mg/kg to about 67.5 mg/kg, or from about 20 mg/kg to about 60 mg/kg).

In some embodiments, the therapeutically effective dose of the anti-CD47 antibody is 20, 30, 45, 60, or 67.5 mg/kg. In some embodiments, the therapeutically effective dose of the anti-CD47 antibody is 20 to 60 mg/kg. In some embodiments, the therapeutically effective dose of the anti-CD47 antibody is 20 to 67.5 mg/kg.

A dose of an anti-CD47 antibody can be a flat dose. For example, a flat dose can be given irrespective of a particular subject's weight. Alternatively a flat dose can be given based on a particular subject's weight falling within a particular weight range, e.g., a first range of less than or equal to 100 kg; or a second range of greater than 100 kg. A flat dose can be, e.g., 1000-5000, 2000-4000, 2000-3500, 2400-3500, 1000, 1100, 1200, 1300, 1400, 1500, 1600,1700, 1800, 1900,2000,2100,2200,2300,2400,2500,2600,2700,2800,2900, 3000,3100,3200,3300,3400,3500,3600,3700,3800,3900,4000,4100,4200,4300,4400, 4500, 4600, 4700, 4800, 4900, 5000 mg, or an interim number of mg thereof.

A therapeutically effective dose of the anti-CD20 antibody can depend on the specific agent used, but can be about 100 mg of antibody per m² of body surface area or more (e.g., about 100 mg/m² or more, about 125 mg/m² or more, about 150 mg/m² or more, about 175 mg/m² or more, about 200 mg/m² or more, about 225 mg/m² or more, about 250 mg/m² or more, about 275 mg/m² or more, about 300 mg/m² or more, about 325 mg/m² or more, about 350 mg/m² or more, about 375 mg/m² or more, about 400 mg/m² or more, about 425 mg/m² or more, about 450 mg/m² or more, about 475 mg/m² or more, or about 500 mg/m² or more), or from about 300 mg/m² to about 450 mg/m² (e.g., from about 325 mg/m² to about 425 mg/m², or from about 350 mg/m² to about 400 mg/m²). In some embodiments, the therapeutically effective dose of the anti-CD20 antibody is 100, 125, 150, 175, 200, 225, 250, 275, 300, 325, 350, 375, 400, 425, 450, 475, or 500 mg/m². In some embodiments, the therapeutically effective dose of the anti-CD20 antibody is preferably 375 mg/m².

A dose of an anti-CD20 antibody can be a flat dose. For example, a flat dose can be given irrespective of a particular subject's weight. Alternatively a flat dose can be given based on a particular subject's gender, e.g., a first range for a male (average body surface area of 1.9 m²) and a second range for a female (average body surface area of 1.6 m²). A flat dose can be, e.g., 500-2000, 600-1900, 700-1800, 800-1700, 900-1600, 1000-1700, 1100-1600, 1200-1500,1300-1400,500,600,700, 800,900,1000,1100, 1200, 1300, 1400,1500,1600, 1700, 1800, 1900, 2000 mg, or an interim number of mg thereof.

The dose needed to achieve and/or maintain a particular serum level of the administered composition is proportional to the amount of time between doses and inversely proportional to the number of doses administered. Thus, as the frequency of dosing increases, the needed dose decreases. The optimization of dosing strategies will be readily understood and practiced by one of ordinary skill in the art. An exemplary treatment regime entails administration once every two weeks or once a month or once every 3 to 6 months. Therapeutic entities of the present invention are usually administered on multiple occasions. Intervals between single dosages can be weekly, monthly or yearly. Intervals can also be irregular as indicated by measuring blood levels of the therapeutic entity in the patient. Alternatively, therapeutic entities of the present invention can be administered as a sustained release formulation, in which case less frequent administration is used. Dosage and frequency vary depending on the half-life of the polypeptide in the patient.

A “maintenance dose” is a dose intended to be a therapeutically effective dose. For example, in experiments to determine the therapeutically effective dose, multiple different maintenance doses may be administered to different subjects. As such, some of the maintenance doses may be therapeutically effective doses and others may be sub-therapeutic doses.

In prophylactic applications, a relatively low dosage may be administered at relatively infrequent intervals over a long period of time. Some patients continue to receive treatment for the rest of their lives. In other therapeutic applications, a relatively high dosage at relatively short intervals is sometimes used until progression of the disease is reduced or terminated, and preferably until the patient shows partial or complete amelioration of symptoms of disease. Thereafter, the patent can be administered a prophylactic regime.

In still other embodiments, methods of the present invention include treating, reducing or preventing tumor growth, tumor metastasis or tumor invasion of cancers including carcinomas, hematologic cancers, melanomas, sarcomas, gliomas, etc. For prophylactic applications, pharmaceutical compositions or medicaments are administered to a patient susceptible to, or otherwise at risk of disease in an amount sufficient to eliminate or reduce the risk, lessen the severity, or delay the outset of the disease, including biochemical, histologic and/or behavioral symptoms of the disease, its complications and intermediate pathological phenotypes presenting during development of the disease.

Toxicity of the combined agents described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in formulating a dosage range that is not toxic for use in human. The dosage of the proteins described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition.

Primer Agents and Priming Dose

In some embodiments of the methods described herein, a primer agent is administered prior to administering a therapeutically effective dose of an anti-CD47 antibody to the individual. Suitable primer agents include an erythropoiesis-stimulating agent (ESA), and/or a priming dose of an anti-CD47 antibody. Following administration of the priming agent, and allowing a period of time effective for an increase in reticulocyte production, a therapeutic dose of an anti-CD47 antibody is administered. Administration may be made in accordance with the methods described in co-pending patent application U.S. Ser. No. 14/769,069, herein specifically incorporated by reference.

In some embodiments, administration of a combination of agents of the invention is combined with an effective dose of an agent that increases patient hematocrit, for example erythropoietin stimulating agents (ESA). Such agents are known and used in the art, including, for example, Aranesp ⋅ ⋅(darbepoetin alfa), Epogen⋅NF/Procrit⋅NF (epoetin alfa), Omontys ⋅(peginesatide), Procrit ⋅, etc.

The term “priming dose” or as used herein refers to a dose of an anti-CD47 agent that primes a subject for administration of a therapeutically effective dose of anti-CD47 agent such that the therapeutically effective dose does not result in a severe loss of RBCs (reduced hematocrit or reduced hemoglobin). The specific appropriate priming dose of an anti-CD47 agent can vary depending on the nature of the agent used and on numerous subject-specific factors (e.g., age, weight, etc.). Examples of suitable priming doses of an anti-CD47 agent include from about 0.5 mg/kg to about 5 mg/kg, from about 0.5 mg/kg to about 4 mg/kg, from about 0.5 mg/kg to about 3 mg/kg, from about 1 mg/kg to about 5 mg/kg, from about 1 mg/kg to about 4 mg/kg, from about 1 mg/kg to about 3 mg/kg, about 1 mg/kg, about 2 mg/kg, about 3 mg/kg, about 4 mg/kg, about 5 mg/kg. In some embodiments, the priming does is preferably 1 mg/kg.

In some embodiments of the methods described herein, the anti-CD47 antibody is administered to the subject as a priming dose ranging from about 0.5 mg to about 10 mg, e.g., from about 0.5 to about 5 mg/kg of antibody, optionally, 4 mg/kg, 3 mg/kg, 2 mg/kg, or 1 mg/kg of antibody. In some embodiments, the anti-CD47 antibody is administered to the subject as a dose ranging from about 20 to about 67.5 mg/kg of antibody, optionally from 15 to 60 mg/kg of antibody, optionally from 30 to 60 mg/kg of antibody, optionally 15 mg/kg of antibody, 20 mg/kg of antibody, 30 mg/kg of antibody, 45 mg/kg of antibody, 60 mg/kg of antibody, or 67.5 mg/kg of antibody.

A priming dose of an anti-CD47 antibody can be a flat priming dose. For example, a flat priming dose can be given irrespective of a particular subject's weight. Alternatively a flat priming dose can be given based on a particular subject's weight falling within a particular weight range, e.g., a first range of less than or equal to 100 kg; or a second range of greater than 100 kg. A flat priming dose can be, e.g., 10-200, 50-100, 80-800, 80-400, 80-200, 70-90, 75-85, 10, 20, 30, 40, 50, 60, 70, 80, 90,100,110, 120, 130, 140,150, 160, 170, 180, 190, 200, 240, 300, 320, 400, 500, 600, 700 or 800 mg, or an interim number of mg thereof.

In some embodiments of the invention, a primer agent is administered prior to administering a therapeutically effective dose of an anti-CD47 agent to the individual. Suitable primer agents include an erythropoiesis-stimulating agent (ESA), and/or a priming dose of an anti-CD47 agent. Following administration of the priming agent, and allowing a period of time effective for an increase in reticulocyte production, a therapeutic dose of an anti-CD47 agent is administered. The therapeutic dose can be administered in number of different ways. In some embodiments, two or more therapeutically effective doses are administered after a primer agent is administered. In some embodiments a therapeutically effective dose of an anti-CD47 agent is administered as two or more doses of escalating concentration, in others the doses are equivalent.

In some embodiments of the invention, an effective priming dose of Hu-5F9G4 is provided, where the effective priming dose for a human is around about 1 mg/kg, e.g. from at least about 0.5 mg/kg up to not more than about 5 mg/kg; from at least about 0.75 mg/kg up to not more than about 1.25 mg/kg; from at least about 0.95 mg/kg up to not more than about 1.05 mg/kg; and may be around about 1 mg/kg

In some embodiments of the invention, an initial dose of a CD47 binding agent is infused over a period of at least about 2 hours, at least about 2.5 hours, at least about 3 hours, at least about 3.5 hours, at least about 4 hours, at least about 4.5 hours, at least about 5 hours, at least about 6 hours or more. In some embodiments an initial dose is infused over a period of time from about 2.5 hours to about 6 hours; for example from about 3 hours to about 4 hours. In some such embodiments, the dose of agent in the infusate is from about 0.05 mg/ml to about 0.5 mg/ml; for example from about 0.1 mg/ml to about 0.25 mg/ml.

In some embodiments a priming dose may be delivered through a sub-cutaneous route, by injection, patch, osmotic pump, and the like as known in the art.

Following administration of the priming agent, and allowing a period of time effective for an increase in reticulocyte production, a therapeutic dose of an anti-CD47 agent is administered. The therapeutic dose can be administered in number of different ways. In some embodiments, two or more therapeutically effective doses are administered after a primer agent is administered, e.g. in a weekly dosing schedule. In some embodiments a therapeutically effective dose of an anti-CD47 agent is administered as two or more doses of escalating concentration, in others the doses are equivalent.

In other embodiments, an initial dose of a CD47 binding agent, e.g. a priming dose, is administered by continuous fusion, e.g. as an osmotic pump, delivery patch, etc., where the dose is administered over a period of at least about 6 hours, at least about 12 hours, at least about 24 hours, at least about 2 days, at least about 3 days. Many such systems are known in the art. For example DUROS technology, provides a bi-compartment system separated by a piston. One of the compartments consists of osmotic engine specifically formulated with an excess of solid NaCl, such that it remains present throughout the delivery period and results in a constant osmotic gradient. It also consists of a semi permeable membrane on one end through which water is drawn into the osmotic engine and establishes a large and constant osmotic gradient between the tissue water and the osmotic engine. Other compartment consists of a drug solution with an orifice from which the drug is released due to the osmotic gradient. This helps to provide site specific and systemic drug delivery when implanted in humans. The preferred site of implantation is subcutaneous placement in the inside of the upper arm.

Following administration of the priming agent, and allowing a period of time effective for an increase in reticulocyte production, a therapeutic dose of the anti-CD47 antibody is administered. The therapeutic dose can be administered in number of different ways. In some embodiments, two or more therapeutically effective doses are administered after a primer agent is administered, e.g. in a weekly dosing schedule. In some embodiments a therapeutically effective dose of the anti-CD47 antibody is administered as two or more doses of escalating concentration, in others the doses are equivalent. There is reduced hemagglutination after the priming dose.

Dosing Cycles

A method of treating a human subject having a CD20+ cancer or reducing the size of the CD20+ cancer in the human subject can include at least one cycle of (a) administering an anti-CD47 antibody to the subject at a dose of greater than or equal to 10 mg of antibody per kg of body weight; and (b) administering an anti-CD20 antibody to the subject. In various embodiments, the methods target one or both of CD47 or SIRPα.

An anti-CD47 antibody can be administered to a subject in a given cycle as a dose ranging from about 20 to about 67.5 mg of antibody per kg of body weight, optionally 20 to 30 mg of antibody per kg of body weight, optionally 20 mg of antibody per kg of body weight, 30 mg of antibody per kg of body weight, 45 mg of antibody per kg of body weight, 60 mg of antibody per kg of body weight, or 67.5 mg of antibody per kg of body weight.

In some embodiments, the interval between each single dose is a week. In some embodiments, the interval between each single dose is two weeks. In some embodiments, the interval between each single dose is three weeks. In some embodiments, the interval between each single dose is four weeks. In some embodiments, the interval between each single dose of anti-CD47 antibody is a week. In some embodiments, the interval between each single dose of anti-CD47 antibody is two weeks. In some embodiments, the interval between each single dose of anti-CD47 antibody is three weeks. In some embodiments, the interval between each single dose of anti-CD47 antibody is four weeks. In some embodiments, the interval between each single dose of Hu5F9 (e.g., Hu5F9-G4) is a week. In some embodiments, the interval between each single dose of Hu5F9 (e.g., Hu5F9-G4) is two weeks. In some embodiments, the interval between each single dose of Hu5F9 (e.g., Hu5F9-G4) is three weeks. In some embodiments, the interval between each single dose of Hu5F9 (e.g., Hu5F9-G4) is four weeks. In some embodiments, the interval between each single dose of anti-CD20 antibody is a week. In some embodiments, the interval between each single dose of anti-CD20 antibody is two weeks. In some embodiments, the interval between each single dose of anti-CD20 antibody is three weeks. In some embodiments, the interval between each single dose of anti-CD20 antibody is four weeks. In some embodiments, the interval between each single dose of anti-CD20 antibody is eight weeks. In some embodiments, the interval between each single dose of rituximab is a week. In some embodiments, the interval between each single dose of rituximab is two weeks. In some embodiments, the interval between each single dose of rituximab is three weeks. In some embodiments, the interval between each single dose of rituximab is four weeks. In some embodiments, the interval between each single dose of rituximab is eight weeks.

In various embodiments, administration of the anti-CD47 antibody and/or administration of the anti-CD20 antibody can occur in one or more cycles, for example, a first cycle can have a first dosing scheme and one or more subsequent cycles can have dosing scheme(s) that are distinct from (or the same as) the first cycle. In various embodiments, the dosing intervals of the first cycle and second cycle are the same (e.g. the anti-CD47 agent is administered once a week) and the dosing intervals of the third cycle and further additional cycles are different from the first and second cycles (e.g., the anti-CD47 agent is administered once every two weeks). The dosing intervals of the third cycle and additional cycles can be the same. For example, an anti-CD47 antibody can be administered in a first cycle comprising a dose of antibody once every week; a second cycle comprising a dose of antibody once every week; a third cycle comprising a dose of antibody once every two weeks; a fourth cycle comprising a dose of antibody once every two weeks; and additional cycles comprising a dose of antibody once every two weeks as needed, e.g., as determined by a physician. The first cycle, second cycle, third cycle, and additional cycles can be 4 weeks in duration.

In some embodiments, an anti-CD47 antibody can be administered to the subject for at least three distinct cycles of four weeks each, the first cycle comprising (1) administering a dose of anti-CD47 antibody once every week; the second cycle comprising (2) administering a dose of anti-CD47 antibody once every week; and the third cycle comprising (3) administering a dose of anti-CD47 antibody once every two weeks.

In various embodiments, an anti-CD20 antibody can be administered to the subject for at least three distinct cycles of four weeks each, the first cycle comprising (1) administering a dose of anti-CD20 antibody once every week; the second cycle comprising (2) administering a dose of anti-CD20 antibody once every 4 weeks; and the third cycle comprising (3) administering a dose of anti-CD20 antibody once every 4 weeks. In various embodiments, an anti-CD20 antibody can be further administered to the subject for additional cycles. In various embodiments, anti-CD20 antibody can be administered once every 4 weeks or administered once every 8 weeks during the additional cycles.

In various embodiments, a priming dose of an anti-CD47 antibody is administered to a subject in a given cycle prior to administering an anti-CD47 antibody to the subject at a dose of greater than or equal to 10 mg of antibody per kg of body weight. A priming dose can be 1 mg of antibody per kg of body weight. A priming dose can be administered to a subject for about 3 hours.

In particular embodiments, anti-CD47 antibody and anti-CD20 antibody are administered to a subject according to the following cycles:

### Cycle 1 (4 Weeks)

Priming dose of 1 mg/kg anti-CD47 on day 1
Weekly dose of 30 mg/kg anti-CD47 starting on day 8
Weekly dose of 375 mg/m² rituximab or anti-CD20 antibody equivalent dose starting on day 8

### Cycle 2 (4 Weeks)

Weekly dose of 30 mg/kg anti-CD47
Monthly dose of 375 mg/m² rituximab or anti-CD20 antibody equivalent dose

### Cycles 3-5

30 mg/kg anti-CD47 every other week
Monthly dose of 375 mg/m² rituximab or anti-CD20 antibody equivalent dose

### Cycle 6+ (Continue Until Loss of Clinical Benefit)

30 mg/kg dose of anti-CD47 every other week
Every other month dosing of 375 mg/m² rituximab or anti-CD20 antibody equivalent dose

In various embodiments, a first cycle comprises providing a priming dose of anti-CD47 antibody, followed by a weekly dose (e.g., once every week) of the anti-CD47 antibody. The weekly dose of anti-CD47 antibody can be administered through a second cycle. Following the second cycle, the anti-CD47 antibody can be administered during a third cycle through every-other-week doses. In various embodiments, the anti-CD47 antibody can be continued to be administered through a fourth and fifth cycle. In various embodiments, the anti-CD47 antibody is administered in subsequent cycles until therapeutic response is achieved. In various embodiments, the anti-CD20 antibody can also be administered during each of the first cycle, second cycle, third cycle, fourth cycle, fifth cycle, and subsequent cycles. In various embodiments, the anti-CD20 antibody is administered through a weekly dose through the first cycle, a monthly dose through the second, third, fourth, and fifth cycles, and an every-other-month dose in subsequent cycles..

As an example, an anti-CD47 antibody can be administered to a subject in a first cycle comprising a priming dose of 1 mg of antibody per kg of body weight on day 1 followed by a dose of 30 mg of antibody per kg of body weight once every week (e.g., day 8, day 15, and so on). The first cycle can be 4 weeks in duration. An anti-CD20 antibody can be administered to the subject in the first cycle once every week at a dose of 375 mg/m² of antibody. In various embodiments, the method targets CD47 or SIRPα.

An anti-CD47 antibody can be administered in a second cycle comprising a dose of 30 mg of antibody per kg of body weight once every week. The second cycle can be 4 weeks in duration. An anti-CD20 antibody can be administered in the second cycle once every four weeks (e.g. monthly) at a dose of 375 mg/m² of antibody. In various embodiments, the method targets CD47 or SIRPα.

In various embodiments, the anti-CD47 antibody and the anti-CD20 antibody can each be administered to the patient on the same day (e.g., weekly doses on day 8, day 15, etc.) In some embodiments, on these days where both therapies are administered to the patient, the anti-CD20 antibody is administered prior to administration of the anti-CD47 antibody. In other embodiments, on these days where both therapies are administered to the patient, the anti-CD47 antibody can be administered prior to administration of the anti-CD20 antibody.

An anti-CD47 antibody can be administered in a third cycle comprising a dose of 30 mg of antibody per kg of body weight once every two weeks. The third cycle can be 4 weeks in duration. An anti-CD20 antibody can be administered in the third cycle once every four weeks (e.g. monthly) at a dose of 375 mg/m² of antibody. In various embodiments, the method targets CD47 or SIRPα.

In various embodiments, the third cycle can be repeated through one or more additional cycles. In one embodiment, the third cycle is repeated twice (e.g., through a fourth cycle and fifth cycle).

In various embodiments, an anti-CD47 antibody can be administered in a sixth cycle comprising a dose of 30 mg of antibody per kg of body weight once every two weeks. In various embodiments, the sixth cycle further comprises administering an anti-CD20 antibody one every other month at a dose of 375 mg/m² of antibody. The sixth cycle can be a set number of weeks or, in some embodiments, can depend on whether the patient responds to the treatment. For example, once the patient responds to the treatment, the sixth cycle can be terminated a number of weeks after the patient exhibits clinical benefit. As another example, the sixth cycle can be terminated if, after providing treatment to the patient in the sixth cycle, the patient fails to clinically respond to the treatment. As another example, the sixth cycle can be terminated if, after providing treatment to the patient in the sixth cycle, the clinical benefit of the treatment is lost. In various embodiments, the method targets CD47 or SIRPα.

Additional cycles can be used. For example, at least one additional cycle, optionally 1, 2, 3, 4, 5, 6, 7, 8, 9, 10, 11, 12, 13, 14, 15, 16, 17, 18, 19, 20, or greater than 20 additional cycles can be used. The dosing regimen of the at least one additional cycle can be the same as the second cycle, optionally wherein the anti-CD20 antibody portion of the dosing regimen is discontinued after completing 6 total cycles. Optionally the anti-CD20 portion of a given cycle can be continued after completing 6 total cycles, e.g., by pursuing a once per month or a once every other month dosing protocol. An at least one additional cycle can be 4 weeks in duration.

Also disclosed herein is a method of treating or reducing the size of a cancer in a human subject, comprising administering an anti-CD47 antibody (e.g., Hu5F9-G4) and an anti-CD20 antibody (e.g., rituximab) to the subject for at least two distinct cycles of four weeks each, the first cycle comprising (1) administering a priming dose of anti-CD47 antibody in the range of 1 mg to 10 mg (e.g., 1 mg to 5 mg, e.g., 1 mg, 2 mg, 3 mg, 4 mg, 5 mg) of antibody per kg of body weight at time 0 (e.g., TO or day 1), (2) administering a dose of at least 30 mg (e.g., 30-50, 30, 35, 40, 45, 50 mg) of anti-CD47 antibody per kg of body weight once every week beginning one week after TO with an additional (optional) loading dose of at least 30 mg/kg (e.g., 30-50, 30, 35, 40, 45, 50 mg) on Day 11 (week 2), and (3) administering a dose of 375 mg/m² of anti-CD20 antibody once every week; and the second cycle comprising (1) administering a dose of at least 30 mg (e.g., 30-50, 30, 35, 40, 45, 50 mg) of anti-CD47 antibody per kg of body weight once every week, and (2) administering a dose of 375 mg/m² of anti-CD20 antibody once every month. Anti-CD47 and anti-CD20 antibodies can be subsequently administered through a third, fourth, and fifth cycles. In various embodiments, the third, fourth, and fifth cycles each comprise (1) administering a dose of at least 30 mg (e.g., 30-50, 30, 35, 40, 45, 50 mg) of anti-CD47 antibody per kg of body weight once every other week and (2) administering a dose of 375 mg/m² of anti-CD20 antibody once every month. Additional cycles (e.g., sixth, seventh, eighth, ninth, tenth, etc.) of anti-CD47 and anti-CD20 antibodies can be provided without limit or, for example, until clinical benefit is reduced or lost or no longer observed. When reached and starting at Cycle 6 and beyond, anti-CD20 antibody can instead be administered to the subject at a dose of 375 mg/m² once every eight weeks. Generally, anti-CD47 antibody and anti-CD20 antibody will continue to be administered to the subject as above until the subject loses clinical benefit, e.g., via CR or death. The anti-CD47 antibody can be Hu5F9-G4. The anti-CD20 antibody can be rituximab. The cancer can be at least one of: a CD20+ cancer, a B cell cancer, Non-Hodgkin's lymphoma (NHL), indolent lymphoma, follicular lymphoma (FL), marginal zone lymphoma, or diffuse large B cell lymphoma (DLBCL). In various embodiments, the method targets CD47 or SIRPα.

Also disclosed herein is a method of treating or reducing the size of a CD20+ cancer in a human subject, comprising administering an anti-CD47 antibody that is Hu5F9-G4 and an anti-CD20 antibody that is rituximab to the subject for at least two distinct cycles of four weeks each, the first cycle comprising (1) administering a priming dose of anti-CD47 antibody in the range of 1 mg to 10 mg (e.g., 1 mg to 5 mg, e.g., 1 mg, 2 mg, 3 mg, 4 mg, 5 mg) of antibody per kg of body weight at time 0 (e.g., TO or day 1), (2) administering a dose of at least 30 mg (e.g., 30-50, 30, 35, 40, 45, 50 mg) of anti-CD47 antibody per kg of body weight once every week beginning one week after TO with an additional (optional) loading dose of at least 30 mg/kg (e.g., 30-50, 30, 35, 40, 45, 50 mg) on Day 11 (week 2), and (3) administering a dose of 375 mg/m² of anti-CD20 antibody once every week; and the second cycle comprising (1) administering a dose of at least 30 mg (e.g., 30-50, 30, 35, 40, 45, 50 mg) of anti-CD47 antibody per kg of body weight once every week, and (2) administering a dose of 375 mg/m² of anti-CD20 antibody once every month. Anti-CD47 and anti-CD20 antibodies can be subsequently administered through a third, fourth, and fifth cycles. In various embodiments, the third, fourth, and fifth cycles each comprise (1) administering a dose of at least 30 mg (e.g., 30-50, 30, 35, 40, 45, 50 mg) of anti-CD47 antibody per kg of body weight once every other week and (2) administering a dose of 375 mg/m² of anti-CD20 antibody once every month. Additional cycles (e.g., sixth, seventh, eighth, ninth, tenth, etc.) of anti-CD47 and anti-CD20 antibodies can be provided without limit or, for example, until clinical benefit is reduced or lost or no longer observed. When reached and starting at Cycle 6 and beyond, anti-CD20 antibody can instead be administered to the subject at a dose of 375 mg/m² once every eight weeks. When reached and starting at Cycle 6 and beyond, anti-CD20 antibody can instead be administered to the subject at a dose of 375 mg/m² once every eight weeks. Generally, anti-CD47 antibody and anti-CD20 antibody will continue to be administered to the subject as above until the subject loses clinical benefit, e.g., via CR or death. The CD20+ cancer can be at least one of: a B cell cancer, Non-Hodgkin's lymphoma (NHL), indolent lymphoma, follicular lymphoma (FL), marginal zone lymphoma, or diffuse large B cell lymphoma (DLBCL). In various embodiments, the method targets CD47 or SIRPα.

Also disclosed herein is a method of treating a human subject having a CD20+ cancer, comprising administering an anti-CD47 antibody (e.g., Hu5F9-G4) and an anti-CD20 antibody (e.g., rituximab) to the subject for at least two distinct cycles of four weeks each, the first cycle comprising (1) administering a priming dose of anti-CD47 antibody in the range of 1 mg to 10 mg (e.g., 1 mg to 5 mg, e.g., 1 mg, 2 mg, 3 mg, 4 mg, 5 mg) of antibody per kg of body weight at time 0 (e.g., TO or day 1), (2) administering a dose of at least 30 mg (e.g., 30-50, 30, 35, 40, 45, 50 mg) of anti-CD47 antibody per kg of body weight once every week beginning one week after TO with an additional (optional) loading dose of at least 30 mg/kg (e.g., 30-50, 30, 35, 40, 45, 50 mg) on Day 11 (week 2), and (3) administering a dose of 375 mg/m² of anti-CD20 antibody once every week; and the second cycle comprising (1) administering a dose of at least 30 mg (e.g., 30-50, 30, 35, 40, 45, 50 mg) of anti-CD47 antibody per kg of body weight once every week, and (2) administering a dose of 375 mg/m² of anti-CD20 antibody once every month. Anti-CD47 and anti-CD20 antibodies can be subsequently administered through a third, fourth, and fifth cycles. In various embodiments, the third, fourth, and fifth cycles each comprise (1) administering a dose of at least 30 mg (e.g., 30-50, 30, 35, 40, 45, 50 mg) of anti-CD47 antibody per kg of body weight once every other week and (2) administering a dose of 375 mg/m² of anti-CD20 antibody once every month. Additional cycles (e.g., sixth, seventh, eighth, ninth, tenth, etc.) of anti-CD47 and anti-CD20 antibodies can be provided without limit or, for example, until clinical benefit is reduced or lost or no longer observed. When reached and starting at Cycle 6 and beyond, anti-CD20 antibody can instead be administered to the subject at a dose of 375 mg/m² once every eight weeks. When reached and starting at Cycle 6 and beyond, anti-CD20 antibody can instead be administered to the subject at a dose of 375 mg/m² once every eight weeks. Generally, anti-CD47 antibody and anti-CD20 antibody will continue to be administered to the subject as above until the subject loses clinical benefit, e.g., via CR or death. The anti-CD47 antibody can be Hu5F9-G4. The anti-CD20 antibody can be rituximab. In various embodiments, the method targets CD47 or SIRPα.

Also disclosed herein is a method of treating a human subject having lymphoma, comprising administering an anti-CD47 antibody (e.g., Hu5F9-G4) and an anti-CD20 antibody (e.g., rituximab) to the subject for at least two distinct cycles of four weeks each, the first cycle comprising (1) administering a priming dose of anti-CD47 antibody in the range of 1 mg to 10 mg (e.g., 1 mg to 5 mg, e.g., 1 mg, 2 mg, 3 mg, 4 mg, 5 mg) of antibody per kg of body weight at time 0 (e.g., TO or day 1), (2) administering a dose of at least 30 mg (e.g., 30-50, 30, 35, 40, 45, 50 mg) of anti-CD47 antibody per kg of body weight once every week beginning one week after TO with an additional (optional) loading dose of at least 30 mg/kg (e.g., 30-50, 30, 35, 40, 45, 50 mg) on Day 11 (week 2), and (3) administering a dose of 375 mg/m² of anti-CD20 antibody once every week; and the second cycle comprising (1) administering a dose of at least 30 mg (e.g., 30-50, 30, 35, 40, 45, 50 mg) of anti-CD47 antibody per kg of body weight once every week, and (2) administering a dose of 375 mg/m² of anti-CD20 antibody once every month. Anti-CD47 and anti-CD20 antibodies can be subsequently administered through a third, fourth, and fifth cycles. In various embodiments, the third, fourth, and fifth cycles each comprise (1) administering a dose of at least 30 mg (e.g., 30-50, 30, 35, 40, 45, 50 mg) of anti-CD47 antibody per kg of body weight once every other week and (2) administering a dose of 375 mg/m² of anti-CD20 antibody once every month. Additional cycles (e.g., sixth, seventh, eighth, ninth, tenth, etc.) of anti-CD47 and anti-CD20 antibodies can be provided without limit or, for example, until clinical benefit is reduced or lost or no longer observed. When reached and starting at Cycle 6 and beyond, anti-CD20 antibody can instead be administered to the subject at a dose of 375 mg/m² once every eight weeks. When reached and starting at Cycle 6 and beyond, anti-CD20 antibody can instead be administered to the subject at a dose of 375 mg/m² once every eight weeks. Generally, anti-CD47 antibody and anti-CD20 antibody will continue to be administered to the subject as above until the subject loses clinical benefit, e.g., via CR or death. The anti-CD47 antibody can be Hu5F9-G4. The anti-CD20 antibody can be rituximab. In various embodiments, the method targets CD47 or SIRPα.

Also disclosed herein is a method of treating a human subject having NHL, comprising administering an anti-CD47 antibody (e.g., Hu5F9-G4) and an anti-CD20 antibody (e.g., rituximab) to the subject for at least two distinct cycles of four weeks each, the first cycle comprising (1) administering a priming dose of anti-CD47 antibody in the range of 1 mg to 10 mg (e.g., 1 mg to 5 mg, e.g., 1 mg, 2 mg, 3 mg, 4 mg, 5 mg) of antibody per kg of body weight at time 0 (e.g., TO or day 1), (2) administering a dose of at least 30 mg (e.g., 30-50, 30, 35, 40, 45, 50 mg) of anti-CD47 antibody per kg of body weight once every week beginning one week after TO with an additional (optional) loading dose of at least 30 mg/kg (e.g., 30-50, 30, 35, 40, 45, 50 mg) on Day 11 (week 2), and (3) administering a dose of 375 mg/m² of anti-CD20 antibody once every week; and the second cycle comprising (1) administering a dose of at least 30 mg (e.g., 30-50, 30, 35, 40, 45, 50 mg) of anti-CD47 antibody per kg of body weight once every week, and (2) administering a dose of 375 mg/m² of anti-CD20 antibody once every month. Anti-CD47 and anti-CD20 antibodies can be subsequently administered through a third, fourth, and fifth cycles. In various embodiments, the third, fourth, and fifth cycles each comprise (1) administering a dose of at least 30 mg (e.g., 30-50, 30, 35, 40, 45, 50 mg) of anti-CD47 antibody per kg of body weight once every other week and (2) administering a dose of 375 mg/m² of anti-CD20 antibody once every month. Additional cycles (e.g., sixth, seventh, eighth, ninth, tenth, etc.) of anti-CD47 and anti-CD20 antibodies can be provided without limit or, for example, until clinical benefit is reduced or lost or no longer observed. When reached and starting at Cycle 6 and beyond, anti-CD20 antibody can instead be administered to the subject at a dose of 375 mg/m² once every eight weeks. When reached and starting at Cycle 6 and beyond, anti-CD20 antibody can instead be administered to the subject at a dose of 375 mg/m² once every eight weeks. Generally, anti-CD47 antibody and anti-CD20 antibody will continue to be administered to the subject as above until the subject loses clinical benefit, e.g., via CR or death. The anti-CD47 antibody can be Hu5F9-G4. The anti-CD20 antibody can be rituximab. In various embodiments, the method targets CD47 or SIRPα.

Also disclosed herein is a method of treating a human subject having diffuse large B cell lymphoma (DLBCL), comprising administering an anti-CD47 antibody (e.g., Hu5F9-G4) and an anti-CD20 antibody (e.g., rituximab) to the subject for at least two distinct cycles of four weeks each, the first cycle comprising (1) administering a priming dose of anti-CD47 antibody in the range of 1 mg to 10 mg (e.g., 1 mg to 5 mg, e.g., 1 mg, 2 mg, 3 mg, 4 mg, 5 mg) of antibody per kg of body weight at time 0 (e.g., TO or day 1), (2) administering a dose of at least 30 mg (e.g., 30-50, 30, 35, 40, 45, 50 mg) of anti-CD47 antibody per kg of body weight once every week beginning one week after TO with an additional (optional) loading dose of at least 30 mg/kg (e.g., 30-50, 30, 35, 40, 45, 50 mg) on Day 11 (week 2), and (3) administering a dose of 375 mg/m² of anti-CD20 antibody once every week; and the second cycle comprising (1) administering a dose of at least 30 mg (e.g., 30-50, 30, 35, 40, 45, 50 mg) of anti-CD47 antibody per kg of body weight once every week, and (2) administering a dose of 375 mg/m² of anti-CD20 antibody once every month. Anti-CD47 and anti-CD20 antibodies can be subsequently administered through a third, fourth, and fifth cycles. In various embodiments, the third, fourth, and fifth cycles each comprise (1) administering a dose of at least 30 mg (e.g., 30-50, 30, 35, 40, 45, 50 mg) of anti-CD47 antibody per kg of body weight once every other week and (2) administering a dose of 375 mg/m² of anti-CD20 antibody once every month. Additional cycles (e.g., sixth, seventh, eighth, ninth, tenth, etc.) of anti-CD47 and anti-CD20 antibodies can be provided without limit or, for example, until clinical benefit is reduced or lost or no longer observed. When reached and starting at Cycle 6 and beyond, anti-CD20 antibody can instead be administered to the subject at a dose of 375 mg/m² once every eight weeks. When reached and starting at Cycle 6 and beyond, anti-CD20 antibody can instead be administered to the subject at a dose of 375 mg/m² once every eight weeks. Generally, anti-CD47 antibody and anti-CD20 antibody will continue to be administered to the subject as above until the subject loses clinical benefit, e.g., via CR or death. The anti-CD47 antibody can be Hu5F9-G4. The anti-CD20 antibody can be rituximab. In various embodiments, the method targets CD47 or SIRPα.

Also disclosed herein is a method of treating a human subject having indolent lymphoma comprising administering an anti-CD47 antibody (e.g., Hu5F9-G4) and an anti-CD20 antibody (e.g., rituximab) to the subject for at least two distinct cycles of four weeks each, the first cycle comprising (1) administering a priming dose of anti-CD47 antibody in the range of 1 mg to 10 mg (e.g., 1 mg to 5 mg, e.g., 1 mg, 2 mg, 3 mg, 4 mg, 5 mg) of antibody per kg of body weight at time 0 (e.g., TO or day 1), (2) administering a dose of at least 30 mg (e.g., 30-50, 30, 35, 40, 45, 50 mg) of anti-CD47 antibody per kg of body weight once every week beginning one week after TO with an additional (optional) loading dose of at least 30 mg/kg (e.g., 30-50, 30, 35, 40, 45, 50 mg) on Day 11 (week 2), and (3) administering a dose of 375 mg/m² of anti-CD20 antibody once every week; and the second cycle comprising (1) administering a dose of at least 30 mg (e.g., 30-50, 30, 35, 40, 45, 50 mg) of anti-CD47 antibody per kg of body weight once every week, and (2) administering a dose of 375 mg/m² of anti-CD20 antibody once every month. Anti-CD47 and anti-CD20 antibodies can be subsequently administered through a third, fourth, and fifth cycles. In various embodiments, the third, fourth, and fifth cycles each comprise (1) administering a dose of at least 30 mg (e.g., 30-50, 30, 35, 40, 45, 50 mg) of anti-CD47 antibody per kg of body weight once every other week and (2) administering a dose of 375 mg/m² of anti-CD20 antibody once every month. Additional cycles (e.g., sixth, seventh, eighth, ninth, tenth, etc.) of anti-CD47 and anti-CD20 antibodies can be provided without limit or, for example, until clinical benefit is reduced or lost or no longer observed. When reached and starting at Cycle 6 and beyond, anti-CD20 antibody can instead be administered to the subject at a dose of 375 mg/m² once every eight weeks. When reached and starting at Cycle 6 and beyond, anti-CD20 antibody can instead be administered to the subject at a dose of 375 mg/m² once every eight weeks. Generally, anti-CD47 antibody and anti-CD20 antibody will continue to be administered to the subject as above until the subject loses clinical benefit, e.g., via CR or death. The anti-CD47 antibody can be Hu5F9-G4. The anti-CD20 antibody can be rituximab. In various embodiments, the method targets CD47 or SIRPα.

Also disclosed herein is a method of treating a human subject having follicular lymphoma (FL), comprising administering an anti-CD47 antibody (e.g., Hu5F9-G4) and an anti-CD20 antibody (e.g., rituximab) to the subject for at least two distinct cycles of four weeks each, the first cycle comprising (1) administering a priming dose of anti-CD47 antibody in the range of 1 mg to 10 mg (e.g., 1 mg to 5 mg, e.g., 1 mg, 2 mg, 3 mg, 4 mg, 5 mg) of antibody per kg of body weight at time 0 (e.g., TO or day 1), (2) administering a dose of at least 30 mg (e.g., 30-50, 30, 35, 40, 45, 50 mg) of anti-CD47 antibody per kg of body weight once every week beginning one week after TO with an additional (optional) loading dose of at least 30 mg/kg (e.g., 30-50, 30, 35, 40, 45, 50 mg) on Day 11 (week 2), and (3) administering a dose of 375 mg/m² of anti-CD20 antibody once every week; and the second cycle comprising (1) administering a dose of at least 30 mg (e.g., 30-50, 30, 35, 40, 45, 50 mg) of anti-CD47 antibody per kg of body weight once every week, and (2) administering a dose of 375 mg/m² of anti-CD20 antibody once every month. Anti-CD47 and anti-CD20 antibodies can be subsequently administered through a third, fourth, and fifth cycles. In various embodiments, the third, fourth, and fifth cycles each comprise (1) administering a dose of at least 30 mg (e.g., 30-50, 30, 35, 40, 45, 50 mg) of anti-CD47 antibody per kg of body weight once every other week and (2) administering a dose of 375 mg/m² of anti-CD20 antibody once every month. Additional cycles (e.g., sixth, seventh, eighth, ninth, tenth, etc.) of anti-CD47 and anti-CD20 antibodies can be provided without limit or, for example, until clinical benefit is reduced or lost or no longer observed. When reached and starting at Cycle 6 and beyond, anti-CD20 antibody can instead be administered to the subject at a dose of 375 mg/m² once every eight weeks. When reached and starting at Cycle 6 and beyond, anti-CD20 antibody can instead be administered to the subject at a dose of 375 mg/m² once every eight weeks. Generally, anti-CD47 antibody and anti-CD20 antibody will continue to be administered to the subject as above until the subject loses clinical benefit, e.g., via CR or death. The anti-CD47 antibody can be Hu5F9-G4. The anti-CD20 antibody can be rituximab. In various embodiments, the method targets CD47 or SIRPα.

Also disclosed herein is a method of treating a human subject having marginal zone lymphoma, comprising administering an anti-CD47 antibody (e.g., Hu5F9-G4) and an anti-CD20 antibody (e.g., rituximab) to the subject for at least two distinct cycles of four weeks each, the first cycle comprising (1) administering a priming dose of anti-CD47 antibody in the range of 1 mg to 10 mg (e.g., 1 mg to 5 mg, e.g., 1 mg, 2 mg, 3 mg, 4 mg, 5 mg) of antibody per kg of body weight at time 0 (e.g., TO or day 1), (2) administering a dose of at least 30 mg (e.g., 30-50, 30, 35, 40, 45, 50 mg) of anti-CD47 antibody per kg of body weight once every week beginning one week after TO with an additional (optional) loading dose of at least 30 mg/kg (e.g., 30-50, 30, 35, 40, 45, 50 mg) on Day 11 (week 2), and (3) administering a dose of 375 mg/m² of anti-CD20 antibody once every week; and the second cycle comprising (1) administering a dose of at least 30 mg (e.g., 30-50, 30, 35, 40, 45, 50 mg) of anti-CD47 antibody per kg of body weight once every week, and (2) administering a dose of 375 mg/m² of anti-CD20 antibody once every month. Anti-CD47 and anti-CD20 antibodies can be subsequently administered through a third, fourth, and fifth cycles. In various embodiments, the third, fourth, and fifth cycles each comprise (1) administering a dose of at least 30 mg (e.g., 30-50, 30, 35, 40, 45, 50 mg) of anti-CD47 antibody per kg of body weight once every other week and (2) administering a dose of 375 mg/m² of anti-CD20 antibody once every month. Additional cycles (e.g., sixth, seventh, eighth, ninth, tenth, etc.) of anti-CD47 and anti-CD20 antibodies can be provided without limit or, for example, until clinical benefit is reduced or lost or no longer observed. When reached and starting at Cycle 6 and beyond, anti-CD20 antibody can instead be administered to the subject at a dose of 375 mg/m² once every eight weeks. When reached and starting at Cycle 6 and beyond, anti-CD20 antibody can instead be administered to the subject at a dose of 375 mg/m² once every eight weeks. Generally, anti-CD47 antibody and anti-CD20 antibody will continue to be administered to the subject as above until the subject loses clinical benefit, e.g., via CR or death. The anti-CD47 antibody can be Hu5F9-G4. The anti-CD20 antibody can be rituximab. In various embodiments, the method targets CD47 or SIRPα.

Administration

In the methods described herein, compositions, e.g., an anti-CD47 antibody and, optionally, an additional agent, are administered to a subject. The compositions can be administered by parenteral, topical, intravenous, intra-abdominal, intra-tumoral, oral, subcutaneous, intra-arterial, intracranial, intraperitoneal, intranasal or intramuscular means. A typical route of administration is intravenous or intra-tumoral, although other routes can be equally effective.

In some embodiments the anti-CD47 antibody and/or the additional agent is administered intra-abdominally. In some embodiments the anti-CD47 antibody and/or the additional agent is administered intravenously. In some embodiments the anti-CD47 antibody and/or the additional agent is administered intra-tumorally. In one embodiment, a priming dose of the anti-CD47 antibody is administered, and the priming dose is delivered subcutaneously. In some embodiments, the anti-CD47 antibody and the additional agent are administered concurrently. In some embodiments, the anti-CD47 antibody and the additional agent are administered sequentially.

The active agents are administered within a period of time to produce an additive or synergistic effect on depletion of cancer cells in the host. Methods of administration include, without limitation, systemic administration, intra-tumoral administration, etc. Usually the anti-CD47 antibody is administered within about a period of about 45 days, about 30 days, about 21 days, about 14 days, about 10 days, about 8 days, about 7 days, about 6 days, about 5 days, about 4 days, about 3 days, about 2 days, about 1 day or substantially the same day as the additional agent. In some embodiments the anti-CD47 antibody is administered prior to the additional agent. In some embodiments the anti-CD47 antibody is administered after the additional agent. The agents can be considered to be combined if administration scheduling is such that the serum level of both agents is at a therapeutic level at the same time. Administration may be repeated as necessary for depletion of the cancer cell population.

One or more antibodies disclosed herein can be administered by a medical professional, optionally a physician.

One or more antibodies disclosed herein can be administered by the subject.

Clinical Endpoints

The methods described herein result in at least one improved endpoint compared to baseline.

A method disclosed herein can result in an objective response (OR) in a subject. An objective response is a partial response or complete remission as defined by Cheson, Lugano, or similar NHL response criteria.

A method disclosed herein can result disease control in a subject. Disease control is stable disease plus objective response.

A method disclosed herein can result in a partial response (PR) in a subject. PR is a shrinkage of the tumor by at least 50% by imaging criteria (CT or PET/CT) without complete disappearance of tumor lesions. By PET/CT criteria, a PR is as described above or by reduced metabolic uptake compared with baseline and residual masses of any size (Lugano criteria, Cheson et al., JCO 2014).

A method disclosed herein can result in a complete response (CR) in a subject. Cheson et al., JCO 2014.

A method disclosed herein can result in stable disease (SD) in a subject. Cheson et al., JCO 2014.

A method disclosed herein can reduce the size of a subject's cancer relative to baseline where baseline is determined prior to administration of anti-CD47 antibody.

A method disclosed herein can result in a reversal of refractoriness to rituximab in a subject.

Pharmaceutical Compositions

The methods described herein include administration of pharmaceutical compositions comprising the anti-CD47 antibody and/or the additional agent. In some embodiments, the pharmaceutical composition includes both the anti-CD47 and the additional agent. In some embodiments, a pharmaceutical composition includes one of the anti-CD47 and additional agent. Therefore, sequential administration of the anti-CD47 and additional agent can be achieved by separately administering a first pharmaceutical composition and then subsequently administering the second pharmaceutical composition.

Typically, the compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S. Food and Drug Administration.

The pharmaceutical compositions can be administered in a variety of unit dosage forms depending upon the method of administration. For example, unit dosage forms suitable for oral administration include, but are not limited to, powder, tablets, pills, capsules and lozenges. It is recognized that compositions of the invention when administered orally, should be protected from digestion. This is typically accomplished either by complexing the molecules with a composition to render them resistant to acidic and enzymatic hydrolysis, or by packaging the molecules in an appropriately resistant carrier, such as a liposome or a protection barrier. Means of protecting agents from digestion are well known in the art.

The compositions for administration will commonly comprise an antibody or other ablative agent dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as needed to approximate physiological conditions such as pH adjusting and buffering agents, toxicity adjusting agents and the like, e.g., sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration selected and the patient's needs (e.g., Remington's Pharmaceutical Science (15th ed., 1980) and Goodman & Gillman, The Pharmacological Basis of Therapeutics (Hardman et al., eds., 1996)).

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

“Pharmaceutically acceptable salts and esters” means salts and esters that are pharmaceutically acceptable and have the desired pharmacological properties. Such salts include salts that can be formed where acidic protons present in the compounds are capable of reacting with inorganic or organic bases. Suitable inorganic salts include those formed with the alkali metals, e.g. sodium and potassium, magnesium, calcium, and aluminum. Suitable organic salts include those formed with organic bases such as the amine bases, e.g., ethanolamine, diethanolamine, triethanolamine, tromethamine, N methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). Pharmaceutically acceptable esters include esters formed from carboxy, sulfonyloxy, and phosphonoxy groups present in the compounds, e.g., C₁₋₆ alkyl esters. When there are two acidic groups present, a pharmaceutically acceptable salt or ester can be a mono-acid-mono-salt or ester or a di-salt or ester; and similarly where there are more than two acidic groups present, some or all of such groups can be salified or esterified. Compounds named in this invention can be present in unsalified or unesterified form, or in salified and/or esterified form, and the naming of such compounds is intended to include both the original (unsalified and unesterified) compound and its pharmaceutically acceptable salts and esters. Also, certain compounds named in this invention may be present in more than one stereoisomeric form, and the naming of such compounds is intended to include all single stereoisomers and all mixtures (whether racemic or otherwise) of such stereoisomers.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

Kits

Also described herein are kits comprising the active agents, e.g., an anti-CD47 antibody and, optionally, an additional agent, and formulations thereof, and instructions for use. The additional agent may be an anti-CD20 agent such as rituximab. Kits typically include a label indicating the intended use of the contents of the kit. The term label includes any writing, or recorded material supplied on or with the kit, or which otherwise accompanies the kit.

Also provided are kits for use in the various methods disclosed herein. The subject kits include a primer agent and an anti-CD47 agent. In some embodiments, a kit comprises two or more primer agents. In some embodiments, a kit comprises two or more anti-CD47 agents. In some embodiments, a primer agent is provided in a dosage form (e.g., a priming dosage form). In some embodiments, a primer agent is provided in two or more different dosage forms (e.g., two or more different priming dosage forms). In some embodiments, an anti-CD47 agent is provided in a dosage form (e.g., a therapeutically effective dosage form). In some embodiments, an anti-CD47 agent is provided in two or more different dosage forms (e.g., two or more different therapeutically effective dosage forms). In the context of a kit, a primer agent and/or an anti-CD47 agent can be provided in liquid or solid form in any convenient packaging (e.g., stick pack, dose pack, etc.).

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), flash drive, and the like, on which the information has been recorded. Yet another form of these instructions that may be present is a website address which may be used via the internet to access the information at a removed site.

Sequences

In some embodiments, the methods described herein include administration of antibodies with sequences described herein; e.g., the heavy chain, light chain, and/or CDR sequences described herein. The sequences of the administered antibodies can be, e.g., at least 95, 96, 97, 98, 99, or 100% identical to the sequences described herein.

The term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (<www.ncbi.nlm.nih.gov/>)

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3^{rd} Ed. (Plenum Press) Vols A and B(1992).

### Example 1: Hu5F9-G4 in Combination with Rituximab in Human Patients with Relapsed/Refractory B-Cell Non-Hodgkin's Lymphoma

Introduction

Non-Hodgkin's lymphoma (NHL) is among the most common cancers in the USA and Europe, with more than 70,000 and 93,000 new cases diagnosed every year, respectively. Diffuse large B-cell lymphoma (DLBCL) is an aggressive subtype of NHL with high relapse rate and poor long-term survival. In addition, few treatment options are available to patients with indolent lymphoma who have relapsed or are refractory to rituximab. Novel and effective therapies are needed to address these high unmet medical needs. Hu5F9-G4 is a monoclonal antibody that targets CD47, an anti-phagocytic cell surface protein. Nonclinical studies have demonstrated that blockade of CD47 signaling through this antibody eliminates human tumor cells including NHL, through facilitating phagocytosis by macrophages. Additional nonclinical studies demonstrate that anti-CD47 antibodies can synergize with Fc receptor-activating anti-cancer antibodies including rituximab. Combination therapy with Hu5F9-G4 and rituximab, an anti-CD20 monoclonal antibody, demonstrated a synergistic anti-cancer response compared to either agent alone in nonclinical models of NHL.

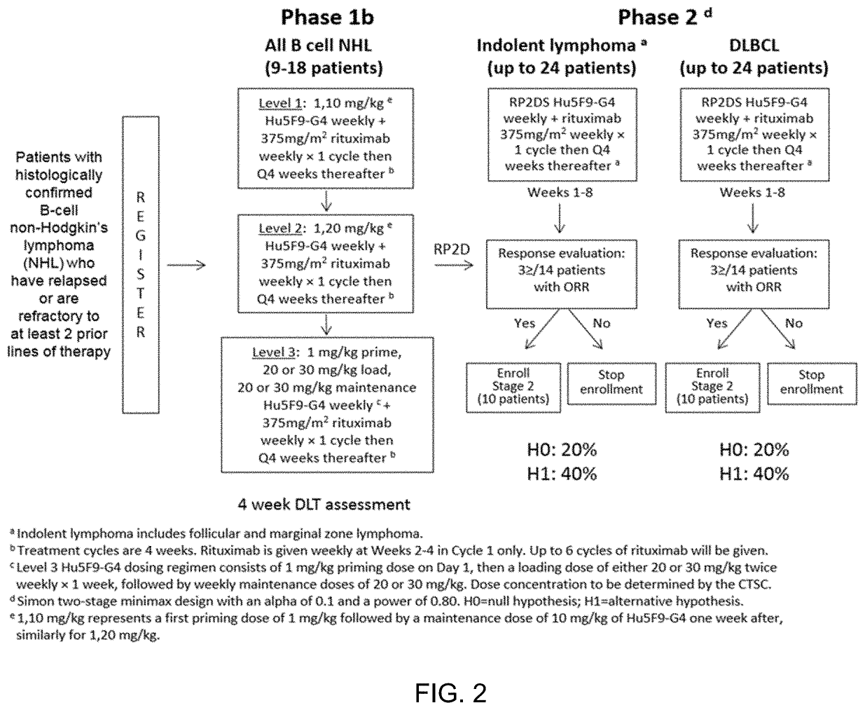

This Phase 1b/2 trial establishes the safety and tolerability and dosing strategy of Hu5F9-G4 in combination with rituximab in patients with relapsed/refractory B-cell NHL. Hu5F9-G4 and rituximab were both administered intravenously. Initially, this trial utilized a reduced starting dose of Hu5F9-G4 in combination with full doses of rituximab. Subsequent dose cohorts escalated the dose of Hu5F9-G4. In addition, preliminary anti-cancer activity was investigated with this antibody combination. FIG. 2 shows a study design schema for Phase 1b/2 Trial of Hu5F9-G4 in Combination with Rituximab in Patients with Relapsed/Refractory B-cell Non-Hodgkin's Lymphoma.

Patient Eligibility

Inclusion Criteria were as follows:
1. Adults ≥18 years
2. Phase 1b only: B-cell NHL expressing CD20 by immunohistochemistry (IHC) or flow cytometry, relapsed or refractory to at least 2 prior lines of therapy
3. DLBCL Phase 2 cohort: Histologically confirmed de novo or transformed DLBCL expressing CD20 by IHC or flow cytometry, refractory to frontline therapy; or relapsed or refractory to second line salvage regimens or autologous hematopoietic cell transplantation
4. Indolent lymphoma Phase 2 cohort: Histologically confirmed marginal zone or follicular lymphoma (Grade 1-3a) expressing CD20 by IHC or flow cytometry, relapsed or refractory to at least 2 prior lines of therapy
5. Eastern Cooperative Oncology Group (ECOG) score 0-2
6. Disease that is measurable or assessable for response per Lugano Classification for lymphomas
7. Laboratory measurements, blood counts:
   Hemoglobin ≥9.5 g/dL
   Absolute neutrophil count (ANC)≥1.0×10⁹/mL
   Platelets ≥50×10⁹/mL
8. Laboratory measurements, hepatic function:
   Aspartate aminotransferase (AST)/alanine aminotransferase (ALT)<5×upper limit of normal (ULN)
   Bilirubin≤1.5× or 3.0×ULN and primarily unconjugated if patient has a documented history of Gilbert's syndrome or a genetic equivalent
9. Laboratory measurements, renal function:
   Serum creatinine ≤1.5×ULN or calculated glomerular filtration rate (GFR) >40 mL/min/1.73 m²
10. Negative urine or serum pregnancy test within 30 days before enrollment and within 72 hours before the first administration of Hu5F9-G4 for women of childbearing potential.
11. Females of childbearing potential should be willing to use 1 highly effective method of contraception during the study and for 12 months after the last dose of rituximab or 4 months after the last dose of Hu5F9-G4, whichever occurs later
12. Males should be willing to use 1 effective method of contraception during the study and for 12 months after the last dose of rituximab or 4 months after the last dose of Hu5F9-G4, whichever occurs later, if the partner is a female of childbearing potential
13. Subject has provided informed consent
14. Should be willing and able to comply with clinic visits and procedures outlined in the study protocol
15. Phase 2 only: Willing to consent to 1 mandatory pre-treatment and 1 on-treatment tumor biopsy, unless not feasible as determined by the Investigator (reasons include but are not limited to lack of accessible tumor tissue to biopsy and patient safety issues)

Exclusion Criteria were as follows:
1. Patients with active brain metastases. (Patients with stable treated central nervous system [CNS] lesions who are off corticosteroid therapy for at least 3 weeks are not considered active.)
2. Prior anti-cancer therapy including chemotherapy, hormonal therapy, or investigational agents within 2 weeks or within at least 4 half-lives prior to Hu5F9-G4 dosing (up to a maximum of 4 weeks), whichever is longer. In all situations, the maximum required washout period will not exceed 4 weeks prior to the day of first treatment with Hu5F9-G4. Low dose steroids (oral prednisone or equivalent ≤20 mg per day), localized non-CNS radiotherapy, pre-existing previous hormonal therapy with LHRH agonists for prostate cancer, and treatment with bisphosphonates and RANKL inhibitors are not criteria for exclusion.
3. Known active or chronic hepatitis B or C infection or human immunodeficiency virus (HIV).
4. Red blood cell (RBC) transfusion dependence, defined as requiring more than 2 units of RBC transfusions during the 4-week period prior to screening. RBC transfusions are permitted during screening and prior to enrollment to meet the hemoglobin inclusion criteria.
5. History of hemolytic anemia or Evans syndrome in the last 3 months.
6. Positive Direct Antiglobulin Test (DAT).
7. Prior treatment with CD47 or signal regulatory protein alpha (SIRP( ) targeting agents.
8. Second malignancy, except treated basal cell or localized squamous skin carcinomas, localized prostate cancer, or other malignancy for which patients are not on active anti-cancer therapy as defined in Exclusion Criterion 2.
9. Hypersensitivity to the active substance, to murine proteins, or to any of the other excipients of rituximab listed at: RITUXAN® (rituximab) prescribing information http://www.gene.com/download/pdf/rituxan_prescribing.pdf, MabThera® (rituximab) prescribing information
http://www.ema.europa.eu/docs/en_GB/document_library/EPAR_-_Product_Information/human/000165/WC500025821.pdf. Each link is as of Apr. 27, 2017.
10. Significant medical diseases or conditions, as assessed by the Investigators and Sponsor that would substantially increase the risk-benefit ratio of participating in the study. This includes but is not limited to acute myocardial infarction within the last 6 months, unstable angina, uncontrolled diabetes mellitus, significant active infections, severely immunocompromised state, and congestive heart failure New York Heart Association (NYHA) Class II-IV.
11. History of psychiatric illness or substance abuse likely to interfere with ability to comply with protocol requirements or give informed consent.
12. Pregnancy or active breastfeeding.

Study Objectives

Primary Objectives

(1) Investigation of the safety and tolerability, and definition of Phase 2 dose for Hu5F9-G4 in combination with rituximab.

(2) In Phase 2, evaluation of efficacy of Hu5F9-G4 in combination with rituximab in patients with indolent lymphoma and DLBCL as measured by the overall response rate (ORR).

Secondary Objectives

(1) In Phase 1b and 2, evaluation of pharmacokinetic (PK) profile of Hu5F9-G4 in combination with rituximab.

(2) In Phase 1b and 2, evaluation of immunogenicity of Hu5F9-G4 in combination with rituximab.

(3) In Phase 2, evaluation of efficacy of Hu5F9-G4 in combination with rituximab in indolent lymphoma and DLBCL as measured by the duration of response, best overall response, progression free survival, and overall survival.

(4) Evaluation of response rates according to LYRIC criteria for lymphomas.

Exploratory Objectives

(1) Assessment of biomarkers of immune cell efficacy and tumor penetration of Hu5F9-G4 in combination with rituximab.

(2) Assessment of efficacy in molecular subtypes of NHL.

Endpoints

Primary

(1) Dose-limiting toxicities (DLTs) (Phase 1b only) and adverse events (AEs) according to NCI CTCAE, Version 4.03.

(2) Phase 2: Objective response according to the Lugano Classification for lymphomas.

Secondary

(1) Phase 1b and 2: Concentration versus time measurements for Hu5F9-G4 in combination with rituximab and PK parameters, including maximum plasma concentration (Cₘₐₓ), time to maximum concentration (Tₘₐₓ), terminal half-life (t_{1/2}), area under the curve (AUC), clearance (CL), and volume of distribution during the terminal phase (V_{z}).

(2) Phase 1b and 2: Anti-drug antibodies to Hu5F9-G4 and rituximab.

(3) Phase 2: Duration of response (DOR), best overall response (BOR), progression-free survival (PFS), and overall survival (OS).

(4) Objective response according to the LYRIC criteria for lymphomas.

Exploratory

(1) CD47 receptor occupancy on peripheral RBCs and white blood cells (WBCs), and lymphoma cells, where applicable.

(2) Pharmacodynamic markers of Hu5F9-G4 biological activity potentially including, but not limited to, circulating cytokine profiles, T-cell receptor sequencing on circulating T cells, mass cytometry (CyTOF)/flow cytometry of circulating leukocytes, and T-cell activation studies.

(3) In patients undergoing tumor biopsies, Hu5F9-G4 saturation of tumor cells and changes in the tumor microenvironment including, but not limited to, macrophage and T-cell tumor infiltration.

(4) In patients undergoing tumor biopsies, correlation of anti-cancer response to molecular subtypes of NHL including, but not limited to, cell-of-origin in DLBCL and BCL2, BCL6, and MYC mutation/expression status.

Intervention and Mode of Delivery

Hu5F9-G4 is a humanized monoclonal antibody against CD47 and rituximab is a chimeric monoclonal antibody against CD20. Both drugs were administered intravenously. Hu5F9-G4 was administered on Days 1, 8, 15, and 22 for all Phase 1b cycles while rituximab was administered on Days 8, 15, and 22 for the first cycle followed by Day 1 for Cycles 2-6.

Duration of Intervention and Evaluation

Phase 1b/2: For the Phase 1b part of the study, patients were treated with Hu5F9-G4 and rituximab in a standard 3+3 dose escalation design. DLT safety evaluation used for determination of the maximum tolerated dose (MTD) occurred within the first 4 weeks. A response assessment occurred every 2 cycles (8 weeks) until disease progression. Rituximab was or is administered for a total of 6 cycles, while Hu5F9-G4 treatment was or is extended beyond 6 cycles for those who do not have disease progression.

Number of Patients

Phase 1b: 9 to 18 patients total

Per dose level:

Level 1: 3-6

Level 2: 3-6

Level 3: 3-6

Phase 2: 48 patients (24 patients for indolent lymphoma; 24 patients for DLBCL)

Study Total: 57-66 patients (assuming progression to Stage 2 of Phase 2)

H-Score

A H-score was calculated as a measure of the presence or absence of B-cells in a patient. A tissue biopsy (e.g., a cancer biopsy) was obtained from a patient and immunostaining for a B-cell marker such as CD19 or CD20. Immunostained tissue slices were imaged for the B-cell marker for determining the H-score. An exemplary method for determining a H-score is described below in reference to the presence of absence of CD20 B-cells.

The H-score can be scored as follows: 1) A score of 2+ or +3 on a scale of 0-3+, whereas 0 represents absent B-cell staining with 3+representing maximal B-cell staining; 2) or using an H score, a semiquantitative score whereby CD20 membrane staining intensity (0, 1+, 2+, or 3+) is determined for each cell in a fixed field. The percentage of cells at each staining intensity level is calculated and an H-score is assigned using the following formula: [1×(% cells scored as 1+)+2×(% cells scored as 2+)+3×(% cells scored as 3+)]. An H-score can range from 0-300. A similar method for deriving the H score may be used. A high H score cut-off would be utilized for presence of B-cells. Low CD20 expression can be scored with a score of 0 or 1+; whereby a low H-score cut-off is utilized for establishing a lack of B-cell presence in the subject.

Retrospective Analysis of Variables Affecting Response Rates

Different variables were analyzed to determine the different response rates between DLBCL patients enrolled in the Phase 1b trial and the DLBCL patients enrolled in the Phase 2 trial. Variables included: CD19 cell count at baseline, percent CD19 cell count over total lymphocytes at baseline, total lymphocyte count at baseline, months from last anti-CD20 therapy, rituximab concentration in a patient at baseline, tumor burden at baseline, hemoglobin count, neutrophil count, platelet count, Eastern Cooperative Oncology Group (ECOG) status, quantity of lactic acid dehydrogenase (LDH) at baseline, and quantity of albumin at baseline.

Additionally, although no clinical CD20+B-cell data was collected, patient data on CD19+ B-cells and rituximab concentration were used as a proxy for the presence or absence of CD20+ B-cells such that the presence/absence of CD20+ B-cells would serve as an additional variable for analysis.

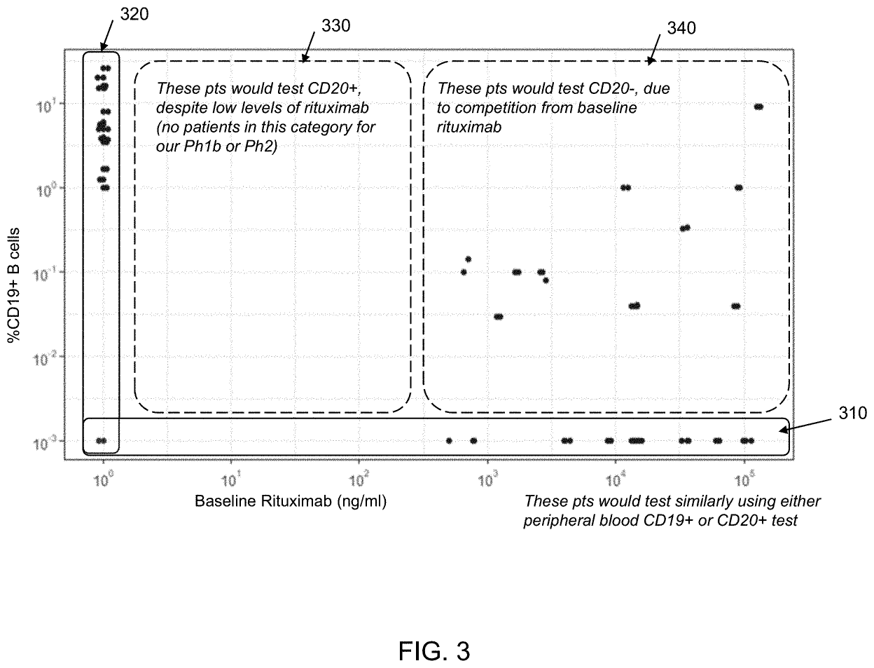

FIG. 3 shows the use of percentage CD19+ B-cells and rituximab as a proxy for presence of CD20+ B-cells. Specifically, patients with high rituximab concentration (e.g., above 103 ng/mL) and limited CD19+B Cells (e.g., below 0.01% CD19+ B-cells), labeled as 310 in FIG. 3, were categorized as not having CD20+ B-cells. Patients with low rituximab concentration (e.g., less than 10 ng/mL) and high CD19+B Cells (e.g., above 1% CD19+B-cells), labeled as 320 in FIG. 3, were categorized as having CD20+ B-cells. Patients with intermediate rituximab concentration (between 1 and 500 ng/mL) and some CD19+B Cells (above 0.01% CD19+ B-cells), labeled as 330 in FIG. 3, were categorized as having CD20+B-cells. Patients with high rituximab concentration (e.g., above 500 ng/mL) and some CD19+B Cells (above 0.01% CD19+ B-cells), labeled as 340 in FIG. 3, were categorized as having CD20+ B-cells.

For each variable, a univariate analysis was conducted using the logistic regression method that modeled the relationship between the variable and an objective response rate (including both complete response and partial response) across the DLBCL patients enrolled in the Phase 1b trial and the Phase 2 trial.

Planned Study with Refined Patient Eligibility Criteria

Revised eligibility criteria will be implemented in a new registration DLBCL patient cohort. An estimated 20 patient interim analysis using the new revised eligibility criteria will be conducted to verify whether to enroll a subsequent 80 patients. Endpoints will be based on objective response rate (complete response or partial response) with duration of response.

DLBCL patients in the interim analysis will have received greater than or equal to 2 prior lines of therapy. Table 5 below documents additional revised protocol criteria for selecting patients to be included in the 20 patient interim analysis and for their subsequent treatment.

TABLE 5
Revised Protocol Criteria

| Current Protocol Criteria | Revised Protocol Criteria |
|---|---|
| Prior therapy washout period | Prior therapy washout period of |
| of 3 weeks | 4 weeks |
| No peripheral B cell | Require patients to have presence of |
| requirement | normal B cells in the peripheral blood |
| Must be CAR-T ineligible | Remove requirement for CAR-T |
| (defined by medical | ineligibility but still collect reasons |
| judgement or lack of | if they are CAR-T ineligible |
| availability) | |
| Rituximab given first on Day | On Day 8 and subsequent combo |
| 8 followed by magrolimab | dosing days, give magrolimab 30 |
| | mg/kg first, followed by rituximab |
| 11 mostly academic sites with | Consider additional sites, such as |
| CAR T programs | community centers, that lack |
| | immediate access to CAR T cells |

### Example 2: Human Results

Patient Response Rate

Generally, DLBCL patients enrolled in the Phase 1b trial responded more favorably to the combination treatment of magrolimab and rituximab in comparison to patients enrolled in the Phase 2 trial. Table 6 shows a summary of Antitumor Activity Observed across patients treated with magrolimab and rituximab combination in both Phase 1b and Phase 2 studies as of May 15, 2019.

TABLE 6

| | Total DLBCL | Phase 1b | Phase 2 |
|---|---|---|---|
| Best overall response | N = 59 | N = 21 (%) | N = 38 (%) |
| Objective Response Rate | 21(36%) | 10 (48%) | 11 (29%) |
| (ORR) | | | |
| Complete Response (CR) | 9 (15%) | 7 (33%) | 2 (5%) |
| Partial Response (PR) | 12 (20%) | 3 (14%) | 9 (24%) |
| Stable Disease (SD) | 7 (12%) | 4 (19%) | 3 (8%) |
| Progressive Disease (PD) | 31 (53%) | 7 (33%) | 24 (63%) |

Specifically, as shown in Table 6, of the DLBCL patients enrolled in the Phase 1b trial, 10 of the 21 patients (48%) exhibited an objective response rate (7 being complete response and 3 being partial response). Comparatively, or the DLBCL patients enrolled in the Phase 2 trial, only 11 of the 38 patients (29%) exhibited an objective response rate (2 being complete response and 9 being partial response).

Additionally, of the DLBCL patients enrolled in the Phase 1b trial, 4 patients (19%) exhibited stable disease and 7 patients (33%) exhibited progressive disease. Three patients (8%) enrolled in the Phase 2 trial exhibited stable disease but a significantly higher percentage of patients enrolled in the Phase 2 trial exhibited progressive disease (24 of the 38 patients, 63%).

Additionally, in the Phase 1b trial, the median follow-up for DLBCL patients was 13.8 months. In contrast, in the phase 2 trial, the median follow-up for DLBCL patients was significantly reduced at 3.7 months.

Retrospective Analysis of Variables Affecting Response Rates

Given the significant difference in response rates among the patients enrolled in the Phase 1b and Phase 2 trials, a univariate analysis was conducted across different variables to determine likely causes that gave rise to the difference in response rates.

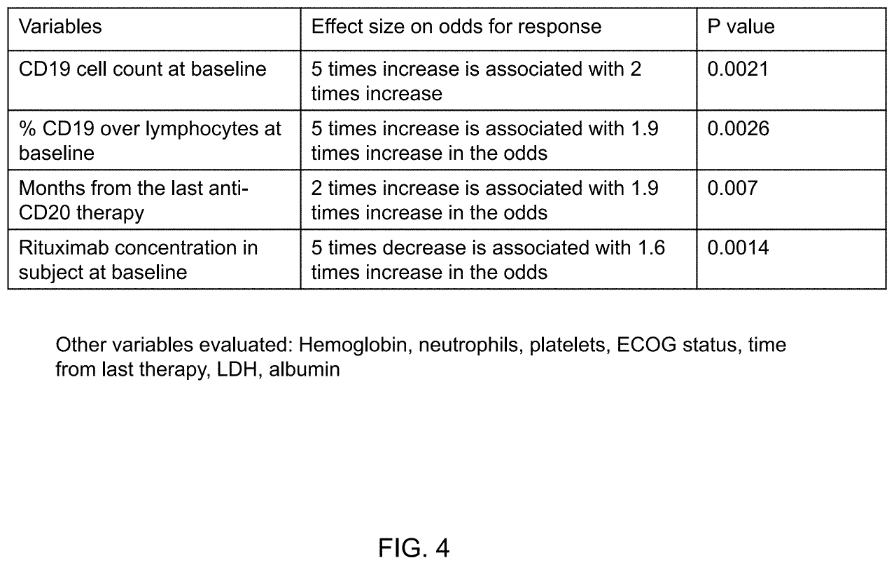

FIG. 4 shows identified variables that affect response rates among patients in the Phase 1b/2 trials. In particular, the variables of CD19 cell count at baseline, percent CD19 cell count over lymphocytes at baseline, the number of months from last anti-CD20 therapy, and rituximab concentration in the subject were found to be statistically significantly related to a patient's response to the combination magrolimab and rituximab therapy.

In particular, each of the variables of CD19 cell count at baseline, percent CD19 cell count over lymphocytes at baseline, and the number of months from last anti-CD20 therapy exhibited a direct correlation with patient objective response rate. Rituximab concentration in the subject at baseline was inversely correlated with patient objective response rate.

Presence of CD19+ B-Cells

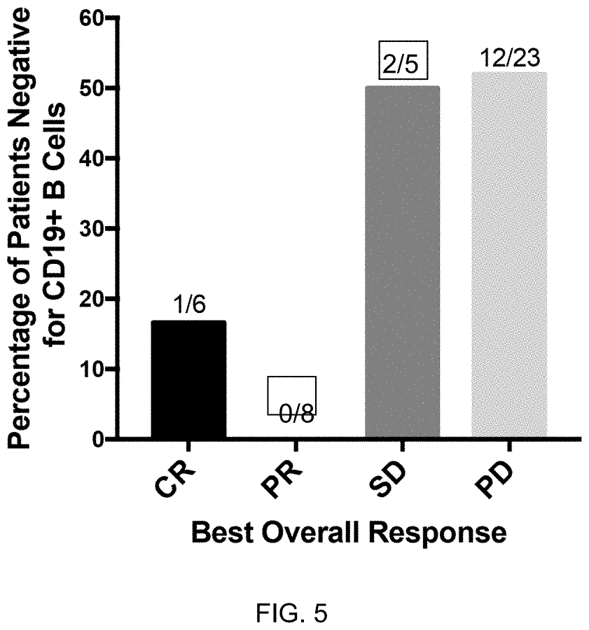

The variable of presence of CD19+ B-cells was further investigated to determine its association with patients that exhibited an objective response rate. FIG. 5 is a bar graph depicting the best overall response across patients that are negative for CD19+ B-cells. Across the patients in which CD19 B cell data was available, 6 patients exhibited CR, 7 patients exhibited PR, 4 patients exhibited SD, and 23 patients exhibited PD. Zero of the 6 patients exhibiting CR were negative for CD19+ B-cells, zero of the 7 patients exhibiting PR were negative for CD19+ B-cells, two of the 4 patients exhibiting SD were negative for CD19+ B-cells, and twelve of the 23 patients exhibiting PD were negative for CD19+B-cells. These results indicate that patients that were negative for B-cells disproportionately exhibited SD or PD.

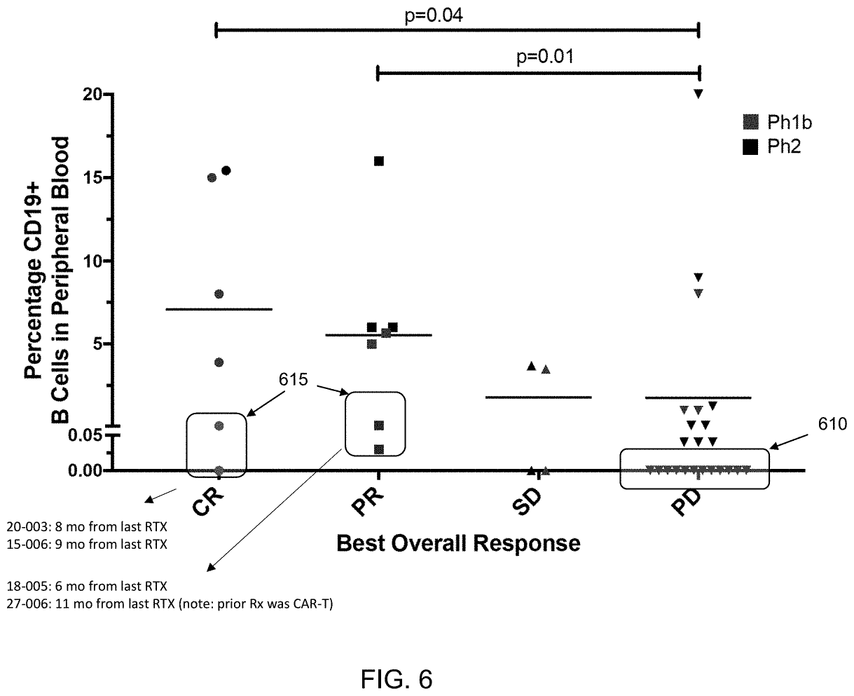

FIG. 6 is a plot depicting the best overall response of patients based on a percentage of CD19+ B-cells in the peripheral blood of the patients. Specifically, FIG. 6 depicts the percentage of CD19+ B-cells over total lymphocytes in the peripheral blood for individual patients enrolled in the Phase 1b or Phase 2 Trials as well as the best overall response for each of those individual patients. Generally, patients with a higher percentage of CD19+ B-cells over total lymphocytes in the peripheral blood responded more favorably (e.g., CR or PR) in comparison to patients with a lower percentage of CD19+ B-cells.

Specifically, patients that exhibited a CR had an average of ˜7.5% CD19+ B-cells out of total lymphocytes. Patients that exhibited a PR had an average of 5.5% CD19+B-cells out of total lymphocytes. Patients that exhibited a SD or PD had an average of ˜2% CD19+ B-cells out of total lymphocytes. The average % CD19+B-cell population in each of CR and PR patients were statistically significant in comparison to the average % CD19+B-cell population in PD patients. In particular, a large population of patients who exhibited PD, labeled as 610 on FIG. 6, had no CD19+ B-cells. Notably, four patients exhibited CR or PR, labeled as 615 on FIG. 6, which can potentially be attributed to prolonged B-cell depletion from prior treatment (rituximab).

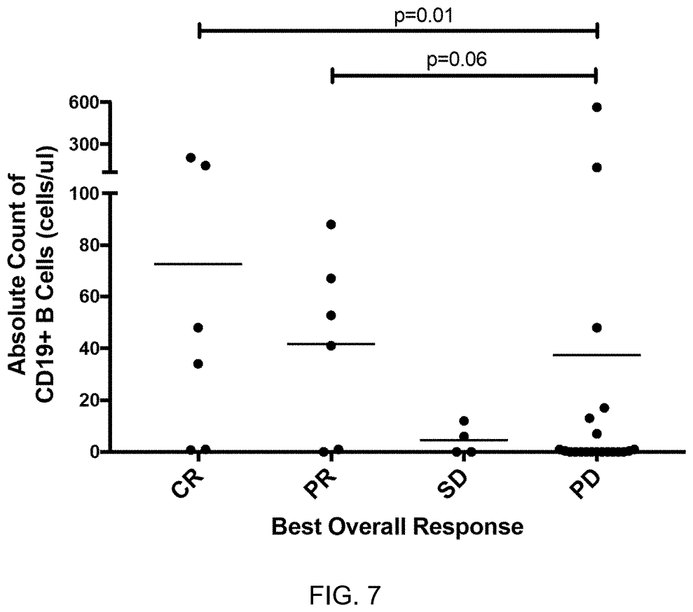

FIG. 7 is a plot depicting the best overall response of patients based on an absolute count of CD19+ B-cells in the peripheral blood of the patients. Specifically, FIG. 7 depicts the absolute count of CD19+ B-cells (cells per microliter) for individual patients enrolled in the Phase 1b or Phase 2 Trials as well as the best overall response for each of those individual patients. Similar to the conclusion drawn from the results shown in FIG. 6, patients with a higher absolute count of CD19+ B-cells responded more favorably (e.g., CR or PR) in comparison to patients with a lower absolute count of CD19+ B-cells.

Specifically, patients that exhibited a CR had an average of ˜75 CD19+ B-cells per microliter. Patients that exhibited a PR had an average of ˜42 CD19+ B-cells per microliter. Patients that exhibited SD had an average of 5% CD19+ B-cells per microliter. Patients that exhibited PD had an average of ˜39% CD19+ B-cells per microliter. Of note, patients that exhibited PD had a wide range of absolute count of CD19+ B-cells (ranging from zero cells per microliter up to 600 cells per microliter). The absolute count of CD19+B-cells in CR patients were statistically significant in comparison to the absolute count of CD19+ B-cells in PD patients.

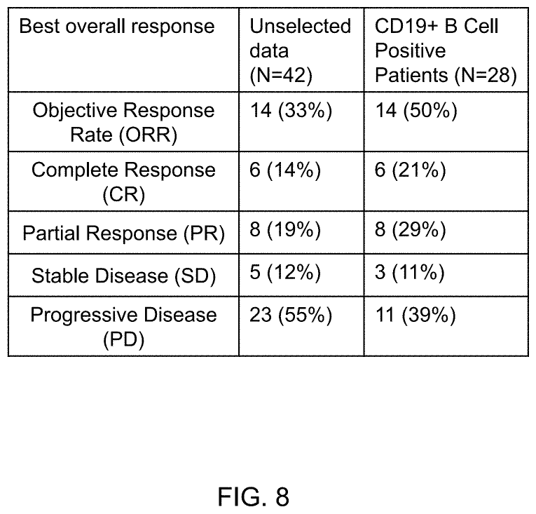

FIG. 8 shows response rates of patients involved in the Phase 1b/2 trials before and after applying an eligibility criteria for presence of CD19+ B-cells. The column titled “Unselected data” refers to the population of patients (N=42) enrolled in the Phase 1b and Phase 2 trials without concern for the presence of CD19+ B-cells. The column entitled “CD19+B cell positive patients” represents a subset (N=28) of the population of patients where CD19+ B-cells were present. Presence of B-cells was defined as detection of B-cells above the limit of detection. Therefore, if an eligibility criteria were retroactively applied to the patients enrolled in the Phase 1b/2 trials, 14 of the 42 patients would be excluded. Of the patients excluded, all exhibited either SD or PD. Specifically, retroactive application of this eligibility criteria for presence of CD19+ B-cells increased the percentage of patients that exhibited an ORR from 33% up to 50%. Additionally, retroactive application of this eligibility criteria for presence of CD19+ B-cells increased the percentage of patients that exhibited a CR and a PR from 14% to 21% and from 19% to 29%, respectively. This suggests that an eligibility criteria requiring patients to have a presence of CD19+ B-cells can be promising for identifying patients that are likely to respond favorably to the combination magrolimab and rituximab therapy.

Presence of CD20+ B-Cells

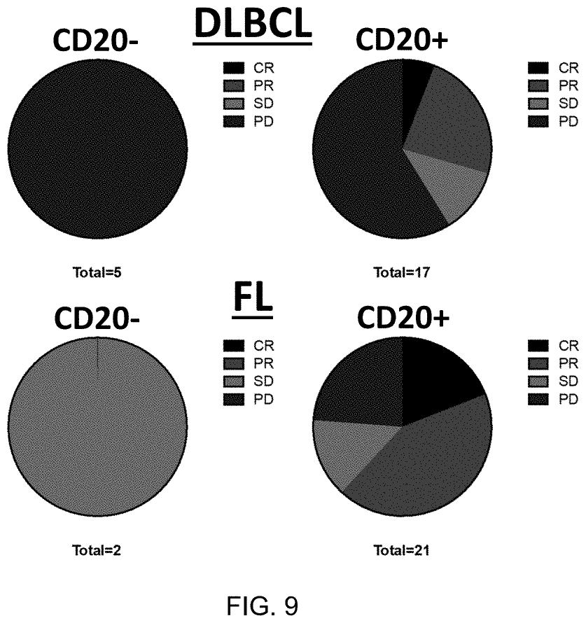

FIG. 9 shows pie charts depicting the best overall response of patients with diffuse large B-cell lymphoma or follicular lymphoma based on a presence or absence of CD20+B-cells in the patients. For both diseases, patients that were lacking CD20 B-cells (denoted as CD20—in FIG. 9) either exhibited SD or PD. On the contrary, patients that had presence of CD20+ B-cells exhibited a more varied response rate.

Specifically, of the 17 DLBCL patients with a presence of CD20+ B-cells, over 25% of those patients exhibited either a CR or PR. Additionally, of the 21 follicular lymphoma patients with a presence of CD20+ B-cells, over 50% of those patients exhibited either a CR or PR. This suggests that an eligibility criteria for the presence of CD20+ B-cells may identify patients that are more likely to respond to the combination magrolimab and rituximab treatment.

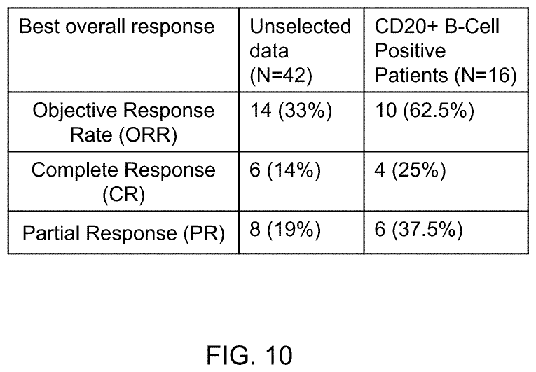

FIG. 10 shows objective response rates of patients involved in the Phase 1b/2 trials, before and after applying an eligibility criteria for presence of CD20+ B-cells. The column titled “Unselected data” refers to the population of patients (N=42) enrolled in the Phase 1b and Phase 2 trials without concern for the presence of CD20+ B-cells. The column entitled “CD20+B cell positive patients” represents a subset (N=16) of the population of patients where CD20+ B-cells were estimated to be present (e.g., estimated through the proxy data of CD19+B-cell and rituximab concentration, as described above). If an eligibility criteria for presence of CD20+ B-cells were retroactively applied to the patients enrolled in the Phase 1b/2 trials, 26 of the 42 patients would be excluded.

Retroactive application of this eligibility criteria for presence of CD20+ B-cells increased the percentage of patients that exhibited an ORR from 33% up to 62.5%. Additionally, retroactive application of this eligibility criteria for presence of CD20+ B-cells increased the percentage of patients that exhibited a CR and a PR from 14% to 25% and from 19% to 37.5%, respectively. This suggests that an eligibility criteria requiring patients to have a presence of CD20+ B-cells can be promising for identifying patients that are likely to respond favorably to the combination magrolimab and rituximab therapy.

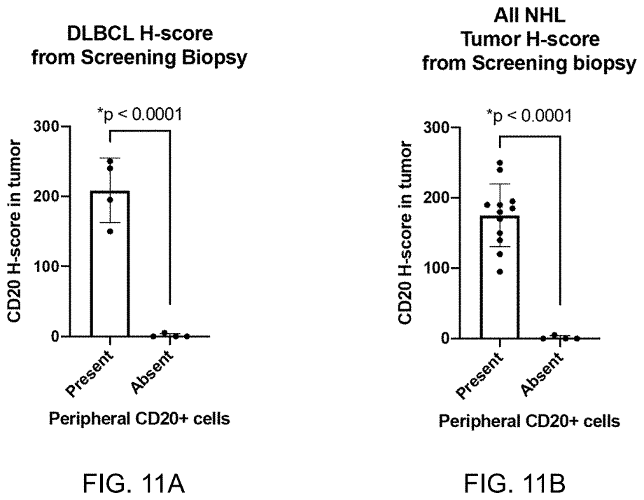

FIGS. 11A and 11B depict results describing a CD20 H-score, which is used as a direct measurement of the presence or absence of CD20+ B-cells. Specifically, FIG. 11A depicts the CD20 H-score for DLBCL patients at the time of their screening (e.g., screening for eligibility prior enrolling in the trial and receiving treatment). FIG. 11B depicts the CD20 H-score across all NHL patients at the time of their screening. A cutoff of CD20+ B-cells less than or equal to 0.2% of total CD45+cells was used to define CD20 negative cases. In both FIGS. 11A and 11B, patients with a high CD20 H-score had a presence of CD20+B-cells whereas patients with a low CD20 H-score had an absence of CD20+ B-cells. More specifically, in FIG. 11A, DLBCL patients with CD20+ B-cells had an average CD20 H-score of ˜200, which was significantly different from the corresponding H-score of DLBCL patients who lacked CD20+ B-cells. In FIG. 11B, all NHL patients with CD20+ B-cells had an average CD20 H-score of ˜180, which was significantly different from the corresponding H-score of NHL patients who lacked CD20+ B-cells. This indicates that if an eligibility criteria for the presence of CD20+ B-cells was implemented, the CD20 H-score can be used to directly predict the presence or absence of CD20+ B-cells.

Presence of Both CD19+ and CD20+ B-Cells

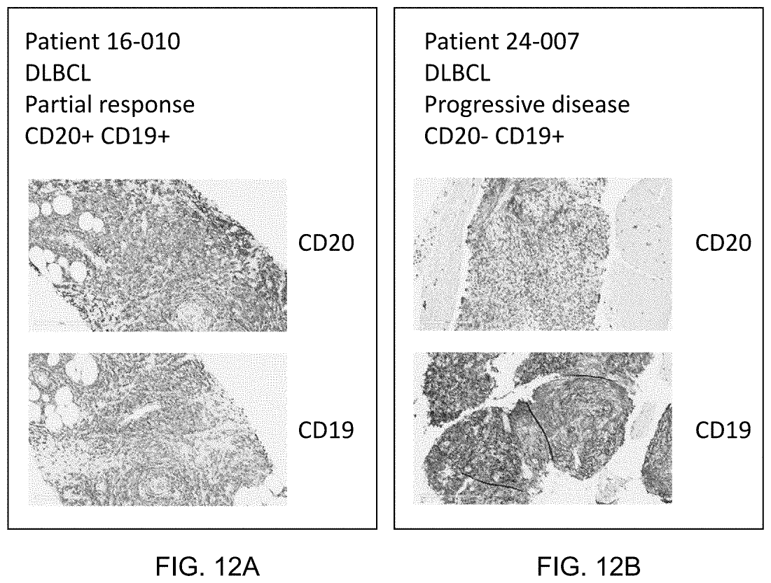

FIGS. 12A and 12B depict the outcome of two patients with either CD20+CD19+ or CD20-CD19+ profiles confirmed using immunohistochemistry (IHC). The presence of CD20 and CD19 B-cells was determined through IHC staining of a tumor biopsy and by calculating an H-score, as described above.

FIG. 12A depicts positive IHC staining of both CD20 and CD19 B-cells in a DLBCL patient who exhibited a partial response to the combination therapy of magrolimab and rituximab. In contrast, FIG. 12B depicts negative IHC staining of CD20 B-cells and positive IHC staining of CD19 B-cells in another DLBCL patient. Here, this DLBCL patient exhibited progressive disease in response to the combination therapy of magrolimab and rituximab. Altogether, FIGS. 12A and 12B indicate that patients with a CD20-/CD19+profile may respond poorly to magrolimab+rituximab combination therapy in comparison to other patients with a different profile.

Time of Last Anti-CD20 Treatment

The time of last anti-CD20 treatment can be used as a direct predictor of a patient's response to the combination therapy and/or it can be used as a surrogate measurement for the presence or absence of either CD19+ B-cells or CD20+ B-cells.

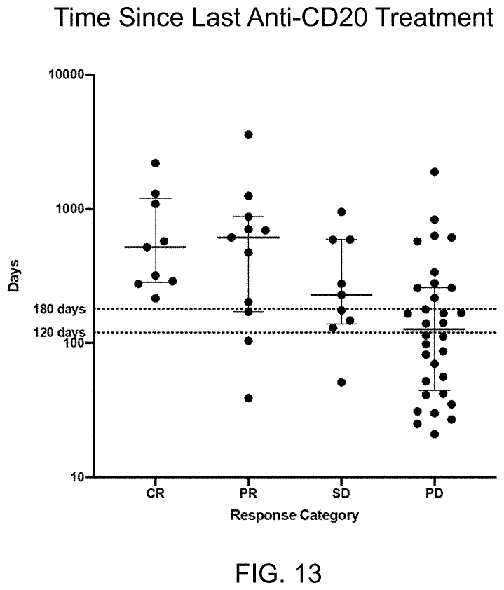

FIG. 13 shows the number of days (log-scale) since last anti-CD20 treatment on the y-axis and the patient response (e.g., CR, PR, SD, PD) on the x-axis. Generally, the higher the number of days since the last anti-CD20 treatment, the more likely the patient would exhibit a CR or PR as opposed to SD or PD. Specifically, as shown in FIG. 13, patients that exhibited a CR or PR averaged ˜750-800 days since last anti-CD20 treatment. In comparison, patients that exhibited SD averaged ˜200 days since last anti-CD20 treatment whereas patients that exhibited PD averaged ˜120 days since last anti-CD20 treatment. This indicates that the time of last anti-CD20 treatment can be a direct predictor of a patient's response to the combination therapy of magrolimab and rituximab.

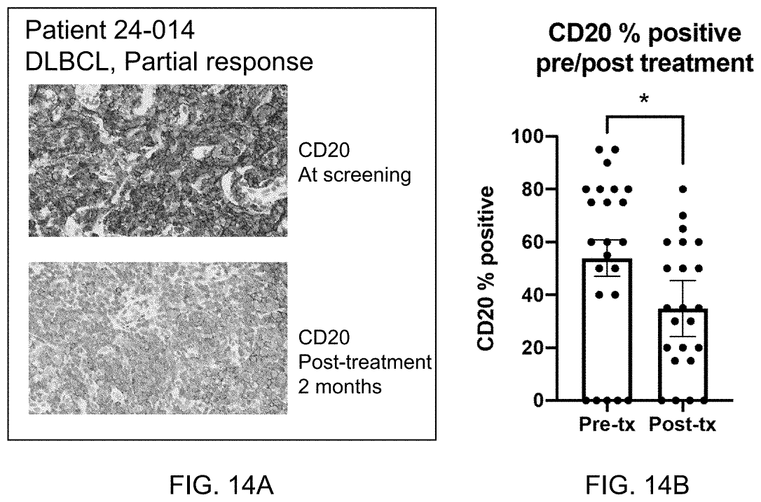

FIGS. 14A and 14B show the reduction of CD20 expression following treatment involving an anti-CD20 treatment e.g., rituximab. Specifically, FIG. 14A depicts CD20 immunohistochemistry staining of tissue slices obtained from a DLBCL patient (patient 24-014) at screening and 2 months post-treatment. The intensity of the CD20 immunohistochemistry staining in the tissue post-treatment is reduced in comparison to the CD20 staining in the tissue at screening, likely due to the anti-CD20 treatment. Additionally, FIG. 14B depicts the quantified percentage of CD20+expression at screening (e.g., “pre-tx”) and post treatment (e.g., “post-tx”). Here, over 50% of cells were CD20+ at screening where less than 40% of cells were CD20+post treatment. This different is statistically significant.

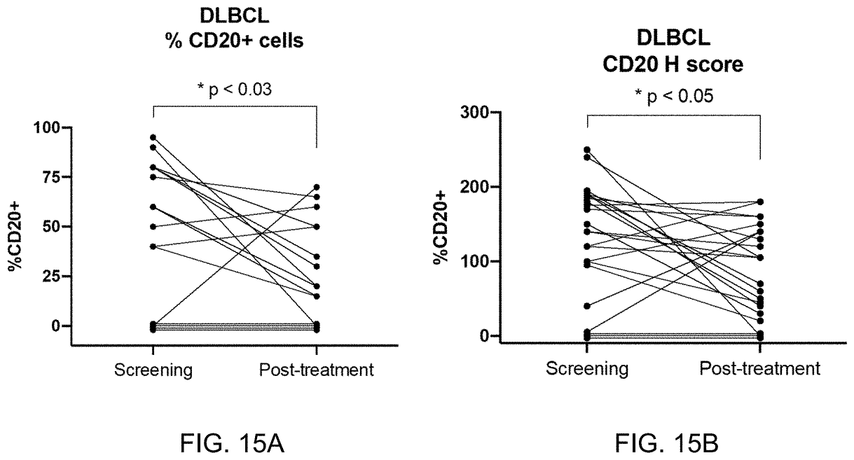

FIGS. 15A and 15B show the change in CD20 expression in individual DLBCL patients at screening and post-treatment. FIG. 15A shows that the majority of DLBCL patients experienced a reduction in the percentage of cells that expressed CD20 following treatment. FIG. 15B similarly shows that the majority of DLBCL patients experienced a reduction in the CD20 H score, which is measurement of the presence of CD20+ B-cells.

Altogether, FIGS. 14A/14B and 15A/15B demonstrate that CD20 expression is reduced as a result of a treatment including anti-CD20 and therefore, a larger number of days since a last anti-CD20 treatment would likely lead to improved outcomes, as shown in FIG. 13, given the replenishment of CD20 B-cells over time.

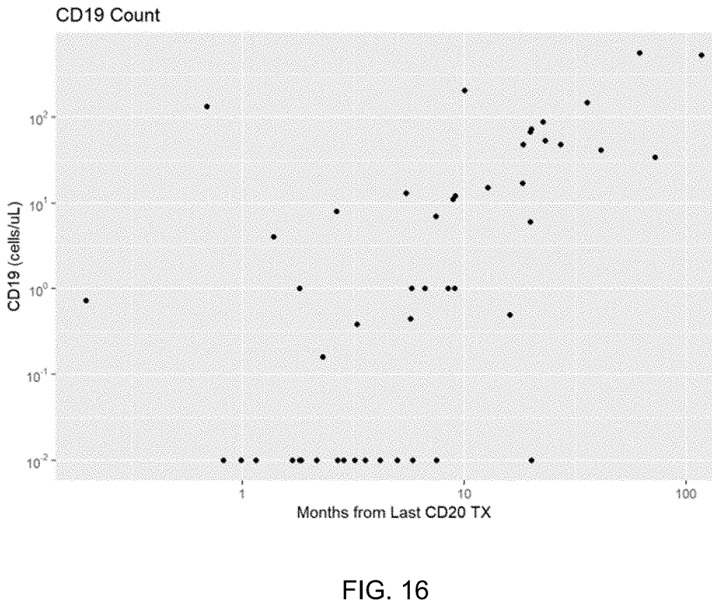

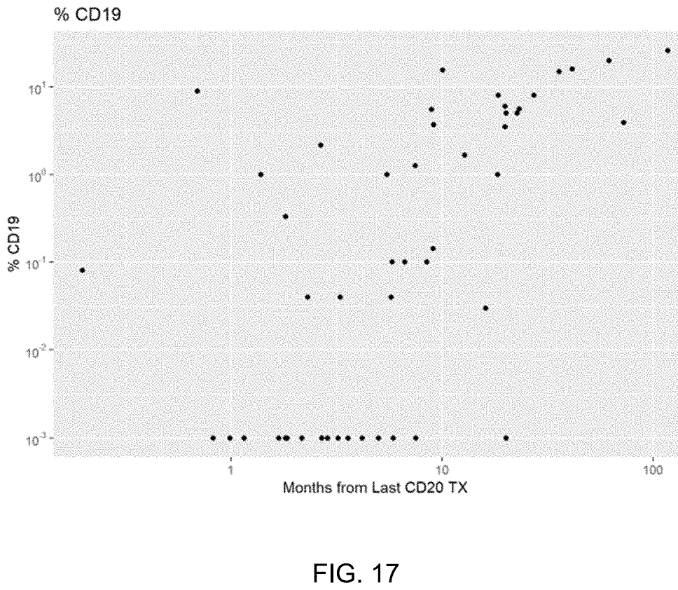

FIG. 16 shows a correlation between the time that a patient last received an anti-CD20 treatment and an absolute count of CD19 B-cells present in the patient. Generally, there is a direct correlation between the number of months from the last anti-CD20 treatment and the absolute number (cells per microliter) of CD19+ B-cells. FIG. 17 shows a correlation between the time that a patient last received an anti-CD20 treatment and a percentage of CD19 B-cells present in the patient. Here, there is a direct correlation between the number of months from the last anti-CD20 treatment and the percentage of CD19+ B-cells over total lymphocytes. Notably, in both FIG. 16 and FIG. 17, there is a subpopulation of patients that between 1 and 10 months, do not recover CD19 B-cells. It may be that these patients require more than 10 months before their CD19 B-cells are replenished. Table 7 below provides statistical data pertaining to the number of months from the last anti-CD20 treatment in patients where CD19 B-cells were determined to be present or absent. A cutoff of the limit of detection was used to differentiate between presence/absence of CD19+ B-cells.

TABLE 7

| | Months from last CD20 | Months from last CD20 |
|---|---|---|
| | treatment in patients | treatment in patients |
| | where CD19 B-cells | where CD19 B-cells |
| | are absent (N = 14) | are present (N = 28) |
| median | 2.7 | 10 |
| mean | 3.9 | 19 |
| Q1, Q3 | 1.7, 4.2 | 6, 21 |
| Min, max | 0.8, 20 | 0, 118 |

Altogether, these results suggest that the time of last anti-CD20 treatment can be implemented as an eligibility criterion to exclude patients that are less likely to respond to the combination treatment of magrolimab and rituximab. As one example, the eligibility criterion can be that the patient last received the anti-CD20 treatment at least 4 weeks ago.

Rituximab Concentration in Subject

The concentration of the anti-CD20 therapy (e.g., rituximab) in the subject at baseline can be used as a surrogate measurement for the presence or absence of B-cells, such as CD19+ B-cells. Each of FIGS. 17-19 describe results that support the use of the concentration of rituximab in the subject as a surrogate measurement for the presence of CD19+ B-cells.

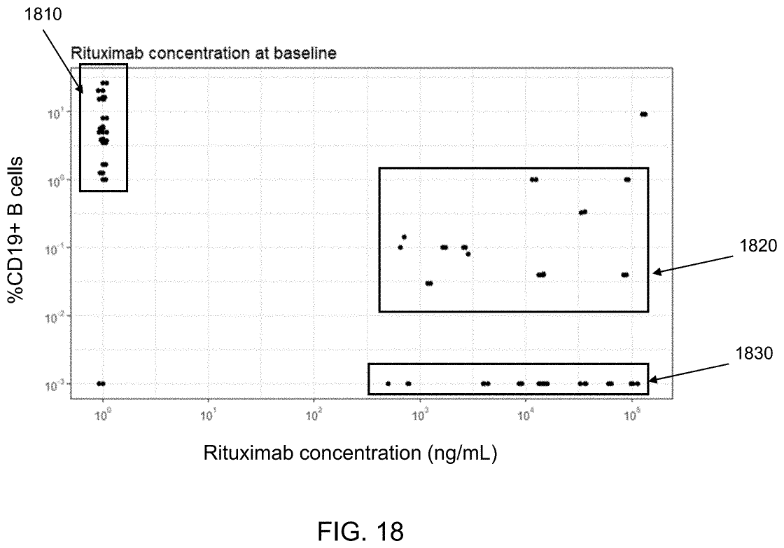

FIG. 18 shows a correlation between a rituximab concentration in a patient (e.g., as a measure of rituximab pharmacokinetics) and a percentage of CD19+ B-cells present in the patient. A significant proportion of patients, labeled as 1810 in FIG. 18, had low serum levels of rituximab (˜1 ng/mL) and higher percentages of CD19+ B-cells (between 100 and 10¹% B-cells). A second population of patients, labeled as 1820 in FIG. 18, had higher serum levels of rituximab (between 10² and 105 ng/mL) with slightly lower percentages of CD19+ B-cells (between 10⁻² and 100% B-cells). Furthermore, a third population of patients, labeled as 1830 in FIG. 18, had higher levels of rituximab (between 10² and 105 ng/mL) and low percentages of CD19+ B-cells (10-3% B-cells). Therefore, across the first population 1810, second population 1820, and third population 1830, a decrease of rituximab concentration results in a substantial increase in the percentage of CD19+ B-cells in the patient.

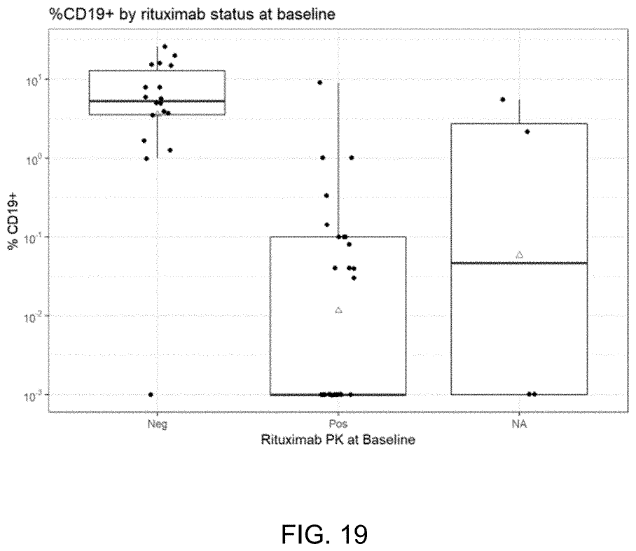

FIG. 19 shows a correlation between a presence or absence of rituximab in a patient and a percentage of CD19 B-cells present in the patient. Specifically, patients were categorized into a “negative” category and a “positive” category based on the serum rituximab levels in each patient. A cutoff of the limit of detection was used to differentiate between presence or absence of CD19 B-cells. Patients that were categorized in the “negative” category had an average percentage of CD19+ B-cells of ˜4% whereas patients that were categorized in the “positive” category had an average percentage of CD19+ B-cells of 0.01%. The N/A category refers to patients without available CD19 measurements.

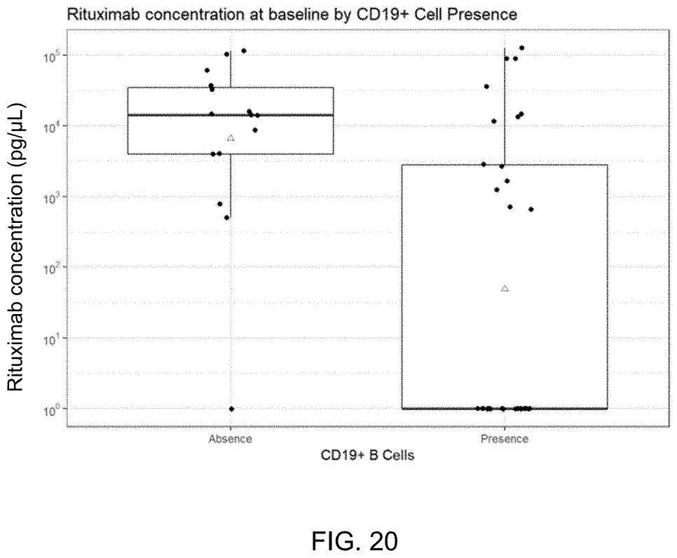

FIG. 20 shows a correlation between a rituximab concentration in a patient and a presence or absence of CD19 B-cells present in the patient. Patients were categorized into an “absent” category and a “present” category. A cutoff of the limit of detection was used to differentiate between presence or absence of CD19 B-cells. Patients that were categorized in the “absent” category had an average rituximab concentration of ˜10⁴ pg/μL whereas patients that were categorized in the “present” category had an average rituximab concentration of ˜10² pg/μL.

Table 8 below documents the categorization of patients in one of either rituximab positive or negative and in one of either CD19+B-cell absent or CD19+B-cell present categories.

TABLE 8

| | CD19 Absence | CD19 Presence |
|---|---|---|
| | (N =15) | (N = 30) |
| Rituximab negative | 1 (2%) | 17 (38%) |
| (N = 18) | | |
| Rituximab positive | 14 (31%) | 13 (29%) |
| (N = 27) | | |

Altogether, these results suggest that the rituximab concentration in the patient can be implemented as an eligibility criterion to exclude patients that are less likely to respond to the combination treatment of magrolimab and rituximab. For example, the eligibility criterion can be that the rituximab concentration in the patient at baseline or screening is less than 1 ng/mL, 10 ng/mL, or 100 ng/mL.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

### Example 3: Antibody Receptor Occupancy in Q1W vs Q2W Hu5F9-G4 Dosing Regime

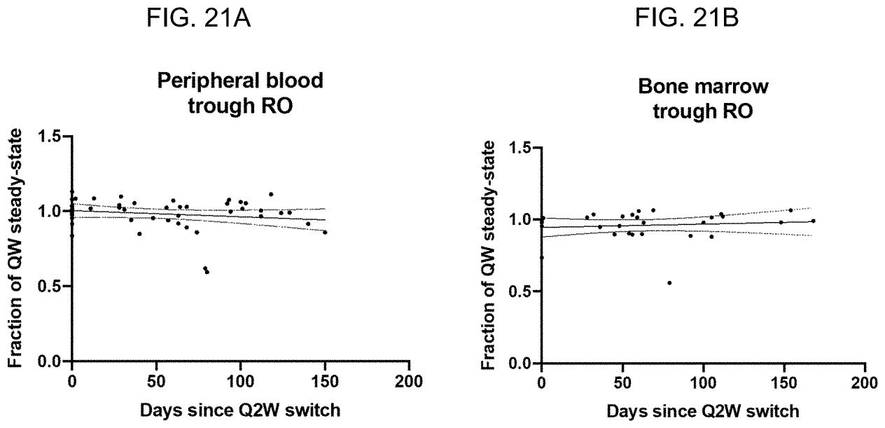

FIG. 21A shows CD47 receptor occupancy by Hu5F9-G4 in CD45+ peripheral blood cells over time after a transition from Hu5F9-G4 dosing (Q1W) to every other week Hu5F9-G4 dosing (Q2W). Receptor occupancy is expressed as a fraction of the steady-state QW level. FIG. 21B shows CD47 receptor occupancy by Hu5F9-G4 in CD45+ bone marrow cells over time after a transition from weekly Hu5F9-G4 dosing (Q1W) to every other week Hu5F9-G4 dosing (Q2W). Receptor occupancy is expressed as a fraction of the steady-state QW level.

Antibody receptor occupancy (RO) was assessed in the once per week dosing (Q1W) and the once every two weeks dosing (Q2W) regime. Patients were dosed with Hu5F9-G4 once a week for all cycles (Q1W throughout) or once per week for cycles 1 and 2 and then once every two weeks (Q2W) in Cycle 3 and beyond. CD47 antibody receptor occupancy (RO) was assessed in the peripheral blood and bone marrow and compared against Q1W vs. Q2W dosing. Primary patient blood or bone marrow cells were stained with a Hu5F9-G4-reactive fluorescent anti-IgG4 antibody, followed by quantitation via flow cytometry. Occupancy levels were calculated as a percent of maximum signal, defined by matched patient sample with saturating quantities of unlabeled Hu5F9-G4 added prior to anti-IgG4 antibody staining. Data for the Q2W dosing were normalized against the Q1W RO levels.

The patients rapidly achieved maximum occupancy during Cycle 1 and Cycle 2 (Q1W dosing, not shown). A similar CD47 antibody RO was observed in both the peripheral blood (FIG. 21A) or bone marrow (FIG. 21B) after Q2W dosing change in Cycle 3 and beyond. For both figures, the dots indicate the antibody occupancy level in patient samples taken over time from Cycle 3 and beyond normalized to the patient Q1W RO levels, while the middle line indicates the linear regression best fit, and the top and bottom lines indicate the 95% confidence intervals. Thus, Hu5F9-G4 Q2W (i.e., dose administration once every two weeks) dosing resulted in a similar CD47 receptor occupancy as Q1W (i.e., dose administration once every week) dosing.

While the invention has been particularly shown and described with reference to a preferred embodiment and various alternate embodiments, it will be understood by persons skilled in the relevant art that various changes in form and details can be made therein without departing from the spirit and scope of the invention.

TABLE 9
SEQUENCES

| SEQ ID | | |
|---|---|---|
| NO | ID | Sequence |
| 1 | 1H9 | SYWIT |
| | CDR-H1 | |
| 2 | 1H9 | DIYPGSGSTNHIEKFKS |
| | CDR-H2 | |
| 3 | 1H9 | GYGSSYGYFDY |
| | CDR-H3 | |
| 4 | 1H9 | RASENIYSYLA |
| | CDR-L1 | |
| 5 | 1H9 | TAKTLAE |
| | CDR-L2 | |
| 6 | 1H9 | QHQYGPPFT |
| | CDR-L3 | |
| 7 | Humanized | QVQLVQSGAE VKKPGASVKV SCKASGYTFT SYWITWVKQA PGQGLEWIGD |
| | 1H9 VH | IYPGSGSTNH IEKFKSKATL TVDTSISTAY MELSRLRSDD TAVYYCATGY |
| | | GSSYGYFDYW GQGTLVTVSS |
| 8 | Humanized | DIQMTQSPSS LSASVGDRVT ITCRASENIY SYLAWYQQKP GKAPKLLIYT |
| | 1H9 VL | AKTLAEGVPS RFSGSGSGTD FTLTISSLQP EDFATYYCQH QYGPPFTFGQ |
| | | GTKLEIK |
| 9 | 3C2 | SYWMH |
| | CDR-H1 | |
| 10 | 3C2 | NIDPSDSDTHYNQKFKD |
| | CDR-H2 | |
| 11 | 3C2 | GYSKYYAMDY |
| | CDR-H3 | |
| 12 | 3C2 | RSSQSIVHSYGNTYLE |
| | CDR-L1 | |
| 13 | 3C2 | KVSNRFS |
| | CDR-L2 | |
| 14 | 3C2 | FQGSHVPYT |
| | CDR-L3 | |
| 15 | Humanized | QVQLVQSGAE VKKPGASVKV SCKASGYTFT SYWMHWVRQA PGQGLEWMGN |
| | 3C2 V_{H} | IDPSDSDTHY NQKFKDRVTM TRDTSTSTVY MELSSLRSED TAVYYCARGY |
| | | SKYYAMDYWG QGTLVTVSS |
| 16 | Humanized | DIVMTQTPLS LSVTPGQPAS ISCRSSQSIV HSYGNTYLEW YLQKPGQSPQ |
| | 3C2 V_{L} | LLIYKVSNRF SGVPDRFSGS GSGTDFTLKI SRVEAEDVGV YYCFQGSHVP |
| | | YTFGQGTKLE IK |
| 17 | Humanized | QVQLVQSGAEVKKPGASVKVSCKASGYTFTSYWITWVKQAPGQGLEWIGDIYPGSG |
| | 1H9HC | STNHIEKFKSKATLTVDTSISTAYMELSRLRSDDTAVYYCATGYGSSYGYFDYWGQ |
| | (full- | GTLVTVSSASTKGPSVFPLAPSSKSTSGGTAALGCLVKDYFPEPVTVSWNSGALTS |
| | length) | GVHTFPAVLQSSGLYSLSSVVTVPSSSLGTQTYICNVNHKPSNTKVDKKVEPKSCD |
| | | KTHTCPPCPAPELLGGPSVFLFPPKPKDTLMISRTPEVTCVVVDVSHEDPEVKFNW |
| | | YVDGVEVHNAKTKPREEQYASTYRVVSVLTVLHQDWLNGKEYKCKVSNKALPAPIE |
| | | KTISKAKGQPREPQVYTLPPSREEMTKNQVSLTCLVKGFYPSDIAVEWESNGQPEN |
| | | NYKTTPPVLDSDGSFFLYSKLTVDKSRWQQGNVFSCSVMHEALHNHYTQKSLSLSP |
| | | G |
| 18 | Humanized | DIQMTQSPSSLSASVGDRVTITCRASENIYSYLAWYQQKPGKAPKLLIYTAKTLAE |
| | 1H9 LC | GVPSRFSGSGSGTDFTLTISSLQPEDFATYYCQHQYGPPFTFGQGTKLEIKRTVAA |
| | (full- | PSVFIFPPSDEQLKSGTASVVCLLNNFYPREAKVQWKVDNALQSGNSQESVTEQDS |
| | length) | KDSTYSLSSTLTLSKADYEKHKVYACEVTHQGLSSPVTKSFNRGEC |
| 19 | Humanized | QVQLVQSGAEVKKPGASVKVSCKASGYTFTSYWMHWVRQAPGQGLEWMGNIDPSDS |
| | 3C2 HC | DTHYNQKFKDRVTMTRDTSTSTVYMELSSLRSEDTAVYYCARGYSKYYAMDYWGQG |
| | (full- | TLVTVSSASTKGPSVFPLAPSSKSTSGGTAALGCLVKDYFPEPVTVSWNSGALTSG |
| | length) | VHTFPAVLQSSGLYSLSSVVTVPSSSLGTQTYICNVNHKPSNTKVDKKVEPKSCDK |
| | | THTCPPCPAPELLGGPSVFLFPPKPKDTLMISRTPEVTCVVVDVSHEDPEVKFNWY |
| | | VDGVEVHNAKTKPREEQYASTYRVVSVLTVLHQDWLNGKEYKCKVSNKALPAPIEK |
| | | TISKAKGQPREPQVYTLPPSREEMTKNQVSLTCLVKGFYPSDIAVEWESNGQPENN |
| | | YKTTPPVLDSDGSFFLYSKLTVDKSRWQQGNVFSCSVMHEALHNHYTQKSLSLSPG |
| 20 | Humanized | DIVMTQTPLSLSVTPGQPASISCRSSQSIVHSYGNTYLEWYLQKPGQSPQLLIYKV |
| | 3C2 LC | SNRFSGVPDRFSGSGSGTDFTLKISRVEAEDVGVYYCFQGSHVPYTFGQGTKLEIK |
| | (full- | RTVAAPSVFIFPPSDEQLKSGTASVVCLLNNFYPREAKVQWKVDNALQSGNSQESV |
| | length) | TEQDSKDSTYSLSSTLTLSKADYEKHKVYACEVTHQGLSSPVTKSFNRGEC |
| 21 | 9B11 | DYYIH |
| | CDR-H1 | |
| 22 | 9B11 | RIDPEDGETKYAPKFQG |
| | CDR-H2 | |
| 23 | 9B11 | GGFAY |
| | CDR-H3 | |
| 24 | 9B11 | ASSSVSSSYLY |
| | CDR-L1 | |
| 25 | 9B11 | STSNLAS |
| | CDR-L2 | |
| 26 | 9B11 | HQWSSHPYT |
| | CDR-L3 | |
| 27 | 9B11 V_{H} | EVQLQQSGAELVKPGASVKLSCTASGFNIKDYYIHWVKQRTEQGLEWIGRIDPEDG |
| | | ETKYAPKFQGKATITADTSSNTAYLQLNSLTSEDTAVYSCAKGGFAYWGQGTLVTV |
| | | SA |
| 28 | 9B11 V_{L} | QIVLTQSPAIMSASPGEKVTLTCSASSSVSSSYLYWYQQKPGSSPKLWIYSTSNLA |
| | | SGVPARFSGSGSGTSYSLTISSMEAEDAASYFCHQWSSHPYTFGGGTKLEIK |
| 29 | 7E11 | SYWMH |
| | CDR-H1 | |
| 30 | 7E11 | NIDPSDSDTHYNQKFKD |
| | CDR-H2 | |
| 31 | 7E11 | SYGNYGENAMDY |
| | CDR-H3 | |
| 32 | 7E11 | RSSQSIVHSYGNTYLE |
| | CDR-L1 | |
| 33 | 7E11 | KVSNRFS |
| | CDR-L2 | |
| 34 | 7E11 | FQGSHVPFT |
| | CDR-L3 | |
| 35 | 7E11 V_{H} | QVKLQESGAELVRPGSSVKLSCKASGYTFTSYWMHWVKQRPIQGLEWIGNIDPSDS |
| | | DTHYNQKFKDKATLTVDNSSSTAYMQLSSLTSEDSAVYYCASYGNYGENAMDYWGQ |
| | | GTSVTVSS |
| 36 | 7E11 V_{L} | DILMTQTPLSLPVSLGDQASISCRSSQSIVHSYGNTYLEWYLQKPGQSPKLLIYKV |
| | | SNRFSGVPDRFSGSGSGTDFTLKISRVEAEDLGVYYCFQGSHVPFTFGSGTKLEIK |
| 37 | Humanized | CAGGTTCAGTTGGTTCAGTCTGGCGCCGAAGTGAAGAAACCTGGCGCCTCTGTGAA |
| | 1H9 heavy | GGTGTCCTGCAAGGCTTCCGGCTACACCTTTACCAGCTACTGGATCACCTGGGTCA |
| | chain | AGCAGGCTCCTGGACAGGGACTCGAGTGGATCGGCGATATCTATCCTGGCTCCGGC |
| | nucleic | TCCACCAACCACATCGAGAAGTTCAAGTCCAAGGCTACCCTGACCGTGGACACCTC |
| | acid | CATCTCCACCGCCTACATGGAACTGTCCCGGCTGAGATCTGACGACACCGCCGTGT |
| | | ACTATTGCGCTACCGGCTACGGCTCCTCCTACGGCTACTTTGATTATTGGGGCCAG |
| | | GGCACCCTGGTCACCGTGTCCTCTGCTTCTACCAAGGGACCCAGCGTGTTCCCTCT |
| | | GGCTCCTTCCAGCAAGTCTACCTCTGGCGGAACAGCTGCTCTGGGCTGCCTGGTCA |
| | | AGGACTACTTTCCTGAGCCTGTGACCGTGTCTTGGAACTCTGGCGCTCTGACATCT |
| | | GGCGTGCACACATTCCCTGCTGTGCTGCAGTCCTCCGGCCTGTACTCTCTGTCCTC |
| | | TGTCGTGACCGTGCCTTCCAGCTCTCTGGGAACCCAGACCTACATCTGCAATGTGA |
| | | ACCACAAGCCTTCCAACACCAAGGTGGACAAGAAGGTGGAACCCAAGTCCTGCGAC |
| | | AAGACCCACACCTGTCCTCCATGTCCTGCTCCAGAACTGCTCGGCGGACCTTCCGT |
| | | GTTTCTGTTCCCTCCAAAGCCTAAGGACACCCTGATGATCTCTCGGACCCCTGAAG |
| | | TGACCTGCGTGGTGGTGGATGTGTCTCACGAGGACCCAGAAGTGAAGTTCAATTGG |
| | | TACGTGGACGGCGTGGAAGTGCACAACGCCAAGACCAAGCCTAGAGAGGAACAGTA |
| | | CGCCTCCACCTACAGAGTGGTGTCCGTGCTGACAGTGCTGCACCAGGATTGGCTGA |
| | | ACGGCAAAGAGTACAAGTGCAAGGTGTCCAACAAGGCCCTGCCTGCTCCTATCGAA |
| | | AAGACCATCTCCAAGGCCAAGGGCCAGCCTAGGGAACCCCAGGTTTACACCCTGCC |
| | | ACCTAGCCGGGAAGAGATGACCAAGAACCAGGTGTCCCTGACCTGCCTCGTGAAGG |
| | | GCTTCTACCCTTCCGATATCGCTGTGGAATGGGAGAGCAACGGCCAGCCTGAGAAC |
| | | AACTACAAGACAACCCCTCCTGTGCTGGACTCCGACGGCTCATTCTTTCTGTACTC |
| | | CAAGCTGACTGTGGACAAGTCCAGATGGCAGCAGGGCAACGTGTTCTCCTGCAGCG |
| | | TGATGCACGAGGCCCTGCACAATCACTACACACAGAAGTCTCTGTCTCTGAGCCCC |
| | | GGC |
| 38 | Humanized | GACATCCAGATGACCCAGTCTCCATCCTCTCTGTCCGCCTCTGTGGGCGACAGAGT |
| | 1H9 light | GACCATCACCTGTCGGGCCTCCGAGAACATCTACTCCTACCTGGCCTGGTATCAGC |
| | chain | AGAAGCCTGGCAAGGCTCCCAAGCTGCTGATCTACACCGCTAAGACACTGGCCGAG |
| | nucleic | GGCGTGCCCTCTAGATTTTCTGGCTCTGGAAGCGGCACCGACTTTACCCTGACAAT |
| | acid | CTCCAGCCTGCAGCCTGAGGACTTCGCCACCTACTACTGCCAGCACCAGTACGGCC |
| | | CTCCATTCACCTTTGGCCAGGGCACCAAGCTGGAAATCAAGCGGACAGTGGCCGCT |
| | | CCTTCCGTGTTCATCTTCCCACCTTCCGACGAGCAGCTGAAGTCTGGCACAGCCTC |
| | | TGTCGTGTGCCTGCTGAACAACTTCTACCCTCGGGAAGCCAAGGTGCAGTGGAAGG |
| | | TGGACAATGCCCTGCAGTCCGGCAACTCCCAAGAGTCTGTGACCGAGCAGGACTCC |
| | | AAGGACAGCACCTACAGCCTGTCCTCCACACTGACCCTGTCCAAGGCCGACTACGA |
| | | GAAGCACAAGGTGTACGCCTGCGAAGTGACCCATCAGGGCCTGTCTAGCCCTGTGA |
| | | CCAAGTCTTTCAACCGGGGCGAGTGC |

| | | |
|---|---|---|
| 39 | Humanized | CAGGTTCAGTTGGTTCAGTCTGGCGCCGAAGTGAAGAAACCTGGCGCCTCTGTGAA |
| | 3C2 heavy | GGTGTCCTGCAAGGCTTCCGGCTACACCTTTACCAGCTACTGGATGCACTGGGTCC |
| | chain | GACAGGCTCCAGGACAAGGCTTGGAGTGGATGGGCAACATCGACCCCTCTGACAGC |
| | nucleic | GACACCCACTACAACCAGAAATTCAAGGACCGCGTGACCATGACCAGAGACACCTC |
| | acid | CACCAGCACCGTGTACATGGAACTGTCCAGCCTGAGATCCGAGGACACCGCCGTGT |
| | | ACTACTGTGCCAGAGGCTACTCCAAGTACTACGCCATGGACTACTGGGGCCAGGGC |
| | | ACACTGGTTACCGTGTCCTCTGCTTCCACCAAGGGACCCTCTGTGTTCCCTCTGGC |
| | | TCCTTCCAGCAAGTCTACCTCTGGCGGAACAGCTGCTCTGGGCTGCCTGGTCAAGG |
| | | ACTACTTTCCTGAGCCTGTGACCGTGTCTTGGAACTCTGGCGCTCTGACATCTGGC |
| | | GTGCACACATTCCCTGCTGTGCTGCAGTCCTCCGGCCTGTACTCTCTGTCCTCTGT |
| | | CGTGACCGTGCCTTCCAGCTCTCTGGGAACCCAGACCTACATCTGCAATGTGAACC |
| | | ACAAGCCTTCCAACACCAAGGTGGACAAGAAGGTGGAACCCAAGTCCTGCGACAAG |
| | | ACCCACACCTGTCCTCCATGTCCTGCTCCAGAACTGCTCGGCGGACCTTCCGTGTT |
| | | TCTGTTCCCTCCAAAGCCTAAGGACACCCTGATGATCTCTCGGACCCCTGAAGTGA |
| | | CCTGCGTGGTGGTGGATGTGTCCCACGAAGATCCAGAAGTGAAGTTCAATTGGTAC |
| | | GTGGACGGCGTGGAAGTGCACAACGCCAAGACCAAGCCTAGAGAGGAACAGTACGC |
| | | CTCCACCTACAGAGTGGTGTCCGTGCTGACAGTGCTGCACCAGGATTGGCTGAACG |
| | | GCAAAGAGTACAAGTGCAAGGTGTCCAACAAGGCCCTGCCTGCTCCTATCGAAAAG |
| | | ACCATCTCCAAGGCCAAGGGCCAGCCTAGGGAACCCCAGGTTTACACCCTGCCTCC |
| | | AAGCCGGGAAGAGATGACCAAGAACCAGGTGTCCCTGACCTGCCTCGTGAAGGGCT |
| | | TCTACCCTTCCGATATCGCCGTGGAATGGGAGAGCAATGGCCAGCCAGAGAACAAC |
| | | TACAAGACAACCCCTCCTGTGCTGGACTCCGACGGCTCATTCTTTCTGTACTCCAA |
| | | GCTGACCGTGGACAAGTCCAGATGGCAGCAGGGCAACGTGTTCTCCTGCAGCGTGA |
| | | TGCACGAGGCCCTGCACAATCACTATACCCAGAAGTCCCTGTCTCTGTCCCCTGGC |
| 40 | Humanized | GACATCGTGATGACCCAGACACCTCTGAGCCTGAGCGTGACACCTGGACAGCCTGC |
| | 3C2 light | CTCCATCTCCTGCAGATCCTCTCAGTCCATCGTGCACTCCTACGGCAACACCTACC |
| | chain | TGGAATGGTATCTGCAGAAGCCCGGCCAGTCTCCTCAGCTGCTGATCTACAAGGTG |
| | nucleic | TCCAACCGGTTCTCTGGCGTGCCCGACAGATTTTCCGGCTCTGGCTCTGGCACCGA |
| | acid | CTTCACCCTGAAGATCTCCAGAGTGGAAGCCGAGGACGTGGGCGTGTACTACTGCT |
| | | TCCAAGGCTCTCACGTGCCCTACACCTTTGGCCAGGGCACCAAGCTGGAAATCAAG |
| | | CGGACAGTGGCCGCTCCTTCCGTGTTCATCTTCCCACCTTCCGACGAGCAGCTGAA GTCCGGCACAGCTTCTGTCGTGTGCCTGCTGAACAACTTCTACCCTCGGGAAGCCA |
| | | AGGTGCAGTGGAAGGTGGACAATGCCCTGCAGTCCGGCAACTCCCAAGAGTCTGTG |
| | | ACCGAGCAGGACTCCAAGGACAGCACCTACAGCCTGTCCAGCACACTGACCCTGTC |
| | | CAAGGCCGACTACGAGAAGCACAAGGTGTACGCCTGCGAAGTGACCCATCAGGGCC |
| | | TGTCTAGCCCTGTGACCAAGTCTTTCAACCGGGGCGAGTGC |
| 41 | 9B11 VH | GAGGTTCAGCTGCAGCAGTCTGGGGCAGAGCTTGTGAAGCCAGGGGCCTCAGTCAA |
| | nucleic | GTTGTCCTGCACAGCTTCTGGCTTCAACATTAAAGACTACTATATACACTGGGTGA |
| | acid | AGCAGAGGACTGAACAGGGCCTGGAGTGGATTGGAAGGATTGATCCTGAGGATGGT |
| | | GAAACTAAATATGCCCCGAAATTCCAGGGCAAGGCCACTATAACAGCAGACACATC |
| | | CTCCAACACAGCCTACCTGCAGCTCAACAGCCTGACATCTGAGGACACTGCCGTCT |
| | | ATTCCTGTGCTAAGGGGGGGTTTGCTTACTGGGGCCAAGGGACTCTGGTCACTGTC |
| | | TCTGCA |
| 42 | 9B11 VL | CAAATTGTTCTCACCCAGTCTCCAGCAATCATGTCTGCATCTCCTGGGGAGAAGGT |
| | nucleic | CACCTTGACCTGCAGTGCCAGTTCAAGTGTAAGTTCCAGCTACTTGTACTGGTACC |
| | acid | AGCAGAAGCCAGGATCCTCCCCCAAACTCTGGATTTATAGCACATCCAACCTGGCT |
| | | TCTGGAGTCCCTGCTCGCTTCAGTGGCAGTGGGTCTGGGACCTCTTACTCTCTCAC |
| | | AATCAGCAGCATGGAGGCTGAAGATGCTGCCTCTTATTTCTGCCATCAGTGGAGTA |
| | | GTCACCCGTACACGTTCGGAGGGGGGACCAAGCTGGAAATAAAA |
| 43 | 7E11 VH | CAGGTCAAGCTGCAGGAGTCTGGGGCTGAGCTGGTGAGGCCTGGGTCTTCAGTGAA |
| | nucleic | GCTGTCCTGCAAGGCTTCTGGCTACACCTTCACCAGCTACTGGATGCATTGGGTGA |
| | acid | AGCAGAGGCCTATACAAGGCCTTGAATGGATTGGTAACATTGACCCTTCTGATAGT |
| | | GATACTCACTACAATCAAAAGTTCAAGGACAAGGCCACATTGACTGTGGACAACTC |
| | | CTCCAGCACAGCCTACATGCAGCTCAGCAGCCTGACCTCTGAGGACTCTGCGGTCT |
| | | ATTACTGTGCAAGCTATGGTAACTACGGGGAGAATGCTATGGACTACTGGGGTCAA |
| | | GGAACCTCAGTCACCGTCTCCTCA |
| 44 | 7E11 VL | GATATTTTGATGACCCAAACTCCACTCTCCCTGCCTGTCAGTCTTGGAGATCAAGC |
| | nucleic | CTCCATCTCTTGCAGATCTAGTCAGAGCATTGTACATAGTTATGGAAACACCTATT |
| | acid | TAGAATGGTACCTGCAGAAACCAGGCCAGTCTCCAAAACTCCTGATCTACAAAGTT |
| | | TCCAACCGATTTTCTGGGGTCCCAGACAGGTTCAGTGGCAGTGGATCAGGTACAGA |
| | | TTTCACACTCAAGATCAGCAGAGTGGAGGCTGAGGATCTGGGAGTTTATTACTGCT |
| | | TTCAAGGTTCACATGTTCCATTCACGTTCGGCTCGGGGACAAAGTTGGAAATAAAA |
| 45 | SIRPa | EEELQVIQPDKSVLVAAGETATLRCTATSLIPVGPIQWFRGAGPGRELIYNQKEGH |
| | | FPRVTTVSDLTKRNNMDFSIRIGNITPADAGTYYCVKFRKGSPDDVEFKSGAGTEL |
| | | SVRA |
| 46 | KWar VH | EVQLVQSGAEVKKPGATVKISCKVSGFNIKDYYIHWVQQAPGKGLEWIGRIDPEDG |
| | | ETKYAPKFQDRATITADTSTDTAYMELSSLRSEDTAVYYCARWGAYWGQGTLVTVS |
| | | S |
| 47 | KWar VL | QIVLTQSPPTLSLSPGERVTLTCSASSSVSSSYLYWYQQKPGQAPKLWIYSTSNLA |
| | | SGVPARFSGSGSGTSYTLTISSLQPEDFAVYFCHQWSSYPRTFGAGTKLEIK |
| 48 | SIRPa V1 | EEELQVIQPDKSVLVAAGETATLRCTATSLIPVGPIQWFRGAGPGRELIYNQKEGH |
| | | FPRVTTVSDLTKRNNMDFSIRIGNITPADAGTYYCVKFRKGSPDDVEFKSGAGTEL |
| | | SVRA |
| 49 | SIRPa V2 | EEELQVIQPDKSVSVAAGESAILHCTVTSLIPVGPIQWFRGAGPARELIYNQKEGH |
| | | FPRVTTVSESTKRENMDFSISISNITPADAGTYYCVKFRKGSPDTEFKSGAGTELS |
| | | VRA |
| 50 | Hu5f9-G4 | QVQLVQSGAEVKKPGASVKVSCKASGYTFTNYNMHWVRQAPGQRLEWMGTIYPGND |
| | Antibody | DTSYNQKFKDRVTITADTSASTAYMELSSLRSEDTAVYYCARGGYRAMDYWGQGTL |
| | Heavy | VTVSSASTKGPSVFPLAPCSRSTSESTAALGCLVKDYFPEPVTVSWNSGALTSGVH |
| | Chain | TFPAVLQSSGLYSLSSVVTVPSSSLGTKTYTCNVDHKPSNTKVDKRVESKYGPPCP |
| | | PCPAPEFLGGPSVFLFPPKPKDTLMISRTPEVTCVVVDVSQEDPEVQFNWYVDGVE |
| | | VHNAKTKPREEQFNSTYRVVSVLTVLHQDWLNGKEYKCKVSNKGLPSSIEKTISKA |
| | | KGQPREPQVYTLPPSQEEMTKNQVSLTCLVKGFYPSDIAVEWESNGQPENNYKTTP |
| | | PVLDSDGSFFLYSRLTVDKSRWQEGNVFSCSVMHEALHNHYTQKSISISLGK |
| 51 | Hu5f9-G4 | DIVMTQSPLSLPVTPGEPASISCRSSQSIVYSNGNTYLGWYLQKPGQSPQLLIYKV |
| | Antibody | SNRFSGVPDRFSGSGSGTDFTLKISRVEAEDVGVYYCFQGSHVPYTFGQGTKLEIK |
| | Light | RTVAAPSVFIFPPSDEQLKSGTASVVCLLNNFYPREAKVQWKVDNALQSGNSQESV |
| | chain | TEQDSKDSTYSLSSTLTLSKADYEKHKVYACEVTHQGLSSPVTKSFNRGEC |
| 52 | Hu5f9-G4 | NYNMH |
| | VH CDR1 | |
| 53 | Hu5f9-G4 | TIYPGNDDTSYNQKFKD |
| | VH CDR2 | |
| 54 | Hu5f9-G4 | GGYRAMDY |
| | VH CDR3 | |
| 55 | Hu5f9-G4 | RSSQSIVYSNGNTYL |
| | VL CDR1 | |
| 56 | Hu5f9-G4 | KVSNRFS |
| | VL CDR2 | |
| 57 | Hu5f9-G4 | FQGSHVPYT |
| | VL CDR3 | |
| 58 | 5F9 | VHQVQLQQPGAELVKPGASVMMSCKASGYTFTNYNMHWVKQTPGQGLEWIGTIYPG |
| | | NDDTSYNQKFKDKATLTADKSSSAAYMQLSSLTSEDSAVYYCARGGYRAMDYWGQT |
| | | SVTVSS |
| 59 | 5F9 VL | DVLMTQTPLSLPVSLGDQASISCRSSQSIVYSNGNTYLGWYLQKPGQSPKLLIYKV |
| | | SNRFSGVPDRFSGSGSGTDFTLKISRVEAEDLGVYHCFQGSHVPYTFGGGTKVEIK |
| 60 | HuB6H12 | EVQLVESGGGLVQPGGSLRLSCAASGFTFSGYGMSWVRQAPGKGLEWVATITSGGT |
| | VH | YTYYPDSVKGRFTISRDNAKNSLYLQMNSLRAEDTAVYYCARSLAGNAMDYWGQGT |
| | | LVTVSS |
| 61 | HuB6H12 | EIVLTQSPATLSLSPGERATLSCRASQTISDYLHWYQQKPGQAPRLLIKFASQSIS |
| | VL | GIPARFSGSGSGTDFTLTISSLEPEDFAVYYCQNGHGFPRTFGQGTKVEIK |
| 62 | 8B6 VH | EVQLVESGGGLVQPGGSLRLSCAASGFTFSSYWMSWVRQAPGKGLEWVANIKQDGS |
| | | EKYYVDSVKGRFTISRDNAKNSLYLQMNSLRAEDTAVYYCARWGQGTLVTVSS |
| 63 | 8B6 VL | DIVMTQSPATLSVTPGDRVSLSCRASQNFSDYLHWYQQKSHESPRLLIKYVSHSIS |
| | | GIPSRFSGSGSGSDFTLSINSVEPEDVGVYYCQNGHSFPPTFGGGTKLEIK |
| 64 | C3 VH | QVQLQQSGAELVKPGASVKLSCKASGYTFTNYYIFWVKERPGQGLEWIGDINPSNG |
| | | DTNFNEKFKIKATLTVDKSSSTTYMQLNSLTSEDSAVYFCTRGGYTMDYWGQGTSV |
| | | TVSS |
| 65 | C3 VL | DVVMTQTPLSLPVSLGDQASISCRSSQSLVHSNGNTYFHWYVQKPGQSPKLLIYKV |
| | | SYRFSGVPDRFSGSGSGTDFTLKISRVEAEDLGVYFCSQSTHVPRTFGGGTKLEIK |
| 66 | HuC3 VH | QVQLVQSGAEVKKPGASVKVSCKASGYTFTNYYIFWVRQAPGQGLEWIGDINPSNG |
| | (A) | DTNFNEKFKIKATLTVDKSTSTTYMELSSLRSEDTAVYYCTRGGYTMDYWGQGTLV |
| | | TVSS |
| 67 | HuC3 VH | QVQLVQSGAEVKKPGASVKVSCKASGYTFTNYYIFWVRQAPGQGLEWMGDINPSNG |
| | (B) | DTNFNEKFKIRVTMTRDTSTSTVYMELSSLRSEDTAVYYCTRGGYTMDYWGQGTLV |
| | | TVSS |
| 68 | HuC3 VL | DVVMTQTPLSLSVTPGQPASISCRSSQSLVHSNGNTYFHWYLQKPGQPPKLLIYKV |
| | (C) | SYRFSGVPDRFSGSGSGTDFTLKISRVEAEDVGVYFCSQSTHVPRTFGQGTKVEIK |
| 69 | HuC3 VL | DIVMTQTPLSLSVTPGQPASISCRSSQSLVHSNGNTYFHWYLQKPGQPPQLLIYKV |
| | (D) | SYRFSGVPDRFSGSGSGTDFTLKISRVEAEDVGVYYCSQSTHVPRTFGQGTKVEIK |
| 70 | Anti-CD47 | EVQLQQSGAELVRSGASVKLSCTASGFNIKDYYLHWVKQRPEQGLEWIGWIDPDNG |
| | VH | DTEFAPKFQGKATMTADTSSNTAYLQLSSLTSEDTAVYYCNAAYGSSSYPMDYWGQ |
| | | GTSVTV |
| 71 | Anti-CD47 | EVQLVQSGAEVKKPGATVKISCKVSGFNIKDYYLHWVQQAPGKGLEWMGWIDPDNG |
| | VH | DTEYAEKFQGRVTITADTSTDTAYMELSSLRSEDTAVYYCNAAYGSSSYPMDYWGQ |
| | | GTTVTV |
| 72 | Anti-CD47 | QMQLVQSGAEVKKTGSSVKVSCKASGFNIKDYYLHWVRQAPGQALEWMGWIDPDNG |
| | VH | DTEYAQKFQDRVTITRDRSMSTAYMELSSLRSEDTAMYYCNAAYGSSSYPMDYWGQ |
| | | GTTVTV |
| 73 | Anti-CD47 | EVQLVQSGAEVKKTGSSVKVSCKASGFNIKDYYLHWVRQAPGQALEWMGWIDPDNG |
| | VH | DTEYAQKFQDRVTITRDRSMSTAYMELSSLRSEDTAMYYCNAAYGSSSYPMDYWGQ |
| | | GTTVTV |
| 74 | Anti-CD47 | QMQLVQSGAEVKKTGSSVKVSCKASGFNIKDYYLHWVRQAPGQALEWMGWIDPDNG |
| | VH | DTEYAQKFQGRVTMTADTSSNTAYMELSSLRSEDTAMYYCNAAYGSSSYPMDYWGQ |
| | | GTTVTV |
| 75 | Anti-CD47 | QMQLVQSGAEVKKTGSSVKVSCKASGFNIKDYYLHWVRQAPGQALEWMGWIDPDNG |
| | VH | DTEYAQKFQGRVTMTEDTSTDTAYMELSSLRSEDTAMYYCNAAYGSSSYPMDYWGQ |
| | | GTTVTV |
| 76 | Anti-CD47 | QMQLVQSGAEVKKTGSSVKVSCKASGFNIKDYYLHWVRQAPGQALEWMGWIDPDQG |
| | VH | DTEYAQKFQDRVTITRDRSMSTAYMELSSLRSEDTAMYYCNAAYGSSSYPMDYWGQ |
| | | GTTVTV |
| 77 | Anti-CD47 | QMQLVQSGAEVKKTGSSVKVSCKASGFNIKDYYLHWVRQAPGQALEWMGWIDPDYG |
| | VH | DTEYAQKFQDRVTITRDRSMSTAYMELSSLRSEDTAMYYCNAAYGSSSYPMDYWGQ |
| | | GTTVTV |
| 78 | Anti-CD47 | QMQLVQSGAEVKKTGSSVKVSCKASGFNIKDYYLHWVRQAPGQALEWMGWIDPDSG |
| | VH | DTEYAQKFQDRVTITRDRSMSTAYMELSSLRSEDTAMYYCNAAYGSSSYPMDYWGQ |
| | | GTTVTV |
| 79 | Anti-CD47 | QMQLVQSGAEVKKTGSSVKVSCKASGFNIKDYYLHWVRQAPGQALEWMGWIDPDNA |
| | VH | DTEYAQKFQDRVTITRDRSMSTAYMELSSLRSEDTAMYYCNAAYGSSSYPMDYWGQ |
| | | GTTVTV |
| 80 | Anti-CD47 | QMQLVQSGAEVKKTGSSVKVSCKASGFNIKDYYLHWVRQAPGQALEWMGWIDPDNT |
| | VH | DTEYAQKFQDRVTITRDRSMSTAYMELSSLRSEDTAMYYCNAAYGSSSYPMDYWGQ |
| | | GTTVTV |
| 81 | Anti-CD47 | QMQLVQSGAEVKKTGSSVKVSCKASGFNIKDYYLHWVRQAPGQALEWMGWIDPDNG |
| | VH | DTEYAQKFQDRVTITRDRSMSTAYMELSSLRSEDTAMYYCNAAYGSSPYPMDYWGQ |
| | | GTTVTV |
| 82 | Anti-CD47 | QMQLVQSGAEVKKTGSSVKVSCKASGYTFTYYYLHWVRQAPGQALEWMGWIDPDNG |
| | VH | DTEYAQKFQDRVTITRDRSMSTAYMELSSLRSEDTAMYYCNAAYGSSSYPMDYWGQ |
| | | GTTVTV |
| 83 | Anti-CD47 | QMQLVQSGAEVKKTGSSVKVSCKASGFTFTYYYLHWVRQAPGQALEWMGWIDPDNG |
| | VH | DTEYAQKFQDRVTITRDRSMSTAYMELSSLRSEDTAMYYCNAAYGSSSYPMDYWGQ |
| | | GTTVTV |
| 84 | Anti-CD47 | QMQLVQSGAEVKKTGSSVKVSCKASGYNFTYYYLHWVRQAPGQALEWMGWIDPDNG |
| | VH | DTEYAQKFQDRVTITRDRSMSTAYMELSSLRSEDTAMYYCNAAYGSSSYPMDYWGQ |
| | | GTTVTV |
| 85 | Anti-CD47 | QMQLVQSGAEVKKTGSSVKVSCKASGYTITYYYLHWVRQAPGQALEWMGWIDPDNG |
| | VH | DTEYAQKFQDRVTITRDRSMSTAYMELSSLRSEDTAMYYCNAAYGSSSYPMDYWGQ |
| | | GTTVTV |
| 86 | Anti-CD47 | QMQLVQSGAEVKKTGSSVKVSCKASGYTFKYYYLHWVRQAPGQALEWMGWIDPDNG |
| | VH | DTEYAQKFQDRVTITRDRSMSTAYMELSSLRSEDTAMYYCNAAYGSSSYPMDYWGQ |
| | | GTTVTV |
| 87 | Anti-CD47 | QMQLVQSGAEVKKTGSSVKVSCKASGYTFTDYYLHWVRQAPGQALEWMGWIDPDNG |
| | VH | DTEYAQKFQDRVTITRDRSMSTAYMELSSLRSEDTAMYYCNAAYGSSSYPMDYWGQ |
| | | GTTVTV |
| 88 | Anti-CD47 | QMQLVQSGAEVKKTGSSVKVSCKASGFTFTDYYLHWVRQAPGQALEWMGWIDPDNG |
| | VH | DTEYAQKFQDRVTITRDRSMSTAYMELSSLRSEDTAMYYCNAAYGSSSYPMDYWGQ |
| | | GTTVTV |
| 89 | Anti-CD47 | QMQLVQSGAEVKKTGSSVKVSCKASGFTITDYYLHWVRQAPGQALEWMGWIDPDNG |
| | VH | DTEYAQKFQDRVTITRDRSMSTAYMELSSLRSEDTAMYYCNAAYGSSSYPMDYWGQ |
| | | GTTVTV |
| 90 | Anti-CD47 | QMQLVQSGAEVKKTGSSVKVSCKASGYTFKDYYLHWVRQAPGQALEWMGWIDPDNG |
| | VH | DTEYAQKFQDRVTITRDRSMSTAYMELSSLRSEDTAMYYCNAAYGSSSYPMDYWGQ |
| | | GTTVTV |
| 91 | Anti-CD47 | QMQLVQSGAEVKKTGSSVKVSCKASGFTFKDYYLHWVRQAPGQALEWMGWIDPDNG |
| | VH | DTEYAQKFQDRVTITRDRSMSTAYMELSSLRSEDTAMYYCNAAYGSSSYPMDYWGQ |
| | | GTTVTV |
| 92 | Anti-CD47 | QMQLVQSGAEVKKTGSSVKVSCKASGFNIKDYYLHWVRQAPGQALEWMGWIDPDNG |
| | VH | DTEYAQKFQDRVTITRDRSMSTAYLQLSSLRSEDTAMYYCNAAYGSSSYPMDYWGQ |
| | | GTTVTV |
| 93 | Anti-CD47 | QMQLVQSGAEVKKTGSSVKVSCKASGFNIKDYYLHWVRQAPGQALEWMGWIDPDNG |
| | VH | DTEYAQKFQDRVTITRDRSMSTAYMELSSLTSEDTAVYYCNAAYGSSSYPMDYWGQ |
| | | GTTVTV |
| 94 | Anti-CD47 | EVQLVQSGAEVKKPGATVKISCKVSGFNIKDYYLHWVRQAPGQALEWMGWIDPDNG |
| | VH | DTEYAQKFQDRVTITRDRSMSTAYMELSSLRSEDTAMYYCNAAYGSSSYPMDYWGQ |
| | | GTTVTV |
| 95 | Anti-CD47 | EVQLVQSGAEVKKPGATVKISCKVSGFNIKDYYLHWVQQAPGKGLEWMGWIDPDNG |
| | VH | DTEYAQKFQDRVTITRDRSMSTAYMELSSLRSEDTAMYYCNAAYGSSSYPMDYWGQ |
| | | GTTVTV |
| 96 | Anti-CD47 | DIKMTQSPSSLYASLGERVTITCKASQDIHRYLSWFQQKPGKSPKILIYRANRLVD |
| | VL | GVPSRFSGSGSGQDYSLTISSLEYEDMGIYYCLQYDEFPYTFGGGTKLEMK |
| 97 | Anti-CD47 | DIKMTQSPSSLYASLGERVTITCKASQDIHRYLSWFQQKPGKSPKILIYRANRLVD |
| | VL | GVPSRFSGSGSGQDYSLTISSLEYEDMGIYYCLQYDEFPYTFGGGTKLEIK |
| 98 | Anti-CD47 | DIQMTQSPSSLSASVGDRVTITCKASQDIHRYLSWYQQKPGKAPKLLIYRANRLVD |
| | VL | GVPSRFSGSGSGTDFTFTISSLQPEDIATYYCLQYDEFPYTFGGGTKVEIK |
| 99 | Anti-CD47 | DIQMTQSPSSLSASVGDRVTITCKASQDIHRYLSWFQQKPGKAPKSLIYRANRLVD |
| | VL | GVPSRFSGSGSGTDFTLTISSLQPEDFATYYCLQYDEFPYTFGGGTKVEIK |
| 100 | Anti-CD47 | NIQMTQSPSAMSASVGDRVTITCKASQDIHRYLSWFQQKPGKVPKHLIYRANRLVD |
| | VL | GVPSRFSGSGSGTEFTLTISSLQPEDFATYYCLQYDEFPYTFGGGTKVEIK |
| 101 | Anti-CD47 | DIQMTQSPSSLSASVGDRVTITCKASQDIHRYLSWYQQKPGKAPKRLIYRANRLVD |
| | VL | GVPSRFSGSGSGTEFTLTISSLQPEDFATYYCLQYDEFPYTFGGGTKVEIK |
| 102 | Anti-CD47 | DIQMTQSPSSLSASVGDRVTITCRASQDIHRYLAWYQQKPGKVPKLLIYRANRLQS |
| | VL | GVPSRFSGSGSGTDFTLTISSLQPEDVATYYCLQYDEFPYTFGQGTKVEIK |
| 103 | Anti-CD47 | EIVLTQSPATLSLSPGERATLSCRASQDIHRYLAWYQQKPGQAPRLLIYRANRRAT |
| | VL | GIPARFSGSGSGTDFTLTISSLEPEDFAVYYCLQYDEFPYTGFQGTRLEIK |
| 104 | Anti-CD47 | DIQMTQSPSAMSASVGDRVTITCKASQDIHRYLSWFQQKPGKVPKHLIYRANRLVD |
| | VL | GVPSRFSGSGSGTEFTLTISSLQPEDFATYYCLQYDEFPYTFGGGTKVEIK |
| 105 | Anti-CD47 | NIQMTQSPSAMSASVGDRVTITCRARQGIHRYLSWFQQKPGKVPKHLIYRANRLVD |
| | VL | GVPSRFSGSGSGTEFTLTISSLQPEDFATYYCLQYDEFPYTFGGGTKVEIK |
| 106 | Anti-CD47 | NIQMTQSPSAMSASVGDRVTITCKASQDIHRYLSWFQQKPGKVPKILIYRANRLVD |
| | VL | GVPSRFSGSGSGTEFTLTISSLQPEDFATYYCLQYDEFPYTFGGGTKVEIK |
| 107 | Anti-CD47 | NIQMTQSPSAMSASVGDRVTITCKASQDIHRYLSWFQQKPGKVPKHLIYRANRLVS |
| | VL | GVPSRFSGSGSGTEFTLTISSLQPEDFATYYCLQYDEFPYTFGGGTKVEIK |
| 108 | Anti-CD47 | NIQMTQSPSAMSASVGDRVTITCRARQGIHRYLSWFQQKPGKVPKILIYRANRLVD |
| | VL | GVPSRFSGSGSGTEFTLTISSLQPEDFATYYCLQYDEFPYTFGGGTKVEIK |
| 109 | Anti-CD47 | NIQMTQSPSAMSASVGDRVTITCRARQGIHRYLSWFQQKPGKVPKHLIYRANRLVS |
| | VL | GVPSRFSGSGSGTEFTLTISSLQPEDFATYYCLQYDEFPYTFGGGTKVEIK |
| 110 | Anti-CD47 | NIQMTQSPSAMSASVGDRVTITCKASQDIHRYLSWFQQKPGKVPKLLIYRANRLVD |
| | VL | GVPSRFSGSGSGTEFTLTISSLQPEDFATYYCLQYDEFPYTFGGGTKVEIK |
| 111 | Anti-CD47 | NIQMTQSPSAMSASVGDRVTITCKASQDIHRYLSWFQQKPGKVPKLLIYRANRLVS |
| | VL | GVPSRFSGSGSGTEFTLTISSLQPEDFATYYCLQYDEFPYTFGGGTKVEIK |
| 112 | Anti-CD47 | NIQMTQSPSAMSASVGDRVTITCRARQGIHRYLSWFQQKPGKVPKLLIYRANRLVS |
| | VL | GVPSRFSGSGSGTEFTLTISSLQPEDFATYYCLQYDEFPYTFGGGTKVEIK |
| 113 | Anti-CD47 | QVQLVQSGAEVKKPGASVKVSCKASGYTFTNYNMHWVRQAPGQGLEWIGTIYPGND |
| | VH | DTSYNQKFKDKATLTADKSTSTAYMELSSLRSEDTAVYYCARGGYRAMDYWGQGTL |
| | | VTVSS |
| 114 | Anti-CD47 | QVQLVQSGAEVKKPGASVKVSCKASGYTFTNYNMHWVRQAPGQRLEWMGTIYPGND |
| | VH | DTSYNQKFKDRVTITADTSASTAYMELSSLRSEDTAVYYCARGGYRAMDYWGQGTL |
| | | VTVSS |
| 115 | Anti-CD47 | QVQLVQSGAEVKKPGASVKVSCKASGYTFTNYNMHWVRQAPGQRLEWIGTIYPGND |
| | VH | DTSYNQKFKDRATLTADKSASTAYMELSSLRSEDTAVYYCARGGYRAMDYWGQGTL |
| | | VTVSS |
| 116 | Anti-CD47 | DVVMTQSPLSLPVTPGEPASISCRSSQSIVYSNGNTYLGWYLQKPGQSPKLLIYKV |
| | VL | SNRFSGVPDRFSGSGSGTDFTLKISRVEAEDVGVYHCFQGSHVPYTFGGGTKVEIK |
| 117 | Anti-CD47 | DIVMTQSPLSLPVTPGEPASISCRSSQSIVYSNGNTYLGWYLQKPGQSPQLLIYKV |
| | VL | SNRFSGVPDRFSGSGSGTDFTLKISRVEAEDVGVYYCFQGSHVPYTFGQGTKLEIK |
| 118 | Anti-CD47 | DVVMTQSPLSLPVTPGEPASISCRSSQSIVYSNGNTYLGWYLQKPGQSPQLLIYKV |
| | VL | SNRFSGVPDRFSGSGSGTDFTLKISRVEAEDVGVYHCFQGSHVPYTFGQGTKLEIK |
| 119 | Anti-SIRPa | QVQLQQPGAELVRPGSSVKLSCKASGYTFTSYWVHWVKQRPIQGLEWIGNIDPSDS |
| | VH | DTHYNQKFKDKASLTVDKSSSTAYMQLSSLTFEDSAVYYCVRGGTGTMAWFAYWGQ |
| | | GTLVTVSA |
| 120 | Anti-SIRPa | EVQLVQSGAEVKKPGESLRISCKASGYTFTSYWVHWVRQMPGKGLEWIGNIDPSDS |
| | VH | DTHYNQKFKDHVTLSVDKSISTAYLQLSSLKASDTAMYYCVRGGTGTMAWFAYWGQ |
| | | GTLVTVSS |
| 121 | Anti-SIRPa | EVQLVQSGAEVKKPGESLRISCKASGYSFTSYWVHWVRQMPGKGLEWMGNIDPSDS |
| | VH | DTHYNQKFKDHVTLSVDKSISTAYLQLSSLKASDTAMYYCVRGGTGTMAWFAYWGQ |
| | | GTLVTVSS |
| 122 | Anti-SIRPa | EVQLVQSGAEVKKPGESLRISCKASGYSFTSYWVHWVRQMPGKGLEWMGNIDPSDS |
| | VH | DTHYSPSFQGHVTLSVDKSISTAYLQLSSLKASDTAMYYCVRGGTGTMAWFAYWGQ |
| | | GTLVTVSS |
| 123 | Anti-SIRPa | EVQLVQSGAEVKKPGESLRISCKASGYSFTSYWVHWVRQMPGKGLEWMGNIDPSDS |
| | VH | DTHYSPSFQGHVTLSVDKSISTAYLQLSSLKASDTAMYYCVRGGTGTLAWFAYWGQ |
| | | GTLVTVSS |
| 124 | Anti-SIRPa | EVQLVQSGAEVKKPGESLRISCKASGYSFTSYWVHWVRQMPGKGLEWMGNIDPSDS |
| | VH | DTHYSPSFQGHVTLSVDKSISTAYLQLSSLKASDTAMYYCVRGGTGTMAYFAYWGQ |
| | | GTLVTVSS |
| 125 | Anti-SIRPa | EVQLVQSGAEVKKPGESLRISCKASGYSFTSYWVHWVRQMPGKGLEWMGNIDPSDS |
| | VH | DTHYSPSFQGHVTLSVDKSISTAYLQLSSLKASDTAMYYCVRGGTGTLAYFAYWGQ |
| | | GTLVTVSS |
| 126 | Anti-SIRPa | DVVMTQTPLSLPVSLGDQASISCRSSQSLVHSYGNTYLYWYLQKPGQSPKLLIYRV |
| | VL | SNRFSGVPDRFSGSGSGTDFTLKISRVEAEDLGVYFCFQGTHVPYTFGSGTKLEIK |
| 127 | Anti-SIRPa | DVVMTQSPLSLPVTLGQPASISCRSSQSLVHSYGNTYLYWYQQRPGQSPRLLIYRV |
| | VL | SNRFSGVPDRFSGSGSGTDFTLKISRVEAEDVGVYFCFQGTHVPYTFGGGTKVEIK |
| 128 | Anti-SIRPa | DVVMTQSPLSLPVTLGQPASISCRSSQSLVHSYGNTYLYWFQQRPGQSPRLLIYRV |
| | VL | SNRFSGVPDRFSGSGSGTDFTLKISRVEAEDVGVYYCFQGTHVPYTFGGGTKVEIK |
| 129 | Rituximab | QVQLQQPGAEINKPGASVMSCKASGYTFTSYNMHVTVKQTPGRGLEWIGAIYPGNG |
| | heavy | DTSYNQKFYGKATLTADKSSSTAYMQLSSLTSEDSAVYYCARSTYYGGDWYENVWG |
| | chain | AGTTVTVSAASTKGPSVEPLAPSSKSTSGGTAALGCLVKDYFPEPVTVSWNSGALT |
| | chimeric | SGVHTFPAVLQSSGLYSLSSVVTVPSSSLGTQTYICNVNHKPSNTKNDKKAEPKSC |
| | | DKTHTCPPCPAPELLGGPSVFLFPPKPKDTLMISRTPEVTCVVVDVSHEDPEVKFN |
| | | WYVDGVEVHNAKTKPREEQYNSTYRVVSVLTVLHQDWLNGKEYKCKVSNKALPAPI |
| | | EKTISKAKGQPREPQVYTLPPSRDELTKNQVSLTCLVKGFYPSDIAVEWESNGQPE |
| | | NNYKTTPPVLDSDGSFFLYSKLTVDKSRWQQGNVFSCSVMHEALHNHYTQKSLSLS |
| | | PGK |
| 130 | Rituximab | QIVLSQSPAILSASPGEKVTMTCRASSSVSYIHWFQQKPGSSPKPWIYATSNLASG |
| | light | VPVRFSGSGSGTSYSLTISRVEAEDAATYYCQQWTSNPPTFGGGTKLEIKRTVAAP |
| | chain | SVFIFPPSDEQLKSGTASVVCLLNNEYPREAKVQWKVDNALQSGNSQESVTEQDSK |
| | chimeric | DSTYSLSSTLTLSKADYEKHKVYACEVTHQGLSSPVTKSFNRGEC |
| 131 | Rituximab | KASGYTFTSYNMH |
| | VH CDR1 | |
| 132 | Rituximab | AIYPGNGDTS |
| | VH CDR2 | |
| 133 | Rituximab | ARSTYYGGDWYFNV |
| | VH CDR3 | |
| 134 | Rituximab | RASSSVSYIH |
| | VL CDR1 | |
| 135 | Rituximab | YATSNLAS |
| | VL CDR2 | |
| 136 | Rituximab | QQWTSNPPT |
| | VL CDR3 | |
| 137 | Rituximab | QVQLQQPGAELVKPGASVKMSCKASGYTFTSYNMHWVKQTPGRGLEWIGAIYPGNG |
| | Variable | DTSYNQKFKGKATLTADKSSSTAYMQLSSLTSEDSAVYYCARSTYYGGDWYFNVWG |
| | Heavy | AGTTVTVSAASTK |
| | chain | |
| | (VH) | |
| 138 | Rituximab | GPSVFPLAPSSKSTSGGTAALGCLVKDYFPEPVTVSWNSGALTSGVHTFPAVLQSS |
| | Constant | GLYSLSSVVTVPSSSLGTQTYICNVNHKPSNTKVDKKA |
| | Heavy | |
| | Chain 1 | |
| | (C_{H}1) | |
| 139 | Rituximab | EPKSCDKTHTCPPCP |
| | Heavy | |
| | Chain | |
| | Hinge | |
| 140 | Rituximab | APELLGGPSVFLFPPKPKDTLMISRTPEVTCVVVDVSHEDPEVKFNWYVDGVEVHN |
| | Constant | AKTKPREEQYNSTYRVVSVLTVLHQDWLNGKEYKCKVSNKALPAPIEKTISKAK |
| | Heavy | |
| | Chain 2 | |
| | (C_{H}2) | |
| 141 | Rituximab | GQPREPQVYTLPPSRDELTKNQVSLTCLVKGFYPSDIAVEWESNGQPENNYKTTPP |
| | Constant | VLDSDGSFFLYSKLTVDKSRWQQGNVFSCSVMHEALHNHYTQKSLSLSPGK |
| | Heavy | |
| | Chain 3 | |
| | (C_{H}3) | |
| 142 | Ritaximab | QIVLSQSPAILSASPGEKVTMTCRASSSVSYIHWFQQKPGSSPKPWIYATSNLASG |
| | Variable | VPVRFSGSGSGTSYSLTISRVEAEDAATYYCQQWTSNPPTFGGGTKLEIK |
| | Light | |
| | Chain | |
| 143 | Ritaximab | RTVAAPSVFIFPPSDEQLKSGTASVVCLLNNFYPREAKVQWKVDNALQSGNSQESV |
| | Constant | TEQDSKDSTYSLSSTLTLSKADYEKHKVYACEVTHQGLSSPVTKSFNRGEC |
| | Light | |
| | Chain | |
| 144 | Hu5f9-G4 | QVQLVQSGAEVKKPGASVKVSCKASGYTFTNYNMHWVRQAPGQRLEWMGTIYPGND |
| | VH | DTSYNQKFKDRVTITADTSASTAYMELSSLRSEDTAVYYCARGGYRAMDYWGQGTL |
| | | VTVSS |
| 145 | Hu5f9-G4 | DIVMTQSPLSLPVTPGEPASISCRSSQSIVYSNGNTYLGWYLQKPGQSPQLLIYKV |
| | VL | SNRFSGVPDRFSGSGSGTDFTLKISRVEAEDVGVYYCFQGSHVPYTFGQGTKLEIK |
| 146 | Hu5f9-G4 | RSSQSIVYSNGNTYLG |
| | VL CDR1 | |
| 147 | Hu5f9-G4 | GYTFTNYN |
| | VH CDR1 | |
| 148 | Hu5f9-G4 | IYPGNDDT |
| | VH CDR2 | |
| 149 | Hu5f9-G4 | ARGGYRAMDY |
| | VH CDR3 | |
| 150 | Hu5f9-G4 | QSIVYSNGNTY |
| | VL CDR1 | |
| 151 | Hu5f9-G4 | KVS |
| | VL CDR2 | |
| 152 | Hu5f9-G4 | FQGSHVPYT |
| | VL CDR3 | |
| 153 | Hu5f9-G4 | GYTFTNY |
| | VH CDR1 | |
| 154 | Hu5f9-G4 | PGND |
| | VH CDR2 | |
| 155 | Hu5f9-G4 | GYRAMD |
| | VH CDR3 | |
| 156 | Hu5f9-G4 | SQSIVYSNGNTY |
| | VL CDR1 | |
| 157 | Hu5f9-G4 | KVS |
| | VL CDR2 | |
| 158 | Hu5f9-G4 | GSHVPY |
| | VL CDR3 | |
| 159 | Hu5f9-G4 | ASGYTFTNYN |
| | VH CDR1 | |
| 160 | Hu5f9-G4 | IYPGNDDTSYNQKFKDR |
| | VH CDR2 | |
| 161 | Hu5f9-G4 | GGYRAMD |
| | VH CDR3 | |
| 162 | Hu5f9-G4 | SSQSIVYSNGNTY |
| | VL CDR1 | |
| 163 | Hu5f9-G4 | KVSNRFSGVPDR |
| | VL CDR2 | |
| 164 | Hu5f9-G4 | GSHVPY |
| | VL CDR3 | |

1. A method of treating a blood cancer in a subject comprising: (a) administering an anti-CD47 agent that inhibits binding between CD47 and SIRPα; and (b) administering an anti-CD20 antibody to the subject, wherein B-cells are determined or have been determined to be present in the subject prior to performing steps (a) and (b).

2. A method of treating a blood cancer in a subject comprising:
determining or having determined that B-cells are present in the subject; and
administering or having administered to the subject (i) an anti-CD47 agent that inhibits binding between CD47 and SIRPα and (ii) an anti-CD20 antibody.

3.-4. (canceled)

5. The method of claim 1, wherein the determination that B-cells are present in the subject comprises determining or having determined that the subject has CD19+ B-cells.

6.-10. (canceled)

11. The method of claim 1, wherein the determination that B-cells are present in the subject comprises determining or having determined that the subject has CD20+ B-cells.

12.-16. (canceled)

17. The method of claim 1, wherein the determination that B-cells are present in the subject comprises determining or having determined that the subject has both CD19+ B-cells and CD20+ B-cells.

18.-20. (canceled)

21. The method of claim 1, wherein the determination that B-cells are present in the subject comprises determining or having determined that the subject previously received an anti-CD20 therapy more than a threshold amount of time ago.

22. The method of claim 21, wherein the threshold amount of time is at least 4 weeks.

23.-34. (canceled)

35. The method of claim 1, wherein the anti-CD47 agent comprises an anti-CD47 antibody or an anti-SIRPα antibody.

36. The method of claim 35, wherein the anti-CD47 agent comprises or is magrolimab.

37. (canceled)

38. The method of claim 1, wherein the blood cancer is diffuse large B-cell lymphoma (DLBCL).

39. The method of claim 38, wherein the subject has relapsed or refractory DLBCL.

40. The method of claim 39, wherein the subject has previously been treated with at least two prior lines of therapy.

41. The method of claim 1, wherein the blood cancer is follicular lymphoma (FL) or marginal zone lymphoma.

42. The method of claim 1, wherein the blood cancer is non-Hodgkin's lymphoma.

43. (canceled)

44. The method of claim 1, wherein the anti-CD47 agent is administered intravenously.

45. The method of claim 1, wherein the anti-CD20 antibody is administered intravenously.

46.-59. (canceled)

60. The method of claim 1, wherein the anti-CD20 antibody comprises or is rituximab.

61.-66. (canceled)

67. The A-method of claim 1,
wherein the anti-CD47 agent comprises or is magrolimab; and
wherein the anti-CD20 antibody comprises or is rituximab,
wherein administering magrolimab comprises (1) administering a priming dose of magrolimab in the range of 1 mg to 10 mg of antibody per kg of body weight on Day 1, (2) administering weekly doses of magrolimab of at least 30 mg per kg of body weight for 8 weeks, and (3) administering every-other-week doses of magrolimab of at least 30 mg per kg of body weight onwards, and
wherein administering rituximab comprises (1) administering a weekly dose of rituximab at about 375 mg per m² of body surface area for 4 weeks and subsequently (2) administering monthly rituximab at about 375 mg per m² of body surface area.

68. (canceled)

69. A method comprising:
obtaining a sample from a subject with a blood cancer;
determining whether B-cells are present in the subject by performing an assay on the obtained sample from the subject,
wherein a presence of B-cells in the subject indicates that the subject is likely to respond to a therapy comprising 1) an anti-CD47 agent that inhibits binding between CD47 and SIRPα and 2) an anti-CD20 antibody,
wherein an absence of B-cells in the subject indicates that the subject is unlikely to respond to a therapy comprising 1) the anti-CD47 agent that inhibits binding between CD47 and SIRPα and 2) the anti-CD20 antibody.

70. The method of claim 69, wherein the sample obtained from the subject is a peripheral blood sample.

71.-133. (canceled)
